(19) United States
(12) Patent Application Publication
(10) Pub. No.: US 2025/0205292 A1
(43) Pub. Date: Jun. 26, 2025

(54) PHARMACEUTICAL COMPOSITIONS AND USES THEREOF

(71) Applicant: Siolta Therapeutics, Inc., San Carlos, CA (US)

(72) Inventors: Nikole KIMES, San Francisco, CA (US); Ricardo VALLADARES, Brookhaven, GA (US)

(21) Appl. No.: 18/967,879

(22) Filed: Dec. 4, 2024

Related U.S. Application Data
(63) Continuation of application No. PCT/US23/68094, filed on Jun. 7, 2023.
(60) Provisional application No. 63/350,666, filed on Jun. 9, 2022.

(51) Int. Cl. A61K 35/747; A61K 35/745; A61P 1/00; A61P 15/02; A61P 31/04

(52) U.S. Cl. CPC A61K 35/747; A61K 35/745; A61P 1/00; A61P 15/02; A61P 31/04

(57) ABSTRACT

Provided herein are pharmaceutical compositions comprising a bacterial population. The bacterial population comprises one bacterial strains or at least two bacterial strains. Such pharmaceutical compositions can be administered to a subject for prevention and/or treatment of dysbiosis and dysbiosis associated conditions and diseases, such as vaginal or infant gastrointestinal diseases.

### CROSS-REFERENCE

This application is a continuation of International Patent Application No. PCT/US23/68094, filed Jun. 7, 2023, which claims priority to and the benefit of U.S. Provisional Application No. 63/350,666, filed Jun. 9, 2022, the entire contents of each of which application is incorporated herein by reference in its entirety.

### SEQUENCE LISTING

The instant application contains a Sequence Listing which has been submitted electronically in XML format and is hereby incorporated by reference in its entirety. Said XML copy, created on Jun. 6, 2023, is named 53206-715_301_SL.xml and is 42.796 megabytes in size.

### BACKGROUND

Recent developments in the areas of microbiome and genome research provide evidence that the microbiome-host relationships influence human health or disease onset and progression. For example, they have been implicated in the inflammatory diseases Bacterial Vaginosis (BV) and Necrotizing Enterocolitis (NEC), playing key roles in the etiology of these disorders. The rising incidence of these diseases is concerning and represents a major public health challenge.

Currently available pharmaceutical compositions, however, can lack effectiveness, scalability, reliability, or stability.

### BRIEF SUMMARY

Restoring the microbiome-host homeostasis can help treating these disorders. Effective treatments can comprise administration of live biotherapeutics.

Disclosed herein, in some embodiments, are methods of treating a disease or disease condition in a subject in need thereof. In an aspect, a method of treating a disease or disease condition in a subject in need thereof comprises: administering to the subject a therapeutically-effective amount of a formulation, wherein the formulation comprises a bacterial population comprising a first bacterial strain and a second bacterial strain different from the first bacterial strain, wherein the first bacterial strain and the second bacterial strain comprise Bifidobacterium sp. or Vertebrate-Associated Lactobacillaceae, and wherein the formulation inhibits: (1) a growth or biofilm formation of a pathogen by at least 0.1% or (2) an immune response signaling pathway by at least 0.1%, relative to a growth or biofilm formation of the pathogen or the immune response signaling pathway when inhibited by a control formulation not comprising the first bacterial strain and the second bacterial strain, respectively.

In some embodiments, the disease or disease condition comprises a vaginal disease or a complication associated with the vaginal disease. In some embodiments, the vaginal disease comprises bacterial vaginosis (BV) or recurrent BV, and wherein the complication associated with the vaginal disease comprises preterm birth, pelvic inflammatory disease (PID), vulvovaginitis or sexually transmitted infections (STIs). In some embodiments, the formulation inhibits a growth or biofilm formation of a vaginal pathogen. In some embodiments, the formulation inhibits the growth or biofilm formation of the vaginal pathogen by at least 0.1% relative to a growth or biofilm formation of the vaginal pathogen when inhibited by the control formulation. In some embodiments, the formulation inhibits the growth or biofilm formation of the vaginal pathogen by at least 5% or by at least 50% relative to the growth or biofilm formation of the vaginal pathogen when inhibited by the control formulation. In some embodiments, the subject has been administered with a medication for treating the vaginal disease. In some embodiments, the medication comprises an antibiotic. In some embodiments, the subject has not been administered with a medication for treating the vaginal disease. In some embodiments, the disease or disease condition comprises an infant gastrointestinal disease. In some embodiments, the infant gastrointestinal disease comprises necrotizing enterocolitis (NEC), infectious gastroenteritis, neonatal cholestasis, pediatric intestinal motility disorders, gastroenteritis, Inflammatory bowel disease (IBD), Irritable bowel syndrome (IBS), or a combination thereof. In some embodiments, the formulation inhibits a growth of an infant gastrointestinal pathogen. In some embodiments, the formulation inhibits the growth of the infant gastrointestinal pathogen by at least 0.1% relative to a growth of the infant gastrointestinal pathogen when inhibited by the control formulation. In some embodiments, the formulation inhibits the growth of the infant gastrointestinal pathogen by at least 5% or by at least 50% relative to the growth of the infant gastrointestinal pathogen when inhibited by the control formulation. In some embodiments, the formulation inhibits a signal of an immune response reporter of the immune response signaling pathway within an engineered cell by at least 0.1% relative to a signal of the immune response reporter of the immune response signaling pathway within the engineered cell when inhibited by the control formulation. In some embodiments, the formulation inhibits the signal of the immune response reporter of the immune response signaling pathway within the engineered cell by at least 1% or by at least 10% relative to the signal of the immune response reporter of the immune response signaling pathway within the engineered cell when inhibited by the control formulation. In some embodiments, the engineered cell comprises a mammalian cell. In some embodiments, the immune response reporter comprises an inflammatory immune response reporter.

Disclosed herein, in some embodiments, are compositions. In an aspect, a composition comprises: a bacterial population comprising a first bacterial strain and a second bacterial strain different from the first bacterial strain for use in treating a disease or disease condition in a subject in need thereof, wherein the first bacterial strain and the second bacterial strain comprise Bifidobacterium sp. or Vertebrate-Associated Lactobacillaceae, and wherein the composition is configured to treat the disease or disease condition at least in part by: (1) inhibiting a growth or biofilm formation of a pathogen in the subject by at least 0.1%, relative to a growth or biofilm formation of the pathogen in the subject when inhibited by a control composition comprising a control bacterial population that does not comprise the first bacterial strain and the second bacterial strain; or (2) inhibiting an immune response signaling pathway of a cell of the subject by at least 0.1%, relative to the immune response signaling pathway of the cell of the subject when inhibited by the control composition.

In some embodiments, the disease or disease condition comprises an infant gastrointestinal disease. In some embodiments, the pathogen comprises an infant gastrointestinal pathogen. In some embodiments, the composition is configured to treat the infant gastrointestinal disease at least in part by inhibiting a growth of the infant gastrointestinal pathogen in the subject by at least 0.1% relative to a growth of the infant gastrointestinal pathogen in the subject when inhibited by the control composition. In some embodiments, the composition is configured to treat the infant gastrointestinal disease at least in part by inhibiting the growth of the infant gastrointestinal pathogen in the subject by at least 5% or by at least 50% relative to the growth of the infant gastrointestinal pathogen in the subject when inhibited by the control composition. In some embodiments, the composition is configured to inhibit the immune response signaling pathway of the cell of the subject by at least 1% or by at least 10% relative to the immune response signaling pathway of the cell of the subject when inhibited by the control formulation. In some embodiments, the disease or disease condition comprises a vaginal disease or a complication associated with the vaginal disease. In some embodiments, the vaginal disease comprises bacterial vaginosis (BV) or recurrent BV, and wherein the complication associated with the vaginal disease comprises preterm birth, pelvic inflammatory disease (PID), vulvovaginitis, or sexually transmitted infections (STIs). In some embodiments, the pathogen comprises a vaginal pathogen. In some embodiments, the composition is configured to treat the vaginal disease or the complication associated with the vaginal disease at least in part by inhibiting a growth or biofilm formation of the vaginal pathogen in the subject by at least 0.10% relative to a growth or biofilm formation of the vaginal pathogen in the subject when inhibited by the control composition. In some embodiments, the composition is configured to treat the vaginal disease or the complication associated with the vaginal disease at least in part by inhibiting the growth or biofilm formation of the vaginal pathogen in the subject by at least 5% or by at least 50% relative to the growth or biofilm formation of the vaginal pathogen in the subject when inhibited by the control composition.

Disclosed herein, in some embodiments, are compositions. In an aspect, a composition comprises: a bacterial population comprising a first bacterial strain and a second bacterial strain different from the first bacterial strain, wherein the first bacterial strain and the second bacterial strain comprise Bifidobacterium sp. or Vertebrate-Associated Lactobacillaceae, and wherein when the first bacterial strain is cultured with (1) the second bacterial strain or (2) a cultured medium of the second bacterial strain: (a) the first bacterial strain inhibits a growth or biofilm formation of a pathogen by at least 0.1% relative to a growth or biofilm formation of the pathogen when inhibited by a control bacterial strain comprising the first bacterial strain not cultured with (1) the second bacterial strain or (2) the cultured medium of the second bacterial strain, respectively, (b) the first bacterial strain inhibits an immune response signaling pathway by at least 0.1% relative to the immune response signaling pathway when inhibited by the control bacterial strain, respectively, (c) a cultured medium of the first bacterial strain inhibits the growth or biofilm formation of the pathogen by at least 0.1% relative to a growth or biofilm formation of the pathogen when inhibited by a cultured medium of the control bacterial strain, respectively, or (d) the cultured medium of the first bacterial strain inhibits the immune response signaling pathway by at least 0.1% relative to the immune response signaling pathway when inhibited by the cultured medium of the control bacterial strain, respectively.

In some embodiments, the pathogen comprises an infant gastrointestinal pathogen. In some embodiments, when the first bacterial strain is cultured with (1) the second bacterial strain or (2) the cultured medium of the second bacterial strain: (a) the first bacterial strain inhibits a growth of the infant gastrointestinal pathogen by at least 0.1% relative to a growth of the infant gastrointestinal pathogen when inhibited by the control bacterial strain, respectively, or (b) the cultured medium of the first bacterial strain inhibits the growth of the infant gastrointestinal pathogen by at least 0.1% relative to a growth of the infant gastrointestinal pathogen when inhibited by the cultured medium of the control bacterial strain, respectively. In some embodiments, when the first bacterial strain is cultured with (1) the second bacterial strain or (2) the cultured medium of the second bacterial strain: (a) the first bacterial strain inhibits the growth of the infant gastrointestinal pathogen by at least 5% or by at least 50% relative to the growth of the infant gastrointestinal pathogen when inhibited by the control bacterial strain, respectively, or (b) the cultured medium of the first bacterial strain inhibits the growth of the infant gastrointestinal pathogen by at least 5% or by at least 50% relative to the growth of the infant gastrointestinal pathogen when inhibited by the cultured medium of the control bacterial strain, respectively. In some embodiments, when the first bacterial strain is cultured with (1) the second bacterial strain or (2) the cultured medium of the second bacterial strain: (a) the first bacterial strain inhibits a signal of an immune response reporter of the immune response signaling pathway within an engineered cell by at least 0.1%, relative to a signal of the immune response reporter of the immune response signaling pathway within the engineered cell when inhibited by the control bacterial strain, respectively, or (b) the cultured medium of the first bacterial strain inhibits the signal of the immune response reporter of the immune response signaling pathway within the engineered cell by at least 0.1%, relative to a signal of the immune response reporter of the immune response signaling pathway within the engineered cell when inhibited by the cultured medium of the control bacterial strain, respectively. In some embodiments, when the first bacterial strain is cultured with (1) the second bacterial strain or (2) the cultured medium of the second bacterial strain: (a) the first bacterial strain inhibits the signal of the immune response reporter of the immune response signaling pathway within the engineered cell by at least 1% or by at least 10% relative to the signal of the immune response reporter of the immune response signaling pathway within the engineered cell when inhibited by the control bacterial strain, respectively, (b) the cultured medium of the first bacterial strain inhibits the signal of the immune response reporter of the immune response signaling pathway within the engineered cell by at least 1% or by at least 10% relative to the signal of the immune response reporter of the immune response signaling pathway within the engineered cell when inhibited by the cultured medium of the control bacterial strain, respectively. In some embodiments, the engineered cell comprises a mammalian cell. In some embodiments, the immune response reporter comprises an inflammatory immune response reporter. In some embodiments, the pathogen comprises a vaginal pathogen. In some embodiments, when the first bacterial strain is cultured with (1) the second bacterial strain or (2) the cultured medium of the second bacterial strain: (a) the first bacterial strain inhibits a growth or biofilm formation of the vaginal pathogen by at least 0.1% relative to a growth or biofilm formation of the vaginal pathogen when inhibited by the control bacterial strain, respectively, (b) the cultured medium of the first bacterial strain inhibits the growth or biofilm formation of the vaginal pathogen by at least 0.1% relative to a growth or biofilm formation of the vaginal pathogen when inhibited by the cultured medium of the control bacterial strain, respectively. In some embodiments, when the first bacterial strain is cultured with (1) the second bacterial strain or (2) the cultured medium of the second bacterial strain: (a) the first bacterial strain inhibits the growth or biofilm formation of the vaginal pathogen by at least 5% or by at least 50% relative to the growth or biofilm formation of the vaginal pathogen when inhibited by the control bacterial strain, respectively, (b) the cultured medium of the first bacterial strain inhibits the growth of the vaginal pathogen by at least 5% or by at least 50% relative to the growth or biofilm formation of the vaginal pathogen when inhibited by the cultured medium of the control bacterial strain, respectively.

Disclosed herein, in some embodiments, are compositions. In an aspect, a composition comprises: a cultured medium or derivative thereof of a bacterial population comprising (1) a first bacterial strain and (2) a second bacterial strain or a supernatant thereof, wherein the second bacterial strain is different from the first bacterial strain, wherein the first bacterial strain and the second bacterial strain comprise Bifidobacterium sp. or Vertebrate-Associated Lactobacillaceae, wherein the cultured medium or derivative thereof is configured to inhibit: (1) a growth or biofilm formation of a pathogen by at least 0.1% relative to a growth or biofilm formation of the pathogen when inhibited by a control cultured medium or derivative thereof of a control bacterial population or (2) an immune response signaling pathway by at least 0.1%, relative to the immune signaling pathway when inhibited by the control cultured medium or derivative thereof of the control bacterial population, and wherein the control bacterial population does not comprise (1) the first bacterial strain and (2) the second bacterial strain or the supernatant thereof.

In some embodiments, the pathogen comprises an infant gastrointestinal pathogen. In some embodiments, the cultured medium or derivative thereof is configured to inhibit a growth of the infant gastrointestinal pathogen by at least 0.1% relative to a growth of the infant gastrointestinal pathogen when inhibited by the control cultured medium or derivative thereof of the control bacterial population. In some embodiments, the cultured medium or derivative thereof is configured to inhibit the growth of the infant gastrointestinal pathogen by at least 5% or by at least 50% relative to the growth of the infant gastrointestinal pathogen when inhibited by the control cultured medium or derivative thereof of the control bacterial population. In some embodiments, the cultured medium or derivative thereof is configured to inhibit a signal of an immune response reporter of the immune response signaling pathway within an engineered cell by at least 0.1% relative to a signal of the immune response reporter of the immune response signaling pathway within the engineered cell when inhibited by the control cultured medium or derivative thereof of the control bacterial population. In some embodiments, the cultured medium or derivative thereof is configured to inhibit the signal of the immune response reporter of the immune response signaling pathway within the engineered cell by at least 1% or by at least 10%, relative to the signal of the immune response reporter of the immune response signaling pathway within the engineered cell when inhibited by the control cultured medium or derivative thereof of the control bacterial population. In some embodiments, the engineered cell comprises a mammalian cell. In some embodiments, the immune response reporter comprises an inflammatory immune response reporter. In some embodiments, the pathogen comprises a vaginal pathogen. In some embodiments, the cultured medium or derivative thereof is configured to inhibit a growth or biofilm formation of the vaginal pathogen by at least 0.1% relative to a growth or biofilm formation of the vaginal pathogen when inhibited by the control cultured medium or derivative thereof of the control bacterial population. In some embodiments, the cultured medium or derivative thereof is configured to inhibit the growth or biofilm formation of the vaginal pathogen by at least 5% or by at least 50% relative to the growth or biofilm formation of the vaginal pathogen when inhibited by the control cultured medium or derivative thereof of the control bacterial population.

Disclosed herein, in some embodiments, are methods of treating a disease or disease condition in a subject in need thereof. In an aspect, a method of treating a disease or disease condition in a subject in need thereof comprises: administering to the subject a therapeutically-effective amount of a formulation comprising a supernatant of a culture or a derivative of the supernatant, wherein the culture comprises (1) a first bacterial strain and (2) a second bacterial strain or a cultured medium thereof, wherein the second bacterial strain is different from the first bacterial strain, wherein the first bacterial strain and the second bacterial strain comprise Bifidobacterium sp. or Vertebrate-Associated Lactobacillaceae, wherein the formulation inhibits (i) a growth or biofilm formation of a pathogen by at least 0.1% or (ii) an immune response signaling pathway by at least 0.1%, relative to a growth or biofilm formation of the pathogen or the immune response signaling pathway when inhibited by a control formulation comprising a control supernatant of a control culture or a derivative of the control supernatant, respectively, and wherein the control culture does not comprise (1) the first bacterial strain and (2) the second bacterial strain or the cultured medium thereof.

In some embodiments, the pathogen comprises an infant gastrointestinal pathogen. In some embodiments, the formulation inhibits a growth of the infant gastrointestinal pathogen by at least 0.1% relative to a growth of the infant gastrointestinal pathogen when inhibited by the control formulation. In some embodiments, the formulation inhibits the growth of the infant gastrointestinal pathogen by at least 5% or by at least 50% relative to the growth of the infant gastrointestinal pathogen when inhibited by the control formulation. In some embodiments, the formulation inhibits a signal of an immune response reporter of the immune response signaling pathway within an engineered cell by at least 0.1% relative to a signal of the immune response reporter of the immune response signaling pathway within the engineered cell when inhibited by the control formulation. In some embodiments, the formulation inhibits signal of the immune response reporter of the immune response signaling pathway within the engineered cell by at least 1% or at least 10% relative to the signal of the immune response reporter of the immune response signaling pathway within the engineered cell when inhibited by the control formulation. In some embodiments, the immune response signaling pathway comprises an inflammatory immune response signaling pathway. In some embodiments, the pathogen comprises a vaginal pathogen. In some embodiments, the formulation inhibits a growth or biofilm formation of the vaginal pathogen by at least 0.1% relative to a growth or biofilm formation of the vaginal pathogen when inhibited by the control formulation. In some embodiments, the formulation inhibits the growth or biofilm formation of the vaginal pathogen by at least 5% or by at least 50% relative to the growth or biofilm formation of the vaginal pathogen when inhibited by the control formulation.

In the compositions disclosed herein: In some embodiments, the composition is formulated in a vaginal dosage form. In some embodiments, the composition further comprises a pharmaceutically acceptable excipient. In some embodiments, the pharmaceutically acceptable excipient comprises a hydrogel. In some embodiments, the first bacterial strain or the second bacterial strain is configured to exhibit an adherence to a vaginal epithelial cell (VEC) by at least 1×10\hat{ }2 [colony-forming units (CFU)/9.5 centimeter square (cm\hat{ }2)] or by at least 1×10\hat{ }6 log CFU/cm\hat{ }2. In some embodiments, the first bacterial strain or the second bacterial strain is configured to exhibit a growth ratio of at least about 0.1 or at least about 0.5 between (1) the first bacterial strain or the second bacterial strain being cultured in a culture medium comprising a carbon source consisting of glycogen and (2) the first bacterial strain or the second bacterial strain being cultured in a culture medium comprising a carbon source consisting of glucose, respectively. In some embodiments, the vaginal pathogen comprises Prevotella bivia, Atopobium vaginae, Sneathia spp., G. vaginalis, L. iners, or a combination thereof. In some embodiments, the first bacterial strain or the second bacterial strain is configured to inhibit a growth of the vaginal pathogen by at least 5% or at least 20%, relative to a growth of the vaginal pathogen not inhibited by the first bacterial strain or the second bacterial strain, respectively. In some embodiments, the composition comprises a bacterial product generated by the first bacterial strain or the second bacterial strain. In some embodiments, the bacterial product is a fermentation product of the first bacterial strain or the second bacterial strain. In some embodiments, the bacterial product is a secreted metabolite of the first bacterial strain or the second bacterial strain. In some embodiments, the first bacterial strain or the second bacterial strain comprises the Vertebrate-Associated Lactobacillaceae. In some embodiments, the first bacterial strain comprises L. jensenii or L. gasseri. In some embodiments, the first bacterial strain comprise L. jensenii ST21 (DSM34525) or L. gasseri ST105 (DSM34528). In some embodiments, the first bacterial strain comprises at least two bacterial strains In some embodiments, the at least two bacterial strains comprise L. jensenii and L. gasseri. In some embodiments, the at least two bacterial strains comprise L. jensenii ST21 (DSM 34525) and L. gasseri ST105 (DSM34528). In some embodiments, the second bacterial strain comprises L. crispatus. In some embodiments, the second bacterial strain comprises L. crispatus ST100 (DSM33187), L. crispatus ST20 (DSM34527), or L. crispatus ST112 (DSM34529). In some embodiments, the second bacterial strain comprises L. crispatus ST100 (DSM33187).

In the methods of treating a disease or disease condition in a subject in need thereof disclosed herein: In some embodiments, the formulation is formulated in a vaginal dosage form. In some embodiments, the formulation further comprises a pharmaceutically acceptable excipient. In some embodiments, the pharmaceutically acceptable excipient comprises a hydrogel. In some embodiments, the first bacterial strain or the second bacterial strain is configured to exhibit an adherence to a vaginal epithelial cell (VEC) by at least 1×10\hat{ }2 [colony-forming units (CFU)/9.5 centimeter square (cm\hat{ }2)] or by at least 1×10\hat{ }6 log CFU/cm\hat{ }2. In some embodiments, the first bacterial strain or the second bacterial strain is configured to exhibit a growth ratio of at least about 0.1 or at least about 0.5 between (1) the first bacterial strain or the second bacterial strain being cultured in a culture medium comprising a carbon source consisting of glycogen and (2) the first bacterial strain or the second bacterial strain being cultured in a culture medium comprising a carbon source consisting of glucose, respectively. In some embodiments, the vaginal pathogen comprises Prevotella bivia, Atopobium vaginae, Sneathia spp., G. vaginalis, L. iners or a combination thereof. In some embodiments, the first bacterial strain or the second bacterial strain is configured to inhibit a growth of the vaginal pathogen by at least 5% or at least 20%, relative to a growth of the vaginal pathogen not inhibited by the first bacterial strain or the second bacterial strain, respectively. In some embodiments, the formulation comprises a bacterial product generated by the first bacterial strain or the second bacterial strain. In some embodiments, the bacterial product is a fermentation product of the first bacterial strain or the second bacterial strain. In some embodiments, the bacterial product is a secreted metabolite of the first bacterial strain or the second bacterial strain. In some embodiments, the first bacterial strain or the second bacterial strain comprises the Vertebrate-Associated Lactobacillaceae. In some embodiments, the first bacterial strain comprises L. jensenii or L. gasseri. In some embodiments, the first bacterial strain comprise L. jensenii ST21 (DSM34525) or L. gasseri ST105 (DSM34528). In some embodiments, the first bacterial strain comprises at least two bacterial strains In some embodiments, the at least two bacterial strains comprise L. jensenii and L. gasseri. In some embodiments, the at least two bacterial strains comprise L. jensenii ST21 (DSM 34525) and L. gasseri ST105 (DSM34528). In some embodiments, the second bacterial strain comprises L. crispatus. In some embodiments, the second bacterial strain comprises L. crispatus ST100 (DSM33187), L. crispatus ST20 (DSM34527), or L. crispatus ST112 (DSM34529). In some embodiments, the second bacterial strain comprises L. crispatus ST100 (DSM33187).

In the compositions disclosed herein: In some embodiments, the immune response signaling pathway comprises an inflammatory immune response signaling pathway or an innate immune response signaling pathway, optionally the immune response signaling pathway comprises a toll-like receptor (TLR) signaling pathway. In some embodiments, the TLR signaling pathway comprises TLR4 signaling pathway. In some embodiments, an inhibition of the TLR4 signaling pathway is measured by a nuclear factor kappa-light-chain-enhancer of activated B cells (NFkB) reporter. In some embodiments, the TLR receptor further comprises a ligand. In some embodiments, the ligand comprises lipopolysaccharide (LPS). In some embodiments, the disease or disease condition comprises NEC. In some embodiments, the composition is formulated in a solid dosage form or liquid dosage form. In some embodiments, the liquid dosage form comprises a suspension. In some embodiments, the composition is formulated in oral dosage form. In some embodiments, the composition further comprises a pharmaceutically acceptable excipient In some embodiments, the first bacterial strain or the second bacterial strain is configured to exhibit an adherence to an intestinal epithelial cell (IEC) by at least 1×10\hat{ }4 [colony-forming units (CFU)/9.5 centimeter square (cm\hat{ }2)], optionally by at least 1×10\hat{ }7 log CFU/cm\hat{ }2. In some embodiments, the first bacterial strain or the second bacterial strain is configured to exhibit a growth ratio of about at least about 0.1 or at least 0.7 between (1) the first bacterial strain or the second bacterial strain being cultured a culture medium comprising a carbon source consisting of lactose or human milk oligosaccharide (HMO) and (2) the first bacterial strain or the second bacterial strain being cultured in a culture medium comprising a carbon source consisting of glucose. In some embodiments, the HMO comprises 2′flucosyllaotase (2′-FL), Lacto-N-neotetraose (LNnT), or a combination thereof. In some embodiments, the second bacterial strain is configured to exhibit the growth ratio of at least about at least about 0.1 or at least 0.7 between (1) the second bacterial strain being cultured in the culture medium comprising the carbon source consisting of the lactose or the HMO and (2) the second bacterial strain being cultured in the culture medium comprising the carbon source consisting of the glucose. In some embodiments, the first bacterial strain is configured to exhibit the growth ratio of at most about 10%, or optionally by 50%, between (1) the first bacterial strain being cultured in the culture medium comprising the carbon source consisting of the lactose or the HMO and (2) the first bacterial strain being cultured in the culture medium comprising the carbon source consisting of the glucose. In some embodiments, the first bacterial strain is configured to not exhibit a growth in the culture medium comprising the carbon source consisting of the lactose or the HMO. In some embodiments, the infant gastrointestinal pathogen comprises an opportunistic pathogen. In some embodiments, the infant gastrointestinal pathogen comprises E. coli, K. pneumoniae, C. perfringens, S. aureus, S. flexneri, or a combination thereof. In some embodiments, the infant gastrointestinal pathogen comprises the E. coli. In some embodiments, the first bacterial strain or the second bacterial strain is configured to inhibit a growth of the infant gastrointestinal pathogen by at least 10%, or optionally by at least 50%, relative to growth of the infant gastrointestinal pathogen not inhibited by the first bacterial strain or the second bacterial strain, respectively. In some embodiments, the composition comprises a bacterial product generated by the first bacterial strain or the second bacterial strain. In some embodiments, the bacterial product is a fermentation product or a secreted metabolite of the first bacterial strain or the second bacterial strain. In some embodiments, the bacterial population, when contacted with a barrier comprising intestinal epithelial cells (IECs), increases an impedance of the barrier by at least 0.1%, relative to an impedance of the barrier contacted with a control bacterial population not comprising the first bacterial strain and the second bacterial strain. In some embodiments, the first bacterial strain or the second bacterial strain comprises Bifidobacterium longum, Bifidobacterium pseudocatenulatum, Bifidobacterium breve, Bifidobacterium bifidum, or Lactobacillus plantarum. In some embodiments, the first bacterial strain or the second bacterial strain comprises Bifidobacterium longum ST81 (DSM 34594), Bifidobacterium longum ST23 (DSM 34590), Bifidobacterium pseudocatenulatum ST37 (DSM 34587), Bifidobacterium breve ST71 (DSM 34607), Bifidobacterium longum ST119 (DSM 34608), Bifidobacterium bifidum ST31 (DSM 34533), or Lactobacillus plantarum ST65 (DSM 34526). In some embodiments, the first bacterial strain or the second bacterial strain comprises Bifidobacterium pseudocatenulatum ST37 (DSM 34587), Bifidobacterium breve ST71 (DSM 34607), Bifidobacterium longum ST119 (DSM 34608), Bifidobacterium bifidum ST31 (DSM 34533), or Lactobacillus plantarum ST65 (DSM 34526). In some embodiments, the first bacterial strain or the second bacterial strain comprises Bifidobacterium pseudocatenulatum ST37 (DSM 34587), Bifidobacterium breve ST71 (DSM 34607), Bifidobacterium longum ST119 (DSM 34608), Bifidobacterium bifidum ST31 (DSM 34533), and Lactobacillus plantarum ST65 (DSM 34526). In some embodiments, the second bacterial strain comprises Bifidobacterium bifidum ST31 (DSM 34533).

In the methods of treating a disease or disease condition in a subject in need thereof disclosed herein: In some embodiments, the immune response signaling pathway comprises an inflammatory immune response signaling pathway or an innate immune response signaling pathway, optionally the immune response signaling pathway comprises a toll-like receptor (TLR) signaling pathway. In some embodiments, the TLR signaling pathway comprises TLR4 signaling pathway. In some embodiments, TLR4 signaling pathway comprises a nuclear factor kappa-light-chain-enhancer of activated B cells (NFkB). In some embodiments, the TLR4 signaling pathway comprises a ligand. In some embodiments, the ligand comprises lipopolysaccharide (LPS). In some embodiments, the disease or disease condition comprises NEC. In some embodiments, the formulation is formulated in a solid dosage form or liquid dosage form. In some embodiments, the liquid dosage form comprises a suspension. In some embodiments, the formulation is formulated in oral dosage form. In some embodiments, the method further comprises a pharmaceutically acceptable excipient In some embodiments, the first bacterial strain or the second bacterial strain is configured to exhibit an adherence to an intestinal epithelial cell (IEC) by at least 1×10\hat{ }4 [colony-forming units (CFU)/9.5 centimeter square (cm\hat{ }2)], optionally by at least 1×10\hat{ }7 log CFU/cm\hat{ }2. In some embodiments, the first bacterial strain or the second bacterial strain is configured to exhibit a growth ratio of about at least about 0.1 or at least 0.7 between (1) the first bacterial strain or the second bacterial strain being cultured a culture medium comprising a carbon source consisting of lactose or human milk oligosaccharide (HMO) and (2) the first bacterial strain or the second bacterial strain being cultured in a culture medium comprising a carbon source consisting of glucose. In some embodiments, the HMO comprises 2′flucosyllaotase (2′-FL), Lacto-N-neotetraose (LNnT), or a combination thereof. In some embodiments, the second bacterial strain is configured to exhibit the growth ratio of at least about at least about 0.1 or at least 0.7 between (1) the second bacterial strain being cultured in the culture medium comprising the carbon source consisting of the lactose or the HMO and (2) the second bacterial strain being cultured in the culture medium comprising the carbon source consisting of the glucose. In some embodiments, the first bacterial strain is configured to exhibit the growth ratio of at most about 0.99 between (1) the first bacterial strain being cultured in the culture medium comprising the carbon source consisting of the lactose or the HMO and (2) the first bacterial strain being cultured in the culture medium comprising the carbon source consisting of the glucose. In some embodiments, the first bacterial strain is configured to not exhibit a growth in the culture medium comprising the carbon source consisting of the lactose or the HMO. In some embodiments, the infant gastrointestinal pathogen comprises an opportunistic pathogen. In some embodiments, the infant gastrointestinal pathogen comprises E. coli, K. pneumoniae, C. perfringens, S. aureus, S. flexneri, or a combination thereof. In some embodiments, the infant gastrointestinal pathogen comprises the E. coli. In some embodiments, the first bacterial strain or the second bacterial strain is configured to inhibit a growth of the infant gastrointestinal pathogen by at least 10%, or optionally by 50%, relative to growth of the infant gastrointestinal pathogen not inhibited by the first bacterial strain or the second bacterial strain, respectively. In some embodiments, the formulation comprises a bacterial product generated by the first bacterial strain or the second bacterial strain. In some embodiments, the bacterial product is a fermentation product or a secreted metabolite of the first bacterial strain or the second bacterial strain. In some embodiments, the bacterial population, when contacted with a barrier comprising intestinal epithelial cells (IECs), increases an impedance of the barrier by at least 0.1%, relative to an impedance of the barrier contacted with a control bacterial population not comprising the first bacterial strain and the second bacterial strain. In some embodiments, the first bacterial strain or the second bacterial strain comprises Bifidobacterium longum, Bifidobacterium pseudocatenulatum, Bifidobacterium breve, Bifidobacterium bifidum, or Lactobacillus plantarum. In some embodiments, the first bacterial strain or the second bacterial strain comprises Bifidobacterium longum ST81 (DSM 34594), Bifidobacterium longum ST23 (DSM 34590), Bifidobacterium pseudocatenulatum ST37 (DSM 34587), Bifidobacterium breve ST71 (DSM 34607), Bifidobacterium longum ST119 (DSM 34608), Bifidobacterium bifidum ST31 (DSM 34533), or Lactobacillus plantarum ST65 (DSM 34526). In some embodiments, the first bacterial strain or the second bacterial strain comprises Bifidobacterium pseudocatenulatum ST37 (DSM 34587), Bifidobacterium breve ST71 (DSM 34607), Bifidobacterium longum ST119 (DSM 34608), Bifidobacterium bifidum ST31 (DSM 34533), or Lactobacillus plantarum ST65 (DSM 34526). In some embodiments, the first bacterial strain or the second bacterial strain comprises Bifidobacterium pseudocatenulatum ST37 (DSM 34587), Bifidobacterium breve ST71 (DSM 34607), Bifidobacterium longum ST119 (DSM 34608), Bifidobacterium bifidum ST31 (DSM 34533), and Lactobacillus plantarum ST65 (DSM 34526). In some embodiments, the second bacterial strain comprises Bifidobacterium bifidum ST31 (DSM 34533).

In some embodiments, the bacterial population is purified In some embodiments, the composition comprises less than 20 bacterial strains. In some embodiments, the composition comprises at least 3 or at least 4 bacterial strains. In some embodiments, the first bacterial strain comprises at least 2 or at least 3 bacterial strains. In some embodiments, the second bacterial strain comprises at least 2 or at least 3 bacterial strains. In some embodiments, the composition comprises at least about 10\hat{ }2 colony-forming units (CFU) of the first bacterial strain and the second bacterial strain. In some embodiments, the composition comprises at most about 10\hat{ }15 colony-forming units (CFU) of the first bacterial strain and the second bacterial strain. In some embodiments, within the composition, an amount of the first bacterial strain is at least about 5%, 10%, 20%, 50% 100%, 2-fold, 10-fold, or 100-fold higher than an amount of the second bacterial strain. In some embodiments, within the composition, an amount of the first bacterial strain is at most about 5%, 10%, 20%, 50% 100%, 2-fold, 10-fold, or 100-fold higher than an amount of the second bacterial strain. In some embodiments, within the composition, an amount of the first bacterial strain is at least about 5%, 10%, 20%, 50% 100%, 2-fold, 10-fold, or 100-fold lower than an amount of the second bacterial strain. In some embodiments, within the composition, an amount of the first bacterial strain is at most about 5%, 10%, 20%, 50% 100%, 2-fold, 10-fold, or 100-fold lower than an amount of the second bacterial strain.

Disclosed herein, in some embodiments, are compositions. In an aspect, a composition comprises: a bacterial population comprising a bacterial strain for use in treating an infant gastrointestinal disease in a subject in need thereof, wherein the composition is configured to treat the infant gastrointestinal disease at least in part by inhibiting a growth of an infant gastrointestinal pathogen in the subject by at least 60%, relative to a growth of the infant gastrointestinal pathogen in the subject when not inhibited by the composition.

Disclosed herein, in some embodiments, are methods of treating an infant gastrointestinal disease in a subject in need thereof. In an aspect, a method of treating an infant gastrointestinal disease in a subject in need thereof comprises: administering to the subject a therapeutically-effective amount of a formulation, wherein the formulation comprises a bacterial population comprising a bacterial strain, wherein (1) the formulation inhibits a growth of an infant gastrointestinal pathogen by at least 60%, relative to a growth of the infant gastrointestinal pathogen when not inhibited by the formulation, or (2) the formulation is configured to treat the infant gastrointestinal disease at least in part by inhibiting a growth of an infant gastrointestinal pathogen in the subject by at least 60%, relative to a growth of the infant gastrointestinal pathogen in the subject formulation not inhibited by the formulation.

Disclosed herein, in some embodiments, are compositions. In an aspect, a composition comprises: a bacterial population comprising a bacterial strain for use in treating a vaginal disease or a complication associated with the vaginal disease in a subject in need thereof, wherein the composition is formulated for application to a vagina of the subject, and wherein the composition is configured to treat the vaginal disease or the complication associated with the vaginal disease at least in part by inhibiting a vaginal pathogen in the subject by at least 30%, relative to a growth or biofilm formation of the vaginal pathogen in the subject when not inhibited by the composition.

Disclosed herein, in some embodiments, are methods of a vaginal disease or a complication associated with the vaginal disease in a subject in need thereof. In an aspect, a method of treating a vaginal disease or a complication associated with the vaginal disease in a subject in need thereof comprises administering to the subject a therapeutically-effective amount of a formulation, wherein the formulation comprises a bacterial population comprising a bacterial strain, wherein (1) the formulation inhibits a growth or biofilm formation of a vaginal pathogen by at least 30%, relative to a growth or biofilm formation of the vaginal pathogen when not inhibited by the formulation, or (2) wherein the formulation is configured to treat the vaginal disease or the complication associated with the vaginal disease at least in part by inhibiting a growth or biofilm formation of the vaginal pathogen in the subject by at least 30%, relative to a growth or biofilm formation of the vaginal pathogen in the subject when not inhibited by the formulation.

Disclosed herein, in some embodiments, are compositions. In an aspect, a composition comprises: a bacterial population comprising a first bacterial strain, wherein when the first bacterial strain is cultured with (1) a second bacterial strain different from the first bacterial strain or (2) a cultured medium of the second bacterial strain: (a) the first bacterial strain inhibits an immune response signaling pathway by at least 10% relative to the immune response signaling pathway when inhibited by a control bacterial strain comprising the first bacterial strain not cultured with (1) the second bacterial strain or (2) the cultured medium of the second bacterial strain, respectively, or (b) a cultured medium of the first bacterial strain inhibits the immune response signaling pathway by at least 10% relative to the immune response signaling pathway when inhibited by a cultured medium of the control bacterial strain, respectively.

Disclosed herein, in some embodiments, are compositions. In an aspect, a composition comprises: a bacterial population comprising a first bacterial strain and a second bacterial strain different from the first bacterial strain for use in treating a vaginal disease or a complication associated with the vaginal disease in a subject in need thereof, wherein the first bacterial strain and the second bacterial strain are derived from a vagina of a donor, wherein the composition is formulated for application to a vagina of the subject, and wherein the composition is configured to treat the vaginal disease or the complication associated with the vaginal disease at least in part by inhibiting a growth or biofilm formation of a vaginal pathogen in the subject by at least 0.1%, relative to a growth or biofilm formation of the vaginal pathogen in the subject when not inhibited by the composition

In some embodiments, the donor is a human. In some embodiments, the human is a healthy individual. In some embodiments, the human does not have the vaginal disease or the complication associated with the vaginal disease. In some embodiments, the first bacterial strain and the second bacterial strain are derived from a microbiota of the vagina of the donor.

Disclosed herein, in some embodiments, are compositions. In an aspect, a composition comprises: a bacterial population comprising a first bacterial strain and a second bacterial strain different from the first bacterial strain, wherein the first bacterial strain and the second bacterial strain are derived from a gastrointestinal tract of a human, and wherein when the first bacterial strain is cultured with (1) the second bacterial strain or (2) a cultured medium of the second bacterial strain: (a) the first bacterial strain inhibits a growth of a gastrointestinal pathogen by at least 0.1% relative to a growth of the gastrointestinal pathogen when inhibited by a control bacterial strain comprising the first bacterial strain not cultured with (1) the second bacterial strain or (2) the cultured medium of the second bacterial strain, respectively, (b) the first bacterial strain inhibits an immune response signaling pathway by at least 0.1% relative to the immune response signaling pathway when inhibited by the control bacterial strain, respectively, (c) a cultured medium of the first bacterial strain inhibits the growth of the gastrointestinal pathogen by at least 0.1% relative to a growth of the gastrointestinal pathogen when inhibited by a cultured medium of the control bacterial strain, respectively, or (d) the cultured medium of the first bacterial strain inhibits the signal of the immune response signaling pathway by at least 0.1% relative to the immune response reporter when inhibited by the cultured medium of the control bacterial strain, respectively.

In some embodiments, the human is a healthy individual. In some embodiments, the human is an infant. In some embodiments, the infant does not have the infant gastrointestinal disease.

Disclosed herein, in some embodiments, are compositions. In an aspect, a composition comprises: a bacterial product originating from a culture of: (1) a first bacterial strain and a second bacterial strain different from the first bacterial strain or (2) the first bacterial strain and a cultured medium of the second bacterial strain, wherein the first bacterial strain and the second bacterial strain comprise Bifidobacterium sp. or Vertebrate-Associated Lactobacillaceae, and wherein the bacterial product is configured to inhibit: (a) a growth of or biofilm formation of a pathogen by at least 0.1% relative to a growth of the pathogen when inhibited by a control bacterial metabolite generated by a control culture; or (b) an immune response signaling pathway by at least 0.1% relative to the immune response signaling when inhibited by the control bacterial product generated by the control culture, and wherein the control culture does not comprise the (1) first bacterial strain and (2) the second bacterial strain or the culture medium of the second bacterial strain.

In some embodiments, the pathogen comprises an infant gastrointestinal pathogen. In some embodiments, the bacterial product is configured to inhibit: (i) a growth of the infant gastrointestinal pathogen by at least 0.1% relative to a growth of the infant gastrointestinal when inhibited by the control bacterial product generated by the control culture; or (ii) a signal of an immune response reporter of the immune response signaling pathway within an engineered cell by at least 0.1% relative to a signal of the immune response reporter of the immune response signaling pathway within the engineered cell when inhibited by the control bacterial product generated by the control culture. In some embodiments, the pathogen comprises a vaginal pathogen. In some embodiments, the bacterial product is configured to inhibit a growth or biofilm formation of the vaginal pathogen by at least 0.1%, relative to a growth or biofilm formation of the vaginal pathogen when inhibited by control bacterial product generated by the control culture. In some embodiments, the bacterial product comprises at least lactic acid hydrogen peroxide, lipoteichoic acid, proteinaceous products, peptide products, short chain fatty acids, immunomodulatory lipids, bacteriocins or a combination thereof. In some embodiments, the bacterial product comprises at least 2, 3, 4, 5 or more bacterial products.

Disclosed herein, in some embodiments, are compositions. In an aspect, a composition comprises: a bacterial population comprising at least one strain of Bifidobacterium sp. or Vertebrate-Associated Lactobacillaceae, wherein the bacterial population is capable of exhibiting: (a) an increase of adhering to an intestinal epithelial cell (IPC) by at least 0.1%; (b) an increase in an impedance of a barrier comprising IPCs by at least 0.1%; (c) an increase of growth in a medium comprising a human milk oligosaccharide by at least 0.1%; (d) an increase of inhibiting a growth of a pathogen by at least 0.1%; (e) a decrease of an immune response reporter by at least 0.1%; or (f) a combination of (a)-(e), relative to a control bacterial strain comprising B. longum EV27 or L. reuteri BG49.

Disclosed herein, in some embodiments, are compositions. In an aspect, a composition comprises: at least one of Bifidobacterium bifidum ST31 (DSM34533), Bifidobacterium bifidum ST80 (DSM 34534), Lactobacillus crispatus ST112 (DSM34529), Lactobacillus crispatus ST20 (DSM34527), Lactobacillus gasseri ST105 (DSM34528), Lactobacillus jensenii ST21 (DSM34525), Lactobacillus plantarum ST65 (DSM34526), Bifidobacterium adolescentis ST101 (DSM34592), Bifidobacterium breve ST56 (DSM34588), Bifidobacterium longum ST19 (DSM34589), Bifidobacterium longum ST81 (DSM34594), Bifidobacterium pseudocatenulatum ST37 (DSM34587), Bifidobacterium pseudocatenulatum ST66 (DSM34591), Lactobacillus crispatus ST100 (DSM33187), Lactobacillus rhamnosus ST116 (DSM34593), Bifidobacterium longum ST23 (DSM34590), Bifidobacterium breve ST71 (DSM34607), or Bifidobacterium longum ST119 (DSM34608).

In some embodiments, the composition comprises at least two of Bifidobacterium bifidum ST31 (DSM34533), Bifidobacterium bifidum ST80 (DSM 34534), Lactobacillus crispatus ST112 (DSM34529), Lactobacillus crispatus ST20 (DSM34527), Lactobacillus gasseri ST105 (DSM34528), Lactobacillus jensenii ST21 (DSM34525), Lactobacillus plantarum ST65 (DSM34526), Bifidobacterium adolescentis ST101 (DSM34592), Bifidobacterium breve ST56 (DSM34588), Bifidobacterium longum ST19 (DSM34589), Bifidobacterium longum ST81 (DSM34594), Bifidobacterium pseudocatenulatum ST37 (DSM34587), Bifidobacterium pseudocatenulatum ST66 (DSM34591), Lactobacillus crispatus ST100 (DSM33187), Lactobacillus rhamnosus ST116 (DSM34593), Bifidobacterium longum ST23 (DSM34590), Bifidobacterium breve ST71 (DSM34607), or Bifidobacterium longum ST119 (DSM34608). In some embodiments, the composition is formulated in an oral or vaginal dosage form. In some embodiments, the composition comprises at least about 10\hat{ }2 colony-forming units per strain.

Disclosed herein, in some embodiments, are methods for generating a bacterial combination. In an aspect, a method for generating a bacterial combination comprises: (a) providing a first bacterial strain; (b) generating a plurality of cultures, each comprising the first bacterial strain and: (1) a given bacterial strain of a plurality of bacterial strains or (2) a metabolite generated by the given bacterial strain; (c) determining a culture of the plurality of cultures of (b) that is capable of (i) inhibiting a growth or biofilm formation of a vaginal pathogen by at least 0.1% relative to a growth of or biofilm formation of the vaginal pathogen inhibited by a control culture; (ii) inhibiting a signal of an immune response reporter by at least 0.1% relative to a signal of the immune response reporter inhibited by the control culture; or (iii) inhibiting a growth of an infant gastrointestinal pathogen by at least 0.1% relative to a growth of the infant gastrointestinal pathogen inhibited by the control culture, wherein the control culture does not comprise the first bacterial strain and (1) the given bacterial strain or (2) the metabolite generated by the given bacterial strain, thereby identifying the given bacterial strain to generate the bacterial combination comprising the first bacterial strain and the given bacterial strain.

Disclosed herein, in some embodiments, are pharmaceutical compositions. In an aspect, a pharmaceutical composition comprises a bacterial population comprising a first bacterial strain and a second bacterial strain, wherein the first bacterial strain and the second bacterial strain are different from one another, wherein the bacterial population comprises Bifidobacterium sp. or Vertebrate-Associated Lactobacillaceae, wherein the first bacterial strain, when present within: (1) a medium comprising an energy source and the second bacterial strain or (2) a supernatant of the medium comprising the energy source and the second bacterial strain, exhibits a growth of at least about 105% by weight as compared to a growth of the first bacterial strain when present in a medium comprising the energy source in an absence of the second bacterial strain or the supernatant of the medium comprising the energy source and the second bacterial strain, and wherein the energy source comprises a human milk oligosaccharide (HMO) or does not comprise starch.

In some embodiments, the supernatant of the medium comprising the energy source and the second bacterial strain is cell-free. In some embodiments, the supernatant of the medium comprising the energy source and the second bacteria strain comprises a fermentation product derived from the second bacterial strain. In some embodiments, the first bacterial strain, when present within (1) the medium comprising the energy source and the second bacterial strain or (2) the supernatant of the medium comprising the energy source and the second bacterial strain, exhibits a growth for at most about 48 hours, at most about 24 hours, or at most about 12 hours of at least about 105% by weight as compared to a growth of the first bacterial strain when present within the medium comprising the energy source in an absence of the second bacterial strain or the supernatant of the medium comprising the energy source and the second bacterial strain for at most about 48 hours, at most about 24 hours, or at most about 12 hours, respectively. In some embodiments, the first bacterial strain, when present within (1) the medium comprising the energy source and the second bacterial strain or (2) the supernatant of the medium comprising the energy source and the second bacteria strain, exhibits the growth of at least about 150%, at least about 1000%, or at least about 10000%, by weight as compared to the growth of the first bacterial strain when present within the medium comprising the energy source in an absence of the second bacterial strain or the supernatant of the medium comprising the energy source and the second bacteria strain. In some embodiments, the first bacterial strain or the second bacterial strain does not comprise a recombinant genetic modification, optionally wherein each of the first bacterial strain and the second bacterial strain does not comprise the recombinant genetic modification. In some embodiments, the pharmaceutical composition is formulated in an enteral dosage form, an injectable dosage form, a parenteral dosage form, a topical dosage form, or a combination thereof, optionally wherein the enteral dosage form comprises an oral dosage form, an intragastric dosage form, or a rectal dosage form. In some embodiments, the Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof. In some embodiments, the Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the Bifidobacterium sp. comprises B. animalis, B. pseudocatenulatum, B. bifidum, B. breve, B. dentium, B. faecale, B. longum, or a combination thereof. In some embodiments, the first bacteria strain comprises the Bifidobacterium sp. or the Vertebrate-Associated Lactobacillaceae. In some embodiments, second bacteria strain comprises the Bifidobacterium sp. or the Vertebrate-Associated Lactobacillaceae. In some embodiments, the energy source does not comprise the starch, optionally wherein the starch is not a modified starch, fermented starch, a dextrin, or a maltodextrin. In some embodiments, the energy source comprises the HMO, optionally wherein the HMO comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, or a combination thereof.

Disclosed herein, in some embodiments, are pharmaceutical compositions. In an aspect, a pharmaceutical composition comprises a bacterial population comprising a bacterial strain, wherein the bacterial population comprises Bifidobacterium sp. or Vertebrate-Associated Lactobacillaceae, and wherein a metabolite or an inviable cell derived from the bacterial strain, when combined with an engineered cell comprising a reporter, decreases a signal of the reporter by at least about 5% as compared to a signal of the reporter when the engineered cell is not combined with the metabolite or the inviable cell derived from the bacterial strain.

In some embodiments, a medium comprising the metabolite or the inviable cells derived from the bacterial strain comprises a supernatant derived from a growth culture of the bacterial strain, optionally wherein the supernatant derived from the growth culture of the bacterial strain is cell-free. In some embodiments, the medium comprising the metabolite or the inviable cells derived from the bacterial strain comprises a fermentation product derived from a growth culture of the bacterial strain. In some embodiments, the bacterial strain does not comprise a recombinant genetic modification. In some embodiments, the metabolite is not a cluster of differentiation 4 (CD4) peptide or a fragment thereof. In some embodiments, the engineered cell comprises an engineered immune cell, optionally wherein the engineered immune cell comprises a macrophage. In some embodiments, the reporter comprises an immune response reporter. In some embodiments, the reporter comprises a nuclear factor kappa-light-chain-enhancer of activated B cells (NFkB) reporter or an interferon-sensitive response element reporter (ISRE), optionally wherein the NFkB reporter comprises a secreted embryonic alkaline phosphatase (SEAP) reporter or the ISRE reporter comprises a Lucia luciferase. In some embodiments, the metabolite or the inviable cell derived from the bacterial strain, when combined with the engineered cell comprising the reporter, decreases the signal of the reporter by at least about 10% or at least about 50% as compared to when the engineered cell is not combined with the metabolite or the inviable cell derived from the bacterial strain. In some embodiments, the pharmaceutical composition is formulated in an enteral dosage form, an injectable dosage form, a parenteral dosage form, a topical dosage form, or a combination thereof. In some embodiments, the enteral dosage form comprises an oral dosage form, an intragastric dosage form, or a rectal dosage form, optionally wherein the intragastric dosage form comprises a dosage form that is configured to pass through a feeding tube. In some embodiments, the bacteria strain comprises the Bifidobacterium sp, optionally wherein the Bifidobacterium sp. comprises B. animalis, B. pseudocatenulatum, B. bifidum, B. breve, B. dentium, B. faecale, B. longum, or a combination thereof. In some embodiments, the bacteria strain comprises the Vertebrate-Associated Lactobacillaceae, optionally wherein the Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof. In some embodiments, the Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.

Disclosed herein, in some embodiments, are methods. In an aspect, a method comprises administering the pharmaceutical composition disclosed herein to a subject. In some embodiments, the subject has bacterial vaginosis (BV) or necrotizing enterocolitis (NEC) or a risk of the BV or a risk of the NEC. In some embodiments, the subject has the BV or the risk of the BV, optionally wherein the subject has a microbial dysbiosis in a vagina of the subject. In some embodiments, the subject is at least about 10 years old or at most about 120 years old. In some embodiments, the subject has the NEC or the risk of the NEC, optionally wherein the subject has a microbial dysbiosis in a gastrointestinal tract of the subject. In some embodiments, the subject is at most about 1 year old or at least about 1 day old, optionally wherein the subject is a premature infant.

Disclosed herein, in some embodiments, are methods. In an aspect, a method comprises: (a) providing a plurality of bacterial strains; (b) culturing a given bacterial strain of the plurality of bacterial strains in a carbon source or a plurality of carbon sources; (c) measuring growth of the plurality of bacterial strains; and (d) selecting a bacterial strain of the plurality of bacteria strains, wherein the plurality of bacterial strains comprises Bifidobacterium sp. or Vertebrate-Associated Lactobacillaceae, wherein the bacterial strain, when present within (1) a medium comprising the carbon source and a second bacterial strain or (2) a supernatant of the medium comprising the carbon source and the second bacteria strain, exhibits a growth of at least about 105% by weight as compared to a growth of the first bacterial strain when present within a medium comprising the carbon source in an absence of the second bacterial strain or the supernatant of the medium comprising the carbon source and the second bacteria strain, and wherein the carbon source comprises human milk oligosaccharide (HMO) or does not comprise starch.

Disclosed herein, in some embodiments, are methods. In an aspect, a method comprises: (a) providing a plurality of bacterial strains; (b) culturing a given bacterial strain of the plurality of bacterial strains in a carbon source of a plurality of carbon sources; (c) measuring growth of the plurality of bacterial strains; and (d) selecting a bacterial strain of the plurality of bacteria strains, wherein the plurality of bacterial strains comprises Bifidobacterium sp. or Vertebrate-Associated Lactobacillaceae, wherein a metabolite or an inviable cell derived from the bacterial strain, when combined with an engineered cell comprising a reporter, decreases a signal of the reporter by at least about 5% as compared to a signal of the reporter when the engineered cell is not combined with the metabolite or the inviable cell derived from the bacterial strain.

Disclosed herein, in some embodiments, are pharmaceutical compositions. In an aspect, a pharmaceutical composition comprises i. a purified bacterial population comprising a bacterial population comprising a first bacterial strain and a second bacterial strain, wherein i. the first bacterial strain and the second bacterial strain are different from one another, ii. the bacterial population comprises Bifidobacterium sp. or Vertebrate-Associated Lactobacillaceae, iii. the first bacterial strain, when present in a medium comprising an energy source and a second bacterial strain or a supernatant of the medium comprising the energy source and the second bacterial strain, exhibits a growth of at least about 105% by weight as compared to growth of the first bacterial strain when present in a medium comprising the energy source in an absence of the second bacterial strain or the supernatant of the medium comprising the energy source and the second bacterial strain, and iv. wherein the energy source does not comprise starch.

In some embodiments, the supernatant of the medium comprising the energy source and the second bacteria can be cell-free, or a fermentation product derived from the second bacterial strain. In some embodiments, the first bacterial strain when present within the medium comprising the energy source and the second bacterial strain or the supernatant of the medium comprising the energy source and the second bacterial strain, exhibits a growth for at most about 48 hours, 24 hours, or 12 hours of at least about 105%, or at least 150%, or at least 1000%, or at least 10000% by weight as compared to a growth of the first bacterial strain when present within the medium comprising the energy source in an absence of the second bacterial strain or the supernatant of the medium comprising the energy source and the second bacterial strain for at most about 48 hours.

In some embodiments, a pharmaceutically acceptable dosage form can include an injectable dosage form, parenteral dosage form, topical dosage form, or a combination thereof. In some embodiments, the enteral dosage form can comprise an oral dosage form, intragastric dosage form, or rectal dosage form. In some embodiments, the intragastric dosage form can comprise a dosage form that is configured to pass through a feeding tube.

In some embodiments, the bacterial genus Lactobacillaceae can comprise strains Vertebrate-Associated Lactobacillaceae, LimosiVertebrate-Associated Lactobacillaceae, LigiVertebrate-Associated Lactobacillaceae, Lacticaseibacillus sp., Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the bacterial genus the Bifidobacterium can comprise strains B. animalis, B. pseudocatenulatum, B. bifidum, B. breve, B. dentium, B. faecale, B. longum, or a combination thereof. In some embodiments, the first bacterial strain can comprise the Bifidobacterium sp. or the Lactobacillaceae sp. In some embodiments, the second bacterial strain can comprise the Bifidobacterium sp. or the Lactobacillaceae sp.

In some embodiments, the starch is not a modified starch. In some embodiments, the starch is not a fermented starch. In some embodiments, the fermented starch is not a dextrin. In some embodiments, the dextrin is not a maltodextrin. In some embodiments, the energy source can comprise fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, or a combination thereof. In some embodiments, the pharmaceutical composition can comprise a pharmaceutically acceptable excipient, cryoprotectant, or combination thereof.

In some embodiments, the pharmaceutical composition can comprise a bacterial population comprising at least one strain of Bifidobacterium sp., at least one strain of Lactobacillus sp., at least one strain of Akkermansia sp., at least one strain of Anaerbutyricum sp., at least one strain of Anaerostipes sp., at least one strain of Anaerotignum sp., at least one strain of Bacillus sp., at least one strain of Bacteroides sp., at least one strain of Blautia sp., at least one strain of Clostridium sp., at least one strain of Coprococcus sp., Dorea sp., Enterococcus sp., at least one strain of Erysipelatoclostridium sp., at least one strain of Escherichia sp., at least one strain of Eubacterium sp., at least one strain of Faecalibacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., at least one strain of Roseburia sp., at least one strain of Ruminococcus sp., or at least one strain of Veillonella sp., or a combination thereof.

Disclosed herein, in some embodiments, are pharmaceutical compositions. In some aspects, the pharmaceutical composition can comprise a bacterial population comprising a first bacterial strain and a second bacterial strain, wherein i. the first bacterial strain and the second bacterial strain are different from one another, ii. wherein the bacterial population comprises Bifidobacterium sp. or Vertebrate-Associated Lactobacillaceae, and iii. wherein the first bacterial strain, when present within a medium comprising a secreted metabolite derived from the second bacterial strain, proliferates for at least about 10 cell divisions.

In some embodiments, the the first bacterial strain can proliferate within a medium comprising a secreted metabolite derived from the second bacterial strain. In some embodiments, the supernatant derived from the growth culture of the bacterial strain can be cell-free. In some embodiments, the first bacterial strain can proliferate within the medium comprising the secreted metabolite derived from the second bacterial strain for at least about 10, 15, 20, or 32 cell divisions in at most 12, 24, or 48 hours. In some embodiments, the secreted metabolite can be a supernatant derived from a growth culture of a second bacterial strain. In some embodiments, a pharmaceutically acceptable dosage form can include an injectable dosage form, parenteral dosage form, topical dosage form, or a combination thereof. In some embodiments, the enteral dosage form can comprise an oral dosage form, intragastric dosage form, or rectal dosage form. In some embodiments, the intragastric dosage form can comprise a dosage form that is configured to pass through a feeding tube.

In some embodiments, the bacterial genus Lactobacillaceae can comprise strains Vertebrate-Associated Lactobacillaceae, LimosiVertebrate-Associated Lactobacillaceae, LigiVertebrate-Associated Lactobacillaceae, Lacticaseibacillus sp., Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the bacterial genus the Bifidobacterium can comprise strains B. animalis, B. pseudocatenulatum, B. bifidum, B. breve, B. dentium, B. faecale, B. longum, or a combination thereof. In some embodiments, the first bacterial strain can comprise the Bifidobacterium sp. or the Lactobacillaceae sp. In some embodiments, the second bacterial strain can comprise the Bifidobacterium sp. or the Lactobacillaceae sp.

In some embodiments, the pharmaceutical composition can comprise a bacterial population comprising at least one strain of Bifidobacterium sp., at least one strain of Lactobacillus sp., at least one strain of Akkermansia sp., at least one strain of Anaerbutyricum sp., at least one strain of Anaerostipes sp., at least one strain of Anaerotignum sp., at least one strain of Bacillus sp., at least one strain of Bacteroides sp., at least one strain of Blautia sp., at least one strain of Clostridium sp., at least one strain of Coprococcus sp., Dorea sp., Enterococcus sp., at least one strain of Erysipelatoclostridium sp., at least one strain of Escherichia sp., at least one strain of Eubacterium sp., at least one strain of Faecalibacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., at least one strain of Roseburia sp., at least one strain of Ruminococcus sp., or at least one strain of Veillonella sp., or a combination thereof.

In some embodiments, the energy source can comprise fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof. In some embodiments, the starch is not a fermented starch. In some embodiments, the starch can comprise a modified starch. In some embodiments, the modified starch can comprise a fermented starch. fermented starch can comprise a dextrin. In some embodiments, the dextrin can comprise a maltodextrin. In some embodiments, the pharmaceutical composition can comprise a pharmaceutically acceptable excipient, cryoprotectant, or combination thereof.

Disclosed herein, in some embodiments, are pharmaceutical compositions. In some aspects, the pharmaceutical composition can comprise a bacterial population comprising a first bacterial strain and a second bacterial strain, wherein i. the first bacterial strain and the second bacterial strain are different from one another, ii. wherein the bacterial population comprises Bifidobacterium sp. or Vertebrate-Associated Lactobacillaceae, iii. wherein the first bacterial strain, when present within a medium comprising a secreted metabolite derived from the second bacterial strain and a layer of epithelial cells, can decrease permeability of the layer of epithelial cells by at least about 5%, as compared to permeability of a layer of epithelial cells present within a medium comprising the first bacterial strain in an absence of the secreted metabolite, and wherein the permeability of the layer of epithelial cells is measured by transport of a Fluorescein isothiocyanate (FITC)-conjugated dextran or by transepithelial electrical resistance across the layer of epithelial cells.

In some embodiments, the medium comprising the secreted metabolite derived from the second bacterial strain can comprise a supernatant derived from a growth culture of the second bacterial strain. In some embodiments, the supernatant derived from a growth culture of a second bacterial strain can be cell-free. In some embodiments, the secreted metabolite supernatant can decrease permeability of epithelial cells by at least about 5%, 10%, 20%, 50%, or more than 50% as compared to permeability of a layer of epithelial cells present within a medium comprising the first bacterial strain in an absence of the secreted metabolite. In some embodiments, the epithelial cells can comprise mammalian epithelial cells. In some embodiments, the mammalian epithelial cells can comprise human epithelial cells.

In some embodiments, the bacterial genus Lactobacillaceae can comprise strains Vertebrate-Associated Lactobacillaceae, LimosiVertebrate-Associated Lactobacillaceae, LigiVertebrate-Associated Lactobacillaceae, Lacticaseibacillus sp., Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the bacterial genus the Bifidobacterium can comprise strains B. animalis, B. pseudocatenulatum, B. bifidum, B. breve, B. dentium, B. faecale, B. longum, or a combination thereof. In some embodiments, the first bacterial strain can comprise the Bifidobacterium sp. or the Lactobacillaceae sp. In some embodiments, the second bacterial strain can comprise the Bifidobacterium sp. or the Lactobacillaceae sp.

In some embodiments, the pharmaceutical composition can comprise a bacterial population comprising at least one strain of Bifidobacterium sp., at least one strain of Lactobacillus sp., at least one strain of Akkermansia sp., at least one strain of Anaerbutyricum sp., at least one strain of Anaerostipes sp., at least one strain of Anaerotignum sp., at least one strain of Bacillus sp., at least one strain of Bacteroides sp., at least one strain of Blautia sp., at least one strain of Clostridium sp., at least one strain of Coprococcus sp., Dorea sp., Enterococcus sp., at least one strain of Erysipelatoclostridium sp., at least one strain of Escherichia sp., at least one strain of Eubacterium sp., at least one strain of Faecalibacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., at least one strain of Roseburia sp., at least one strain of Ruminococcus sp., or at least one strain of Veillonella sp., or a combination thereof.

In some embodiments, the energy source can comprise fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof. In some embodiments, the starch is not a fermented starch. In some embodiments, the starch can comprise a modified starch. In some embodiments, the modified starch can comprise a fermented starch. fermented starch can comprise a dextrin. In some embodiments, the dextrin can comprise a maltodextrin. In some embodiments, the pharmaceutical composition can comprise a pharmaceutically acceptable excipient, cryoprotectant, or combination thereof.

In some embodiments, a pharmaceutically acceptable dosage form can include an injectable dosage form, parenteral dosage form, topical dosage form, or a combination thereof. In some embodiments, the enteral dosage form can comprise an oral dosage form, intragastric dosage form, or rectal dosage form. In some embodiments, the intragastric dosage form can comprise a dosage form that is configured to pass through a feeding tube.

Disclosed herein, in some embodiments, are pharmaceutical compositions. In some aspects, the pharmaceutical composition can comprise a bacterial population comprising a first bacterial strain and a second bacterial strain, wherein i. the first bacterial strain and the second bacterial strain are different from one another, ii. wherein the bacterial population comprises Bifidobacterium sp. or Vertebrate-Associated Lactobacillaceae, iii. wherein a secreted metabolite or an inviable cell derived from the bacterial strain, when combined with an engineered cell comprising a reporter, decreases a signal of the reporter by at least about 5% as compared to a signal of the reporter when the engineered cell is not combined with the metabolite or the inviable cell derived from the bacterial strain.

In some embodiments, the medium comprising the secreted metabolite or the inviable cells derived from the bacterial strain can comprise a supernatant derived from a growth culture of the bacterial strain. In some embodiments, the supernatant can be cell-free. In some embodiments, the medium comprising the secreted metabolite or the inviable cells derived from the bacterial strain can comprise a fermentation product derived from a growth culture of the bacterial strain. In some embodiments, the secreted metabolite derived from the bacterial strain can comprise a fermentation product derived from the bacterial strain. In some embodiments, the engineer cell can comprise a macrophage. In some embodiments, the inviable cell can comprise a pasteurized cell.

In some embodiments, the reporter can comprise a nuclear factor kappa-light-chain-enhancer of activated B cells (NFkB) reporter or an interferon-sensitive response element reporter (ISRE). In some embodiments, the NFkB reporter may comprise secreted embryonic alkaline phosphatase (SEAP) reporter. In some embodiments, the ISRE reporter may comprise a Lucia luciferase. In some embodiments, the secreted metabolite or inviable cell derived from the bacterial strain, when combined with the engineered cell, can decrease a signal of the reporter by at least about 5%, at least about 10%, or at least about 50%, or at least about more than 50% as compared to when the engineered cell is not combined with the metabolite or the inviable cell derived from the bacterial strain.

In some embodiments, a pharmaceutically acceptable dosage form can include an injectable dosage form, parenteral dosage form, topical dosage form, or a combination thereof. In some embodiments, the enteral dosage form can comprise an oral dosage form, intragastric dosage form, or rectal dosage form. In some embodiments, the intragastric dosage form can comprise a dosage form that is configured to pass through a feeding tube.

In some embodiments, the bacterial genus Lactobacillaceae can comprise strains Vertebrate-Associated Lactobacillaceae, LimosiVertebrate-Associated Lactobacillaceae, LigiVertebrate-Associated Lactobacillaceae, Lacticaseibacillus sp., Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the bacterial genus the Bifidobacterium can comprise strains B. animalis, B. pseudocatenulatum, B. bifidum, B. breve, B. dentium, B. faecale, B. longum, or a combination thereof. In some embodiments, the first bacterial strain can comprise the Bifidobacterium sp. or the Lactobacillaceae sp. In some embodiments, the second bacterial strain can comprise the Bifidobacterium sp. or the Lactobacillaceae sp.

In some embodiments, the pharmaceutical composition can comprise a bacterial population comprising at least one strain of Bifidobacterium sp., at least one strain of Lactobacillus sp., at least one strain of Akkermansia sp., at least one strain of Anaerbutyricum sp., at least one strain of Anaerostipes sp., at least one strain of Anaerotignum sp., at least one strain of Bacillus sp., at least one strain of Bacteroides sp., at least one strain of Blautia sp., at least one strain of Clostridium sp., at least one strain of Coprococcus sp., Dorea sp., Enterococcus sp., at least one strain of Erysipelatoclostridium sp., at least one strain of Escherichia sp., at least one strain of Eubacterium sp., at least one strain of Faecalibacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., at least one strain of Roseburia sp., at least one strain of Ruminococcus sp., or at least one strain of Veillonella sp., or a combination thereof.

In some embodiments, the energy source can comprise fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof. In some embodiments, the starch is not a fermented starch. In some embodiments, the starch can comprise a modified starch. In some embodiments, the modified starch can comprise a fermented starch. fermented starch can comprise a dextrin. In some embodiments, the dextrin can comprise a maltodextrin. In some embodiments, the pharmaceutical composition can comprise a pharmaceutically acceptable excipient, cryoprotectant, or combination thereof.

Disclosed herein, in some embodiments, are pharmaceutical compositions. In some aspects, the pharmaceutical composition can comprise a bacterial population comprising a first bacterial strain and a second bacterial strain, wherein i. the first bacterial strain and the second bacterial strain are different from one another, ii. wherein the bacterial population comprises Bifidobacterium sp. or Vertebrate-Associated Lactobacillaceae, and iii. wherein the first bacterial strain, when present within a medium comprising the second bacterial strain or a supernatant thereof for at most about 15 hours, can exhibit a growth of at least about 105% by colony-forming unit (CFU) as compared to a growth of the first bacterial strain when present within a medium not comprising the second bacterial strain or the supernatant thereof for at most about 15 hours.

In some embodiments, the medium comprising the second bacterial strain or the supernatant thereof can comprise a secreted metabolite derived from the second bacterial strain. In some embodiments, the secreted metabolite derived from the second bacterial strain can comprise a fermentation product of derived from the second bacterial strain. In some embodiments, the supernatant of the medium comprising the second bacterial strain can be cell-free.

In some embodiments, the first bacterial strain, when present within a medium comprising the second bacterial strain or a supernatant thereof for at most about 15, 10, or 8 hours, can exhibit a growth of at least about 105%, at least about 110%, at least about 120%, at least about 150%, at least 200%, or at least about more than 200% by CFU as compared to a growth of the first bacterial strain when present within a medium not comprising the second bacterial strain or the supernatant thereof.

In some embodiments, the bacterial genus Lactobacillaceae can comprise strains Vertebrate-Associated Lactobacillaceae, LimosiVertebrate-Associated Lactobacillaceae, LigiVertebrate-Associated Lactobacillaceae, Lacticaseibacillus sp., Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the bacterial genus the Bifidobacterium can comprise strains B. animalis, B. pseudocatenulatum, B. bifidum, B. breve, B. dentium, B. faecale, B. longum, or a combination thereof. In some embodiments, the first bacterial strain can comprise the Bifidobacterium sp. or the Lactobacillaceae sp. In some embodiments, the second bacterial strain can comprise the Bifidobacterium sp. or the Lactobacillaceae sp.

In some embodiments, the pharmaceutical composition can comprise a bacterial population comprising at least one strain of Bifidobacterium sp., at least one strain of Lactobacillus sp., at least one strain of Akkermansia sp., at least one strain of Anaerbutyricum sp., at least one strain of Anaerostipes sp., at least one strain of Anaerotignum sp., at least one strain of Bacillus sp., at least one strain of Bacteroides sp., at least one strain of Blautia sp., at least one strain of Clostridium sp., at least one strain of Coprococcus sp., Dorea sp., Enterococcus sp., at least one strain of Erysipelatoclostridium sp., at least one strain of Escherichia sp., at least one strain of Eubacterium sp., at least one strain of Faecalibacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., at least one strain of Roseburia sp., at least one strain of Ruminococcus sp., or at least one strain of Veillonella sp., or a combination thereof.

In some embodiments, the energy source can comprise fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof. In some embodiments, the starch is not a fermented starch. In some embodiments, the starch can comprise a modified starch. In some embodiments, the modified starch can comprise a fermented starch. fermented starch can comprise a dextrin. In some embodiments, the dextrin can comprise a maltodextrin. In some embodiments, the pharmaceutical composition can comprise a pharmaceutically acceptable excipient, cryoprotectant, or combination thereof.

Disclosed herein, in some embodiments, are pharmaceutical compositions. In some aspects, the pharmaceutical composition can comprise a bacterial population comprising at least one strain of Bifidobacterium sp. or Vertebrate-Associated Lactobacillaceae, wherein the bacterial population, when present within a medium comprising sialic acid, does not exhibit a growth of at least about 105% by weight as compared to a growth of a reference bacterial population when present within a medium comprising glucose.

In some embodiments, the bacterial population, when present within the medium comprising the sialic acid, may not exhibit the growth of at least about 105%, at least about 120%, at least about 150%, at least about 1000%, or at least about more than 1000% by weight as compared to the growth of the reference bacterial population when present within the medium comprising the glucose as a carbon source or sole carbon source.

In some embodiments, a pharmaceutically acceptable dosage form can include an injectable dosage form, parenteral dosage form, topical dosage form, or a combination thereof. In some embodiments, the enteral dosage form can comprise an oral dosage form, intragastric dosage form, or rectal dosage form. In some embodiments, the intragastric dosage form can comprise a dosage form that is configured to pass through a feeding tube.

In some embodiments, the bacterial genus Lactobacillaceae can comprise strains Vertebrate-Associated Lactobacillaceae, LimosiVertebrate-Associated Lactobacillaceae, LigiVertebrate-Associated Lactobacillaceae, Lacticaseibacillus sp., Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof. In some embodiments, the bacterial genus the Bifidobacterium can comprise strains B. animalis, B. pseudocatenulatum, B. bifidum, B. breve, B. dentium, B. faecale, B. longum, or a combination thereof. In some embodiments, the first bacterial strain can comprise the Bifidobacterium sp. or the Lactobacillaceae sp. In some embodiments, the second bacterial strain can comprise the Bifidobacterium sp. or the Lactobacillaceae sp.

In some embodiments, the pharmaceutical composition can comprise a bacterial population comprising at least one strain of Bifidobacterium sp., at least one strain of Lactobacillus sp., at least one strain of Akkermansia sp., at least one strain of Anaerbutyricum sp., at least one strain of Anaerostipes sp., at least one strain of Anaerotignum sp., at least one strain of Bacillus sp., at least one strain of Bacteroides sp., at least one strain of Blautia sp., at least one strain of Clostridium sp., at least one strain of Coprococcus sp., Dorea sp., Enterococcus sp., at least one strain of Erysipelatoclostridium sp., at least one strain of Escherichia sp., at least one strain of Eubacterium sp., at least one strain of Faecalibacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., at least one strain of Roseburia sp., at least one strain of Ruminococcus sp., or at least one strain of Veillonella sp., or a combination thereof.

In some embodiments, the energy source can comprise fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof. In some embodiments, the starch is not a fermented starch. In some embodiments, the starch can comprise a modified starch. In some embodiments, the modified starch can comprise a fermented starch. fermented starch can comprise a dextrin. In some embodiments, the dextrin can comprise a maltodextrin. In some embodiments, the pharmaceutical composition can comprise a pharmaceutically acceptable excipient, cryoprotectant, or combination thereof.

Disclosed herein, in some embodiments, are methods for treating a subject having or suspected of having a disease. In an aspect, a method for treating a subject having or suspected of having a disease comprises administering to the subject a pharmaceutical composition of any pharmaceutical compositions disclosed thereof.

In some embodiments, the disease is an inflammatory disease. In some embodiments, the inflammatory disease is a bacterial vaginosis (BV) or a necrotizing enterocolitis (NEC) disease. In some embodiments, the subject can have BV or NEC or be at risk of BV or NEC. In some embodiments, the subject can have a microbial dysbiosis in a gastrointestinal (GI) tract or vagina of the subject. In some embodiments, the subject at risk for BV can be at least about 10 years old, at least about 15 years old, at least about more than 15 years old, or at most about 120 years old. In some embodiments, the subject at risk for NEC can be a premature infant. In some embodiments, the subject at risk for NEC can be at most about 1 year old, or at least about 1 day old.

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the bacterial strain, when present within a medium comprising the carbon source and a second bacterial strain or a supernatant of the medium comprising the carbon source and the second bacterial strain, exhibits a growth of at least about 105% by weight as compared to a growth of the first bacterial strain when present within a medium comprising the carbon source in an absence of the second bacterial strain or the supernatant of the medium comprising the carbon source and the second bacterial strain and wherein the carbon source may not comprise starch.

In some embodiments, the supernatant of the medium can be cell-free. In some embodiments, the supernatant of the medium can comprise a fermentation product derived from the second bacterial strain.

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the plurality of bacterial strains comprises Bifidobacterium sp. or Vertebrate-Associated Lactobacillaceae, wherein the bacterial strain, when present within a medium comprising a secreted metabolite derived from a second bacterial strain, proliferates for at least about 10 cell divisions.

In some embodiments, the medium comprising the secreted metabolite derived from the bacterial strain can comprise a supernatant derived from a growth culture of the second bacterial strain. In some embodiments, the supernatant can be cell-free.

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the plurality of bacterial strains comprises Bifidobacterium sp. or Vertebrate-Associated Lactobacillaceae, wherein i. the bacterial strain, when present within a medium comprising a secreted metabolite derived from the second bacterial strain and a layer of epithelial cells, can decrease permeability of the layer of epithelial cells by at least about 5%, as compared to permeability of a layer of epithelial cells present within a medium comprising the bacterial strain in an absence of the secreted metabolite, and ii. wherein the permeability of the layer of epithelial cells can be measured by transport of a Fluorescein isothiocyanate (FITC)-conjugated dextran or by transepithelial electrical resistance across the layer of epithelial cells.

In some embodiments, the medium comprising the secreted metabolite derived from the bacterial strain can comprise a supernatant derived from a growth culture of the second bacterial strain. In some embodiments, the supernatant can be cell-free. In some embodiments, the supernatant can comprise a fermentation product derived from the second bacterial strain. In some embodiments, the epithelial cells can comprise mammalian epithelial cells. In some embodiments, the mammalian epithelial cells can comprise human epithelial cells.

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the plurality of bacterial strains comprises Bifidobacterium sp. or Vertebrate-Associated Lactobacillaceae, wherein a secreted metabolite or an inviable cell derived from the bacterial strain, when combined with an engineered cell comprising a reporter, decreases a signal of the reporter by at least about 5% as compared to a signal of the reporter when the engineered cell is not combined with the metabolite or the inviable cell derived from the bacterial strain.

In some embodiments, the medium comprising the secreted metabolite derived from the bacterial strain can comprise a supernatant derived from a growth culture of the second bacterial strain. In some embodiments, the supernatant can be cell-free. In some embodiments, the supernatant or the metabolite can comprise a fermentation product derived from the second bacterial strain. In some embodiments, the engineered cell comprises a macrophage. In some embodiments, the inviable cell comprises as pasteurized cell. In some embodiments, the reporter can comprise NFkB or ISRE. In some embodiments, the NFkB reporter can comprise a SEAP reporter. In some embodiments, the ISRE reporter can comprise a Lucia Luciferase.

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the plurality of bacterial strains comprises Bifidobacterium sp. or Vertebrate-Associated Lactobacillaceae, wherein i. the bacterial strain, when present within a medium comprising the second bacterial strain or a supernatant thereof for at most about hours, exhibits a growth of at least about 105% by colony-forming unit (CFU) as compared to a growth of the bacterial strain when present within a medium not comprising the second bacterial strain or the supernatant thereof for at most about 15 hours.

In some embodiments, the medium comprising the second bacterial strain or the supernatant thereof can comprise a secreted metabolite derived from the second bacterial strain. In some embodiments, the metabolite can comprise a fermentation product derived from the second bacterial strain. In some embodiments, the supernatant can be cell-free.

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the plurality of bacterial strains comprises Bifidobacterium sp. or Vertebrate-Associated Lactobacillaceae, wherein the first bacterial strain is i. configured to utilize a metabolite derived from the second bacterial strain as a growth promoter, and ii. wherein the metabolite is not a butyrate, a vitamin B12, or an ammonia (NH3).

In some embodiments, the metabolite can be a secreted metabolite derived from the second bacterial strain, and cannot be a derivative or a combination of the butyrate, the vitamin B12, or the ammonia (NH3).

Disclosed herein, in some embodiments, are methods for producing a pharmaceutical composition. In an aspect, a method for producing a pharmaceutical composition comprises i. providing a plurality of bacterial strains, wherein the plurality of bacterial strains comprises Bifidobacterium sp. or Vertebrate-Associated Lactobacillaceae, ii. Culturing a given bacterial strain of the plurality of bacterial strains in a carbon source or a plurality of carbon sources, iii. Measuring growth of the plurality of bacterial strains, and iv. Selecting a bacterial strain of the plurality of bacterial strains wherein the plurality of bacterial strains comprises Bifidobacterium sp. or Vertebrate-Associated Lactobacillaceae, wherein the bacterial strain is incapable of using sialic acid as a carbon source.

In some embodiments, the bacterial population, when present within the medium comprising the sialic acid, does not exhibit a growth of at least about 105% by weight as compared to the growth of a reference bacterial population when present within the medium comprising glucose as a carbon source or sole carbon source for the reference bacterial population.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The features of the disclosure are set forth with particularity in the appended claims. A better understanding of the features and the disclosure will be obtained by reference to the following detailed description that sets forth illustrative embodiments, in which the principles of the disclosure are utilized, and the accompanying drawings (also “Figure” and “FIG.” herein), of which:

FIG. 1 shows that the growth curve of the L. crispatus and L. jensenii colonies, species in the Lactobacilli genus on MRS plates over the course of 32 hours (L. crispatus) and 20 hours (L. jensenii).

FIG. 2 shows a schematic of the Direct Carbohydrate Utilization Screen method for monitoring the growth of Bifidobacteria and Lactobacilli cultures in various carbohydrate-rich mediums. The data analysis method for visualizing the growth rates is also shown.

FIG. 3 shows the layout of the 96-well plate used for the Direct Carbohydrate Utilization Screen method.

FIG. 4 shows an example of the growth ratio to glucose for each bacterial strain of interest in each carbohydrate stock solution media as a clustermap.

FIG. 5 shows an example of a principal coordinates analysis visualizing the AUC ratio of each strain of interest cultured in each carbohydrate stock solution media.

FIG. 6 shows an example of the predicted Carbohydrate Utilization Screen AUC results for Lactobacillus strains.

FIG. 7 shows the method for executing Bifidobacteria screening, a technique to isolate and identify Bifidobacterial strains via DNA sequencing.

FIGS. 8A-8B show an example of the optical density measurement results for Bifidobacteria DNA. FIG. 8A shows an example of measurement data for the Qubit and Nanodrop, creating an identification profile for the Bifidobacterial strain. FIG. 8B shows an example of the assay results, creating a visual indication for identification of the Bifidobacterial strain.

FIGS. 9A-9G show examples of the nanopore sequencing measurement results for Bifidobacteria DNA, including the length of the DNA and the quality of the DNA. FIG. 9A shows an example of the nanodrop measurement data including examples of the average genome size for each strain of interest. FIG. 9B shows an example of the nanodrop measurement and analysis data including completeness and contamination measurements for various strains of interest. FIG. 9C shows an example of the average data for various strains of interest including completeness, contamination, and genome size averages. FIG. 9D shows an example of the nanodrop measurement and analysis data including the assembly error per million base pairs and the consensus quality score (QV). FIG. 9E shows an example of the gene number data plotted for each species of interest. FIG. 9F shows an example of the gene length data plotted for each species of interest. FIG. 9G shows an example of nanodrop data analyzed for various strains of interest, including polished average gene length ratios.

FIGS. 10A-10D show the method for membrane integrity screening using a monolayer of Caco-2 cells as an in vitro model for epithelial tissue. FIG. 10A shows the general method for membrane integrity screening. FIG. 10B shows an example of the microscopy visualization of the monolayer of Caco-2 cells in room air and a CO₂ incubator. FIG. 10C diagrams a 24-well plate setup in which groups consisting of 4 wells per group were subject to different treatments. FIG. 10D shows an example of the data involving measurement of TNFα induced membrane permeability over a number of hours.

FIGS. 11A-11B show examples of the results of the TEER membrane permeability analysis. FIG. 11A shows an example of TEER and FD4 fluorescence results for samples treated with various levels of IFNγ and/or IFNg treatment over the span of 4 hours for TEER and 48 hours for FD4 results. FIG. 11B shows examples of TEER and FD4 fluorescence results for individual plates over a period of 4 hours for TEER and 24 hours for FD4 results.

FIGS. 12A-12D show examples of the results of TEER membrane permeability analysis in room air incubation and alternatively CO₂ incubation, as well as with and without the addition of HCO₃. FIG. 12A shows examples of TEER results measured with the room air incubation condition with HCO₃ as well as without HCO₃ for a span of 4 hours post-treatment. FIG. 12B shows examples of TEER results measured with the room air incubation condition with HCO₃ as well as without HCO₃ for a span of 24 hours post-treatment. FIG. 12C shows examples of TEER results measured with the CO₂ incubation condition with HCO₃ as well as without HCO₃ for a span of 4 hours post-treatment. FIG. 12D shows examples of TEER results measured with the CO₂ incubation condition with HCO₃ as well as without HCO₃ for a span of 24 hours post-treatment.

FIGS. 13A-13F show the pathogen growth inhibition assay plate layout and examples of the spectrophotometry data results. FIG. 13A shows the layout of the 96-well plate for the in vitro growth inhibition assay. FIG. 13B shows an example of the cluster map data analysis for the OD₆₀₀ measurements of various pathogenic strains of interest and various bacterial strains of interest. FIG. 13C shows examples of the OD₆₀₀ curve of cultured pathogenic E. coli is in media with metabolites of the bacterial strains of interest, for example L. rhamnosus ST34, A. muciniphila ST7, and F. prausnitzii ST38 (Fp ST38). FIG. 13D shows examples of the OD₆₀₀ curve of cultured pathogenic E. coli in media with metabolites of the Bifidobacterium strains of interest, for example B. longum ST59, B. breve ST56, B. longum infantis ST19, B. stercoris ST24, B. dentium ST40, B. stercoris ST101, B. longum ST27, B. bifidum_1 ST50, and B. breve_2 ST30. FIG. 13E shows examples of the OD₆₀₀ curve for growth of cultured pathogenic K. pneumoniae in media with metabolites of the bacterial strains of interest, for example L. rhamnosus ST34. FIG. 13F shows examples of the OD₆₀₀ curve for growth of cultured pathogenic K. pneumoniae in media with metabolites of the Bifidobacterium strains of interest.

FIG. 14 shows the OD₆₀₀ curve for dose-dependent inhibition of E. coli growth.

FIGS. 15A-15J show the plate setup and examples of results of the SEAP detection assay in detecting inflammatory transcription factor NFκB in RAW-Dual cells after treatment with potential therapeutic strains. FIG. 15A shows an example of the 96-well plate setup for the SEAP assay. FIG. 15B shows examples of SEAP test results at various MOIs. FIG. 15C shows examples of SEAP test results for various metabolite factors of the bacterial strains of interest, for example A. muciniphila ST7 and B. bifidum ST80. FIG. 15D shows examples of NFκB SEAP activity results from the metabolites of various Bifidobacterium strains of interest. FIG. 15E shows examples of basal NFκB SEAP activity results, as well as examples of results from the metabolites of various other species of interest, for example Blautia and Roseburia. FIG. 15F shows examples of NFκB SEAP activity results for various metabolite factors of the bacterial strains of interest on E. coli cell growth. FIG. 15G shows examples of NFκB SEAP activity results at various supernatant dilution factors for various pathogens of interest including E. coli. FIG. 15H shows examples of NFκB SEAP activity results at various supernatant dilution factors deriving from various bacterial strains of interest including B. bifidum ST80. FIG. 15I shows examples of NFκB SEAP activity results at various supernatant dilution factors deriving from various bacterial strains of interest including B. longum EV27. FIG. 15J shows examples of NFκB SEAP activity results at various supernatant dilution factors deriving from various bacterial strains of interest including Fp ST38.

FIGS. 16A-16H show examples of the results of LUC testing in detecting inflammatory transcription factor IRF in RAW-Dual cells after treatment with potential therapeutic strains. FIG. 16A shows examples of LUC test result data for IRF LUC activity at various concentrations of lipopolysaccharides (LPS). FIG. 16B shows examples of LUC test result data for various MOIs. FIG. 16C shows examples of the results of LUC testing for metabolite factors of the bacterial strains of interest, for example A. muciniphila ST7 and B. bifidum ST80 at various dilutions. FIG. 16D shows examples of the results of LUC testing for IRF LUC activity at various dilution factors for the metabolites of various strains of interest, including B. longum EV27. FIG. 16E shows examples of the results of LUC testing for IRF LUC activity at various dilution factors for the metabolites of various strains of interest, including B. theta ST8. FIG. 16F shows examples of the results of LUC testing for IRF LUC activity at various dilution factors for the metabolites of various strains of interest, including Fp ST38. FIG. 16G shows examples of the results of LUC testing for IRF LUC activity for various metabolites of the strains of interest when interacting with pathogens of interest, for example E. coli. FIG. 16H shows examples of the results of LUC testing for IRF LUC activity for the metabolites of various strains of interest at various LPS concentrations.

FIG. 17 depicts a cartoon schematic of an exemplary strain selection method as described herein.

FIG. 18 depicts an exemplary clustermap of vaginally relevant carbohydrate utilization screening results of the exemplary bacterial strains described herein, depicting the growth ratio between the carbohydrate (listed on y-axis) and glucose.

FIG. 19 depicts an exemplary clustermap of BV pathogen biofilm inhibition results represented by the Biofilm Remaining Ratio.

FIG. 20 depicts a cartoon schematic of an exemplary method for identify a therapeutic consortium.

FIG. 21 depicts an exemplary clustermap of NEC pathogen growth inhibition results represented by the Pathogen Growth Ratio (area under the growth curve of treated culture/area under the curve of a media control).

### DETAILED DESCRIPTION

### Microbial Dysbiosis and Diseases

Microbial dysbiosis, the imbalance of microbiome/microbiota (the group or community of microbes or microorganism residing within a subject), can cause or be associated in various diseases and disease/pathological conditions. In some cases, while the microbial dysbiosis may not cause the diseases or disease conditions, it can affect the development and/or progression of the symptoms of the diseases and disease/pathological conditions. In some cases, restoring the microbial dysbiosis can treat/prevent the diseases or disease conditions and improve/alleviate the development/progression of the symptoms of the diseases or disease conditions. One way to restore the microbial dysbiosis can comprise administration of microbes to a subject or patient.

A microbial dysbiosis-associated disease can comprise diseases that affect genital organ, such as vaginal diseases. Bacterial vaginosis (BV) is a vaginal disease that affects about 21 million females annually in the U.S. BV can increase the risk of acquiring sexually transmitted infections (STI), genital tract infections, miscarriage, pelvic inflammatory disease (PID), vulvovaginitis, lower success in fertility, preterm labor, preterm delivery, morbidity, and/or postpartum. Among the patients that have been treated with current standard of care (SOC), such as the uses of antibiotics, about 30% of them can experience recurrence of BV (as short as within 3-months). Treatments for recurrent BV are currently not available. Symptomatic BV can result in itching, unpleasant odor, and discharges.

BV can occur during the dysbiosis of the vaginal microbiota (microbiome). Bacterial diversity can be the primary clinical score for diagnosis of BV. The healthy vaginal microbiota is a community containing a small number of dominant lactic acid-producing Lactobacillus species. BV is characterized by a loss of beneficial Lactobacillus species, increases in pathobionts/pathogens (microbes not normally present in a healthy subject or microbes that present in an amount exceeding those in the normal healthy subject or the subject without the disease), microbial diversity, and/or an increase in vaginal pH. As used herein, a pathogen comprises a microorganism that: (1) causes a disease or disease condition; (2) associated with a disease or disease condition; and/or (3) contributes to the symptoms of the disease or disease condition. In some instances, inhibiting or eliminating a pathogen can treat or prevent the disease or disease condition or alleviate the symptoms of the disease or disease condition. In some cases, a pathogen can be an opportunistic pathogen that is not virulent in healthy subjects but can become virulent with immunocompromised and unhealthy subjects). In some cases, a pathogen can be a pathobiont.

In some cases, dominant Lactobacillus species within a healthy subject can protect the healthy vagina by preventing colonization/engraftment by pathogens and decrease inflammation through resource (such as nutrients) utilization, metabolic shifts, direct microbe-host interactions with the epithelial barrier, and/or vaginal tract acidification. For examples, the dominant Lactobacillus species of a healthy vagina can inhibit the engraftment of the pathogens at least by competitive exclusion of the pathogens; releasing bacterial products such as lactic acid or reactive oxidative species; and/or proteinaceous or peptidic antimicrobial products. During the progression of BV, the dominant Lactobacillus species in the healthy vagina can first be depleted, resulting in an increase of vaginal pH and/or loss of lactic acids and ROS (and other bacterial products produced by the dominant Lactobacillus species). The vagina can then be colonized by pathogens such as G. vaginalis and/or P. bivia, resulting in the muscle degradation, releases of the ammonia (via the conversion from the amino acid generated by the degraded muscles), increased growth and biofilm formation of G. vaginalis, P. bivia, and others, disruption of vaginal epithelial barrier, and/or releases of sialidase. Subsequently, the immune system of the subject can release pro-inflammatory cytokines/chemokines, causing inflammation and further complicating the muscle degradation, the release of the ammonia, the growth and biofilm formation of various vaginal pathogens, disruption of vaginal epithelial barrier, and/or releases of sialidase, resulting in BV. As used herein, a vaginal pathogen comprises a microorganism that: (1) causes a vaginal disease or disease condition; (2) associated with a vaginal disease or disease condition; and/or (3) contributes to the symptoms of the vaginal disease or disease condition. In some instances, inhibiting or eliminating a vaginal pathogen can treat or prevent the vaginal disease or disease condition or alleviate the symptoms of the vaginal disease or disease condition.

Another microbial dysbiosis-associated disease can comprise gastrointestinal diseases that affect gastrointestinal tract, such as infant gastrointestinal diseases or disease conditions. Necrotizing enterocolitis (NEC) is an infant gastrointestinal disease or disease condition that affects about 100,000 infants annually in the U.S. NEC is one of the leading causes of illness and death among preterm infants. About 5-12% of preterm infants (i.e., born before the 37 weeks of pregnancy) can develop NEC. About 40-50% of infants with NEC die from the disease.

NEC can result from intestinal inflammation in preterm infants. NEC is a gastrointestinal disease or disease condition characterized by inflammation, ischemia, and tissue necrosis. Infants with NEC display impaired epithelial barrier integrity with decreased mucus; decreased intracellular junction integrity (such as tight junction); increased intestinal permeability; reduced peristaltic movement; and impaired epithelial cell regeneration; decreased immunoglobulin A, and/or altered microbiota, leading to inflamed intestine (inflamed wall with gas bubbles). In infants with NEC, opportunistic pathogens can displace healthy microbiota and dominate the gut of preterm infants. Additionally, preterm birth can negatively impact gut microbiota development. Multiple opportunistic pathogens in the hospital setting can colonize the preterm infant gut, including E. coli, K. pneumoniae, E. clocae, Salmonella, and E. faecalis. Colonization by these bacteria can contribute to inflammation, infection, antibiotic resistance, and sepsis in preterm infants with no protective gut microbiota. These preterm infant colonizing pathogens can also contain antimicrobial resistance genes, rendering treatments with antibiotics ineffective. Additionally, NEC can be characterized by a breakdown of epithelial barrier integrity in the gastrointestinal tract.

NEC can be driven by a pathogenic microbiome. Opportunistic bacterial pathogens can colonize the intestinal tract in preterm infants. In these infants, beneficial bacteria found in the full-term infant gut are absent. Pathogens can drive the activation of immune response (such as innate immune responses including those regulated by TLR4), resulting in secretion of inflammatory cytokines and drives Th17 polarization. Uncontrolled intestinal inflammation can increase intestinal epithelial permeability, leading to a cycle of inflammation, infection, and necrosis.

Currently available therapeutics can lack efficiency, efficacy, and/or applicability to wide population of patients. Currently available therapeutics can be ineffective for not sufficient to inhibit growth or biofilm by the pathogen, thereby unable to prevent engraftment of the pathogens. Currently available therapeutics can be inefficient for not facilitating the engraftment of the vagina/infant gastrointestinal tract by healthy microbiota (which can prevent the development or progression of BV, as described herein). For example, currently available therapeutics (such as when comprising a microbe for facilitating the engraftment of the microbe to the vagina or gastrointestinal (GI) tract) may not sufficiently adhere to the vaginal or intestinal epithelial cells. Currently available therapeutics may not sufficiently increase barrier integrity. Additionally, these currently available therapeutics may not sufficiently utilize vaginal or infant gastrointestinally relevant carbohydrates (for example, as a nutrient source), rendering them insufficient to proliferate within these organs/tissues and unable to reduce the engraftment by the pathogen microbes. A nutrient source, as used herein, refers to a substance that is metabolized by a microbe or microorganism. In addition, current available therapeutics may not be sufficient to reduce inflammation that contributes to the diseases as described herein, thereby unable to reduce or improve the symptoms associated with the diseases. While antibiotics can be effective in inhibiting or eliminating pathogens in some cases of BV/NEC, it also inhibits and eliminates healthy microbiota, which in turn can prevent the engraftment of the vagina/infant gastrointestinal tract by healthy microbiota and increase the risk of the tissue being engrafted by pathogens or losing the healthy microbiota. In some cases, the uses of antibiotics can also increase the risk of recurrent BV, since antibiotics can also inhibit the growth of all microbes, including those within the normal microbiota of a normal subject. In other cases, the uses of antibiotics lack effectiveness, due to the antimicrobial resistance acquired by some of the pathogens (such as in NEC). The heterogeneity (such as genetic, epigenetic, environmental, and/microbiome differences) can present difficulty to design therapeutics that can treat or prevent BV (such as recurrent BV) or NEC. Currently available therapeutics are also incapable of preventing the disease, for the same reasons as described herein.

Provided herein, are compositions for treating diseases or disease conditions associated with microbial dysbiosis and methods of using the compositions for treating the diseases or disease conditions. The compositions can comprise a bacteria or a plurality of bacteria. Also provided herein are methods for identify the bacteria or the plurality of bacteria.

The compositions provided herein have higher effectiveness, efficiencies, and applicability to subjects' population, relative to the currently available therapeutics. In some cases, the compositions provided herein can be sufficient to inhibit growth or biofilm by the pathogen, thereby preventing the engraftment of the pathogens. In some cases, the compositions provided herein can be efficient in facilitating the engraftment of the vagina/infant gastrointestinal tract by healthy microbiota (which can prevent the development or progression of BV, as described herein). In some cases, the compositions provided herein can sufficiently increase barrier integrity that is comprised in the subjects with the disease. For example, the compositions provided herein can sufficiently adhere to the vaginal or intestinal epithelial cells. In some cases, the compositions provided herein can sufficiently utilize vaginal or infant gastrointestinally relevant carbohydrates (for example, as a nutrient source), rendering them sufficient to proliferate within these organs and reduce the engraftment by the pathogen microbes. In some cases, the compositions provided herein can be sufficient to reduce inflammation that contributes to the diseases as described herein, thereby reducing or improving the symptoms associated with the diseases. In some cases, the compositions provided herein may not inhibit the growth of microbiota associated with the healthy vagina or infant gastrointestinal tract, thereby reducing the risk of the subject being suffered from the same disease (such as recurrent BV). In some cases, the compositions provided herein can be based on selection of bacteria (as described herein) that can be sufficient to reduce or prevent the disease described herein in a wide population with heterogenicity. For the same reasons as described herein, the compositions provided herein can be used to prevent the development of the diseases.

### Compositions

Provided herein, are compositions and/or formulations. The compositions and/or formulations can be used for treating disease or disease conditions associated with microbial dysbiosis, such as those described herein. The compositions and/or formulations can comprise a bacterial population. The compositions and/or formulations can further comprise a pharmaceutically-acceptable excipient, nutrients for the bacterial population, and other components for administrating to a subject. The compositions and/or formulations described herein can thus also comprise pharmaceutical compositions and/or formulations.

### Bacterial Populations

The bacterial population provided herein can comprise a bacterial strain or a plurality bacterial strains. The term “strain” or “bacterial strain” as used herein refers to a group of bacterial cells, isolates, progenies thereof, or derivatives thereof comprising at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the genome sequences. For example, two bacterial cells, isolates, progenies thereof, derivatives thereof, or any combinations thereof may be the same strain if they share at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the genome sequences. In some cases, a strain as used herein can also refer to a group of bacterial cells, isolates, progenies thereof, or derivatives thereof comprising at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in the 16S rRNA gene sequences. For example, two bacterial cells, isolates, progenies thereof, derivatives thereof, or any combinations thereof may be the same strain if they share at least 50%, at least 60%, at least 70%, at least 80%, at least 90%, at least 91%, at least 92%, at least 93%, at least 94%, at least 95%, at least 96%, at least 97%, at least 98%, at least 99% or more sequence identity in their 16S rRNA or rDNA sequences. The bacterial population can be used for treating a disease or disease condition as described herein. A bacteria or bacterial strain of the bacterial population can comprise a sufficient ability in a disease or disease condition associated function as described herein (i.e., the bacteria or bacterial strain is sufficiently capable of carrying out a particular disease or disease condition associated function). For example, the disease or disease condition associated function may comprise any of those described in EXAMPLEs 2-3, 5, 7-8, and 10-11. In some cases, the bacterial population may comprise a plurality of bacterial strains or at least two bacterial strains. In some case, the plurality of bacterial strains or at least two bacterial strains of the bacterial population can have a collective effect in the disease or disease condition associated function as described herein. In some cases, the plurality of bacterial strains or at least two bacterial strains of the bacterial population may comprise at least one donor bacterial strain and at least one recipient bacterial strain.

### Microbial Taxonomy

The bacterial strain described herein can comprise a bacterial strain of Firmicutes or Actinomycetota. The bacterial strain described herein can comprise a bacterial strain of Firmicutes. The bacterial strain described herein can comprise a bacterial strain of Actinomycetota. The bacterial strain described herein can comprise a bacterial strain of Bacilli or Actinomycetia. The bacterial strain described herein can comprise a bacterial strain of Bacilli. The bacterial strain described herein can comprise a bacterial strain of Actinomycetia. The bacterial strain described herein can comprise a bacterial strain of Lactobacillales or Bifidobacteriales. The bacterial strain described herein can comprise a bacterial strain of Lactobacillales. The bacterial strain described herein can comprise a bacterial strain of Bifidobacteriales. The bacterial strain described herein can comprise a bacterial strain of Lactobacillaceae or Bifidobacteriaceae. The bacterial strain described herein can comprise a bacterial strain of Lactobacillaceae. The bacterial strain described herein can comprise a bacterial strain of Bifidobacteriaceae. The bacterial strain described herein can comprise a bacterial strain of Lactobacillus sp. (or Vertebrate-Associated Lactobacillaceae) or Bifidobacterium sp. The bacterial strain described herein can comprise a bacterial strain of Lactobacillus sp. (or Vertebrate-Associated Lactobacillaceae). The bacterial strain described herein can comprise a bacterial strain of Bifidobacterium sp. In some cases, a bacterial strain described herein can be isolated from a vertebrate (or the bacterial strain is vertebrate-associated). In some cases, a bacterial strain described herein can be isolated from a human subject. In some cases, a bacterial strain described herein can be isolated from a healthy human subject. The healthy human subject can be a female. The healthy human subject can be a male. The healthy human subject can be an infant. For example, the bacterial strain can be vertebrate-associated Bifidobacterium sp. The healthy human subject may not have a disease or disease condition as described herein.

In some instances, a species of Lactobacillus family may comprise a species of the Lactobacillus genus proposed in 1901, which is described in Zheng, J., et. al. Int. J. Syst. Evol. Microbiol. 2020; 70:2782-2858 and is entirely incorporated herein by reference. The Lactobacillus genus may comprise Gram-positive, fermentative, facultatively anaerobic, and/or non-spore forming microorganisms. In some cases, the number of microorganisms that can be classified as Lactobacillus genus may increase, compared to those classified in 1901, with the broad definition of the 1901 classification. Lactobacillus genus may comprise about 261 species that comprise distinctive phenotypic, ecological, and/or genotypic characteristics. The number of species in the genus and/or the level of diversity within the Lactobacillus genus may exceed those of other bacterial genera and/or bacterial families. In this case, Lactobacillus can be reclassified. For example, the average nucleotide identity (ANI), average amino acid identity (AAI), core-gene average amino acid identity (cAAI), core genome phylogeny, signature genes, and metabolic, and/or ecological criteria of the bacterial species in the Lactobacillus genus and its sister taxa in the Lactobacillaceae and Leuconostocacae families are used to reclassify the Lactobacillus genus classified using the definition of 1901 (1901 classification).

In some cases, under the reclassification system, the species of the Lactobacillaceae family may comprise about 26 different genera (Lactobacillus, Paralactobacillus, Pediococcus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus, and Lentilactobacillus), as well as merging the Leuconostocacae family into the Lactobacillaceae family. A comparison of the reclassified Lactobacillus species can be found using the Lactotax database, which can be found in the link: http://Lactobacillus.ualberta.ca/ and is entirely incorporated herein by reference. The classification of Lactobacillus described herein, is also provided in Parks, D H et. al. Nat Biotechnol. 2018 November; 36(10):996-1004; Salvetti, E, et. al. Appl Environ Microbio. 2018 Aug. 17; 84(17). Print 2018 Sep. 1 Erratum in: Appl Environ Microbio. 2018 Oct. 1; 84(20); Markets and Markets: https://www.marketsandmarkets.com/Market-Reports/probiotic-market-advanced-technologies-and-global-market-69.html); Parker, C T, et. al. Int. J. Syst. Evol. Microbiol. 68:1825-1829; Duar, D M, et. al. FEMS Microbiol Rev. 2017 Aug. 1; 41 (Supp_1): S27-S48; or Pane and Vinot 2019: https://www.microbiometimes.com/the-Lactobacillus-taxonomy-change-is-coming-why-and-how-to-make-the-most-of-it/, each of which is entirely incorporated herein by reference.

TABLE 1 below shows the names of various Lactobacillus sp. under the 1901 classification and the reclassification.

TABLE 1
Lactobacillus sp. names before and after reclassification

| Name of Lactobacillus species | Name of Lactobacillus species |
|---|---|
| classified in 1901 | under reclassification |
| Lactobacillus acidipiscis | Ligilactobacillus acidipiscis |
| Lactobacillus acidophilus | Lactobacillus acidophilus |
| Lactobacillus agilis | Ligilactobacillus agilis |
| Lactobacillus aviarius | Ligilactobacillus aviarius |
| Lactobacillus brevis | Levilactobacillus brevis |
| Lactobacillus coleohominis | Limosilactobacillus coleohominis |
| Lactobacillus crispatus | Lactobacillus crispatus |
| Lactobacillus crustorum | Companilactobacillus crustorum |
| Lactobacillus curvatus | Latilactobacillus curvatus |
| Lactobacillus diolivorans | Lentilactobacillus diolivorans |
| Lactobacillus farraginis | Lentilactobacillus farraginis |
| Lactobacillus fermentum | Limosilactobacillus fermentum |
| Lactobacillus fuchuensis | Latilactobacillus fuchuensis |
| Lactobacillus harbinensis | Schleiferilactobacillus harbinensis |
| Lactobacillus helveticus | Lactobacillus helveticus |
| Lactobacillus hilgardii | Lentilactobacillus hilgardii |
| Lactobacillus intestinalis | Lactobacillus intestinalis |
| Lactobacillus jensenii | Lactobacillus jensenii |
| Lactobacillus johnsonii | Lactobacillus johnsonii |
| Lactobacillus kefiranofaciens | Lactobacillus kefiranofaciens |
| Lactobacillus kefiri | Lentilactobacillus kefiri |
| Lactobacillus lindneri | Fructilactobacillus lindneri |
| Lactobacillus mali | Liquorilactobacillus mali |
| Lactobacillus manihotivorans | Lacticaseibacillus manihotivorans |
| Lactobacillus mucosae | Limosilactobacillus mucosae |
| Lactobacillus oeni | Liquorilactobacillus oeni |
| Lactobacillus oligofermentans | Paucilactobacillus oligofermentans |
| Lactobacillus panis | Limosilactobacillus panis |
| Lactobacillus pantheris | Lacticaseibacillus pantheris |
| Lactobacillus parabrevis | Levilactobacillus parabrevis |
| Lactobacillus paracollinoides | Secundilactobacillus paracollinoides |
| Lactobacillus parakefiri | Lentilactobacillus parakefiri |
| Lactobacillus paraplantarum | Lactiplantibacillus paraplantarum |
| Lactobacillus pentosus | Lactiplantibacillus pentosus |
| Lactobacillus pontis | Limosilactobacillus pontis |
| Lactobacillus reuteri | Limosilactobacillus reuteri |
| Lactobacillus rhamnosus | Lacticaseibacillus rhamnosus |
| Lactobacillus rossiae | Furfurilactobacillus rossiae |
| Lactobacillus salivarius | Ligilactobacillus salivarius |
| Lactobacillus siliginis | Furfurilactobacillus siliginis |
| Lactobacillus sucicola | Liquorilactobacillus sucicola |
| Lactobacillus vaccinostercus | Paucilactobacillus vaccinostercus |
| Lactobacillus vaginalis | Limosilactobacillus vaginalis |
| Lactobacillus vini | Liquorilactobacillus vini |
| Lactobacillus zeae | Lacticaseibacillus zeae |

The naming used in this application can be determined using the 1901 classification or the reclassification as described herein, interchangeably. As used herein, Vertebrate-Associated Lactobacillaceae refers to bacterial genera in the Lactobacillaceae family that are associated with vertebrates, which includes Lactobacillus, Limosilactobacillus, Ligilactobacillus, and Lacticaseibacillus. In some instances, the bacterial population may comprise at least one strain of Vertebrate-Associated Lactobacillaceae, or at least one strain of Bifidobacterium sp. In some instances, a bacterial population may comprise at least one strain of Bifidobacterium sp. In some instances, a bacterial population may comprise at least one strain of Vertebrate-Associated Lactobacillaceae.

The bacterial strain described herein can comprise a bacterial strain of Bifidobacterium adolescentis, Bifidobacterium bifidum, Bifidobacterium breve, Bifidobacterium longum, Bifidobacterium pseudocatenulatum, Lactobacillus crispatus, Lactobacillus gasseri, Lactobacillus jensenii, Lactobacillus plantarum, or Lactobacillus rhamnosus. The bacterial strain described herein for treating or preventing a vaginal disease or a complication associated with a vaginal disease can comprise a bacterial strain of Lactobacillus crispatus, Lactobacillus gasseri, Lactobacillus jensenii. The bacterial strain described herein for treating or preventing a vaginal disease or a complication associated with a vaginal disease can comprise a bacterial strain of Lactobacillus crispatus. The bacterial strain described herein for treating or preventing a vaginal disease or a complication associated with a vaginal disease can comprise a bacterial strain of Lactobacillus gasseri. The bacterial strain described herein for treating or preventing a vaginal disease or a complication associated with a vaginal disease can comprise a bacterial strain of Lactobacillus jensenii. The bacterial strain described herein for treating or preventing infant gastrointestinal disease can comprise Bifidobacterium adolescentis, Bifidobacterium bifidum, Bifidobacterium breve, Bifidobacterium longum, Bifidobacterium pseudocatenulatum, Lactobacillus plantarum, or Lactobacillus rhamnosus. The bacterial strain described herein for treating or preventing infant gastrointestinal disease can comprise Bifidobacterium adolescentis. The bacterial strain described herein for treating or preventing infant gastrointestinal disease can comprise Bifidobacterium bifidum. The bacterial strain described herein for treating or preventing infant gastrointestinal disease can comprise Bifidobacterium breve. The bacterial strain described herein for treating or preventing infant gastrointestinal disease can comprise Bifidobacterium longum. The bacterial strain described herein for treating or preventing infant gastrointestinal disease can comprise Bifidobacterium pseudocatenulatum. The bacterial strain described herein for treating or preventing infant gastrointestinal disease can comprise Lactobacillus plantarum. The bacterial strain described herein for treating or preventing infant gastrointestinal disease can comprise Lactobacillus rhamnosus.

The bacterial strain described herein may comprise Lactobacillus crispatus ST100 (or “ST100”), Bifidobacterium bifidum ST31 (or “ST31”), Bifidobacterium bifidum ST80 (or “ST80”), Lactobacillus crispatus ST20 (or “ST20”), Lactobacillus crispatus ST112 (or “ST112”), Lactobacillus gasseri ST105 (or “ST105”), Lactobacillus jensenii ST21 (or “ST21”), Lactobacillus plantarum ST65 (or “ST65”), Bifidobacterium adolescentis ST101 (or “ST101”), Bifidobacterium breve ST56 (or “ST56”), Bifidobacterium longum ST19 (or “ST19”), Bifidobacterium longum ST81 (or “ST81”), Bifidobacterium pseudocatenulatum ST37 (or “ST37”), Bifidobacterium pseudocatenulatum ST66 (or “ST66”), Lactobacillus rhamnosus ST116 (or “ST116”), Bifidobacterium breve ST71 (or “ST71”), Bifidobacterium longum ST23 (or “ST23”), Bifidobacterium longum ST119 (or “ST119”). The bacterial strain described herein may comprises ST100. The bacterial strain described herein may comprises ST31. The bacterial strain described herein may comprises ST80. The bacterial strain described herein may comprises ST20. The bacterial strain described herein may comprises ST112. The bacterial strain described herein may comprises ST105. The bacterial strain described herein may comprises ST21. The bacterial strain described herein may comprises ST65. The bacterial strain described herein may comprises ST101. The bacterial strain described herein may comprises ST56. The bacterial strain described herein may comprises ST19. The bacterial strain described herein may comprises ST81. The bacterial strain described herein may comprises ST37. The bacterial strain described herein may comprises ST66. The bacterial strain described herein may comprises ST116. The bacterial strain described herein may comprises ST71. The bacterial strain described herein may comprises ST23. The bacterial strain described herein may comprises ST119.

The bacterial strain of Lactobacillus crispatus described herein may comprise ST100, ST112, or ST20. The bacterial strain of Lactobacillus crispatus described herein may comprise ST100. The bacterial strain of Lactobacillus crispatus described herein may comprise ST20. The bacterial strain of Lactobacillus crispatus described herein may comprise ST112. The bacterial strain of Lactobacillus gasseri described herein may comprise ST105. The bacterial strain of Lactobacillus jensenii described herein may comprise ST21. The bacterial strain of Lactobacillus plantarum described herein may comprise ST65. The bacterial strain of Lactobacillus rhamnosus described herein may comprise ST116. The bacterial strain of Bifidobacterium adolescentis described herein may comprise ST101. The bacterial strain of Bifidobacterium bifidum described herein may comprise ST31 or ST80. The bacterial strain of Bifidobacterium bifidum described herein may comprise ST31. The bacterial strain of Bifidobacterium bifidum described herein may comprise ST80. The bacterial strain of Bifidobacterium breve described herein may comprise ST56 or ST71. The bacterial strain of Bifidobacterium breve described herein may comprise ST56. The bacterial strain of Bifidobacterium breve described herein may comprise ST71. The bacterial strain of Bifidobacterium pseudocatenulatum described herein may comprise ST37 or ST66. The bacterial strain of Bifidobacterium pseudocatenulatum described herein may comprise ST37. The bacterial strain of Bifidobacterium pseudocatenulatum described herein may comprise ST66. The bacterial strain of Bifidobacterium longum described herein may comprise ST19, ST81, ST23, or ST119. The bacterial strain of Bifidobacterium longum described herein may comprise ST19. The bacterial strain of Bifidobacterium longum described herein may comprise ST81. The bacterial strain of Bifidobacterium longum described herein may comprise ST23. The bacterial strain of Bifidobacterium longum described herein may comprise ST119.

In some instances, a bacterial population may comprise one or more Bifidobacterium sp. The one or more Bifidobacterium sp. may include Bifidobacterium adolescentis, Bifidobacterium aerophilum, Bifidobacterium angulatum, Bifidobacterium animalis, Bifidobacterium asteroids, Bifidobacterium bifidum, Bifidobacterium boum, Bifidobacterium breve, Bifidobacterium catenulatum, Bifidobacterium choerinum, Bifidobacterium coryneforme, Bifidobacterium cuniculi, Bifidobacterium dentium, Bifidobacterium faecale, Bifidobacterium gallicum, Bifidobacterium globosum, Bifidobacterium indicum, Bifidobacterium infantis, Bifidobacterium longum, Bifidobacterium magnum, Bifidobacterium minimum, Bifidobacterium pseudocatenulatum, Bifidobacterium seudolongum, Bifidobacterium pullorum, Bifidobacterium stercoris. Bifidobacterium subtile, Bifidobacterium suis, or Bifidobacterium thermophilum, or a combination thereof. In some instances, a bacterial population may comprise one or more Lactobacillus sp. The one or more Lactobacillus sp. may include Lactobacillus johnsonii, Lactocaseibacillus rhamnosus, Lactocaseibacillus zeae, Ligilactobacillus acidipiscis, Lactobacillus acidophilus, Ligilactobacillus agilis, Ligilactobacillus aviarius, Levilactobacillus brevis, Limosilactobacillus coleohominis, Lactobacillus crispatus, Companilactobacillus crustorum, Latilactobacillus curvatus, Lentilactobacillus diolivorans, Lentilactobacillus farraginis, Limosilactobacillus fermentum, Latilactobacillus fuchuensis, Schleiferilactobacillus harbinensis, Lactobacillus helveticus, Lentilactobacillus hilgardii, Lactobacillus intestinalis, Lactobacillus jensenii, Lactobacillus kefiranofaciens, Lentilactobacillus kefiri, Fructilactobacillus lindneri, Liquorilactobacillus mali, Lactocaseibacillus manihotivorans, Limosilactobacillus mucosae, Liquorilactobacillus oeni, Paucilactobacillus oligofermentans, Limosilactobacillus panis, Lactocaseibacillus pantheris, Levilactobacillus parabrevis, Secundilactobacillus paracollinoides, Lentilactobacillus parakefiri, Lactoplantibacillus paraplantarum, Lactoplantibacillus pentosus, Limosiactobacillus pontis, Limosilactobacillus reuteri, Furfurilactobacillus rossiae, Ligilactobacillus salivarius, Furfurilactobacillus siliginis, Liquorilactobacillus sucicola, Paucilactobacillus vaccinostercus, Limosilactobacillus vaginalis, Liquorilactobacillus vini, Laclococcus garvieae, or Lactococcus lactis, or a combination thereof.

In some cases, the bacterial population may comprise at least one strain of Akkermansia sp., at least one strain of Blautia sp., at least one strain of Clostridium sp., at least one strain of Coprococcus sp., at least one strain of Dorea sp., at least one strain of Faecalibacterium sp., at least one strain of Roseburia sp., at least one strain of Ruminococcus sp., at least one strain of Anaerbutyricum sp., at least one strain of Anaerostipes sp., at least one strain of Anaerotignum sp., at least one strain of Bacillus sp., at least one strain of Bacteroides sp., at least one strain of Clostridium sp., at least one strain of Collinsella sp., at least one strain of Enterococcus sp., at least one strain of Erysipelatoclostridium sp., at least one strain of Escherichia sp., at least one strain of Eubacterium sp., at least one strain of Faecalicatena sp., at least one strain of Holdemanella sp., at least one strain of Lachnospira sp., at least one strain of Longibaculum sp., at least one strain of Paraprevotella sp., at least one strain of Parabacteroides sp., at least one strain of Pediococcus sp., or at least one strain of Veillonella sp. In some instances, a bacterial population may comprise at least one strain of Akkermansia sp. In some instances, a bacterial population may comprise at least one strain of Blautia sp. In some instances, a bacterial population may comprise at least one strain of Clostridium sp. In some instances, a bacterial population may comprise at least one strain of Coprococcus sp. In some instances, a bacterial population may comprise at least one strain of Dorea sp. In some instances, a bacterial population may comprise at least one strain of Faecalibacterium sp. In some instances, a bacterial population may comprise at least one strain of Roseburia sp. In some instances, a bacterial population may comprise at least one strain of Ruminococcus sp. In some instances, a bacterial population may comprise at least one strain of Anaerbutyricum sp. In some instances, a bacterial population may comprise at least one strain of Anaerostipes sp. In some instances, a bacterial population may comprise at least one strain of Anaerotignum sp. In some instances, a bacterial population may comprise at least one strain of Bacillus sp. In some instances, a bacterial population may comprise at least one strain of Bacteroides sp. In some instances, a bacterial population may comprise at least one strain of Clostridium sp. In some instances, a bacterial population may comprise at least one strain of Collinsella sp. In some instances, a bacterial population may comprise at least one strain of Enterococcus sp. In some instances, a bacterial population may comprise at least one strain of Erysipelatoclostridium sp. In some instances, a bacterial population may comprise at least one strain of Escherichia sp. In some instances, a bacterial population may comprise at least one strain of Eubacterium sp. In some instances, a bacterial population may comprise at least one strain of Faecalicatena sp. In some instances, a bacterial population may comprise at least one strain of Holdemanella sp. In some instances, a bacterial population may comprise at least one strain of Lachnospira sp. In some instances, a bacterial population may comprise at least one strain of Longibaculum sp. In some instances, a bacterial population may comprise at least one strain of Paraprevotella sp. In some instances, a bacterial population may comprise at least one strain of Parabacteroides sp. In some instances, a bacterial population may comprise at least one strain of Pediococcus sp. In some instances, a bacterial population may comprise at least one strain of Veillonella sp.

In some instances, a bacterial population may comprise at least two strains of Vertebrate-Associated Lactobacillaceae, and/or at least two strains of Bifidobacterium sp., and/or at least two strains of Akkermansia sp., and/or at least two strains of Blautia sp. and/or at least two strains of Clostridium sp., and/or at least two strains of Coprococcus sp., and/or at least two strains of Dorea sp. and/or at least two strains of Faecalibacterium sp., and/or at least two strains of Roseburia sp., and/or at least two strains of Ruminococcus sp., and/or at least two strains of Anaerbutyricum sp., and/or at least two strains of Anaerostipes sp., and/or at least two strains of Anaerotignum sp., and/or at least two strains of Bacillus sp., and/or at least two strains of Bacteroides sp., and/or at least two strains of Clostridium sp., and/or at least two strains of Collinsella sp., and/or at least two strains of Enterococcus sp., and/or at least two strains of Erysipelatoclostridium sp., and/or at least two strains of Escherichia sp., and/or at least two strains of Eubacterium sp., and/or at least two strains of Faecalicatena sp., and/or at least two strains of Holdemanella sp., and/or at least two strains of Lachnospira sp., and/or at least two strains of Longibaculum sp., and/or at least two strains of Paraprevotella sp., and/or at least two strains of Parabacteroides sp., and/or at least two strains of Pediococcus sp., and/or at least two strains of Veillonella sp. In some cases, a bacterial population may comprise at least three strains of Vertebrate-Associated Lactobacillaceae, and/or at least three strains of Bifidobacterium sp., and/or at least three strains of Akkermansia sp., and/or at least three strains of Blautia sp. and/or at least three strains of Clostridium sp., and/or at least three strains of Coprococcus sp., and/or at least three strains of Dorea sp. and/or at least three strains of Faecalibacterium sp., and/or at least three strains of Roseburia sp., and/or at least three strains of Ruminococcus sp., and/or at least three strains of Anaerbutyricum sp., and/or at least three strains of Anaerostipes sp., and/or at least three strains of Anaerotignum sp., and/or at least three strains of Bacillus sp., and/or at least three strains of Bacteroides sp., and/or at least three strains of Clostridium sp., and/or at least three strains of Collinsella sp., and/or at least three strains of Enterococcus sp., and/or at least three strains of Erysipelatoclostridium sp., and/or at least three strains of Escherichia sp., and/or at least three strains of Eubacterium sp., and/or at least three strains of Faecalicatena sp., and/or at least three strains of Holdemanella sp., and/or at least three strains of Lachnospira sp., and/or at least three strains of Longibaculum sp., and/or at least three strains of Paraprevotella sp., and/or at least three strains of Parabacteroides sp., and/or at least three strains of Pediococcus sp., and/or at least three strains of Veillonella sp. In some cases, a bacterial population may comprise at least more than three strains of Vertebrate-Associated Lactobacillaceae, or at least more than three strains of Bifidobacterium sp., and/or at least more than three strains of Akkermansia sp., and/or at least more than three strains of Blautia sp. and/or at least more than three strains of Clostridium sp., and/or at least more than three strains of Coprococcus sp., and/or at least more than three strains of Dorea sp. and/or at least more than three strains of Faecalibacterium sp., and/or at least more than three strains of Roseburia sp., and/or at least more than three strains of Ruminococcus sp., and/or at least more than three strains of Anaerbutyricum sp., and/or at least more than three strains of Anaerostipes sp., and/or at least more than three strains of Anaerotignum sp., and/or at least more than three strains of Bacillus sp., and/or at least more than three strains of Bacteroides sp., and/or at least more than three strains of Clostridium sp., and/or at least more than three strains of Collinsella sp., and/or at least more than three strains of Enterococcus sp., and/or at least more than three strains of Erysipelatoclostridium sp., and/or at least more than three strains of Escherichia sp., and/or at least more than three strains of Eubacterium sp., and/or at least more than three strains of Faecalicatena sp., and/or at least more than three strains of Holdemanella sp., and/or at least more than three strains of Lachnospira sp., and/or at least more than three strains of Longibaculum sp., and/or at least more than three strains of Paraprevotella sp., and/or at least more than three strains of Parabacteroides sp., and/or at least more than three strains of Pediococcus sp., and/or at least more than three strains of Veillonella sp.

In some instances, a pharmaceutical composition that can comprise a bacterial population. Such bacterial population can comprise one or more different bacterial species and/or strains. Such bacterial species and/or strains can belong to one or more different bacterial phyla.

In some instances, a bacterial population may comprise one or more Akkermansia sp. The one or more Akkermansia sp. may include Akkermansia glycaniphila, or Akkermansia muciniphila, or a combination thereof.

In some instances, a bacterial population may comprise one or more Blautia sp. The one or more Blautia sp. may include Blautia acetigignens, Blautia ammoniilytica, Blautia argi, Blautia caecimuris, Blautia coccoides, Blautia faecicola, Blautia faecis, Blautia glucerasea, Blautia hansenii, Blautia honinis, Blautia hydrogenotrophica, Blautia intestinalis, Blautia liquoris, Blautia luti, Blautia obeum, Blautia producta, Blautia schinkii, Blautia stercoris, or Blautia wexlerae or a combination thereof.

In some instances, a bacterial population may comprise one or more Coprococcus sp. The one or more Coprococcus sp. may include Coprococcus ammoniilyticus, Coprococcus catus, Coprococcus comes, or Coprococcus eutactus or a combination thereof.

In some instances, a bacterial population may comprise one or more Dorea sp. The one or more Dorea sp. may include Dorea acetigenes, Dorea ammoniilytica, Dorea formicigenerans, or Dorea longicatena, or a combination thereof

In some instances, a bacterial population may comprise one or more Faecalibacterium sp. The one or more Faecalibacterium sp. may include Faecalibacterium butyricigenerans, Faecalibacterium duncaniae, Faecalibacterium gallinarum, Faecalibacterium hattorii, Faecalibacterium longum, or Faecalibacterium prausnitzii, or a combination thereof.

In some instances, a bacterial population may comprise one or more Roseburia sp. The one or more Roseburia sp. may include Roseburia cecicola, Roseburia faecis, Roseburia hominis, Roseburia intestinalis, or Roseburia inulinivorans or a combination thereof.

In some instances, a bacterial population may comprise one or more Ruminococcus sp. The one or more Ruminococcus sp. may include Ruminococcus albus, Ruminococcus bovis, Ruminococcus bromii, Ruminococcus callidus, Ruminococcus champanellensis, Ruminococcus faecis, Ruminococcus flavefaciens, Ruminococcus gauvreauii, Ruminococcus gnavus, Ruminococcus hansenii, Ruminococcus hydrogenotrophicus, Ruminococcus lactaris, Ruminococcus luti, Ruminococcus obeum, Ruminococcus palustris, Ruminococcus pasteurii, Ruminococcus productus, Ruminococcus schinkii, or Ruminococcus torques, or a combination thereof.

In some instances, a bacterial population may comprise one or more Anaerbutyricum sp. The one or more Anaerbutyricum sp. may include Anaerbutyricum hallii, Anaerbutyricum soehngenii, or a combination thereof.

In some instances, a bacterial population may comprise one or more Anaerostipes sp. The one or more Anaerostipes sp. may include Anaerostipes amylophilus, Anaerostipes butyraticus, Anaerostipes caccae, Anaerostipes faecalis, Anaerostipes hadrus, Anaerostipes hominis, or Anaerostipes rhamnosivorans, or a combination thereof.

In some instances, a bacterial population may comprise one or more Anaerotignum sp. The one or more Anaerotignum sp. may include Anaerotignum aminivorans, Anaerotignum faecicola, Anaerotignum lactatifermentans, Anaerotignum neopropionicum, or Anaerotignum propionicum, or a combination thereof.

In some instances, a bacterial population may comprise one or more Bacteroides sp. The one or more Bacteroides sp. may include Bacteroides acidifaciens, Bacteroides caccae, Bacteroides caecicola, Bacteroides caecigallinarum, Bacteroides caecimuris, Bacteroides cellulolyticus, Bacteroides cellulosilyticus, Bacteroides clarus, Bacteroides corporis, Bacteroides eggerthii, Bacteroides facilis, Bacteroides faecalis, Bacteroides faecichinchillae, Bacteroides faecis, Bacteroides finegoldii, Bacteroides fluxus, Bacteroides fragilis, Bacteroides galacturonicus, Bacteroides gallinaceum, Bacteroides gallinarum, Bacteroides graminisolvens, Bacteroides helcogenes, Bacteroides hominis, Bacteroides intestinalis, Bacteroides koreensis, Bacteroides kribbi, Bacteroides luhongzhouii, Bacteroides luti, Bacteroides nordii, Bacteroides oleiciplenus, Bacteroides ovatus, Bacteroides parvus, Bacteroides pectinophilus, Bacteroides polypragmatus, Bacteroides propionicifaciens, Bacteroides proionicigenes, Bacteroides pyogenes, Bacteroides reticulotermitis, Bacteroides rodentium, Bacteroides salyersiae, Bacteroides stercorirosoris, Bacteroides stercoris, Bacteroides thetaiotaomicron, Bacteroides uniformis, Bacteroides xylanisolvens, or Bacteroides zhangwengongii, or a combination thereof.

In some instances, a bacterial population may comprise one or more Clostridium sp. The one or more Clostridium sp. may include Clostridium aerotolerans, Clostridium aminophilum, Clostridium coccoides, Clostridium nexile, Clostridium polysaccharolyticum, Clostridium symbiosum, Clostridium sphenoides, Clostridium xylanolyticum, Clostridium leptum, Clostridium cellulosi, Clostridium sordelli, or Clostridium scindens, or a combination thereof.

In some instances, a bacterial population may comprise one or more Collinsella sp. The one or more Collinsella sp. may include Collinsella aerofaciens, Collinsella intestinalis, Collinsella massiliensis, Collinsella stercoris, Collinsella tanakaei, or Collinsella vaginalis, or a combination thereof.

In some instances, a bacterial population may comprise one or more Enterococcus sp. The one or more Enterococcus sp. may include Enterococcus alcedinis, Enterococcus alishanensis, Enterococcus aquimarinus, Enterococcus asini, Enterococcus avium, Enterococcus bulliens, Enterococcus caccae, Enterococcus camelliae, Enterococcus canintestini, Enterococcus canis, Enterococcus casseliflavus, Enterococcus cecorum, Enterococcus columbae, Enterococcus crotali, Enterococcus devriesei, Enterococcus diestrammenae, Enterococcus dispar, Enterococcus dongliensis, Enterococcus durans, Enterococcus eurekensis, Enterococcus faecalis, Enterococcus faeciu, Enterococcus florum, Enterococcus gallinarum, Enterococcus gilvus, Enterococcus haemoperoxidus, Enterococcus hermanniensis, Enterococcus hirae, Enterococcus hulanensis, Enterococcus innesii, Enterococcus italicus, Enterococcus lactis, Enterococcus larvae, Enterococcus lemanii, Enterococcus malodoratus, Enterococcus moraviensis, Enterococcus mundtii, Enterococcus nangangensis, Enterococcus olivae, Enterococcus pallens, Enterococcus phoeniculicola, Enterococcus pingfangensis, Enterococcus plantarum, Enterococcus pseudoavium, Enterococcus quebecensis, Enterococcus raffinosus, Enterococcus ratti, Enterococcus rivorum, Enterococcus rotai, Enterococcus saccharolyticus, Enterococcus saigonensis, Enterococcus silesiacus, Enterococcus songbeiensis, Enterococcus sulfureus, Enterococcus termitis, Enterococcus thilandicus, Enterococcus ureasiticus, Enterococcus ureilyticus, Enterococcus viikkiensis, Enterococcus villorum, Enterococcus wangshanyuanii, or Enterococcus xiangfangensis, or a combination thereof.

In some instances, a bacterial population may comprise one or more Escherichia sp. The one or more Escherichia sp. may include Escherichia albertii, Escherichia coli, Escherichia fergusonii, Escherichia hermanni, Escherichia marmotae, or Escherichia ruysiae, or a combination thereof.

In some instances, a bacterial population may comprise one or more Eubacterium sp. The one or more Eubacterium sp. may Eubacterium aggregans, Eubacterium barkeri, Eubacterium brachy, Eubacterium callanderi, Eubacterium cellulosolvens, Eubacterium coprostanoligenes, Eubacterium hominis, Eubacterium infirmum, Eubacterium limosum, Eubacterium maltosivorans, Eubacterium minutum, Eubacterium multiforme, Eubacterium nodatum, Eubacterium oxidoreducens, Eubacterium plexicaudatum, Eubacterium pyruvativorans, Eubacterium ramulus, Eubacterium ruminantium, Eubacterium saphenum, Eubacterium siraeum, Eubacterium tenue, Eubacterium tortuosum, Eubacterium uniforme, Eubacterium ventriosum, Eubacterium xylanophilum, or Eubacterium yurii, or a combination thereof.

In some instances, a bacterial population may comprise one or more Faecalicatena sp. The one or more Faecalicatena sp. may include Faecalicatena absiana, Faecalicatena acetigenes, Faecalicatena contorta, Faecalicatena fissicatena, or Faecalicatena orotica, or a combination thereof.

In some instances, a bacterial population may comprise one or more Holdemanella sp. The one or more Holdemanella sp. may include Holdemanella biformi or Holdemanella proci, or a combination thereof.

In some instances, a bacterial population may comprise one or more Lachnospira sp. The one or more Lachnospira sp. may include Lachnospira eligens, Lachnospira multipara, or Lachnospira pectinoschiza, or a combination thereof.

In some instances, a bacterial population may comprise one or more Longibaculum sp. The one or more Longibaculum sp. may include Longibaculum muris.

In some instances, a bacterial population may comprise one or more Paraprevotella sp. The one or more Paraprevotella sp. may include Paraprevotella clara, or Paraprevotella xylaniphila, or a combination thereof.

In some instances, a bacterial population may comprise one or more Parabacteroides a sp. The one or more Parabacteroides sp. may include Parabacteroides acidifaciens, Parabacteroides chartae, Parabacteroides chinchilla, Parabacteroides chongii, Parabacteroides distasonis, Parabacteroides faecis, Parabacteroides goldsteinii, Parabacteroides gordonii, Parabacteroides hominis, Parabacteroides johnsonii, or Parabacteroides merdae, or a combination thereof.

In some instances, a bacterial population may comprise one or more Pediococcus sp. The one or more Pediococcus sp. may include Pediococcus acidilactici, Pediococcus argentinicus, Pediococcus cellicola, Pediococcus claussenii, Pediococcus damnosus, Pediococcus ethanolidurans, Pediococcus inopinatus, Pediococcus parvulus, Pediococcus pentosaceus, Pediococcus siamensis, or Pediococcus stilesii, or a combination thereof.

In some instances, a bacterial population may comprise one or more Veillonella sp. The one or more Veillonella sp. may include Veillonella atypica, Veillonella caviae, Veillonella cricetid, Veillonella denticariosi, Veillonella dispar, Veillonella hominis, Veillonellan infantium, Veillonella magna, Veillonella montpellierensis, Veillonella nakazawae, Veillonella parvula, Veillonella ratti, Veillonella rodentium, Veillonella rogosae, Veillonella seminalis, or Veillonella tobetseunsis, or a combination thereof.

In some instances, a bacterial strain described herein may be derived from a subject of an organ or tissue thereof. As used herein, when referring to a microbial organism being “derived from,” a subject or an organ/tissue thereof, is equivalent to the microbial organism being obtained originates from the microbiota of the subject of the organ/tissue thereof.

In some instances, a bacterial strain described herein may be derived from a vagina. In some instances, a bacterial strain described herein may be derived from a mammalian vagina. In some instances, a bacterial strain described herein may be derived from a human vagina. In some instances, a bacterial strain described herein may be derived from a human that does not have a disease or disease condition. In some instances, a bacterial strain described herein may be derived from a human that does not have a vaginal disease or disease condition, complication of the vaginal disease or disease condition or a risk thereof. In some instances, a bacterial strain described herein may be derived from a human that does not have BV or a risk thereof. In some instances, a bacterial strain described herein may be derived from a gastrointestinal tract. In some instances, a bacterial strain described herein may be derived from a mammalian gastrointestinal tract. In some instances, a bacterial strain described herein may be derived from a human gastrointestinal tract. In some instances, a bacterial strain described herein may be derived from a human infant gastrointestinal tract. In some instances, a bacterial strain described herein may be derived from a human infant gastrointestinal tract, wherein the infant is a preterm infant. In some instances, a bacterial strain described herein may be derived from a human that does not have a gastrointestinal disease or disease condition or a risk thereof. In some instances, a bacterial strain described herein may be derived from a human that does not have an infant gastrointestinal disease or disease condition or a risk thereof. In some instances, a bacterial strain described herein may be derived from an infant that does not have NEC or a risk thereof. In some instances, a bacterial strain described herein may be derived from a human that does not have a disease or disease condition or a risk thereof. In some instances, a bacterial strain described herein may be derived from a human that is healthy. In some cases, when deriving the bacterial strain as described herein, the bacterial strain can be derived from the microbiota of the subjects, organs, or tissues as described herein. In some instances, a bacterial population may comprise purified bacterial strains. In some cases, In some instances, a bacterial strain described herein may not comprise a recombinant genetic modification. In some cases, a bacterial strain described herein may not be genetically engineered.

### Sequences

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to any one of SEQ ID NOs: 1-30. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.999999% to any one of SEQ ID NOs: 1-30. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to any one of SEQ ID NOs: 1-30. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to any one of SEQ ID NOs: 1-30. The bacterial strain described herein can comprise a sequence having a sequence identity that is 100% to any one of SEQ ID NOs: 1-30.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 1. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 1. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 1. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 1. The bacterial strain described herein can comprise a sequence having a sequence identity that is 100% to SEQ ID NO: 1.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 2. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 2. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 2. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 2. The bacterial strain described herein can comprise a sequence having a sequence identity that is 100% to SEQ ID NO: 2.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 3. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 3. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 3. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 3. The bacterial strain described herein can comprise a sequence having a sequence identity that is 100% to SEQ ID NO: 3.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 4. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 4. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 4. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 4. The bacterial strain described herein can comprise a sequence having a sequence identity that is 100% to SEQ ID NO: 4.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 5. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 5. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 5. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 5. The bacterial strain described herein can comprise a sequence having a sequence identity that is 100% to SEQ ID NO: 5.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 6. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 6. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 6. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 6. The bacterial strain described herein can comprise a sequence having a sequence identity that is 100% to SEQ ID NO: 6.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 7. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 7. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 7. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 7. The bacterial strain described herein can comprise a sequence having a sequence identity that is 100% to SEQ ID NO: 7.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 8. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 8. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 8. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 8. The bacterial strain described herein can comprise a sequence having a sequence identity that is 100% to SEQ ID NO: 8.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 9. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 9. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 9. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 9. The bacterial strain described herein can comprise a sequence having a sequence identity that is 100% to SEQ ID NO: 9.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 10. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 10. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 10. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 10. The bacterial strain described herein can comprise a sequence having a sequence identity that is 100% to SEQ ID NO: 10.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 11. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 11. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 11. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 11. The bacterial strain described herein can comprise a sequence having a sequence identity that is 100% to SEQ ID NO: 11.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 12. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 12. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 12. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 12. The bacterial strain described herein can comprise a sequence having a sequence identity that is 100% to SEQ ID NO: 12.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 13. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 13. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 13. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 13. The bacterial strain described herein can comprise a sequence having a sequence identity that is 100% to SEQ ID NO: 13.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 14. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 14. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 14. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 14. The bacterial strain described herein can comprise a sequence having a sequence identity that is 100% to SEQ ID NO: 14.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 15. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 15. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 15. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 15. The bacterial strain described herein can comprise a sequence having a sequence identity that is 100% to SEQ ID NO: 15.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 16. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 16. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 16. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 16. The bacterial strain described herein can comprise a sequence having a sequence identity that is 100% to SEQ ID NO: 16.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 17. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 17. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 17. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 17. The bacterial strain described herein can comprise a sequence having a sequence identity that is 100% to SEQ ID NO: 17.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 18. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 18. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 18. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 18. The bacterial strain described herein can comprise a sequence having a sequence identity that is 100% to SEQ ID NO: 18.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 20. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 20. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 20. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 20. The bacterial strain described herein can comprise a sequence having a sequence identity that is 100% to SEQ ID NO: 20.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 21. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 21. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 21. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 21. The bacterial strain described herein can comprise a sequence having a sequence identity that is 100% to SEQ ID NO: 21.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 22. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 22. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 22. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 22. The bacterial strain described herein can comprise a sequence having a sequence identity that is 100% to SEQ ID NO: 22.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 23. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 23. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 23. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 23. The bacterial strain described herein can comprise a sequence having a sequence identity that is 100% to SEQ ID NO: 23.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 24. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 24. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 24. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 24. The bacterial strain described herein can comprise a sequence having a sequence identity that is 100% to SEQ ID NO: 24.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 25. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 25. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 25. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 25. The bacterial strain described herein can comprise a sequence having a sequence identity that is 100% to SEQ ID NO: 25.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 26. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 26. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 26. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 26. The bacterial strain described herein can comprise a sequence having a sequence identity that is 100% to SEQ ID NO: 26.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 27. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 27. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 27. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 27. The bacterial strain described herein can comprise a sequence having a sequence identity that is 100% to SEQ ID NO: 27.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 28. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 28. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 28. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 28. The bacterial strain described herein can comprise a sequence having a sequence identity that is 100% to SEQ ID NO: 28.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 29. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 29. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 29. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 29. The bacterial strain described herein can comprise a sequence having a sequence identity that is 100% to SEQ ID NO: 29.

The bacterial strain described herein can comprise a sequence having a sequence identity that is at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.9%, at least about 99.99%, at least about 99.999%, at least about 99.9999%, at least about 99.99999% or more to SEQ ID NO: 30. The bacterial strain described herein can comprise a sequence having a sequence identity that is at most about 80%, at most about 85%, at most about 90%, at most about 91%, at most about 92%, at most about 93%, at most about 94%, at most about 95%, at most about 96%, at most about 97%, at most about 98%, at most about 99%, at most about 99.9%, at most about 99.99%, at most about 99.999%, at most about 99.9999%, or at most about 99.99999% to SEQ ID NO: 30. The bacterial strain described herein can comprise a sequence that has at least about 1, at least about 2, at least about 3, at least about 4, at least about 5, at least about 10, at least about 100, at least about 1000, at least about 10000, at least about 100000, at least about 1000000 or more nucleotide differences relative to SEQ ID NO: 30. The bacterial strain described herein can comprise a sequence that has at most about 1 nucleotide, at most about 2, at most about 3, at most about 4, at most about 5, at most about 10, at most about 100, at most about 1000, at most about 10000, at most about 100000, or at most about 1000000 nucleotide differences relative to SEQ ID NO: 30. The bacterial strain described herein can comprise a sequence having a sequence identity that is 100% to SEQ ID NO: 30.

Lactobacillus crispatus may have a genome that has a sequence that has a sequence identity that is about 95%-100% to any one of SEQ ID NOs: 1-4 and 12-15. Lactobacillus crispatus may have a genome that has a sequence that has a sequence identity that is about 95%-100% to any one of SEQ ID NOs: 1-2. Lactobacillus crispatus may have a genome that has a sequence that has a sequence identity that is about 95%-100% to a combination of SEQ ID NOs: 1-2. Lactobacillus crispatus may have a genome that has a sequence that has a sequence identity that is about 95%-100% to any one of SEQ ID NOs: 3-4. Lactobacillus crispatus may have a genome that has a sequence that has a sequence identity that is about 95%-100% to a combination of SEQ ID NOs: 3-4. Lactobacillus crispatus may have a genome that has a sequence that has a sequence identity that is about 95%-100% to any one of SEQ ID NOs: 12-15. Lactobacillus crispatus may have a genome that has a sequence that has a sequence identity that is about 95%-100% to a combination of SEQ ID NOs: 12-15. Lactobacillus gasseri may have a genome that has a sequence that has a sequence identity that is about 95%-100% to any one of SEQ ID NOs: 5-6. Lactobacillus gasseri may have a genome that has a sequence that has a sequence identity that is about 95%-100% to a combination of SEQ ID NOs: 5-6. Lactobacillus jensenii may have a genome that has a sequence that has a sequence identity that is about 95%-100% to SEQ ID NO: 7. Bifidobacterium bifidum may have a genome that has a sequence that has a sequence identity that is about 95%-100% to any one of SEQ ID NOs: 8-9. Bifidobacterium bifidum may have a genome that has a sequence that has a sequence identity that is about 95%-100% to SEQ ID NO: 8. Bifidobacterium bifidum may have a genome that has a sequence that has a sequence identity that is about 95%-100% to SEQ ID NO: 9. Lactobacillus plantarum may have a genome that has a sequence that has a sequence identity that is about 95%-100% to any one of SEQ ID NOs: 10-11. Lactobacillus plantarum may have a genome that has a sequence that has a sequence identity that is about 95%-100% to a combination of SEQ ID NOs: 10-11. Bifidobacterium adolescentis may have a genome that has a sequence that has a sequence identity that is about 95%-100% to SEQ ID NO: 16. Bifidobacterium breve may have a genome that has a sequence that has a sequence identity that is about 95%-100% to any one of SEQ ID NOs: 17 and 30. Bifidobacterium breve may have a genome that has a sequence that has a sequence identity that is about 95%-100% to SEQ ID NO: 17. Bifidobacterium breve may have a genome that has a sequence that has a sequence identity that is about 95%-100% to SEQ ID NO: 30. Bifidobacterium longum may have a genome that has a sequence that has a sequence identity that is about 95%-100% to any one of SEQ ID NOs: 18-26. Bifidobacterium longum may have a genome that has a sequence that has a sequence identity that is about 95%-100% to any one of SEQ ID NOs: 18-19. Bifidobacterium longum may have a genome that has a sequence that has a sequence identity that is about 95%-100% to a combination of SEQ ID NOs: 18-19. Bifidobacterium longum may have a genome that has a sequence that has a sequence identity that is about 95%-100% to SEQ ID NO: 20. Bifidobacterium longum may have a genome that has a sequence that has a sequence identity that is about 95%-100% to any one of SEQ ID NOs: 21-24. Bifidobacterium longum may have a genome that has a sequence that has a sequence identity that is about 95%-100% to a combination of SEQ ID NOs: 21-24. Bifidobacterium longum may have a genome that has a sequence that has a sequence identity that is about 95%-100% to any one of SEQ ID NOs: 25-26. Bifidobacterium longum may have a genome that has a sequence that has a sequence identity that is about 95%-100% to a combination of SEQ ID NOs: 25-26. Bifidobacterium pseudocatenulatum may have a genome that has a sequence that has a sequence identity that is about 95%-100% to any one of SEQ ID NOs: 27-28. Bifidobacterium pseudocatenulatum may have a genome that has a sequence that has a sequence identity that is about 95%-100% to SEQ ID NO: 27. Bifidobacterium pseudocatenulatum may have a genome that has a sequence that has a sequence identity that is about 95%-100% to SEQ ID NO: 28. Lactobacillus rhamnosus may have a genome that has a sequence that has a sequence identity that is about 95%-100% to SEQ ID NO: 29.

Bifidobacterium bifidum ST31 (or “ST31”) may have a genome sequence that has a sequence identity that is about 100% to SEQ ID NO: 8. Bifidobacterium bifidum ST80 (or “ST80”) may have a genome sequence that has a sequence identity that is about 100% to SEQ ID NO: 9. Lactobacillus crispatus ST100 (or “ST100”) may have a genome sequence that has a sequence identity that is about 100% to a combination of SEQ ID NOs: 12-15. Lactobacillus crispatus ST20 (or “ST20”) may have a genome sequence that has a sequence identity that is about 100% to a combination of SEQ ID NOs: 1-2. Lactobacillus crispatus ST112 (or “ST112”) may have a genome sequence that has a sequence identity that is about 100% to a combination of SEQ ID NO: 3-4. Lactobacillus gasseri ST105 (or “ST105”) may have a genome sequence that has a sequence identity that is about 100% to a combination of SEQ ID NO: 5-6. Lactobacillus jensenii ST21 (or “ST21”) may have a genome sequence that has a sequence identity that is about 100% to SEQ ID NO: 7. Lactobacillus plantarum ST65 (or “ST65”) may have a genome sequence that has a sequence identity that is about 100% to a combination of SEQ ID NOs: 10-11. Bifidobacterium adolescentis ST101 (or “ST101”) may have a genome sequence that has a sequence identity that is about 100% to SEQ ID NO: 16. Bifidobacterium breve ST56 (or “ST56”) may have a genome sequence that has a sequence identity that is about 100% to SEQ ID NO: 17. Bifidobacterium longum ST19 (or “ST19”) may have a genome sequence that has a sequence identity that is about 100% to a combination of SEQ ID NOs: 18-19. Bifidobacterium longum ST81 (or “ST81”) may have a genome sequence that has a sequence identity that is about 100% to a combination of SEQ ID NOs: 21-24. Bifidobacterium pseudocatenulatum ST37 (or “ST37”) may have a genome sequence that has a sequence identity that is about 100% to SEQ ID NO: 27. Bifidobacterium pseudocatenulatum ST66 (or “ST66”) may have a genome sequence that has a sequence identity that is about 100% to SEQ ID NO: 28. Lactobacillus rhamnosus ST116 (or “ST116”) may have a genome sequence that has a sequence identity that is about 100% to SEQ ID NO: 29. Bifidobacterium longum ST23 (or “ST23”) may have a genome sequence that has a sequence identity that is about 100% to SEQ ID NO: 20. Bifidobacterium longum ST119 (or “ST119”) may have a genome sequence that has a sequence identity that is about 100% to a combination of SEQ ID NOs: 25-26. Bifidobacterium breve ST71 (or “ST71”) may have a genome sequence that has a sequence identity that is about 100% to SEQ ID NO: 30.

### Sufficient Abilities

In some instances, the bacterial strain (or the cultured medium thereof; or a bacterial product encompassed within that cultured medium) can have a sufficient ability in a disease associated functions. A disease-associated function may comprise a function of a bacterial strain or bacterial population that is related to a disease or disease condition as described herein. The disease associated function may be related to a complication associated with the disease or disease condition. The disease-associated function may comprise an alteration of the cellular process of the subject or the pathogen associated with the disease or disease condition. The disease-associated function may comprise an alteration of the cellular process of the bacterial strain or bacterial population as described herein. A sufficient ability, as used herein, when referring to a bacterial strain (or the cultured medium thereof; or a bacterial product encompassed within that cultured medium), refers to an ability of the bacterial strain (or the cultured medium thereof; or a bacterial product encompassed within that cultured medium) to alter the disease-associated function. The sufficient ability of a bacterial strain (or a bacterial population) can be mediated by a bacterial product generated by the bacterial strain. The bacterial product may be present within a medium used to culture the bacterial strain. The sufficient ability of a bacterial strain (or a bacterial population) can be mediated by the metabolic ability of a bacterial strain as described herein. The metabolic ability can convert a a substance of a nutrient source to a different substance.

A vaginal disease-associated function may comprise any BV-specific MOAs and functionalities described in this disclosure. In some cases, a vaginal disease-associated function may comprise an adherence to a vaginal epithelial cell (VEC), an inhibition of growth of a vaginal pathogen, an inhibition of a biofilm formation of a vaginal pathogen, a utilization of a vaginally relevant carbohydrate, a growth in a vaginal pH, or a combination thereof. A vaginal disease-associated function may comprise an adherence to a VEC. A vaginal disease-associated function may comprise an inhibition of growth of a vaginal pathogen. A vaginal disease-associated function may comprise an inhibition of a biofilm formation of a vaginal pathogen. A vaginal disease-associated function may comprise a utilization of a vaginally relevant carbohydrate. In some cases, a vaginal disease-associated function may comprise in a growth in a vaginal pH. In some cases, a vaginal disease-associated function may comprise an adherence to a VEC, an inhibition of growth of a vaginal pathogen, an inhibition of a biofilm formation of a vaginal pathogen, a growth in a vaginal pH, and a utilization of a vaginally relevant carbohydrate.

In some instances, the bacterial strain can have a sufficient ability in an infant gastrointestinal disease-associated function. The infant gastrointestinal disease-associated function may comprise any NEC-specific MOAs and functionalities described in this disclosure. In some cases, an infant gastrointestinal disease-associated function may comprise an adherence to an intestinal epithelial cell (IEC), an integrity of a barrier comprising IEC, an inhibition of an infant gastrointestinal pathogen, a utilization of an infant-relevant carbohydrate, an inhibition of an immune response signaling pathway, or a combination thereof. As used herein, an infant gastrointestinal pathogen comprises a microorganism that: (1) causes an infant gastrointestinal disease; (2) associated with an infant gastrointestinal; and/or (3) contributes to the symptoms of the infant gastrointestinal disease. In some instances, inhibiting or eliminating an infant gastrointestinal pathogen can treat or prevent the infant gastrointestinal disease or alleviate the symptoms of the infant gastrointestinal disease. In some cases, an infant gastrointestinal disease-associated function may comprise an adherence to an IEC. In some cases, an infant gastrointestinal disease-associated function may comprise an integrity of a barrier comprising IEC. In some cases, an infant gastrointestinal disease-associated function may comprise an inhibition of an infant gastrointestinal pathogen. In some cases, an infant gastrointestinal disease-associated function may comprise a utilization of an infant-relevant carbohydrate. In some cases, an infant gastrointestinal disease-associated function may comprise an inhibition of an immune response signaling pathway. In some instances, the bacterial strain can have a sufficient ability in a NEC-associated function. In some cases, an infant gastrointestinal disease-associated function may comprise an adherence to an intestinal epithelial cell (IES), an integrity of a barrier comprising IEC, an inhibition of an infant gastrointestinal pathogen, a utilization of an infant-relevant carbohydrate, and an inhibition of an immune response signaling pathway.

### 1. BV-Associated Functions

A bacterial strain described herein (or the cultured medium thereof; or a bacterial product encompassed within that cultured medium) may have a sufficient ability in the adherence to a VEC. In some cases, the bacterial strain may exhibit an adherence to the VEC by at least about 1×10\hat{ }1 CFU per 9.5 cm\hat{ }2 of VEC, at least about 2×10\hat{ }1 CFU per 9.5 cm\hat{ }2 of VEC, at least about 5×10\hat{ }1 CFU per 9.5 cm\hat{ }2 of VEC, at least about 1×10\hat{ }2 CFU per 9.5 cm\hat{ }2 of VEC, at least about 2×10\hat{ }2 CFU per 9.5 cm\hat{ }2 of VEC, at least about 5×10\hat{ }2 CFU per 9.5 cm\hat{ }2 of VEC, at least about 1×10\hat{ }3 CFU per 9.5 cm\hat{ }2 of VEC, at least about 2×10\hat{ }3 CFU per 9.5 cm\hat{ }2 of VEC, at least about 5×10\hat{ }3 CFU per 9.5 cm\hat{ }2 of VEC, at least about 1×10\hat{ }4 CFU per 9.5 cm\hat{ }2 of VEC, at least about 2×10\hat{ }4 CFU per 9.5 cm\hat{ }2 of VEC, at least about 5×10\hat{ }4 CFU per 9.5 cm\hat{ }2 of VEC, at least about 1×10\hat{ }5 CFU per 9.5 cm\hat{ }2 of VEC, at least about 2×10\hat{ }5 CFU per 9.5 cm\hat{ }2 of VEC, at least about 5×10\hat{ }5 CFU per 9.5 cm\hat{ }2 of VEC, at least about 1×10\hat{ }6 CFU per 9.5 cm\hat{ }2 of VEC, at least about 2×10\hat{ }6 CFU per 9.5 cm\hat{ }2 of VEC, at least about 5×10\hat{ }6 CFU per 9.5 cm\hat{ }2 of VEC, at least about 1×10\hat{ }7 CFU per 9.5 cm\hat{ }2 of VEC, at least about 2×10\hat{ }7 CFU per 9.5 cm\hat{ }2 of VEC, at least about 5×10\hat{ }7 CFU per 9.5 cm\hat{ }2 of VEC, at least about 1×10\hat{ }8 CFU per 9.5 cm\hat{ }2 of VEC, at least about 2×10\hat{ }8 CFU per 9.5 cm\hat{ }2 of VEC, at least about 5×10\hat{ }8 CFU per 9.5 cm\hat{ }2 of VEC, at least about 1×10\hat{ }9 CFU per 9.5 cm\hat{ }2 of VEC, at least about 2×10\hat{ }9 CFU per 9.5 cm\hat{ }2 of VEC, at least about 5×10\hat{ }9 CFU per 9.5 cm\hat{ }2 of VEC, at least about 1×10\hat{ }10 CFU per 9.5 cm\hat{ }2 of VEC, at least about 2×10\hat{ }10 CFU per 9.5 cm\hat{ }2 of VEC, at least about 5×10\hat{ }10 CFU per 9.5 cm\hat{ }2 of VEC, at least about 1×10\hat{ }11 CFU per 9.5 cm\hat{ }2 of VEC, at least about 2×10\hat{ }11 CFU per 9.5 cm\hat{ }2 of VEC, at least about 5×10\hat{ }11 CFU per 9.5 cm\hat{ }2 of VEC, at least about 1×10\hat{ }12 CFU per 9.5 cm\hat{ }2 of VEC, at least about 2×10\hat{ }12 CFU per 9.5 cm\hat{ }2 of VEC, at least about 5×10\hat{ }12 CFU per 9.5 cm\hat{ }2 of VEC or more. In some cases, the bacterial strain (or the cultured medium thereof; or a bacterial product encompassed within that cultured medium) may exhibit an adherence to the VEC by at most about 1×10\hat{ }1 CFU per 9.5 cm\hat{ }2 of VEC, at most about 2×10\hat{ }1 CFU per 9.5 cm\hat{ }2 of VEC, at most about 5×10\hat{ }1 CFU per 9.5 cm\hat{ }2 of VEC, at most about 1×10\hat{ }2 CFU per 9.5 cm\hat{ }2 of VEC, at most about 2×10\hat{ }2 CFU per 9.5 cm\hat{ }2 of VEC, at most about 5×10\hat{ }2 CFU per 9.5 cm\hat{ }2 of VEC, at most about 1×10\hat{ }3 CFU per 9.5 cm\hat{ }2 of VEC, at most about 2×10\hat{ }3 CFU per 9.5 cm\hat{ }2 of VEC, at most about 5×10\hat{ }3 CFU per 9.5 cm\hat{ }2 of VEC, at most about 1×10\hat{ }4 CFU per 9.5 cm\hat{ }2 of VEC, at most about 2×10\hat{ }4 CFU per 9.5 cm\hat{ }2 of VEC, at most about 5×10\hat{ }4 CFU per 9.5 cm\hat{ }2 of VEC, at most about 1×10\hat{ }5 CFU per 9.5 cm\hat{ }2 of VEC, at most about 2×10\hat{ }5 CFU per 9.5 cm\hat{ }2 of VEC, at most about 5×10\hat{ }5 CFU per 9.5 cm\hat{ }2 of VEC, at most about 1×10\hat{ }6 CFU per 9.5 cm\hat{ }2 of VEC, at most about 2×10\hat{ }6 CFU per 9.5 cm\hat{ }2 of VEC, at most about 5×10\hat{ }6 CFU per 9.5 cm\hat{ }2 of VEC, at most about 1×10\hat{ }7 CFU per 9.5 cm\hat{ }2 of VEC, at most about 2×10\hat{ }7 CFU per 9.5 cm\hat{ }2 of VEC, at most about 5×10\hat{ }7 CFU per 9.5 cm\hat{ }2 of VEC, at most about 1×10\hat{ }8 CFU per 9.5 cm\hat{ }2 of VEC, at most about 2×10\hat{ }8 CFU per 9.5 cm\hat{ }2 of VEC, at most about 5×10\hat{ }8 CFU per 9.5 cm\hat{ }2 of VEC, at most about 1×10\hat{ }9 CFU per 9.5 cm\hat{ }2 of VEC, at most about 2×10\hat{ }9 CFU per 9.5 cm\hat{ }2 of VEC, at most about 5×10\hat{ }9 CFU per 9.5 cm\hat{ }2 of VEC, at most about 1×10\hat{ }10 CFU per 9.5 cm\hat{ }2 of VEC, at most about 2×10\hat{ }10 CFU per 9.5 cm\hat{ }2 of VEC, at most about 5×10\hat{ }10 CFU per 9.5 cm\hat{ }2 of VEC, at most about 1×10\hat{ }11 CFU per 9.5 cm\hat{ }2 of VEC, at most about 2×10\hat{ }11 CFU per 9.5 cm\hat{ }2 of VEC, at most about 5×10\hat{ }11 CFU per 9.5 cm\hat{ }2 of VEC, at most about 1×10\hat{ }12 CFU per 9.5 cm\hat{ }2 of VEC, at most about 2×10\hat{ }12 CFU per 9.5 cm\hat{ }2 of VEC, or at most about 5×10\hat{ }12 CFU per 9.5 cm\hat{ }2 of VEC. In some cases, the bacterial strain described herein may have an adherence to a VEC that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain. In some cases, the bacterial strain described herein may have an adherence to a VEC that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain. The control strain may comprise LP01 (described in Luigi et al., Acta Biomed. 2019; 90(Suppl 7): 13-17; which is herein incorporated by reference in its entirety); LBV96 (described in U.S. Pat. No. 8,846,027 and Marschalek et al., Breast Care (Basel). 2017 October; 12(5):335-339; which is herein incorporated by reference in its entirety); LBV88 (described in U.S. Pat. No. 8,846,027 and Marschalek et al.); or LBV116 (described in U.S. Pat. No. 8,846,027 and Marschalek et al.). The adherence to the VEC can be measured by contacting a population of a bacterial strains to a VEC and measuring the number of the bacteria cells attached or adhered to the VEC. For example, the adherence to the VEC can be measured by the methods described herein, such as those described in EXAMPLE 2. In some cases, a bacterial strain described herein (or the cultured medium thereof; or a bacterial product encompassed within that cultured medium) may not have a sufficient ability in the adherence to a VEC

A bacterial strain described herein (or the cultured medium thereof; or a bacterial product encompassed within that cultured medium) may have a sufficient ability in inhibiting a growth or biofilm formation of a vaginal pathogen. The vaginal pathogen may comprise G. vaginalis, L. iners, Prevotella bivia, Atopobium vaginae, Sneathia spp., or a combination thereof. The vaginal pathogen may comprise G. vaginalis. The vaginal pathogen may comprise L. iners. The vaginal pathogen may comprise Prevotella bivia. The vaginal pathogen may comprise Atopobium vaginae. The vaginal pathogen may comprise Sneathia spp. The vaginal pathogen may comprise Prevotella bivia, Atopobium vaginae, Sneathia spp., G. vaginalis, and L. iners. In some cases, a bacterial strain described herein (or the cultured medium thereof; or a bacterial product encompassed within that cultured medium) may not have a sufficient ability in inhibiting a growth or biofilm formation of a vaginal pathogen.

In some cases, the bacterial strain (or the cultured medium thereof; or a bacterial product encompassed within that cultured medium) may inhibit the growth of a vaginal pathogen by at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, relative to a growth of the pathogen when inhibited by a control. The control may comprise growing the vaginal pathogen with a media control or without the bacterial strain (such as those described in EXAMPLE 2). The control may comprise growing the vaginal pathogen without the bacterial strain. In some cases, the bacterial strain (or the cultured medium thereof; or a bacterial product encompassed within that cultured medium) may inhibit the growth of a vaginal pathogen by at most about 1%, at most about 2%, at most about 3%, at most about 4%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or at most about 100%, relative to a growth of the pathogen when inhibited by the control.

In some cases, the bacterial strain described herein (or the cultured medium thereof; or a bacterial product encompassed within that cultured medium) may exhibit an inhibition of the growth of a vaginal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain (or the cultured medium thereof; or a bacterial product encompassed within that cultured medium). In some cases, the bacterial strain described herein (or the cultured medium thereof; or a bacterial product encompassed within that cultured medium) may exhibit an inhibition of the growth of a vaginal pathogen that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8% at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain (or the cultured medium thereof; or a bacterial product encompassed within that cultured medium). The control strain may comprise LP01; LBV96; LBV88; or LBV116. The inhibition of the growth of the pathogen can be measured by contacting the bacterial strains (or the cultured medium thereof; or a bacterial product encompassed within that cultured medium) to the vaginal pathogen and measuring the growth of the vaginal pathogen. For example, the inhibition of the growth of the vaginal pathogen can be measured by the methods described herein, such as those described in EXAMPLE 2.

In some cases, the bacterial strain (or the cultured medium thereof; or a bacterial product encompassed within that cultured medium) may inhibit the biofilm formation of a vaginal pathogen by at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, relative to a biofilm formation of the pathogen when inhibited by a control. The control may comprise growing the vaginal pathogen with a media control or without the bacterial strain (such as those described in EXAMPLE 2). The control may comprise growing the vaginal pathogen without the bacterial strain. In some cases, the bacterial strain (or the cultured medium thereof; or a bacterial product encompassed within that cultured medium) may inhibit the biofilm formation of a vaginal pathogen by at most about 1%, at most about 2%, at most about 3%, at most about 4%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or at most about 100%, relative to a biofilm formation of the pathogen when inhibited by the control.

In some cases, the bacterial strain described herein (or the cultured medium thereof; or a bacterial product encompassed within that cultured medium) may exhibit an inhibition of the biofilm formation of a vaginal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain (or the cultured medium thereof; or a bacterial product encompassed within that cultured medium). In some cases, the bacterial strain described herein (or the cultured medium thereof; or a bacterial product encompassed within that cultured medium) may exhibit an inhibition of the biofilm formation of a vaginal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain (or the cultured medium thereof; or a bacterial product encompassed within that cultured medium). The control strain may comprise LP01; LBV96; LBV88; or LBV116. The inhibition of the biofilm formation of the pathogen can be measured by contacting the bacterial strains (or the cultured medium thereof; or a bacterial product encompassed within that cultured medium) to the vaginal pathogen and measuring the biofilm formation of the vaginal pathogen. For example, the inhibition of the biofilm formation of the vaginal pathogen can be measured by the methods described herein, such as those described in EXAMPLE 2.

A bacterial strain described herein may have a sufficient ability in utilizing a vaginally relevant carbohydrate. The term “carbohydrate” as used herein when referring to a naturally occurring compound or derivative consisting of a combination of carbon, hydrogen, and oxygen molecules. The term “carbohydrate” can refer to an aldehyde or ketone with additional various hydroxyl groups including monosaccharides, disaccharides, and polysaccharides. Carbohydrates can be synthetically produced or derived from naturally occurring elements. A vaginally relevant carbohydrate can comprise a carbohydrate present in a vagina. In some cases, the vaginally relevant carbohydrate may comprise glycogen, glucose, dextrin (such as maltodextrin), maltose, mucin, sialic acid, or any combination thereof. In some cases, bacterial strain described herein may have a sufficient ability in utilizing glycogen. bacterial strain described herein may have a sufficient ability in utilizing dextrin. bacterial strain described herein may have a sufficient ability in utilizing maltodextrin. Glycogen can comprise bioglycogen. When a bacterial strain is capable of utilizing a particular nutrient substance (any substance used by an organism to survive, grow, and/or reproduce), it is also capable of converting the nutrient substance into another substance (for example via metabolic activity). For example, when a bacterial strain is capable of utilizing a particular carbohydrate, it is also capable of metabolizing the carbohydrate and/or converting the carbohydrate into a bacterial product or metabolite of that carbohydrate. In some cases, a bacterial strain capable of utilizing a particular substance is also capable of proliferating in an environment comprising that substance as a nutrient source. In some cases, a bacterial strain described herein may not have a sufficient ability in utilizing a vaginally relevant carbohydrate.

In some cases, utilization of vaginal-relevant can be measured by the growth ratio between the bacterial strain grown in a culture having a carbon source consisting of the vaginally relevant carbohydrate and the bacterial strain grown in a culture having a carbon source consisting of glucose (referred to as vaginally relevant carbohydrate growth ratio). A carbon source, as used herein, is a purified substance that acts as a source of carbon—for generating the biomass and/or energy of a microbial organism—that is added into the culture medium for culturing the microbial organism. For example, methods to determine the vaginally relevant carbohydrate growth ratio are described herein, such as those described in EXAMPLE 2.

In some cases, the bacterial strain described herein may have a vaginally relevant carbohydrate growth ratio of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have a vaginally relevant carbohydrate growth ratio of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5.

In some cases, the bacterial strain described herein may have a vaginally relevant carbohydrate growth ratio that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain. In some cases, the bacterial strain described herein may have a vaginally relevant carbohydrate growth ratio that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain. The control strain may comprise LP01; LBV96; LBV88; or LBV116.

In some cases, the bacterial strain described herein may have a vaginally relevant carbohydrate growth ratio (for glycogen) of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have a vaginally relevant carbohydrate growth ratio (for glycogen) of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5. In some cases, the bacterial strain described herein may have a vaginally relevant carbohydrate growth ratio (for bioglycogen) of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have a vaginally relevant carbohydrate growth ratio (for bioglycogen) of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5. In some cases, the bacterial strain described herein may have a vaginally relevant carbohydrate growth ratio (for dextrin) of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have a vaginally relevant carbohydrate growth ratio (for dextrin) of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5. In some cases, the bacterial strain described herein may have a vaginally relevant carbohydrate growth ratio (for maltodextrin) of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have a vaginally relevant carbohydrate growth ratio (for maltodextrin) of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5.

In some cases, the bacterial strain described herein may have a vaginally relevant carbohydrate growth (for example, for glycogen, bioglycogen, dextrin, or maltodextrin) ratio that is at least about 0.001%, at least about 0.001%, at least about 0.010%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain. In some cases, the bacterial strain described herein may have a vaginally relevant carbohydrate growth (for glycogen, bioglycogen, dextrin, or maltodextrin) ratio that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain. The control strain may comprise LP01; LBV96; LBV88; or LBV116.

A bacterial strain described herein may have a sufficient ability in growing in a vaginal-relevant pH (a pH lower than a physiological pH). A vaginal pH may be at least about 1, at least about 1.5, at least about 2, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, or at least about 6. A vaginal pH may be at most about 1, at most about 1.5, at most about 2, at most about 2.5, at most about 2.6, at most about 2.7, at most about 2.8, at most about 2.9, at most about 3, at most about 3.1, at most about 3.2, at most about 3.3, at most about 3.4, at most about 3.5, at most about 3.6, at most about 3.7, at most about 3.8, at most about 3.9, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, or at most about 6. A physiological pH is at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8. A physiological pH is at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, or at most about 8.

In some cases, the growth of a bacterial strain with various pH conditions can be measured by the growth ratio between the bacterial strain grown in a culture having a vaginal pH and the bacterial strain grown in a culture having a physiological pH (also referred to as “vaginal pH/physiological pH growth ratio”). The methods for measuring vaginal pH/physiological pH growth ratio of a bacterial strain can comprise those described in EXAMPLE 2. A bacterial strain described herein may not have a sufficient ability in growing in a vaginal-relevant pH.

In some cases, the bacterial strain described herein may have a vaginal pH/physiological pH growth ratio of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have a vaginal pH/physiological pH growth ratio of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5.

In some cases, the bacterial strain described herein may have a vaginal pH/physiological pH growth ratio that is at least about 0.001%, at least about 0.001%, at least about 0.010%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain. In some cases, the bacterial strain described herein may have a vaginal pH/physiological pH growth ratio that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain. The control strain may comprise LP01; LBV96; LBV88; or LBV116.

A bacterial strain described herein may have a sufficient ability in generating a bacterial product for treating or preventing a vaginal disease or a complication associated with the vaginal disease. For example, the bacterial products for treating or preventing BV can comprise lactic acids or hydrogen peroxide. The methods for measuring the amounts of the bacterial products can comprise those described in EXAMPLE 2. A bacterial strain described herein may not have a sufficient ability in generating a bacterial product for treating or preventing a vaginal disease or a complication associated with the vaginal disease.

In some cases, the bacterial strain described herein generate at least about at least about 1×10\hat{ }-3 micromolar (μM), at least about 2×10\hat{ }-3 μM, at least about 5×10\hat{ }-3 μM, at least about 1×10\hat{ }-2 μM, at least about 2×10\hat{ }-2 μM, at least about 5×10\hat{ }-2 μM, at least about 1×10\hat{ }-1 μM, at least about 2×10\hat{ }-1 μM, at least about 5×10\hat{ }-1 μM, at least about 1×10\hat{ }0 μM, at least about 2×10\hat{ }0 μM, at least about 5×10\hat{ }0 μM, at least about 1×10\hat{ }1 μM, at least about 2×10\hat{ }1 μM, at least about 5×10\hat{ }1 μM, at least about 1×10\hat{ }2 μM, at least about 2×10\hat{ }2 μM, at least about 5×10\hat{ }2 μM, at least about 1×10\hat{ }3 μM, at least about 2×10\hat{ }3 μM, at least about 5×10\hat{ }3 μM, at least about 1×10\hat{ }4 μM, at least about 2×10\hat{ }4 μM, at least about 5×10\hat{ }4 μM, at least about 1×10\hat{ }5 μM, at least about 2×10\hat{ }5 μM, at least about 5×10\hat{ }5 μM, at least about 1×10\hat{ }6 μM, at least about 2×10\hat{ }6 μM, at least about 5×10\hat{ }6 μM hydrogen peroxide, as measured the method described in EXAMPLE 2. In some cases, the bacterial strain described herein generate at most about at most about 1×10\hat{ }-3 micromolar (μM), at most about 2×10\hat{ }-3 μM, at most about 5×10\hat{ }-3 μM, at most about 1×10\hat{ }-2 μM, at most about 2×10\hat{ }-2 μM, at most about 5×10\hat{ }-2 μM, at most about 1×10\hat{ }-1 μM, at most about 2×10\hat{ }-1 μM, at most about 5×10\hat{ }-1 μM, at most about 1×10\hat{ }0 μM, at most about 2×10\hat{ }0 μM, at most about 5×10\hat{ }0 μM, at most about 1×10\hat{ }1 μM, at most about 2×10\hat{ }1 μM, at most about 5×10\hat{ }1 μM, at most about 1×10\hat{ }2 μM, at most about 2×10\hat{ }2 μM, at most about 5×10\hat{ }2 μM, at most about 1×10\hat{ }3 μM, at most about 2×10\hat{ }3 μM, at most about 5×10\hat{ }3 μM, at most about 1×10\hat{ }4 μM, at most about 2×10\hat{ }4 μM, at most about 5×10\hat{ }4 μM, at most about 1×10\hat{ }5 μM, at most about 2×10\hat{ }5 μM, at most about 5×10\hat{ }5 μM, at most about 1×10\hat{ }6 μM, at most about 2×10\hat{ }6 μM, at most about 5×10\hat{ }6 μM hydrogen peroxide, as measured the method described in EXAMPLE 2.

In some cases, the bacterial strain described herein generate at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold more hydrogen peroxide than that of a control strain. In some cases, the bacterial strain described herein generate at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold more hydrogen peroxide than that of a control strain. The control strain may comprise LP01; LBV96; LBV88; or LBV116.

In some cases, the bacterial strain described herein generate at least about 1×10\hat{ }1 RLU of lactic acids at least about 2×10\hat{ }1 RLU of lactic acids at least about 5×10\hat{ }1 RLU of lactic acids at least about 1×10\hat{ }2 RLU of lactic acids at least about 2×10\hat{ }2 RLU of lactic acids at least about 5×10\hat{ }2 RLU of lactic acids at least about 1×10\hat{ }3 RLU of lactic acids at least about 2×10\hat{ }3 RLU of lactic acids at least about 5×10\hat{ }3 RLU of lactic acids at least about 1×10\hat{ }4 RLU of lactic acids at least about 2×10\hat{ }4 RLU of lactic acids at least about 5×10\hat{ }4 RLU of lactic acids at least about 1×10\hat{ }5 RLU of lactic acids at least about 2×10\hat{ }5 RLU of lactic acids at least about 5×10\hat{ }5 RLU of lactic acids at least about 1×10\hat{ }6 RLU of lactic acids at least about 2×10\hat{ }6 RLU of lactic acids at least about 5×10\hat{ }6 RLU of lactic acids, as measured the method described in EXAMPLE 2. In some cases, the bacterial strain described herein generate at most about at most about 1×10\hat{ }1 RLU of lactic acids at most about 2×10\hat{ }1 RLU of lactic acids at most about 5×10\hat{ }1 RLU of lactic acids at most about 1×10\hat{ }2 RLU of lactic acids at most about 2×10\hat{ }2 RLU of lactic acids at most about 5×10\hat{ }2 RLU of lactic acids at most about 1×10\hat{ }3 RLU of lactic acids at most about 2×10\hat{ }3 RLU of lactic acids at most about 5×10\hat{ }3 RLU of lactic acids at most about 1×10\hat{ }4 RLU of lactic acids at most about 2×10\hat{ }4 RLU of lactic acids at most about 5×10\hat{ }4 RLU of lactic acids at most about 1×10\hat{ }5 RLU of lactic acids at most about 2×10\hat{ }5 RLU of lactic acids at most about 5×10\hat{ }5 RLU of lactic acids at most about 1×10\hat{ }6 RLU of lactic acids at most about 2×10\hat{ }6 RLU of lactic acids at most about 5×10\hat{ }6 RLU of lactic acids, as measured the method described in EXAMPLE 2.

In some cases, the bacterial strain described herein generate at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold more lactic acids than that of a control strain. In some cases, the bacterial strain described herein generate at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold more lactic acids than that of a control strain. The control strain may comprise LP01; LBV96; LBV88; or LBV116.

### 2. NEC-Associated Functions

A bacterial strain described herein (or the cultured medium thereof; or a bacterial product encompassed within that cultured medium) may have a sufficient ability in the adherence to an IEC. In some cases, the bacterial strain (or the cultured medium thereof; or a bacterial product encompassed within that cultured medium) may exhibit an adherence to the IEC by at least about 1×10\hat{ }1 CFU per 9.5 cm\hat{ }2 of IEC, at least about 2×10\hat{ }1 CFU per 9.5 cm\hat{ }2 of IEC, at least about 5×10\hat{ }1 CFU per 9.5 cm\hat{ }2 of IEC, at least about 1×10\hat{ }2 CFU per 9.5 cm\hat{ }2 of IEC, at least about 2×10\hat{ }2 CFU per 9.5 cm\hat{ }2 of IEC, at least about 5×10\hat{ }2 CFU per 9.5 cm\hat{ }2 of IEC, at least about 1×10\hat{ }3 CFU per 9.5 cm\hat{ }2 of IEC, at least about 2×10\hat{ }3 CFU per 9.5 cm\hat{ }2 of IEC, at least about 5×10\hat{ }3 CFU per 9.5 cm\hat{ }2 of IEC, at least about 1×10\hat{ }4 CFU per 9.5 cm\hat{ }2 of IEC, at least about 2×10\hat{ }4 CFU per 9.5 cm\hat{ }2 of IEC, at least about 5×10\hat{ }4 CFU per 9.5 cm\hat{ }2 of IEC, at least about 1×10\hat{ }5 CFU per 9.5 cm\hat{ }2 of IEC, at least about 2×10\hat{ }5 CFU per 9.5 cm\hat{ }2 of IEC, at least about 5×10\hat{ }5 CFU per 9.5 cm\hat{ }2 of IEC, at least about 1×10\hat{ }6 CFU per 9.5 cm\hat{ }2 of IEC, at least about 2×10\hat{ }6 CFU per 9.5 cm\hat{ }2 of IEC, at least about 5×10\hat{ }6 CFU per 9.5 cm\hat{ }2 of IEC, at least about 1×10\hat{ }7 CFU per 9.5 cm\hat{ }2 of IEC, at least about 2×10\hat{ }7 CFU per 9.5 cm\hat{ }2 of IEC, at least about 5×10\hat{ }7 CFU per 9.5 cm\hat{ }2 of IEC, at least about 1×10\hat{ }8 CFU per 9.5 cm\hat{ }2 of IEC, at least about 2×10\hat{ }8 CFU per 9.5 cm\hat{ }2 of IEC, at least about 5×10\hat{ }8 CFU per 9.5 cm\hat{ }2 of IEC, at least about 1×10\hat{ }9 CFU per 9.5 cm\hat{ }2 of IEC, at least about 2×10\hat{ }9 CFU per 9.5 cm\hat{ }2 of IEC, at least about 5×10\hat{ }9 CFU per 9.5 cm\hat{ }2 of IEC, at least about 1×10\hat{ }10 CFU per 9.5 cm\hat{ }2 of IEC, at least about 2×10\hat{ }10 CFU per 9.5 cm\hat{ }2 of IEC, at least about 5×10\hat{ }10 CFU per 9.5 cm\hat{ }2 of IEC, at least about 1×10\hat{ }11 CFU per 9.5 cm\hat{ }2 of IEC, at least about 2×10\hat{ }11 CFU per 9.5 cm\hat{ }2 of IEC, at least about 5×10\hat{ }11 CFU per 9.5 cm\hat{ }2 of IEC, at least about 1×10\hat{ }12 CFU per 9.5 cm\hat{ }2 of IEC, at least about 2×10\hat{ }12 CFU per 9.5 cm\hat{ }2 of IEC, at least about 5×10\hat{ }12 CFU per 9.5 cm\hat{ }2 of IEC or more. In some cases, the bacterial strain (or the cultured medium thereof; or a bacterial product encompassed within that cultured medium) may exhibit an adherence to the IEC by at most about 1×10\hat{ }1 CFU per 9.5 cm\hat{ }2 of IEC, at most about 2×10\hat{ }1 CFU per 9.5 cm\hat{ }2 of IEC, at most about 5×10\hat{ }1 CFU per 9.5 cm\hat{ }2 of IEC, at most about 1×10\hat{ }2 CFU per 9.5 cm\hat{ }2 of IEC, at most about 2×10\hat{ }2 CFU per 9.5 cm\hat{ }2 of IEC, at most about 5×10\hat{ }2 CFU per 9.5 cm\hat{ }2 of IEC, at most about 1×10\hat{ }3 CFU per 9.5 cm\hat{ }2 of IEC, at most about 2×10\hat{ }3 CFU per 9.5 cm\hat{ }2 of IEC, at most about 5×10\hat{ }3 CFU per 9.5 cm\hat{ }2 of IEC, at most about 1×10\hat{ }4 CFU per 9.5 cm\hat{ }2 of IEC, at most about 2×10\hat{ }4 CFU per 9.5 cm\hat{ }2 of IEC, at most about 5×10\hat{ }4 CFU per 9.5 cm\hat{ }2 of IEC, at most about 1×10\hat{ }5 CFU per 9.5 cm\hat{ }2 of IEC, at most about 2×10\hat{ }5 CFU per 9.5 cm\hat{ }2 of IEC, at most about 5×10\hat{ }5 CFU per 9.5 cm\hat{ }2 of IEC, at most about 1×10\hat{ }6 CFU per 9.5 cm\hat{ }2 of IEC, at most about 2×10\hat{ }6 CFU per 9.5 cm\hat{ }2 of IEC, at most about 5×10\hat{ }6 CFU per 9.5 cm\hat{ }2 of IEC, at most about 1×10\hat{ }7 CFU per 9.5 cm\hat{ }2 of IEC, at most about 2×10\hat{ }7 CFU per 9.5 cm\hat{ }2 of IEC, at most about 5×10\hat{ }7 CFU per 9.5 cm\hat{ }2 of IEC, at most about 1×10\hat{ }8 CFU per 9.5 cm\hat{ }2 of IEC, at most about 2×10\hat{ }8 CFU per 9.5 cm\hat{ }2 of IEC, at most about 5×10\hat{ }8 CFU per 9.5 cm\hat{ }2 of IEC, at most about 1×10\hat{ }9 CFU per 9.5 cm\hat{ }2 of IEC, at most about 2×10\hat{ }9 CFU per 9.5 cm\hat{ }2 of IEC, at most about 5×10\hat{ }9 CFU per 9.5 cm\hat{ }2 of IEC, at most about 1×10\hat{ }10 CFU per 9.5 cm\hat{ }2 of IEC, at most about 2×10\hat{ }10 CFU per 9.5 cm\hat{ }2 of IEC, at most about 5×10\hat{ }10 CFU per 9.5 cm\hat{ }2 of IEC, at most about 1×10\hat{ }11 CFU per 9.5 cm\hat{ }2 of IEC, at most about 2×10\hat{ }11 CFU per 9.5 cm\hat{ }2 of IEC, at most about 5×10\hat{ }11 CFU per 9.5 cm\hat{ }2 of IEC, at most about 1×10\hat{ }12 CFU per 9.5 cm\hat{ }2 of IEC, at most about 2×10\hat{ }12 CFU per 9.5 cm\hat{ }2 of IEC, or at most about 5×10\hat{ }12 CFU per 9.5 cm\hat{ }2 of IEC. In some cases, the bacterial strain described herein may have an adherence to an IEC that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain. In some cases, the bacterial strain described herein may have an adherence to an IEC that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain. The control strain may comprise EV27 (or referred to as evc001; described in BMC Pediatr. 2017; 17: 133); which is herein incorporated by reference in its entirety); BG49 (or referred to as IBP-9414; described in U.S. clinical trial no. NCT03978000; which is herein incorporated by reference in its entirety. The adherence to the IEC can be measured by contacting a population of a bacterial strains to an IEC and measuring the number of the bacteria cells attached or adhered to the IEC. For example, the adherence to the IEC can be measured by the methods described herein, such as those described in EXAMPLE 3. A bacterial strain described herein (or the cultured medium thereof; or a bacterial product encompassed within that cultured medium) may not have a sufficient ability in the adherence to an IEC.

A bacterial strain described herein (or the cultured medium thereof; or a bacterial product encompassed within that cultured medium) may have a sufficient ability in inhibiting a growth of an infant gastrointestinal pathogen. The infant gastrointestinal pathogen may comprise E. coli, K. pneumoniae, C. perfringens, S. aureus, S. flexneri, or a combination thereof. The infant gastrointestinal pathogen may comprise E. coli. The infant gastrointestinal pathogen may comprise K. pneumoniae. The infant gastrointestinal pathogen may comprise C. perfringens. The infant gastrointestinal pathogen may comprise S. aureus. The infant gastrointestinal pathogen may comprise S. flexneri. The infant gastrointestinal pathogen may comprise E. coli, K. pneumoniae, C. perfringens, S. aureus, and S. flexneri. A bacterial strain described herein (or the cultured medium thereof; or a bacterial product encompassed within that cultured medium) may not have a sufficient ability in inhibiting a growth of an infant gastrointestinal pathogen.

In some cases, the bacterial strain (or the cultured medium thereof; or a bacterial product encompassed within that cultured medium) may inhibit the growth of an infant gastrointestinal pathogen by at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, relative to a growth of the pathogen when inhibited by a control. The control may comprise growing the infant gastrointestinal pathogen with a media control or without the bacterial strain (such as those described in EXAMPLE 3). The control may comprise growing the infant gastrointestinal pathogen without the bacterial strain. In some cases, the bacterial strain (or the cultured medium thereof; or a bacterial product encompassed within that cultured medium) may inhibit the growth of an infant gastrointestinal pathogen by at most about 1%, at most about 2%, at most about 3%, at most about 4%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or at most about 100%, relative to a growth of the pathogen when inhibited by the control.

In some cases, the bacterial strain described herein (or the cultured medium thereof; or a bacterial product encompassed within that cultured medium) may exhibit an inhibition of the growth of an infant gastrointestinal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain (or the cultured medium thereof; or a bacterial product encompassed within that cultured medium). In some cases, the bacterial strain described herein (or the cultured medium thereof; or a bacterial product encompassed within that cultured medium) may exhibit an inhibition of the growth of an infant gastrointestinal pathogen that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain (or the cultured medium thereof; or a bacterial product encompassed within that cultured medium). The control strain may comprise EV27 or BG49. The inhibition of the growth of the pathogen can be measured by contacting the bacterial strains to the infant gastrointestinal pathogen and measuring the growth of the infant gastrointestinal pathogen. For example, the inhibition of the growth of the infant gastrointestinal pathogen can be measured by the methods described herein, such as those described in EXAMPLE 3.

A bacterial strain described herein (or the cultured medium thereof; or a bacterial product encompassed within that cultured medium) may have a sufficient ability in inhibiting a signaling pathway of a cell. Alteration of a signaling pathway may comprise altering an output of that signaling pathway. The output of that signaling pathway can comprise a transcriptional, translational, post-transcriptional, post-translational, metabolic, or cellular output; or a combination thereof. Inhibition (or activation) of a signaling pathway may comprise decreasing (or increasing) an output of that signaling pathway. In some cases, the bacterial strain described herein (or the cultured medium thereof; or a bacterial product encompassed within that cultured medium) having a sufficient ability in inhibiting (or activating) a signaling pathway can inhibit (or activate) a signal of a reporter of the signaling pathway. The reporter can comprise a transcriptional, translational, post-transcriptional, post-translational, metabolic, or cellular reporter; or a combination thereof. The reporter can be genetically engineered. The reporter can generate or facilitate a generation of the signal. The signal can be optical. The signal can be fluorescence. The signaling pathway may comprise an immune response signaling pathway. In some cases, the immune response signaling pathway may comprise an innate immune response signaling pathway. In some cases, the immune response signaling pathway may comprise an adaptive immune response signaling pathway. In some cases, the immune response signaling pathway may comprise an inflammatory immune response signaling pathway. The innate immune response signaling pathway may comprise Recognition Receptor (PRR) signaling pathway. The PPR signaling pathway may recognize molecules of by pathogens (Pathogen-Associated Molecular Patterns/PAMPs) or by damaged cells (the Damage-Associated Molecular Patterns/DAMPs). In some cases, the innate immune response signaling pathway may comprise toll-like receptor (TLR) signaling pathway. The TLR signaling pathway may comprise signaling pathways mediated by TLR1, TLR2, TLR3, TLR4, TLR5, TLR6, TLR7, TLR8, TLR9, TLR10, TLR11, TLR12, TLR13, or a combination thereof. In some cases, the immune response signaling pathway may comprise TLR1 signaling pathway. In some cases, the immune response signaling pathway may comprise TLR2 signaling pathway. In some cases, the immune response signaling pathway may comprise TLR3 signaling pathway. In some cases, the immune response signaling pathway may comprise TLR4 signaling pathway. In some cases, the immune response signaling pathway may comprise TLR5 signaling pathway. In some cases, the immune response signaling pathway may comprise TLR6 signaling pathway. In some cases, the immune response signaling pathway may comprise TLR7 signaling pathway. In some cases, the immune response signaling pathway may comprise TLR8 signaling pathway. In some cases, the immune response signaling pathway may comprise TLR9 signaling pathway. In some cases, the immune response signaling pathway may comprise TLR10 signaling pathway. In some cases, the immune response signaling pathway may comprise TLR11 signaling pathway. In some cases, the immune response signaling pathway may comprise TLR12 signaling pathway. In some cases, the immune response signaling pathway may comprise TLR13 signaling pathway. In some cases, the immune response signaling pathway may comprise nuclear factor kappa-light-chain-enhancer of activated B cells (NF-κB). In some cases, the immune response signaling pathway may comprise a ligand. The ligand can comprise a PAMP. The ligand can comprise a DAMP. The ligand can comprise a ligand of a gram-positive bacteria. The ligand can comprise a ligand of a gram-negative bacteria. The ligand of a gram-negative bacteria can comprise lipopolysaccharide (LPS). In some cases, the immune response signaling pathway may comprise nuclear factor kappa-light-chain-enhancer of activated B cells (NF-κB) signaling pathway. In some cases, the immune response signaling pathway may comprise interferon signaling pathway. In some cases, the bacterial strain or bacterial population described herein can inhibit the immune response pathway of a cell of a subject being administered with the composition or formulation comprising the bacterial strain or bacterial population. The cell may comprise an immune cell. The immune cell may comprise a macrophage, B-cell, or a T-cell. A bacterial strain described herein (or the cultured medium thereof; or a bacterial product encompassed within that cultured medium) may not have a sufficient ability in inhibiting a signaling pathway of a cell.

In some cases, the bacterial strain (or the cultured medium thereof; or a bacterial product encompassed within that cultured medium) may inhibit the immune response signaling pathway by at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, relative to immune response signaling pathway when inhibited by a control. The control may comprise inhibiting the immune response signaling pathway with a media control or without the bacterial strain (such as those described in EXAMPLE 3). In some cases, the bacterial strain (or the cultured medium thereof; or a bacterial product encompassed within that cultured medium) may inhibit the immune response signaling pathway by at most about 1%, at most about 2%, at most about 3%, at most about 4%, at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or at most about 100%, relative to the immune response signaling pathway when inhibited by the control.

In some cases, the bacterial strain described herein (or the cultured medium thereof; or a bacterial product encompassed within that cultured medium) may exhibit an inhibition of the immune response signaling pathway that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain (or the cultured medium thereof; or a bacterial product encompassed within that cultured medium). In some cases, the bacterial strain described herein (or the cultured medium thereof; or a bacterial product encompassed within that cultured medium) may exhibit an inhibition of the immune response signaling pathway that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain (or the cultured medium thereof; or a bacterial product encompassed within that cultured medium). The control strain may comprise EV27 or BG49. The inhibition (or activation) of a signaling pathway or the immune response signaling pathway can be measured by contacting the bacterial strains (or the cultured medium thereof; or a bacterial product encompassed within that cultured medium) to a cell comprising a reporter or an immune response reporter of the immune response signaling pathway and measuring the reporter or immune response reporter. The cell may comprise an engineered cell. The engineered cell may comprise a mammalian cell. The mammalian cell may comprise an immune cell. The immune cell may comprise a macrophage, B-cell, or a T-cell. In some cases, when measuring the immune response signaling pathway using the immune response reporter, the immune response reporter may be activated by a ligand (by contacting the ligand to the cell comprising the reporter), and the cell is then contacted with the bacterial strain. The activation (or inhibition or lack of activation) is then measured. The ligand can comprise a PAMP. The ligand can comprise a DAMP. The ligand can comprise a ligand of a gram-positive bacteria. The ligand can comprise a ligand of a gram-negative bacteria. The ligand of a gram-negative bacteria can comprise lipopolysaccharide (LPS). The immune response reporter may comprise a reporter for the TLR. In some cases, the immune response reporter may comprise a TLR4 reporter. In some embodiments, the engineered cell can comprise a macrophage. In some embodiments, the reporter can comprise a NFkB reporter or an interferon-sensitive response element reporter (ISRE). In some embodiments, the TLR4 reporter can comprise a NFkB reporter or an interferon-sensitive response element reporter (ISRE). In some embodiments, the TLR4 reporter can comprise a NFkB reporter. In some embodiments, the TLR4 reporter can comprise a ISRE reporter In some embodiments, the NFkB reporter can comprise a secreted embryonic alkaline phosphatase (SEAP) reporter. In some embodiments, the ISRE reporter can comprise a Lucia luciferase. For example, the inhibition of the growth of the vaginal pathogen can be measured by the methods described herein, such as those described in EXAMPLE 3.

A bacterial strain described herein (or the cultured medium thereof; or a bacterial product encompassed within that cultured medium) may have a sufficient ability in increasing the integrity of a barrier comprising IEC. The method to measure the integrity of the barrier comprising IEC may comprise contacting a bacterial strain with a barrier comprising IEC and measuring the cellular impedance of the barrier. The methods for measuring the barrier integrity are described herein, for example, in EXAMPLE 3. In some cases, a bacterial strain described herein may increase the cellular impedance of a barrier comprising IEC by at least at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control. The control may comprise growing the infant gastrointestinal pathogen with a media control or without the bacterial strain (such as those described in EXAMPLE 3). In some cases, a bacterial strain described herein may increase the cellular impedance of a barrier comprising IEC by at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control. A bacterial strain described herein (or the cultured medium thereof; or a bacterial product encompassed within that cultured medium) may not have a sufficient ability in increasing the integrity of a barrier comprising IEC.

A bacterial strain described herein may have a sufficient ability in utilizing an infant gastrointestinally relevant carbohydrate. An infant gastrointestinally relevant carbohydrate can comprise a carbohydrate present in an infant gastrointestinal tract. In some cases, the infant gastrointestinally relevant carbohydrate may comprise human milk oligosaccharides (HMOs). HMOs may comprise 2′-Fucosyllactose (2′FL), Lacto-N-difucohexaose I (LNDFH-I), Lacto-N-fucopentaose I (LNFP-I), Lacto-N-fucopentaose II (LNFP-II), Lacto-N-tetraose (LNT), 3-Fucosyllactose (3-FL), 6′-Sialyllactose (6′-SL), Disialyllacto-N-tetraose (DSLNT), Lacto-N-neotetraose (LNnT), Difucosyllactose (DFL), Fucosyldisialyllacto-N-hexose I (FDS-LNH), Lacto-N-fucopentaose III (LNFP-III), 3′-Sialyllactose (3′SL), or any combination thereof. In some cases, bacterial strain described herein may have a sufficient ability in utilizing HMOs as described herein. In some cases, bacterial strain described herein may have a sufficient ability in utilizing 2′FL. In some cases, bacterial strain described herein may have a sufficient ability in utilizing LNT. In some cases, bacterial strain described herein may have a sufficient ability in utilizing LNDFH-I. In some cases, bacterial strain described herein may have a sufficient ability in utilizing LNFP-I. In some cases, bacterial strain described herein may have a sufficient ability in utilizing LNFP-II. In some cases, bacterial strain described herein may have a sufficient ability in utilizing 3-FL. In some cases, bacterial strain described herein may have a sufficient ability in utilizing 6′-SL. In some cases, bacterial strain described herein may have a sufficient ability in utilizing DSLNT. In some cases, bacterial strain described herein may have a sufficient ability in utilizing LNnT. In some cases, bacterial strain described herein may have a sufficient ability in utilizing DFL. In some cases, bacterial strain described herein may have a sufficient ability in utilizing FDS-LNH. In some cases, bacterial strain described herein may have a sufficient ability in utilizing LNFP-III. In some cases, bacterial strain described herein may have a sufficient ability in utilizing 3′SL. A bacterial strain described herein may not have a sufficient ability in utilizing an infant gastrointestinally relevant carbohydrate.

In some cases, utilization of infant gastrointestinal-relevant can be measured by the growth ratio between the bacterial strain grown in a culture having a carbon source comprising the infant gastrointestinally relevant carbohydrate and the bacterial strain grown in a culture having a carbon source comprising glucose (referred to as infant gastrointestinally relevant carbohydrate growth ratio). For example, methods to determine the infant gastrointestinally relevant carbohydrate growth ratio are described herein, such as those described in EXAMPLE 3.

In some cases, the bacterial strain described herein may have an infant gastrointestinally relevant carbohydrate growth ratio of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have an infant gastrointestinally relevant carbohydrate growth ratio of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5.

In some cases, the bacterial strain described herein may have an infant gastrointestinally relevant carbohydrate growth ratio (for 2′FL) of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have an infant gastrointestinally relevant carbohydrate growth ratio (for 2′FL) of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5. In some cases, the bacterial strain described herein may have an infant gastrointestinally relevant carbohydrate growth ratio (for LNT) of at least about 0.01, at least about 0.02, at least about 0.05, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 3.5, at least about 4, at least about 4.5, at least about 5 or more. In some cases, the bacterial strain described herein may have an infant gastrointestinally relevant carbohydrate growth ratio (for LNT) of at most about 0.01, at most about 0.02, at most about 0.05, at most about 0.1, at most about 0.2, at most about 0.3, at most about 0.4, at most about 0.5, at most about 0.6, at most about 0.7, at most about 0.8, at most about 0.9, at most about 1, at most about 1.1, at most about 1.2, at most about 1.3, at most about 1.4, at most about 1.5, at most about 1.6, at most about 1.7, at most about 1.8, at most about 1.9, at most about 2, at most about 2.5, at most about 3, at most about 3.5, at most about 4, at most about 4.5, or at most about 5.

In some cases, the bacterial strain described herein may have an infant gastrointestinally relevant carbohydrate growth (for example, for 2′FL or LNT) ratio that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of a control strain. In some cases, the bacterial strain described herein may have a vaginally relevant carbohydrate growth (for glycogen, bioglycogen, dextrin, or maltodextrin) ratio that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of a control strain. The control strain may comprise EV27 or BG49.

A control composition or control formulation may comprise any control or control strain as described herein.

### Microbial Combinations

The bacterial population can comprise a plurality or at least two bacterial strains as described herein. The bacterial population can comprise a plurality or at least two bacterial species as described herein. The term “plurality” can comprise at 1, 2, 3, 4, 5, 6, 7, 8, 9, 10, or more. The plurality of bacterial strains can comprise any bacterial strains as descried herein. For a bacterial population comprising at least two bacterial strains, each of the at least two bacterial strains are different bacterial strains. In some cases, each of the at least two bacterial strains may comprise the bacterial species as described herein. In some cases, the bacterial population can comprise a plurality of bacterial strains of Bifidobacterium sp. or Lactobacillus sp. (or Vertebrate-Associated Lactobacillaceae). In some cases, the bacterial population can comprise a plurality of bacterial strains of Bifidobacterium adolescentis, Bifidobacterium bifidum, Bifidobacterium breve, Bifidobacterium longum, Bifidobacterium pseudocatenulatum, Lactobacillus crispatus, Lactobacillus gasseri, Lactobacillus jensenii, Lactobacillus plantarum, or Lactobacillus rhamnosus. A plurality of bacterial strains of Bifidobacterium adolescentis, Bifidobacterium bifidum, Bifidobacterium breve, Bifidobacterium longum, Bifidobacterium pseudocatenulatum, Lactobacillus crispatus, Lactobacillus gasseri, Lactobacillus jensenii, Lactobacillus plantarum, or Lactobacillus rhamnosus can comprise any individual bacterial strain or combinations of ST101, ST31, ST80, ST56, ST71, ST23, ST19, ST81, ST119, ST37, ST66, ST20, ST100, ST112, ST105, ST21, ST65, and/or ST116. In some cases, the bacterial population can comprise any of the aforementioned bacterial strains in addition to one or more additional bacterial strains from Bifidobacterium sp. or Lactobacillus sp. (or Vertebrate-Associated Lactobacillaceae).

The bacterial population can comprise any combinations of at least two bacterial strains of the following bacterial strains: a bacterial of Lactobacillus crispatus as described herein, a bacterial of Lactobacillus gasseri as described herein, a bacterial of Lactobacillus jensenii as described herein, a bacterial of Bifidobacterium bifidum as described herein, a bacterial of Lactobacillus plantarum as described herein, a bacterial of Bifidobacterium adolescentis as described herein, a bacterial of Bifidobacterium breve as described herein, a bacterial of Bifidobacterium longum as described herein, a bacterial of Bifidobacterium pseudocatenulatum as described herein, and/or a bacterial of Lactobacillus rhamnosus as described herein. The bacterial population can comprise any combinations of at least two bacterial strains of the following bacterial strains: a bacterial of Lactobacillus crispatus as described herein, a bacterial of Lactobacillus gasseri as described herein, and/or a bacterial of Lactobacillus jensenii as described herein. In some cases, the composition may be used for treating or preventing a disease or disease condition. A disease or disease condition may comprise a disease or complication associated with the disease. In some cases, the composition for treating or preventing a vaginal disease as described herein (e.g., BV or recurrent BV or a complication associated with the vaginal disease (e.g., pre-term birth, birth/pregnancy complications, STIs (such as HIV, Chlamydia, Gonorrhea, Suphilis, Trichomoniasis), pelvic inflammatory disease (PID), vulvovaginitis, or a combination thereof can comprise a bacterial population comprising any combinations of at least two bacterial strains of the following bacterial strains: a bacterial of Lactobacillus crispatus as described herein, a bacterial of Lactobacillus gasseri as described herein, and/or a bacterial of Lactobacillus jensenii as described herein. The bacterial population can comprise any combinations of at least two bacterial strains of the following bacterial strains: a bacterial of Bifidobacterium bifidum as described herein, a bacterial of Lactobacillus plantarum as described herein, a bacterial of Bifidobacterium adolescentis as described herein, a bacterial of Bifidobacterium breve as described herein, a bacterial of Bifidobacterium longum as described herein, a bacterial of Bifidobacterium pseudocatenulatum as described herein, and/or a bacterial of Lactobacillus rhamnosus as described herein. In some cases, the composition for treating or preventing an infant gastrointestinal disease as described herein (e.g., NEC, infectious gastroenteritis, neonatal cholestasis, or pediatric intestinal motility disorder) can comprise a bacterial population comprising any combinations of at least two bacterial strains of the following bacterial strains: a bacterial of Bifidobacterium bifidum as described herein, a bacterial of Lactobacillus plantarum as described herein, a bacterial of Bifidobacterium adolescentis as described herein, a bacterial of Bifidobacterium breve as described herein, a bacterial of Bifidobacterium longum as described herein, a bacterial of Bifidobacterium pseudocatenulatum as described herein, and/or a bacterial of Lactobacillus rhamnosus as described herein.

The bacterial population can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more bacterial strain(s). The bacterial population can comprise at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, or at most about 20 bacterial strain(s). The bacterial population can comprise at least about 1 bacterial strain. The bacterial population can comprise at least about 2 bacterial strains. The bacterial population can comprise at least about 3 bacterial strains. The bacterial population can comprise at least about 4 bacterial strains. The bacterial population can comprise at least about 5 bacterial strains. The bacterial population can comprise at least about 6 bacterial strains. The bacterial population can comprise at least about 7 bacterial strains. The bacterial population can comprise at least about 8 bacterial strains. The bacterial population can comprise at least about 9 bacterial strains. The bacterial population can comprise at least about 10 bacterial strains. The bacterial population can comprise at least about 11 bacterial strains. The bacterial population can comprise at least about 12 bacterial strains. The bacterial population can comprise at least about 13 bacterial strains. The bacterial population can comprise at least about 14 bacterial strains. The bacterial population can comprise at least about 15 bacterial strains. The bacterial population can comprise at least about 16 bacterial strains. The bacterial population can comprise at least about 17 bacterial strains. The bacterial population can comprise at least about bacterial strains. The bacterial population can comprise at least about 19 bacterial strains. The bacterial population can comprise at least about 20 bacterial strains. The bacterial population can comprise more than 20 bacterial strains. The bacterial population can comprise at most about 1 bacterial strain. The bacterial population can comprise at most about 2 bacterial strains. The bacterial population can comprise at most about 3 bacterial strains. The bacterial population can comprise at most about 4 bacterial strains. The bacterial population can comprise at most about 5 bacterial strains. The bacterial population can comprise at most about 6 bacterial strains. The bacterial population can comprise at most about 7 bacterial strains. The bacterial population can comprise at most about 8 bacterial strains. The bacterial population can comprise at most about 9 bacterial strains. The bacterial population can comprise at most about 10 bacterial strains. The bacterial population can comprise at most about 11 bacterial strains. The bacterial population can comprise at most about 12 bacterial strains. The bacterial population can comprise at most about 13 bacterial strains. The bacterial population can comprise at most about 14 bacterial strains. The bacterial population can comprise at most about 15 bacterial strains. The bacterial population can comprise at most about 16 bacterial strains. The bacterial population can comprise at most about 17 bacterial strains. The bacterial population can comprise at most about 18 bacterial strains. The bacterial population can comprise at most about 19 bacterial strains. The bacterial population can comprise at most about 20 bacterial strains. In some cases, the plurality of bacterial strains may not comprise strains from a same bacterial species. In some cases, the plurality of bacterial strains may comprise strains from a same bacterial species.

The bacterial population can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more donor bacterial strain(s). The bacterial population can comprise at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, or at most about 20 donor bacterial strain(s). The bacterial population can comprise at least about 1 donor bacterial strain. The bacterial population can comprise at least about 2 donor bacterial strains. The bacterial population can comprise at least about 3 donor bacterial strains. The bacterial population can comprise at least about 4 donor bacterial strains. The bacterial population can comprise at least about 5 donor bacterial strains. The bacterial population can comprise at least about 6 donor bacterial strains. The bacterial population can comprise at least about 7 donor bacterial strains. The bacterial population can comprise at least about 8 donor bacterial strains. The bacterial population can comprise at least about 9 donor bacterial strains. The bacterial population can comprise at least about 10 donor bacterial strains. The bacterial population can comprise at least about 11 donor bacterial strains. The bacterial population can comprise at least about 12 donor bacterial strains. The bacterial population can comprise at least about 13 donor bacterial strains. The bacterial population can comprise at least about 14 donor bacterial strains. The bacterial population can comprise at least about 15 donor bacterial strains. The bacterial population can comprise at least about 16 donor bacterial strains. The bacterial population can comprise at least about 17 donor bacterial strains. The bacterial population can comprise at least about 18 donor bacterial strains. The bacterial population can comprise at least about 19 donor bacterial strains. The bacterial population can comprise at least about 20 donor bacterial strains. The bacterial population can comprise more than 20 donor bacterial strains. The bacterial population can comprise at most about 1 donor bacterial strain. The bacterial population can comprise at most about 2 donor bacterial strains. The bacterial population can comprise at most about 3 donor bacterial strains. The bacterial population can comprise at most about 4 donor bacterial strains. The bacterial population can comprise at most about 5 donor bacterial strains. The bacterial population can comprise at most about 6 donor bacterial strains. The bacterial population can comprise at most about 7 donor bacterial strains. The bacterial population can comprise at most about 8 donor bacterial strains. The bacterial population can comprise at most about 9 donor bacterial strains. The bacterial population can comprise at most about 10 donor bacterial strains. The bacterial population can comprise at most about 11 donor bacterial strains. The bacterial population can comprise at most about 12 donor bacterial strains. The bacterial population can comprise at most about 13 donor bacterial strains. The bacterial population can comprise at most about 14 donor bacterial strains. The bacterial population can comprise at most about 15 donor bacterial strains. The bacterial population can comprise at most about 16 donor bacterial strains. The bacterial population can comprise at most about 17 donor bacterial strains. The bacterial population can comprise at most about 18 donor bacterial strains. The bacterial population can comprise at most about 19 donor bacterial strains. The bacterial population can comprise at most about 20 donor bacterial strains.

The bacterial population can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more recipient bacterial strain(s). The bacterial population can comprise at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, or at most about 20 recipient bacterial strain(s). The bacterial population can comprise at least about 1 recipient bacterial strain. The bacterial population can comprise at least about 2 recipient bacterial strains. The bacterial population can comprise at least about 3 recipient bacterial strains. The bacterial population can comprise at least about 4 recipient bacterial strains. The bacterial population can comprise at least about 5 recipient bacterial strains. The bacterial population can comprise at least about 6 recipient bacterial strains. The bacterial population can comprise at least about 7 recipient bacterial strains. The bacterial population can comprise at least about 8 recipient bacterial strains. The bacterial population can comprise at least about 9 recipient bacterial strains. The bacterial population can comprise at least about 10 recipient bacterial strains. The bacterial population can comprise at least about 11 recipient bacterial strains. The bacterial population can comprise at least about 12 recipient bacterial strains. The bacterial population can comprise at least about 13 recipient bacterial strains. The bacterial population can comprise at least about 14 recipient bacterial strains. The bacterial population can comprise at least about 15 recipient bacterial strains. The bacterial population can comprise at least about 16 recipient bacterial strains. The bacterial population can comprise at least about 17 recipient bacterial strains. The bacterial population can comprise at least about 18 recipient bacterial strains. The bacterial population can comprise at least about 19 recipient bacterial strains. The bacterial population can comprise at least about 20 recipient bacterial strains. The bacterial population can comprise more than 20 recipient bacterial strains. The bacterial population can comprise at most about 1 recipient bacterial strain. The bacterial population can comprise at most about 2 recipient bacterial strains. The bacterial population can comprise at most about 3 recipient bacterial strains. The bacterial population can comprise at most about 4 recipient bacterial strains. The bacterial population can comprise at most about 5 recipient bacterial strains. The bacterial population can comprise at most about 6 recipient bacterial strains. The bacterial population can comprise at most about 7 recipient bacterial strains. The bacterial population can comprise at most about 8 recipient bacterial strains. The bacterial population can comprise at most about 9 recipient bacterial strains. The bacterial population can comprise at most about 10 recipient bacterial strains. The bacterial population can comprise at most about 11 recipient bacterial strains. The bacterial population can comprise at most about 12 recipient bacterial strains. The bacterial population can comprise at most about 13 recipient bacterial strains. The bacterial population can comprise at most about 14 recipient bacterial strains. The bacterial population can comprise at most about 15 recipient bacterial strains. The bacterial population can comprise at most about 16 recipient bacterial strains. The bacterial population can comprise at most about 17 recipient bacterial strains. The bacterial population can comprise at most about 18 recipient bacterial strains. The bacterial population can comprise at most about 19 recipient bacterial strains. The bacterial population can comprise at most about 20 recipient bacterial strains.

A bacterial population can comprise ST100 and ST20. A bacterial population can comprise ST100 and ST112. A bacterial population can comprise ST100 and ST105. A bacterial population can comprise ST100 and ST21. A bacterial population can comprise ST100 and a bacterial strain of Lactobacillus crispatus. A bacterial population can comprise ST100 and a bacterial strain of Lactobacillus gasseri. A bacterial population can comprise ST100 and a bacterial strain of Lactobacillus jensenii. A bacterial population can comprise ST20 and ST112. A bacterial population can comprise ST20 and ST105. A bacterial population can comprise ST20 and ST21. A bacterial population can comprise ST20 and a bacterial strain of Lactobacillus crispatus. A bacterial population can comprise ST20 and a bacterial strain of Lactobacillus gasseri. A bacterial population can comprise ST20 and a bacterial strain of Lactobacillus jensenii. A bacterial population can comprise ST112 and ST105. A bacterial population can comprise ST112 and ST21. A bacterial population can comprise ST112 and a bacterial strain of Lactobacillus crispatus. A bacterial population can comprise ST112 and a bacterial strain of Lactobacillus gasseri. A bacterial population can comprise ST112 and a bacterial strain of Lactobacillus jensenii. A bacterial population can comprise ST105 and ST21. A bacterial population can comprise ST105 and a bacterial strain of Lactobacillus crispatus. A bacterial population can comprise ST105 and a bacterial strain of Lactobacillus gasseri. A bacterial population can comprise ST105 and a bacterial strain of Lactobacillus jensenii. A bacterial population can comprise ST21 and a bacterial strain of Lactobacillus crispatus. A bacterial population can comprise ST21 and a bacterial strain of Lactobacillus gasseri. A bacterial population can comprise ST21 and a bacterial strain of Lactobacillus jensenii. A bacterial population can comprise a bacterial strain of Lactobacillus crispatus and a bacterial strain of Lactobacillus gasseri. A bacterial population can comprise a bacterial strain of Lactobacillus crispatus and a bacterial strain of Lactobacillus jensenii. A bacterial population can comprise a bacterial strain of Lactobacillus gasseri and a bacterial strain of Lactobacillus jensenii.

A bacterial population can comprise ST100, ST20, and ST112. A bacterial population can comprise ST100, ST20, and ST105. A bacterial population can comprise ST100, ST20, and ST21. A bacterial population can comprise ST100, ST20, and a bacterial strain of Lactobacillus crispatus. A bacterial population can comprise ST100, ST20, and a bacterial strain of Lactobacillus gasseri. A bacterial population can comprise ST100, ST20, and a bacterial strain of Lactobacillus jensenii. A bacterial population can comprise ST100, ST112, and ST105. A bacterial population can comprise ST100, ST112, and ST21. A bacterial population can comprise ST100, ST112, and a bacterial strain of Lactobacillus crispatus. A bacterial population can comprise ST100, ST112, and a bacterial strain of Lactobacillus gasseri. A bacterial population can comprise ST100, ST112, and a bacterial strain of Lactobacillus jensenii. A bacterial population can comprise ST100, ST105, and ST21. A bacterial population can comprise ST100, ST105, and a bacterial strain of Lactobacillus crispatus. A bacterial population can comprise ST100, ST105, and a bacterial strain of Lactobacillus gasseri. A bacterial population can comprise ST100, ST105, and a bacterial strain of Lactobacillus jensenii. A bacterial population can comprise ST100, ST21, and a bacterial strain of Lactobacillus crispatus. A bacterial population can comprise ST100, ST21, and a bacterial strain of Lactobacillus gasseri. A bacterial population can comprise ST100, ST21, and a bacterial strain of Lactobacillus jensenii. A bacterial population can comprise ST100, a bacterial strain of Lactobacillus crispatus, and a bacterial strain of Lactobacillus gasseri. A bacterial population can comprise ST100, a bacterial strain of Lactobacillus crispatus, and a bacterial strain of Lactobacillus jensenii. A bacterial population can comprise ST100, a bacterial strain of Lactobacillus gasseri, and a bacterial strain of Lactobacillus jensenii. A bacterial population can comprise ST20, ST112, and ST105. A bacterial population can comprise ST20, ST112, and ST21. A bacterial population can comprise ST20, ST112, and a bacterial strain of Lactobacillus crispatus. A bacterial population can comprise ST20, ST112, and a bacterial strain of Lactobacillus gasseri. A bacterial population can comprise ST20, ST112, and a bacterial strain of Lactobacillus jensenii. A bacterial population can comprise ST20, ST105, and ST21. A bacterial population can comprise ST20, ST105, and a bacterial strain of Lactobacillus crispatus. A bacterial population can comprise ST20, ST105, and a bacterial strain of Lactobacillus gasseri. A bacterial population can comprise ST20, ST105, and a bacterial strain of Lactobacillus jensenii. A bacterial population can comprise ST20, ST21, and a bacterial strain of Lactobacillus crispatus. A bacterial population can comprise ST20, ST21, and a bacterial strain of Lactobacillus gasseri. A bacterial population can comprise ST20, ST21, and a bacterial strain of Lactobacillus jensenii. A bacterial population can comprise ST20, a bacterial strain of Lactobacillus crispatus, and a bacterial strain of Lactobacillus gasseri. A bacterial population can comprise ST20, a bacterial strain of Lactobacillus crispatus, and a bacterial strain of Lactobacillus jensenii. A bacterial population can comprise ST20, a bacterial strain of Lactobacillus gasseri, and a bacterial strain of Lactobacillus jensenii. A bacterial population can comprise ST112, ST105, and ST21. A bacterial population can comprise ST112, ST105, and a bacterial strain of Lactobacillus crispatus. A bacterial population can comprise ST112, ST105, and a bacterial strain of Lactobacillus gasseri. A bacterial population can comprise ST112, ST105, and a bacterial strain of Lactobacillus jensenii. A bacterial population can comprise ST112, ST21, and a bacterial strain of Lactobacillus crispatus. A bacterial population can comprise ST112, ST21, and a bacterial strain of Lactobacillus gasseri. A bacterial population can comprise ST112, ST21, and a bacterial strain of Lactobacillus jensenii. A bacterial population can comprise ST112, a bacterial strain of Lactobacillus crispatus, and a bacterial strain of Lactobacillus gasseri. A bacterial population can comprise ST112, a bacterial strain of Lactobacillus crispatus, and a bacterial strain of Lactobacillus jensenii. A bacterial population can comprise ST112, a bacterial strain of Lactobacillus gasseri, and a bacterial strain of Lactobacillus jensenii. A bacterial population can comprise ST105, ST21, and a bacterial strain of Lactobacillus crispatus. A bacterial population can comprise ST105, ST21, and a bacterial strain of Lactobacillus gasseri. A bacterial population can comprise ST105, ST21, and a bacterial strain of Lactobacillus jensenii. A bacterial population can comprise ST105, a bacterial strain of Lactobacillus crispatus, and a bacterial strain of Lactobacillus gasseri. A bacterial population can comprise ST105, a bacterial strain of Lactobacillus crispatus, and a bacterial strain of Lactobacillus jensenii. A bacterial population can comprise ST105, a bacterial strain of Lactobacillus gasseri, and a bacterial strain of Lactobacillus jensenii. A bacterial population can comprise ST21, a bacterial strain of Lactobacillus crispatus, and a bacterial strain of Lactobacillus gasseri. A bacterial population can comprise ST21, a bacterial strain of Lactobacillus crispatus, and a bacterial strain of Lactobacillus jensenii. A bacterial population can comprise ST21, a bacterial strain of Lactobacillus gasseri, and a bacterial strain of Lactobacillus jensenii. A bacterial population can comprise a bacterial strain of Lactobacillus crispatus, a bacterial strain of Lactobacillus gasseri, and a bacterial strain of Lactobacillus jensenii.

A bacterial population can comprise a bacterial strain of each of the following: Bifidobacterium bifidum and Lactobacillus plantarum. A bacterial population can comprise a bacterial strain of each of the following: Bifidobacterium bifidum and Bifidobacterium adolescentis. A bacterial population can comprise a bacterial strain of each of the following: Bifidobacterium bifidum and Bifidobacterium breve. A bacterial population can comprise a bacterial strain of each of the following: Bifidobacterium bifidum and Bifidobacterium longum. A bacterial population can comprise a bacterial strain of each of the following: Bifidobacterium bifidum and Bifidobacterium pseudocatenulatum. A bacterial population can comprise a bacterial strain of each of the following: Bifidobacterium bifidum and Lactobacillus rhamnosus. A bacterial population can comprise a bacterial strain of each of the following: Lactobacillus plantarum and Bifidobacterium adolescentis. A bacterial population can comprise a bacterial strain of each of the following: Lactobacillus plantarum and Bifidobacterium breve. A bacterial population can comprise a bacterial strain of each of the following: Lactobacillus plantarum and Bifidobacterium longum. A bacterial population can comprise a bacterial strain of each of the following: Lactobacillus plantarum and Bifidobacterium pseudocatenulatum. A bacterial population can comprise a bacterial strain of each of the following: Lactobacillus plantarum and Lactobacillus rhamnosus. A bacterial population can comprise a bacterial strain of each of the following: Bifidobacterium adolescentis and Bifidobacterium breve. A bacterial population can comprise a bacterial strain of each of the following: Bifidobacterium adolescentis and Bifidobacterium longum. A bacterial population can comprise a bacterial strain of each of the following: Bifidobacterium adolescentis and Bifidobacterium pseudocatenulatum. A bacterial population can comprise a bacterial strain of each of the following: Bifidobacterium adolescentis and Lactobacillus rhamnosus. A bacterial population can comprise a bacterial strain of each of the following: Bifidobacterium breve and Bifidobacterium longum. A bacterial population can comprise a bacterial strain of each of the following: Bifidobacterium breve and Bifidobacterium pseudocatenulatum. A bacterial population can comprise a bacterial strain of each of the following: Bifidobacterium breve and Lactobacillus rhamnosus. A bacterial population can comprise a bacterial strain of each of the following: Bifidobacterium longum and Bifidobacterium pseudocatenulatum. A bacterial population can comprise a bacterial strain of each of the following: Bifidobacterium longum and Lactobacillus rhamnosus. A bacterial population can comprise a bacterial strain of each of the following: Bifidobacterium pseudocatenulatum and Lactobacillus rhamnosus. A bacterial population can comprise a bacterial strain of each of the following: Bifidobacterium bifidum, Lactobacillus plantarum, and Bifidobacterium adolescentis. A bacterial population can comprise a bacterial strain of each of the following: Bifidobacterium bifidum, Lactobacillus plantarum, and Bifidobacterium breve. A bacterial population can comprise a bacterial strain of each of the following: Bifidobacterium bifidum, Lactobacillus plantarum, and Bifidobacterium longum. A bacterial population can comprise a bacterial strain of each of the following: Bifidobacterium bifidum, Lactobacillus plantarum, and Bifidobacterium pseudocatenulatum. A bacterial population can comprise a bacterial strain of each of the following: Bifidobacterium bifidum, Lactobacillus plantarum, and Lactobacillus rhamnosus. A bacterial population can comprise a bacterial strain of each of the following: Bifidobacterium bifidum, Bifidobacterium adolescentis, and Bifidobacterium breve. A bacterial population can comprise a bacterial strain of each of the following: Bifidobacterium bifidum, Bifidobacterium adolescentis, and Bifidobacterium longum. A bacterial population can comprise a bacterial strain of each of the following: Bifidobacterium bifidum, Bifidobacterium adolescentis, and Bifidobacterium pseudocatenulatum. A bacterial population can comprise a bacterial strain of each of the following: Bifidobacterium bifidum, Bifidobacterium adolescentis, and Lactobacillus rhamnosus. A bacterial population can comprise a bacterial strain of each of the following: Bifidobacterium bifidum, Bifidobacterium breve, and Bifidobacterium longum. A bacterial population can comprise a bacterial strain of each of the following: Bifidobacterium bifidum, Bifidobacterium breve, and Bifidobacterium pseudocatenulatum. A bacterial population can comprise a bacterial strain of each of the following: Bifidobacterium bifidum, Bifidobacterium breve, and Lactobacillus rhamnosus. A bacterial population can comprise a bacterial strain of each of the following: Bifidobacterium bifidum, Bifidobacterium longum, and Bifidobacterium pseudocatenulatum. A bacterial population can comprise a bacterial strain of each of the following: Bifidobacterium bifidum, Bifidobacterium longum, and Lactobacillus rhamnosus. A bacterial population can comprise a bacterial strain of each of the following: Bifidobacterium bifidum, Bifidobacterium pseudocatenulatum, and Lactobacillus rhamnosus. A bacterial population can comprise a bacterial strain of each of the following: Lactobacillus plantarum, Bifidobacterium adolescentis, and Bifidobacterium breve. A bacterial population can comprise a bacterial strain of each of the following: Lactobacillus plantarum, Bifidobacterium adolescentis, and Bifidobacterium longum. A bacterial population can comprise a bacterial strain of each of the following: Lactobacillus plantarum, Bifidobacterium adolescentis, and Bifidobacterium pseudocatenulatum. A bacterial population can comprise a bacterial strain of each of the following: Lactobacillus plantarum, Bifidobacterium adolescentis, and Lactobacillus rhamnosus. A bacterial population can comprise a bacterial strain of each of the following: Lactobacillus plantarum, Bifidobacterium breve, and Bifidobacterium longum. A bacterial population can comprise a bacterial strain of each of the following: Lactobacillus plantarum, Bifidobacterium breve, and Bifidobacterium pseudocatenulatum. A bacterial population can comprise a bacterial strain of each of the following: Lactobacillus plantarum, Bifidobacterium breve, and Lactobacillus rhamnosus. A bacterial population can comprise a bacterial strain of each of the following: Lactobacillus plantarum, Bifidobacterium longum, and Bifidobacterium pseudocatenulatum. A bacterial population can comprise a bacterial strain of each of the following: Lactobacillus plantarum, Bifidobacterium longum, and Lactobacillus rhamnosus. A bacterial population can comprise a bacterial strain of each of the following: Lactobacillus plantarum, Bifidobacterium pseudocatenulatum, and Lactobacillus rhamnosus. A bacterial population can comprise a bacterial strain of each of the following: Bifidobacterium adolescentis, Bifidobacterium breve, and Bifidobacterium longum. A bacterial population can comprise a bacterial strain of each of the following: Bifidobacterium adolescentis, Bifidobacterium breve, and Bifidobacterium pseudocatenulatum. A bacterial population can comprise a bacterial strain of each of the following: Bifidobacterium adolescentis, Bifidobacterium breve, and Lactobacillus rhamnosus. A bacterial population can comprise a bacterial strain of each of the following: Bifidobacterium adolescentis, Bifidobacterium longum, and Bifidobacterium pseudocatenulatum. A bacterial population can comprise a bacterial strain of each of the following: Bifidobacterium adolescentis, Bifidobacterium longum, and Lactobacillus rhamnosus. A bacterial population can comprise a bacterial strain of each of the following: Bifidobacterium adolescentis, Bifidobacterium pseudocatenulatum, and Lactobacillus rhamnosus. A bacterial population can comprise a bacterial strain of each of the following: Bifidobacterium breve, Bifidobacterium longum, and Bifidobacterium pseudocatenulatum. A bacterial population can comprise a bacterial strain of each of the following: Bifidobacterium breve, Bifidobacterium longum, and Lactobacillus rhamnosus. A bacterial population can comprise a bacterial strain of each of the following: Bifidobacterium breve, Bifidobacterium pseudocatenulatum, and Lactobacillus rhamnosus. A bacterial population can comprise a bacterial strain of each of the following: Bifidobacterium longum, Bifidobacterium pseudocatenulatum, and Lactobacillus rhamnosus. A bacterial population can comprise a bacterial strain of each of the following: Bifidobacterium bifidum, Lactobacillus plantarum, Bifidobacterium adolescentis, and Bifidobacterium breve. A bacterial population can comprise a bacterial strain of each of the following: Bifidobacterium bifidum, Lactobacillus plantarum, Bifidobacterium adolescentis, and Bifidobacterium longum. A bacterial population can comprise a bacterial strain of each of the following: Bifidobacterium bifidum, Lactobacillus plantarum, Bifidobacterium adolescentis, and Bifidobacterium pseudocatenulatum. A bacterial population can comprise a bacterial strain of each of the following: Bifidobacterium bifidum, Lactobacillus plantarum, Bifidobacterium adolescentis, and Lactobacillus rhamnosus. A bacterial population can comprise a bacterial strain of each of the following: Bifidobacterium bifidum, Lactobacillus plantarum, Bifidobacterium breve, and Bifidobacterium longum. A bacterial population can comprise a bacterial strain of each of the following: Bifidobacterium bifidum, Lactobacillus plantarum, Bifidobacterium breve, and Bifidobacterium pseudocatenulatum. A bacterial population can comprise a bacterial strain of each of the following: Bifidobacterium bifidum, Lactobacillus plantarum, Bifidobacterium breve, and Lactobacillus rhamnosus. A bacterial population can comprise a bacterial strain of each of the following: Bifidobacterium bifidum, Lactobacillus plantarum, Bifidobacterium longum, and Bifidobacterium pseudocatenulatum. A bacterial population can comprise a bacterial strain of each of the following: Bifidobacterium bifidum, Lactobacillus plantarum, Bifidobacterium longum, and Lactobacillus rhamnosus. A bacterial population can comprise a bacterial strain of each of the following: Bifidobacterium bifidum, Lactobacillus plantarum, Bifidobacterium pseudocatenulatum, and Lactobacillus rhamnosus. A bacterial population can comprise a bacterial strain of each of the following: Bifidobacterium bifidum, Bifidobacterium adolescentis, Bifidobacterium breve, and Bifidobacterium longum. A bacterial population can comprise a bacterial strain of each of the following: Bifidobacterium bifidum, Bifidobacterium adolescentis, Bifidobacterium breve, and Bifidobacterium pseudocatenulatum. A bacterial population can comprise a bacterial strain of each of the following: Bifidobacterium bifidum, Bifidobacterium adolescentis, Bifidobacterium breve, and Lactobacillus rhamnosus. A bacterial population can comprise a bacterial strain of each of the following: Bifidobacterium bifidum, Bifidobacterium adolescentis, Bifidobacterium longum, and Bifidobacterium pseudocatenulatum. A bacterial population can comprise a bacterial strain of each of the following: Bifidobacterium bifidum, Bifidobacterium adolescentis, Bifidobacterium longum, and Lactobacillus rhamnosus. A bacterial population can comprise a bacterial strain of each of the following: Bifidobacterium bifidum, Bifidobacterium adolescentis, Bifidobacterium pseudocatenulatum, and Lactobacillus rhamnosus. A bacterial population can comprise a bacterial strain of each of the following: Bifidobacterium bifidum, Bifidobacterium breve, Bifidobacterium longum, and Bifidobacterium pseudocatenulatum. A bacterial population can comprise a bacterial strain of each of the following: Bifidobacterium bifidum, Bifidobacterium breve, Bifidobacterium longum, and Lactobacillus rhamnosus. A bacterial population can comprise a bacterial strain of each of the following: Bifidobacterium bifidum, Bifidobacterium breve, Bifidobacterium pseudocatenulatum, and Lactobacillus rhamnosus. A bacterial population can comprise a bacterial strain of each of the following: Bifidobacterium bifidum, Bifidobacterium longum, Bifidobacterium pseudocatenulatum, and Lactobacillus rhamnosus. A bacterial population can comprise a bacterial strain of each of the following: Lactobacillus plantarum, Bifidobacterium adolescentis, Bifidobacterium breve, and Bifidobacterium longum. A bacterial population can comprise a bacterial strain of each of the following: Lactobacillus plantarum, Bifidobacterium adolescentis, Bifidobacterium breve, and Bifidobacterium pseudocatenulatum. A bacterial population can comprise a bacterial strain of each of the following: Lactobacillus plantarum, Bifidobacterium adolescentis, Bifidobacterium breve, and Lactobacillus rhamnosus. A bacterial population can comprise a bacterial strain of each of the following: Lactobacillus plantarum, Bifidobacterium adolescentis, Bifidobacterium longum, and Bifidobacterium pseudocatenulatum. A bacterial population can comprise a bacterial strain of each of the following: Lactobacillus plantarum, Bifidobacterium adolescentis, Bifidobacterium longum, and Lactobacillus rhamnosus. A bacterial population can comprise a bacterial strain of each of the following: Lactobacillus plantarum, Bifidobacterium adolescentis, Bifidobacterium pseudocatenulatum, and Lactobacillus rhamnosus. A bacterial population can comprise a bacterial strain of each of the following: Lactobacillus plantarum, Bifidobacterium breve, Bifidobacterium longum, and Bifidobacterium pseudocatenulatum. A bacterial population can comprise a bacterial strain of each of the following: Lactobacillus plantarum, Bifidobacterium breve, Bifidobacterium longum, and Lactobacillus rhamnosus. A bacterial population can comprise a bacterial strain of each of the following: Lactobacillus plantarum, Bifidobacterium breve, Bifidobacterium pseudocatenulatum, and Lactobacillus rhamnosus. A bacterial population can comprise a bacterial strain of each of the following: Lactobacillus plantarum, Bifidobacterium longum, Bifidobacterium pseudocatenulatum, and Lactobacillus rhamnosus. A bacterial population can comprise a bacterial strain of each of the following: Bifidobacterium adolescentis, Bifidobacterium breve, Bifidobacterium longum, and Bifidobacterium pseudocatenulatum. A bacterial population can comprise a bacterial strain of each of the following: Bifidobacterium adolescentis, Bifidobacterium breve, Bifidobacterium longum, and Lactobacillus rhamnosus. A bacterial population can comprise a bacterial strain of each of the following: Bifidobacterium adolescentis, Bifidobacterium breve, Bifidobacterium pseudocatenulatum, and Lactobacillus rhamnosus. A bacterial population can comprise a bacterial strain of each of the following: Bifidobacterium adolescentis, Bifidobacterium longum, Bifidobacterium pseudocatenulatum, and Lactobacillus rhamnosus. A bacterial population can comprise a bacterial strain of each of the following: Bifidobacterium breve, Bifidobacterium longum, Bifidobacterium pseudocatenulatum, and Lactobacillus rhamnosus.

A bacterial population can comprise ST31 and ST80. A bacterial population can comprise ST31 and ST65. A bacterial population can comprise ST31 and ST101. A bacterial population can comprise ST31 and ST56. A bacterial population can comprise ST31 and ST71. A bacterial population can comprise ST31 and ST19. A bacterial population can comprise ST31 and ST23. A bacterial population can comprise ST31 and ST81. A bacterial population can comprise ST31 and ST119. A bacterial population can comprise ST31 and ST37. A bacterial population can comprise ST31 and ST66. A bacterial population can comprise ST31 and ST116. A bacterial population can comprise ST80 and ST65. A bacterial population can comprise ST80 and ST101. A bacterial population can comprise ST80 and ST56. A bacterial population can comprise ST80 and ST71. A bacterial population can comprise ST80 and ST19. A bacterial population can comprise ST80 and ST23. A bacterial population can comprise ST80 and ST81. A bacterial population can comprise ST80 and ST119. A bacterial population can comprise ST80 and ST37. A bacterial population can comprise ST80 and ST66. A bacterial population can comprise ST80 and ST116. A bacterial population can comprise ST65 and ST101. A bacterial population can comprise ST65 and ST56. A bacterial population can comprise ST65 and ST71. A bacterial population can comprise ST65 and ST19. A bacterial population can comprise ST65 and ST23. A bacterial population can comprise ST65 and ST81. A bacterial population can comprise ST65 and ST119. A bacterial population can comprise ST65 and ST37. A bacterial population can comprise ST65 and ST66. A bacterial population can comprise ST65 and ST116. A bacterial population can comprise ST101 and ST56. A bacterial population can comprise ST101 and ST71. A bacterial population can comprise ST101 and ST19. A bacterial population can comprise ST101 and ST23. A bacterial population can comprise ST101 and ST81. A bacterial population can comprise ST101 and ST119. A bacterial population can comprise ST101 and ST37. A bacterial population can comprise ST101 and ST66. A bacterial population can comprise ST101 and ST116. A bacterial population can comprise ST56 and ST71. A bacterial population can comprise ST56 and ST19. A bacterial population can comprise ST56 and ST23. A bacterial population can comprise ST56 and ST81. A bacterial population can comprise ST56 and ST119. A bacterial population can comprise ST56 and ST37. A bacterial population can comprise ST56 and ST66. A bacterial population can comprise ST56 and ST116. A bacterial population can comprise ST71 and ST19. A bacterial population can comprise ST71 and ST23. A bacterial population can comprise ST71 and ST81. A bacterial population can comprise ST71 and ST119. A bacterial population can comprise ST71 and ST37. A bacterial population can comprise ST71 and ST66. A bacterial population can comprise ST71 and ST116. A bacterial population can comprise ST19 and ST23. A bacterial population can comprise ST19 and ST81. A bacterial population can comprise ST19 and ST119. A bacterial population can comprise ST19 and ST37. A bacterial population can comprise ST19 and ST66. A bacterial population can comprise ST19 and ST116. A bacterial population can comprise ST23 and ST81. A bacterial population can comprise ST23 and ST119. A bacterial population can comprise ST23 and ST37. A bacterial population can comprise ST23 and ST66. A bacterial population can comprise ST23 and ST116. A bacterial population can comprise ST81 and ST119. A bacterial population can comprise ST81 and ST37. A bacterial population can comprise ST81 and ST66. A bacterial population can comprise ST81 and ST116. A bacterial population can comprise ST119 and ST37. A bacterial population can comprise ST119 and ST66. A bacterial population can comprise ST119 and ST116. A bacterial population can comprise ST37 and ST66. A bacterial population can comprise ST37 and ST116. A bacterial population can comprise ST66 and ST116. A bacterial population can comprise ST31, ST80, and ST65. A bacterial population can comprise ST31, ST80, and ST101. A bacterial population can comprise ST31, ST80, and ST56. A bacterial population can comprise ST31, ST80, and ST71. A bacterial population can comprise ST31, ST80, and ST19. A bacterial population can comprise ST31, ST80, and ST23. A bacterial population can comprise ST31, ST80, and ST81. A bacterial population can comprise ST31, ST80, and ST119. A bacterial population can comprise ST31, ST80, and ST37. A bacterial population can comprise ST31, ST80, and ST66. A bacterial population can comprise ST31, ST80, and ST116. A bacterial population can comprise ST31, ST65, and ST101. A bacterial population can comprise ST31, ST65, and ST56. A bacterial population can comprise ST31, ST65, and ST71. A bacterial population can comprise ST31, ST65, and ST19. A bacterial population can comprise ST31, ST65, and ST23. A bacterial population can comprise ST31, ST65, and ST81. A bacterial population can comprise ST31, ST65, and ST119. A bacterial population can comprise ST31, ST65, and ST37. A bacterial population can comprise ST31, ST65, and ST66. A bacterial population can comprise ST31, ST65, and ST116. A bacterial population can comprise ST31, ST101, and ST56. A bacterial population can comprise ST31, ST101, and ST71. A bacterial population can comprise ST31, ST101, and ST19. A bacterial population can comprise ST31, ST101, and ST23. A bacterial population can comprise ST31, ST101, and ST81. A bacterial population can comprise ST31, ST101, and ST119. A bacterial population can comprise ST31, ST101, and ST37. A bacterial population can comprise ST31, ST101, and ST66. A bacterial population can comprise ST31, ST101, and ST116. A bacterial population can comprise ST31, ST56, and ST71. A bacterial population can comprise ST31, ST56, and ST19. A bacterial population can comprise ST31, ST56, and ST23. A bacterial population can comprise ST31, ST56, and ST81. A bacterial population can comprise ST31, ST56, and ST119. A bacterial population can comprise ST31, ST56, and ST37. A bacterial population can comprise ST31, ST56, and ST66. A bacterial population can comprise ST31, ST56, and ST116. A bacterial population can comprise ST31, ST71, and ST19. A bacterial population can comprise ST31, ST71, and ST23. A bacterial population can comprise ST31, ST71, and ST81. A bacterial population can comprise ST31, ST71, and ST119. A bacterial population can comprise ST31, ST71, and ST37. A bacterial population can comprise ST31, ST71, and ST66. A bacterial population can comprise ST31, ST71, and ST116. A bacterial population can comprise ST31, ST19, and ST23. A bacterial population can comprise ST31, ST19, and ST81. A bacterial population can comprise ST31, ST19, and ST119. A bacterial population can comprise ST31, ST19, and ST37. A bacterial population can comprise ST31, ST19, and ST66. A bacterial population can comprise ST31, ST19, and ST116. A bacterial population can comprise ST31, ST23, and ST81. A bacterial population can comprise ST31, ST23, and ST119. A bacterial population can comprise ST31, ST23, and ST37. A bacterial population can comprise ST31, ST23, and ST66. A bacterial population can comprise ST31, ST23, and ST116. A bacterial population can comprise ST31, ST81, and ST119. A bacterial population can comprise ST31, ST81, and ST37. A bacterial population can comprise ST31, ST81, and ST66. A bacterial population can comprise ST31, ST81, and ST116. A bacterial population can comprise ST31, ST119, and ST37. A bacterial population can comprise ST31, ST119, and ST66. A bacterial population can comprise ST31, ST119, and ST116. A bacterial population can comprise ST31, ST37, and ST66. A bacterial population can comprise ST31, ST37, and ST116. A bacterial population can comprise ST31, ST66, and ST116. A bacterial population can comprise ST80, ST65, and ST101. A bacterial population can comprise ST80, ST65, and ST56. A bacterial population can comprise ST80, ST65, and ST71. A bacterial population can comprise ST80, ST65, and ST19. A bacterial population can comprise ST80, ST65, and ST23. A bacterial population can comprise ST80, ST65, and ST81. A bacterial population can comprise ST80, ST65, and ST119. A bacterial population can comprise ST80, ST65, and ST37. A bacterial population can comprise ST80, ST65, and ST66. A bacterial population can comprise ST80, ST65, and ST116. A bacterial population can comprise ST80, ST101, and ST56. A bacterial population can comprise ST80, ST101, and ST71. A bacterial population can comprise ST80, ST101, and ST19. A bacterial population can comprise ST80, ST101, and ST23. A bacterial population can comprise ST80, ST101, and ST81. A bacterial population can comprise ST80, ST101, and ST119. A bacterial population can comprise ST80, ST101, and ST37. A bacterial population can comprise ST80, ST101, and ST66. A bacterial population can comprise ST80, ST101, and ST116. A bacterial population can comprise ST80, ST56, and ST71. A bacterial population can comprise ST80, ST56, and ST19. A bacterial population can comprise ST80, ST56, and ST23. A bacterial population can comprise ST80, ST56, and ST81. A bacterial population can comprise ST80, ST56, and ST119. A bacterial population can comprise ST80, ST56, and ST37. A bacterial population can comprise ST80, ST56, and ST66. A bacterial population can comprise ST80, ST56, and ST116. A bacterial population can comprise ST80, ST71, and ST19. A bacterial population can comprise ST80, ST71, and ST23. A bacterial population can comprise ST80, ST71, and ST81. A bacterial population can comprise ST80, ST71, and ST119. A bacterial population can comprise ST80, ST71, and ST37. A bacterial population can comprise ST80, ST71, and ST66. A bacterial population can comprise ST80, ST71, and ST116. A bacterial population can comprise ST80, ST19, and ST23. A bacterial population can comprise ST80, ST19, and ST81. A bacterial population can comprise ST80, ST19, and ST119. A bacterial population can comprise ST80, ST19, and ST37. A bacterial population can comprise ST80, ST19, and ST66. A bacterial population can comprise ST80, ST19, and ST116. A bacterial population can comprise ST80, ST23, and ST81. A bacterial population can comprise ST80, ST23, and ST119. A bacterial population can comprise ST80, ST23, and ST37. A bacterial population can comprise ST80, ST23, and ST66. A bacterial population can comprise ST80, ST23, and ST116. A bacterial population can comprise ST80, ST81, and ST119. A bacterial population can comprise ST80, ST81, and ST37. A bacterial population can comprise ST80, ST81, and ST66. A bacterial population can comprise ST80, ST81, and ST116. A bacterial population can comprise ST80, ST119, and ST37. A bacterial population can comprise ST80, ST119, and ST66. A bacterial population can comprise ST80, ST119, and ST116. A bacterial population can comprise ST80, ST37, and ST66. A bacterial population can comprise ST80, ST37, and ST116. A bacterial population can comprise ST80, ST66, and ST116. A bacterial population can comprise ST65, ST101, and ST56. A bacterial population can comprise ST65, ST101, and ST71. A bacterial population can comprise ST65, ST101, and ST19. A bacterial population can comprise ST65, ST101, and ST23. A bacterial population can comprise ST65, ST101, and ST81. A bacterial population can comprise ST65, ST101, and ST119. A bacterial population can comprise ST65, ST101, and ST37. A bacterial population can comprise ST65, ST101, and ST66. A bacterial population can comprise ST65, ST101, and ST116. A bacterial population can comprise ST65, ST56, and ST71. A bacterial population can comprise ST65, ST56, and ST19. A bacterial population can comprise ST65, ST56, and ST23. A bacterial population can comprise ST65, ST56, and ST81. A bacterial population can comprise ST65, ST56, and ST119. A bacterial population can comprise ST65, ST56, and ST37. A bacterial population can comprise ST65, ST56, and ST66. A bacterial population can comprise ST65, ST56, and ST116. A bacterial population can comprise ST65, ST71, and ST19. A bacterial population can comprise ST65, ST71, and ST23. A bacterial population can comprise ST65, ST71, and ST81. A bacterial population can comprise ST65, ST71, and ST119. A bacterial population can comprise ST65, ST71, and ST37. A bacterial population can comprise ST65, ST71, and ST66. A bacterial population can comprise ST65, ST71, and ST116. A bacterial population can comprise ST65, ST19, and ST23. A bacterial population can comprise ST65, ST19, and ST81. A bacterial population can comprise ST65, ST19, and ST119. A bacterial population can comprise ST65, ST19, and ST37. A bacterial population can comprise ST65, ST19, and ST66. A bacterial population can comprise ST65, ST19, and ST116. A bacterial population can comprise ST65, ST23, and ST81. A bacterial population can comprise ST65, ST23, and ST119. A bacterial population can comprise ST65, ST23, and ST37. A bacterial population can comprise ST65, ST23, and ST66. A bacterial population can comprise ST65, ST23, and ST116. A bacterial population can comprise ST65, ST81, and ST119. A bacterial population can comprise ST65, ST81, and ST37. A bacterial population can comprise ST65, ST81, and ST66. A bacterial population can comprise ST65, ST81, and ST116. A bacterial population can comprise ST65, ST119, and ST37. A bacterial population can comprise ST65, ST119, and ST66. A bacterial population can comprise ST65, ST119, and ST116. A bacterial population can comprise ST65, ST37, and ST66. A bacterial population can comprise ST65, ST37, and ST116. A bacterial population can comprise ST65, ST66, and ST116. A bacterial population can comprise ST101, ST56, and ST71. A bacterial population can comprise ST101, ST56, and ST19. A bacterial population can comprise ST101, ST56, and ST23. A bacterial population can comprise ST101, ST56, and ST81. A bacterial population can comprise ST101, ST56, and ST119. A bacterial population can comprise ST101, ST56, and ST37. A bacterial population can comprise ST101, ST56, and ST66. A bacterial population can comprise ST101, ST56, and ST116. A bacterial population can comprise ST101, ST71, and ST19. A bacterial population can comprise ST101, ST71, and ST23. A bacterial population can comprise ST101, ST71, and ST81. A bacterial population can comprise ST101, ST71, and ST119. A bacterial population can comprise ST101, ST71, and ST37. A bacterial population can comprise ST101, ST71, and ST66. A bacterial population can comprise ST101, ST71, and ST116. A bacterial population can comprise ST101, ST19, and ST23. A bacterial population can comprise ST101, ST19, and ST81. A bacterial population can comprise ST101, ST19, and ST119. A bacterial population can comprise ST101, ST19, and ST37. A bacterial population can comprise ST101, ST19, and ST66. A bacterial population can comprise ST101, ST19, and ST116. A bacterial population can comprise ST101, ST23, and ST81. A bacterial population can comprise ST101, ST23, and ST119. A bacterial population can comprise ST101, ST23, and ST37. A bacterial population can comprise ST101, ST23, and ST66. A bacterial population can comprise ST101, ST23, and ST116. A bacterial population can comprise ST101, ST81, and ST119. A bacterial population can comprise ST101, ST81, and ST37. A bacterial population can comprise ST101, ST81, and ST66. A bacterial population can comprise ST101, ST81, and ST116. A bacterial population can comprise ST101, ST119, and ST37. A bacterial population can comprise ST101, ST119, and ST66. A bacterial population can comprise ST101, ST119, and ST116. A bacterial population can comprise ST101, ST37, and ST66. A bacterial population can comprise ST101, ST37, and ST116. A bacterial population can comprise ST101, ST66, and ST116. A bacterial population can comprise ST56, ST71, and ST19. A bacterial population can comprise ST56, ST71, and ST23. A bacterial population can comprise ST56, ST71, and ST81. A bacterial population can comprise ST56, ST71, and ST119. A bacterial population can comprise ST56, ST71, and ST37. A bacterial population can comprise ST56, ST71, and ST66. A bacterial population can comprise ST56, ST71, and ST116. A bacterial population can comprise ST56, ST19, and ST23. A bacterial population can comprise ST56, ST19, and ST81. A bacterial population can comprise ST56, ST19, and ST119. A bacterial population can comprise ST56, ST19, and ST37. A bacterial population can comprise ST56, ST19, and ST66. A bacterial population can comprise ST56, ST19, and ST116. A bacterial population can comprise ST56, ST23, and ST81. A bacterial population can comprise ST56, ST23, and ST119. A bacterial population can comprise ST56, ST23, and ST37. A bacterial population can comprise ST56, ST23, and ST66. A bacterial population can comprise ST56, ST23, and ST116. A bacterial population can comprise ST56, ST81, and ST119. A bacterial population can comprise ST56, ST81, and ST37. A bacterial population can comprise ST56, ST81, and ST66. A bacterial population can comprise ST56, ST81, and ST116. A bacterial population can comprise ST56, ST119, and ST37. A bacterial population can comprise ST56, ST119, and ST66. A bacterial population can comprise ST56, ST119, and ST116. A bacterial population can comprise ST56, ST37, and ST66. A bacterial population can comprise ST56, ST37, and ST116. A bacterial population can comprise ST56, ST66, and ST116. A bacterial population can comprise ST71, ST19, and ST23. A bacterial population can comprise ST71, ST19, and ST81. A bacterial population can comprise ST71, ST19, and ST119. A bacterial population can comprise ST71, ST19, and ST37. A bacterial population can comprise ST71, ST19, and ST66. A bacterial population can comprise ST71, ST19, and ST116. A bacterial population can comprise ST71, ST23, and ST81. A bacterial population can comprise ST71, ST23, and ST119. A bacterial population can comprise ST71, ST23, and ST37. A bacterial population can comprise ST71, ST23, and ST66. A bacterial population can comprise ST71, ST23, and ST116. A bacterial population can comprise ST71, ST81, and ST119. A bacterial population can comprise ST71, ST81, and ST37. A bacterial population can comprise ST71, ST81, and ST66. A bacterial population can comprise ST71, ST81, and ST116. A bacterial population can comprise ST71, ST119, and ST37. A bacterial population can comprise ST71, ST119, and ST66. A bacterial population can comprise ST71, ST119, and ST116. A bacterial population can comprise ST71, ST37, and ST66. A bacterial population can comprise ST71, ST37, and ST116. A bacterial population can comprise ST71, ST66, and ST116. A bacterial population can comprise ST19, ST23, and ST81. A bacterial population can comprise ST19, ST23, and ST119. A bacterial population can comprise ST19, ST23, and ST37. A bacterial population can comprise ST19, ST23, and ST66. A bacterial population can comprise ST19, ST23, and ST116. A bacterial population can comprise ST19, ST81, and ST119. A bacterial population can comprise ST19, ST81, and ST37. A bacterial population can comprise ST19, ST81, and ST66. A bacterial population can comprise ST19, ST81, and ST116. A bacterial population can comprise ST19, ST119, and ST37. A bacterial population can comprise ST19, ST119, and ST66. A bacterial population can comprise ST19, ST119, and ST116. A bacterial population can comprise ST19, ST37, and ST66. A bacterial population can comprise ST19, ST37, and ST116. A bacterial population can comprise ST19, ST66, and ST116. A bacterial population can comprise ST23, ST81, and ST119. A bacterial population can comprise ST23, ST81, and ST37. A bacterial population can comprise ST23, ST81, and ST66. A bacterial population can comprise ST23, ST81, and ST116. A bacterial population can comprise ST23, ST119, and ST37. A bacterial population can comprise ST23, ST119, and ST66. A bacterial population can comprise ST23, ST119, and ST116. A bacterial population can comprise ST23, ST37, and ST66. A bacterial population can comprise ST23, ST37, and ST116. A bacterial population can comprise ST23, ST66, and ST116. A bacterial population can comprise ST81, ST119, and ST37. A bacterial population can comprise ST81, ST119, and ST66. A bacterial population can comprise ST81, ST119, and ST116. A bacterial population can comprise ST81, ST37, and ST66. A bacterial population can comprise ST81, ST37, and ST116. A bacterial population can comprise ST81, ST66, and ST116. A bacterial population can comprise ST119, ST37, and ST66. A bacterial population can comprise ST119, ST37, and ST116. A bacterial population can comprise ST119, ST66, and ST116. A bacterial population can comprise ST37, ST66, and ST116. A bacterial population can comprise ST31, ST80, ST65, and ST101. A bacterial population can comprise ST31, ST80, ST65, and ST56. A bacterial population can comprise ST31, ST80, ST65, and ST71. A bacterial population can comprise ST31, ST80, ST65, and ST19. A bacterial population can comprise ST31, ST80, ST65, and ST23. A bacterial population can comprise ST31, ST80, ST65, and ST81. A bacterial population can comprise ST31, ST80, ST65, and ST119. A bacterial population can comprise ST31, ST80, ST65, and ST37. A bacterial population can comprise ST31, ST80, ST65, and ST66. A bacterial population can comprise ST31, ST80, ST65, and ST116. A bacterial population can comprise ST31, ST80, ST101, and ST56. A bacterial population can comprise ST31, ST80, ST101, and ST71. A bacterial population can comprise ST31, ST80, ST101, and ST19. A bacterial population can comprise ST31, ST80, ST101, and ST23. A bacterial population can comprise ST31, ST80, ST101, and ST81. A bacterial population can comprise ST31, ST80, ST101, and ST119. A bacterial population can comprise ST31, ST80, ST101, and ST37. A bacterial population can comprise ST31, ST80, ST101, and ST66. A bacterial population can comprise ST31, ST80, ST101, and ST116. A bacterial population can comprise ST31, ST80, ST56, and ST71. A bacterial population can comprise ST31, ST80, ST56, and ST19. A bacterial population can comprise ST31, ST80, ST56, and ST23. A bacterial population can comprise ST31, ST80, ST56, and ST81. A bacterial population can comprise ST31, ST80, ST56, and ST119. A bacterial population can comprise ST31, ST80, ST56, and ST37. A bacterial population can comprise ST31, ST80, ST56, and ST66. A bacterial population can comprise ST31, ST80, ST56, and ST116. A bacterial population can comprise ST31, ST80, ST71, and ST19. A bacterial population can comprise ST31, ST80, ST71, and ST23. A bacterial population can comprise ST31, ST80, ST71, and ST81. A bacterial population can comprise ST31, ST80, ST71, and ST119. A bacterial population can comprise ST31, ST80, ST71, and ST37. A bacterial population can comprise ST31, ST80, ST71, and ST66. A bacterial population can comprise ST31, ST80, ST71, and ST116. A bacterial population can comprise ST31, ST80, ST19, and ST23. A bacterial population can comprise ST31, ST80, ST19, and ST81. A bacterial population can comprise ST31, ST80, ST19, and ST119. A bacterial population can comprise ST31, ST80, ST19, and ST37. A bacterial population can comprise ST31, ST80, ST19, and ST66. A bacterial population can comprise ST31, ST80, ST19, and ST116. A bacterial population can comprise ST31, ST80, ST23, and ST81. A bacterial population can comprise ST31, ST80, ST23, and ST119. A bacterial population can comprise ST31, ST80, ST23, and ST37. A bacterial population can comprise ST31, ST80, ST23, and ST66. A bacterial population can comprise ST31, ST80, ST23, and ST116. A bacterial population can comprise ST31, ST80, ST81, and ST119. A bacterial population can comprise ST31, ST80, ST81, and ST37. A bacterial population can comprise ST31, ST80, ST81, and ST66. A bacterial population can comprise ST31, ST80, ST81, and ST116. A bacterial population can comprise ST31, ST80, ST119, and ST37. A bacterial population can comprise ST31, ST80, ST119, and ST66. A bacterial population can comprise ST31, ST80, ST119, and ST116. A bacterial population can comprise ST31, ST80, ST37, and ST66. A bacterial population can comprise ST31, ST80, ST37, and ST116. A bacterial population can comprise ST31, ST80, ST66, and ST116. A bacterial population can comprise ST31, ST65, ST101, and ST56. A bacterial population can comprise ST31, ST65, ST101, and ST71. A bacterial population can comprise ST31, ST65, ST101, and ST19. A bacterial population can comprise ST31, ST65, ST101, and ST23. A bacterial population can comprise ST31, ST65, ST101, and ST81. A bacterial population can comprise ST31, ST65, ST101, and ST119. A bacterial population can comprise ST31, ST65, ST101, and ST37. A bacterial population can comprise ST31, ST65, ST101, and ST66. A bacterial population can comprise ST31, ST65, ST101, and ST116. A bacterial population can comprise ST31, ST65, ST56, and ST71. A bacterial population can comprise ST31, ST65, ST56, and ST19. A bacterial population can comprise ST31, ST65, ST56, and ST23. A bacterial population can comprise ST31, ST65, ST56, and ST81. A bacterial population can comprise ST31, ST65, ST56, and ST119. A bacterial population can comprise ST31, ST65, ST56, and ST37. A bacterial population can comprise ST31, ST65, ST56, and ST66. A bacterial population can comprise ST31, ST65, ST56, and ST116. A bacterial population can comprise ST31, ST65, ST71, and ST19. A bacterial population can comprise ST31, ST65, ST71, and ST23. A bacterial population can comprise ST31, ST65, ST71, and ST81. A bacterial population can comprise ST31, ST65, ST71, and ST119. A bacterial population can comprise ST31, ST65, ST71, and ST37. A bacterial population can comprise ST31, ST65, ST71, and ST66. A bacterial population can comprise ST31, ST65, ST71, and ST116. A bacterial population can comprise ST31, ST65, ST19, and ST23. A bacterial population can comprise ST31, ST65, ST19, and ST81. A bacterial population can comprise ST31, ST65, ST19, and ST119. A bacterial population can comprise ST31, ST65, ST19, and ST37. A bacterial population can comprise ST31, ST65, ST19, and ST66. A bacterial population can comprise ST31, ST65, ST19, and ST116. A bacterial population can comprise ST31, ST65, ST23, and ST81. A bacterial population can comprise ST31, ST65, ST23, and ST119. A bacterial population can comprise ST31, ST65, ST23, and ST37. A bacterial population can comprise ST31, ST65, ST23, and ST66. A bacterial population can comprise ST31, ST65, ST23, and ST116. A bacterial population can comprise ST31, ST65, ST81, and ST119. A bacterial population can comprise ST31, ST65, ST81, and ST37. A bacterial population can comprise ST31, ST65, ST81, and ST66. A bacterial population can comprise ST31, ST65, ST81, and ST116. A bacterial population can comprise ST31, ST65, ST119, and ST37. A bacterial population can comprise ST31, ST65, ST119, and ST66. A bacterial population can comprise ST31, ST65, ST119, and ST116. A bacterial population can comprise ST31, ST65, ST37, and ST66. A bacterial population can comprise ST31, ST65, ST37, and ST116. A bacterial population can comprise ST31, ST65, ST66, and ST116. A bacterial population can comprise ST31, ST101, ST56, and ST71. A bacterial population can comprise ST31, ST101, ST56, and ST19. A bacterial population can comprise ST31, ST101, ST56, and ST23. A bacterial population can comprise ST31, ST101, ST56, and ST81. A bacterial population can comprise ST31, ST101, ST56, and ST119. A bacterial population can comprise ST31, ST101, ST56, and ST37. A bacterial population can comprise ST31, ST101, ST56, and ST66. A bacterial population can comprise ST31, ST101, ST56, and ST116. A bacterial population can comprise ST31, ST101, ST71, and ST19. A bacterial population can comprise ST31, ST101, ST71, and ST23. A bacterial population can comprise ST31, ST101, ST71, and ST81. A bacterial population can comprise ST31, ST101, ST71, and ST119. A bacterial population can comprise ST31, ST101, ST71, and ST37. A bacterial population can comprise ST31, ST101, ST71, and ST66. A bacterial population can comprise ST31, ST101, ST71, and ST116. A bacterial population can comprise ST31, ST101, ST19, and ST23. A bacterial population can comprise ST31, ST101, ST19, and ST81. A bacterial population can comprise ST31, ST101, ST19, and ST119. A bacterial population can comprise ST31, ST101, ST19, and ST37. A bacterial population can comprise ST31, ST101, ST19, and ST66. A bacterial population can comprise ST31, ST101, ST19, and ST116. A bacterial population can comprise ST31, ST101, ST23, and ST81. A bacterial population can comprise ST31, ST101, ST23, and ST119. A bacterial population can comprise ST31, ST101, ST23, and ST37. A bacterial population can comprise ST31, ST101, ST23, and ST66. A bacterial population can comprise ST31, ST101, ST23, and ST116. A bacterial population can comprise ST31, ST101, ST81, and ST119. A bacterial population can comprise ST31, ST101, ST81, and ST37. A bacterial population can comprise ST31, ST101, ST81, and ST66. A bacterial population can comprise ST31, ST101, ST81, and ST116. A bacterial population can comprise ST31, ST101, ST119, and ST37. A bacterial population can comprise ST31, ST101, ST119, and ST66. A bacterial population can comprise ST31, ST101, ST119, and ST116. A bacterial population can comprise ST31, ST101, ST37, and ST66. A bacterial population can comprise ST31, ST101, ST37, and ST116. A bacterial population can comprise ST31, ST101, ST66, and ST116. A bacterial population can comprise ST31, ST56, ST71, and ST19. A bacterial population can comprise ST31, ST56, ST71, and ST23. A bacterial population can comprise ST31, ST56, ST71, and ST81. A bacterial population can comprise ST31, ST56, ST71, and ST119. A bacterial population can comprise ST31, ST56, ST71, and ST37. A bacterial population can comprise ST31, ST56, ST71, and ST66. A bacterial population can comprise ST31, ST56, ST71, and ST116. A bacterial population can comprise ST31, ST56, ST19, and ST23. A bacterial population can comprise ST31, ST56, ST19, and ST81. A bacterial population can comprise ST31, ST56, ST19, and ST119. A bacterial population can comprise ST31, ST56, ST19, and ST37. A bacterial population can comprise ST31, ST56, ST19, and ST66. A bacterial population can comprise ST31, ST56, ST19, and ST116. A bacterial population can comprise ST31, ST56, ST23, and ST81. A bacterial population can comprise ST31, ST56, ST23, and ST119. A bacterial population can comprise ST31, ST56, ST23, and ST37. A bacterial population can comprise ST31, ST56, ST23, and ST66. A bacterial population can comprise ST31, ST56, ST23, and ST116. A bacterial population can comprise ST31, ST56, ST81, and ST119. A bacterial population can comprise ST31, ST56, ST81, and ST37. A bacterial population can comprise ST31, ST56, ST81, and ST66. A bacterial population can comprise ST31, ST56, ST81, and ST116. A bacterial population can comprise ST31, ST56, ST119, and ST37. A bacterial population can comprise ST31, ST56, ST119, and ST66. A bacterial population can comprise ST31, ST56, ST119, and ST116. A bacterial population can comprise ST31, ST56, ST37, and ST66. A bacterial population can comprise ST31, ST56, ST37, and ST116. A bacterial population can comprise ST31, ST56, ST66, and ST116. A bacterial population can comprise ST31, ST71, ST19, and ST23. A bacterial population can comprise ST31, ST71, ST19, and ST81. A bacterial population can comprise ST31, ST71, ST19, and ST119. A bacterial population can comprise ST31, ST71, ST19, and ST37. A bacterial population can comprise ST31, ST71, ST19, and ST66. A bacterial population can comprise ST31, ST71, ST19, and ST116. A bacterial population can comprise ST31, ST71, ST23, and ST81. A bacterial population can comprise ST31, ST71, ST23, and ST119. A bacterial population can comprise ST31, ST71, ST23, and ST37. A bacterial population can comprise ST31, ST71, ST23, and ST66. A bacterial population can comprise ST31, ST71, ST23, and ST116. A bacterial population can comprise ST31, ST71, ST81, and ST119. A bacterial population can comprise ST31, ST71, ST81, and ST37. A bacterial population can comprise ST31, ST71, ST81, and ST66. A bacterial population can comprise ST31, ST71, ST81, and ST116. A bacterial population can comprise ST31, ST71, ST119, and ST37. A bacterial population can comprise ST31, ST71, ST119, and ST66. A bacterial population can comprise ST31, ST71, ST119, and ST116. A bacterial population can comprise ST31, ST71, ST37, and ST66. A bacterial population can comprise ST31, ST71, ST37, and ST116. A bacterial population can comprise ST31, ST71, ST66, and ST116. A bacterial population can comprise ST31, ST19, ST23, and ST81. A bacterial population can comprise ST31, ST19, ST23, and ST119. A bacterial population can comprise ST31, ST19, ST23, and ST37. A bacterial population can comprise ST31, ST19, ST23, and ST66. A bacterial population can comprise ST31, ST19, ST23, and ST116. A bacterial population can comprise ST31, ST19, ST81, and ST119. A bacterial population can comprise ST31, ST19, ST81, and ST37. A bacterial population can comprise ST31, ST19, ST81, and ST66. A bacterial population can comprise ST31, ST19, ST81, and ST116. A bacterial population can comprise ST31, ST19, ST119, and ST37. A bacterial population can comprise ST31, ST19, ST119, and ST66. A bacterial population can comprise ST31, ST19, ST119, and ST116. A bacterial population can comprise ST31, ST19, ST37, and ST66. A bacterial population can comprise ST31, ST19, ST37, and ST116. A bacterial population can comprise ST31, ST19, ST66, and ST116. A bacterial population can comprise ST31, ST23, ST81, and ST119. A bacterial population can comprise ST31, ST23, ST81, and ST37. A bacterial population can comprise ST31, ST23, ST81, and ST66. A bacterial population can comprise ST31, ST23, ST81, and ST116. A bacterial population can comprise ST31, ST23, ST119, and ST37. A bacterial population can comprise ST31, ST23, ST119, and ST66. A bacterial population can comprise ST31, ST23, ST119, and ST116. A bacterial population can comprise ST31, ST23, ST37, and ST66. A bacterial population can comprise ST31, ST23, ST37, and ST116. A bacterial population can comprise ST31, ST23, ST66, and ST116. A bacterial population can comprise ST31, ST81, ST119, and ST37. A bacterial population can comprise ST31, ST81, ST119, and ST66. A bacterial population can comprise ST31, ST81, ST119, and ST116. A bacterial population can comprise ST31, ST81, ST37, and ST66. A bacterial population can comprise ST31, ST81, ST37, and ST116. A bacterial population can comprise ST31, ST81, ST66, and ST116. A bacterial population can comprise ST31, ST119, ST37, and ST66. A bacterial population can comprise ST31, ST119, ST37, and ST116. A bacterial population can comprise ST31, ST119, ST66, and ST116. A bacterial population can comprise ST31, ST37, ST66, and ST116. A bacterial population can comprise ST80, ST65, ST101, and ST56. A bacterial population can comprise ST80, ST65, ST101, and ST71. A bacterial population can comprise ST80, ST65, ST101, and ST19. A bacterial population can comprise ST80, ST65, ST101, and ST23. A bacterial population can comprise ST80, ST65, ST101, and ST81. A bacterial population can comprise ST80, ST65, ST101, and ST119. A bacterial population can comprise ST80, ST65, ST101, and ST37. A bacterial population can comprise ST80, ST65, ST101, and ST66. A bacterial population can comprise ST80, ST65, ST101, and ST116. A bacterial population can comprise ST80, ST65, ST56, and ST71. A bacterial population can comprise ST80, ST65, ST56, and ST19. A bacterial population can comprise ST80, ST65, ST56, and ST23. A bacterial population can comprise ST80, ST65, ST56, and ST81. A bacterial population can comprise ST80, ST65, ST56, and ST119. A bacterial population can comprise ST80, ST65, ST56, and ST37. A bacterial population can comprise ST80, ST65, ST56, and ST66. A bacterial population can comprise ST80, ST65, ST56, and ST116. A bacterial population can comprise ST80, ST65, ST71, and ST19. A bacterial population can comprise ST80, ST65, ST71, and ST23. A bacterial population can comprise ST80, ST65, ST71, and ST81. A bacterial population can comprise ST80, ST65, ST71, and ST119. A bacterial population can comprise ST80, ST65, ST71, and ST37. A bacterial population can comprise ST80, ST65, ST71, and ST66. A bacterial population can comprise ST80, ST65, ST71, and ST116. A bacterial population can comprise ST80, ST65, ST19, and ST23. A bacterial population can comprise ST80, ST65, ST19, and ST81. A bacterial population can comprise ST80, ST65, ST19, and ST119. A bacterial population can comprise ST80, ST65, ST19, and ST37. A bacterial population can comprise ST80, ST65, ST19, and ST66. A bacterial population can comprise ST80, ST65, ST19, and ST116. A bacterial population can comprise ST80, ST65, ST23, and ST81. A bacterial population can comprise ST80, ST65, ST23, and ST119. A bacterial population can comprise ST80, ST65, ST23, and ST37. A bacterial population can comprise ST80, ST65, ST23, and ST66. A bacterial population can comprise ST80, ST65, ST23, and ST116. A bacterial population can comprise ST80, ST65, ST81, and ST119. A bacterial population can comprise ST80, ST65, ST81, and ST37. A bacterial population can comprise ST80, ST65, ST81, and ST66. A bacterial population can comprise ST80, ST65, ST81, and ST116. A bacterial population can comprise ST80, ST65, ST119, and ST37. A bacterial population can comprise ST80, ST65, ST119, and ST66. A bacterial population can comprise ST80, ST65, ST119, and ST116. A bacterial population can comprise ST80, ST65, ST37, and ST66. A bacterial population can comprise ST80, ST65, ST37, and ST116. A bacterial population can comprise ST80, ST65, ST66, and ST116. A bacterial population can comprise ST80, ST101, ST56, and ST71. A bacterial population can comprise ST80, ST101, ST56, and ST19. A bacterial population can comprise ST80, ST101, ST56, and ST23. A bacterial population can comprise ST80, ST101, ST56, and ST81. A bacterial population can comprise ST80, ST101, ST56, and ST119. A bacterial population can comprise ST80, ST101, ST56, and ST37. A bacterial population can comprise ST80, ST101, ST56, and ST66. A bacterial population can comprise ST80, ST101, ST56, and ST116. A bacterial population can comprise ST80, ST101, ST71, and ST19. A bacterial population can comprise ST80, ST101, ST71, and ST23. A bacterial population can comprise ST80, ST101, ST71, and ST81. A bacterial population can comprise ST80, ST101, ST71, and ST119. A bacterial population can comprise ST80, ST101, ST71, and ST37. A bacterial population can comprise ST80, ST101, ST71, and ST66. A bacterial population can comprise ST80, ST101, ST71, and ST116. A bacterial population can comprise ST80, ST101, ST19, and ST23. A bacterial population can comprise ST80, ST101, ST19, and ST81. A bacterial population can comprise ST80, ST101, ST19, and ST119. A bacterial population can comprise ST80, ST101, ST19, and ST37. A bacterial population can comprise ST80, ST101, ST19, and ST66. A bacterial population can comprise ST80, ST101, ST19, and ST116. A bacterial population can comprise ST80, ST101, ST23, and ST81. A bacterial population can comprise ST80, ST101, ST23, and ST119. A bacterial population can comprise ST80, ST101, ST23, and ST37. A bacterial population can comprise ST80, ST101, ST23, and ST66. A bacterial population can comprise ST80, ST101, ST23, and ST116. A bacterial population can comprise ST80, ST101, ST81, and ST119. A bacterial population can comprise ST80, ST101, ST81, and ST37. A bacterial population can comprise ST80, ST101, ST81, and ST66. A bacterial population can comprise ST80, ST101, ST81, and ST116. A bacterial population can comprise ST80, ST101, ST119, and ST37. A bacterial population can comprise ST80, ST101, ST119, and ST66. A bacterial population can comprise ST80, ST101, ST119, and ST116. A bacterial population can comprise ST80, ST101, ST37, and ST66. A bacterial population can comprise ST80, ST101, ST37, and ST116. A bacterial population can comprise ST80, ST101, ST66, and ST116. A bacterial population can comprise ST80, ST56, ST71, and ST19. A bacterial population can comprise ST80, ST56, ST71, and ST23. A bacterial population can comprise ST80, ST56, ST71, and ST81. A bacterial population can comprise ST80, ST56, ST71, and ST119. A bacterial population can comprise ST80, ST56, ST71, and ST37. A bacterial population can comprise ST80, ST56, ST71, and ST66. A bacterial population can comprise ST80, ST56, ST71, and ST116. A bacterial population can comprise ST80, ST56, ST19, and ST23. A bacterial population can comprise ST80, ST56, ST19, and ST81. A bacterial population can comprise ST80, ST56, ST19, and ST119. A bacterial population can comprise ST80, ST56, ST19, and ST37. A bacterial population can comprise ST80, ST56, ST19, and ST66. A bacterial population can comprise ST80, ST56, ST19, and ST116. A bacterial population can comprise ST80, ST56, ST23, and ST81. A bacterial population can comprise ST80, ST56, ST23, and ST119. A bacterial population can comprise ST80, ST56, ST23, and ST37. A bacterial population can comprise ST80, ST56, ST23, and ST66. A bacterial population can comprise ST80, ST56, ST23, and ST116. A bacterial population can comprise ST80, ST56, ST81, and ST119. A bacterial population can comprise ST80, ST56, ST81, and ST37. A bacterial population can comprise ST80, ST56, ST81, and ST66. A bacterial population can comprise ST80, ST56, ST81, and ST116. A bacterial population can comprise ST80, ST56, ST119, and ST37. A bacterial population can comprise ST80, ST56, ST119, and ST66. A bacterial population can comprise ST80, ST56, ST119, and ST116. A bacterial population can comprise ST80, ST56, ST37, and ST66. A bacterial population can comprise ST80, ST56, ST37, and ST116. A bacterial population can comprise ST80, ST56, ST66, and ST116. A bacterial population can comprise ST80, ST71, ST19, and ST23. A bacterial population can comprise ST80, ST71, ST19, and ST81. A bacterial population can comprise ST80, ST71, ST19, and ST119. A bacterial population can comprise ST80, ST71, ST19, and ST37. A bacterial population can comprise ST80, ST71, ST19, and ST66. A bacterial population can comprise ST80, ST71, ST19, and ST116. A bacterial population can comprise ST80, ST71, ST23, and ST81. A bacterial population can comprise ST80, ST71, ST23, and ST119. A bacterial population can comprise ST80, ST71, ST23, and ST37. A bacterial population can comprise ST80, ST71, ST23, and ST66. A bacterial population can comprise ST80, ST71, ST23, and ST116. A bacterial population can comprise ST80, ST71, ST81, and ST119. A bacterial population can comprise ST80, ST71, ST81, and ST37. A bacterial population can comprise ST80, ST71, ST81, and ST66. A bacterial population can comprise ST80, ST71, ST81, and ST116. A bacterial population can comprise ST80, ST71, ST119, and ST37. A bacterial population can comprise ST80, ST71, ST119, and ST66. A bacterial population can comprise ST80, ST71, ST119, and ST116. A bacterial population can comprise ST80, ST71, ST37, and ST66. A bacterial population can comprise ST80, ST71, ST37, and ST116. A bacterial population can comprise ST80, ST71, ST66, and ST116. A bacterial population can comprise ST80, ST19, ST23, and ST81. A bacterial population can comprise ST80, ST19, ST23, and ST119. A bacterial population can comprise ST80, ST19, ST23, and ST37. A bacterial population can comprise ST80, ST19, ST23, and ST66. A bacterial population can comprise ST80, ST19, ST23, and ST116. A bacterial population can comprise ST80, ST19, ST81, and ST119. A bacterial population can comprise ST80, ST19, ST81, and ST37. A bacterial population can comprise ST80, ST19, ST81, and ST66. A bacterial population can comprise ST80, ST19, ST81, and ST116. A bacterial population can comprise ST80, ST19, ST119, and ST37. A bacterial population can comprise ST80, ST19, ST119, and ST66. A bacterial population can comprise ST80, ST19, ST119, and ST116. A bacterial population can comprise ST80, ST19, ST37, and ST66. A bacterial population can comprise ST80, ST19, ST37, and ST116. A bacterial population can comprise ST80, ST19, ST66, and ST116. A bacterial population can comprise ST80, ST23, ST81, and ST119. A bacterial population can comprise ST80, ST23, ST81, and ST37. A bacterial population can comprise ST80, ST23, ST81, and ST66. A bacterial population can comprise ST80, ST23, ST81, and ST116. A bacterial population can comprise ST80, ST23, ST119, and ST37. A bacterial population can comprise ST80, ST23, ST119, and ST66. A bacterial population can comprise ST80, ST23, ST119, and ST116. A bacterial population can comprise ST80, ST23, ST37, and ST66. A bacterial population can comprise ST80, ST23, ST37, and ST116. A bacterial population can comprise ST80, ST23, ST66, and ST116. A bacterial population can comprise ST80, ST81, ST119, and ST37. A bacterial population can comprise ST80, ST81, ST119, and ST66. A bacterial population can comprise ST80, ST81, ST119, and ST116. A bacterial population can comprise ST80, ST81, ST37, and ST66. A bacterial population can comprise ST80, ST81, ST37, and ST116. A bacterial population can comprise ST80, ST81, ST66, and ST116. A bacterial population can comprise ST80, ST119, ST37, and ST66. A bacterial population can comprise ST80, ST119, ST37, and ST116. A bacterial population can comprise ST80, ST119, ST66, and ST116. A bacterial population can comprise ST80, ST37, ST66, and ST116. A bacterial population can comprise ST65, ST101, ST56, and ST71. A bacterial population can comprise ST65, ST101, ST56, and ST19. A bacterial population can comprise ST65, ST101, ST56, and ST23. A bacterial population can comprise ST65, ST101, ST56, and ST81. A bacterial population can comprise ST65, ST101, ST56, and ST119. A bacterial population can comprise ST65, ST101, ST56, and ST37. A bacterial population can comprise ST65, ST101, ST56, and ST66. A bacterial population can comprise ST65, ST101, ST56, and ST116. A bacterial population can comprise ST65, ST101, ST71, and ST19. A bacterial population can comprise ST65, ST101, ST71, and ST23. A bacterial population can comprise ST65, ST101, ST71, and ST81. A bacterial population can comprise ST65, ST101, ST71, and ST119. A bacterial population can comprise ST65, ST101, ST71, and ST37. A bacterial population can comprise ST65, ST101, ST71, and ST66. A bacterial population can comprise ST65, ST101, ST71, and ST116. A bacterial population can comprise ST65, ST101, ST19, and ST23. A bacterial population can comprise ST65, ST101, ST19, and ST81. A bacterial population can comprise ST65, ST101, ST19, and ST119. A bacterial population can comprise ST65, ST101, ST19, and ST37. A bacterial population can comprise ST65, ST101, ST19, and ST66. A bacterial population can comprise ST65, ST101, ST19, and ST116. A bacterial population can comprise ST65, ST101, ST23, and ST81. A bacterial population can comprise ST65, ST101, ST23, and ST119. A bacterial population can comprise ST65, ST101, ST23, and ST37. A bacterial population can comprise ST65, ST101, ST23, and ST66. A bacterial population can comprise ST65, ST101, ST23, and ST116. A bacterial population can comprise ST65, ST101, ST81, and ST119. A bacterial population can comprise ST65, ST101, ST81, and ST37. A bacterial population can comprise ST65, ST101, ST81, and ST66. A bacterial population can comprise ST65, ST101, ST81, and ST116. A bacterial population can comprise ST65, ST101, ST119, and ST37. A bacterial population can comprise ST65, ST101, ST119, and ST66. A bacterial population can comprise ST65, ST101, ST119, and ST116. A bacterial population can comprise ST65, ST101, ST37, and ST66. A bacterial population can comprise ST65, ST101, ST37, and ST116. A bacterial population can comprise ST65, ST101, ST66, and ST116. A bacterial population can comprise ST65, ST56, ST71, and ST19. A bacterial population can comprise ST65, ST56, ST71, and ST23. A bacterial population can comprise ST65, ST56, ST71, and ST81. A bacterial population can comprise ST65, ST56, ST71, and ST119. A bacterial population can comprise ST65, ST56, ST71, and ST37. A bacterial population can comprise ST65, ST56, ST71, and ST66. A bacterial population can comprise ST65, ST56, ST71, and ST116. A bacterial population can comprise ST65, ST56, ST19, and ST23. A bacterial population can comprise ST65, ST56, ST19, and ST81. A bacterial population can comprise ST65, ST56, ST19, and ST119. A bacterial population can comprise ST65, ST56, ST19, and ST37. A bacterial population can comprise ST65, ST56, ST19, and ST66. A bacterial population can comprise ST65, ST56, ST19, and ST116. A bacterial population can comprise ST65, ST56, ST23, and ST81. A bacterial population can comprise ST65, ST56, ST23, and ST119. A bacterial population can comprise ST65, ST56, ST23, and ST37. A bacterial population can comprise ST65, ST56, ST23, and ST66. A bacterial population can comprise ST65, ST56, ST23, and ST116. A bacterial population can comprise ST65, ST56, ST81, and ST119. A bacterial population can comprise ST65, ST56, ST81, and ST37. A bacterial population can comprise ST65, ST56, ST81, and ST66. A bacterial population can comprise ST65, ST56, ST81, and ST116. A bacterial population can comprise ST65, ST56, ST119, and ST37. A bacterial population can comprise ST65, ST56, ST119, and ST66. A bacterial population can comprise ST65, ST56, ST119, and ST116. A bacterial population can comprise ST65, ST56, ST37, and ST66. A bacterial population can comprise ST65, ST56, ST37, and ST116. A bacterial population can comprise ST65, ST56, ST66, and ST116. A bacterial population can comprise ST65, ST71, ST19, and ST23. A bacterial population can comprise ST65, ST71, ST19, and ST81. A bacterial population can comprise ST65, ST71, ST19, and ST119. A bacterial population can comprise ST65, ST71, ST19, and ST37. A bacterial population can comprise ST65, ST71, ST19, and ST66. A bacterial population can comprise ST65, ST71, ST19, and ST116. A bacterial population can comprise ST65, ST71, ST23, and ST81. A bacterial population can comprise ST65, ST71, ST23, and ST119. A bacterial population can comprise ST65, ST71, ST23, and ST37. A bacterial population can comprise ST65, ST71, ST23, and ST66. A bacterial population can comprise ST65, ST71, ST23, and ST116. A bacterial population can comprise ST65, ST71, ST81, and ST119. A bacterial population can comprise ST65, ST71, ST81, and ST37. A bacterial population can comprise ST65, ST71, ST81, and ST66. A bacterial population can comprise ST65, ST71, ST81, and ST116. A bacterial population can comprise ST65, ST71, ST119, and ST37. A bacterial population can comprise ST65, ST71, ST119, and ST66. A bacterial population can comprise ST65, ST71, ST119, and ST116. A bacterial population can comprise ST65, ST71, ST37, and ST66. A bacterial population can comprise ST65, ST71, ST37, and ST116. A bacterial population can comprise ST65, ST71, ST66, and ST116. A bacterial population can comprise ST65, ST19, ST23, and ST81. A bacterial population can comprise ST65, ST19, ST23, and ST119. A bacterial population can comprise ST65, ST19, ST23, and ST37. A bacterial population can comprise ST65, ST19, ST23, and ST66. A bacterial population can comprise ST65, ST19, ST23, and ST116. A bacterial population can comprise ST65, ST19, ST81, and ST119. A bacterial population can comprise ST65, ST19, ST81, and ST37. A bacterial population can comprise ST65, ST19, ST81, and ST66. A bacterial population can comprise ST65, ST19, ST81, and ST116. A bacterial population can comprise ST65, ST19, ST119, and ST37. A bacterial population can comprise ST65, ST19, ST119, and ST66. A bacterial population can comprise ST65, ST19, ST119, and ST116. A bacterial population can comprise ST65, ST19, ST37, and ST66. A bacterial population can comprise ST65, ST19, ST37, and ST116. A bacterial population can comprise ST65, ST19, ST66, and ST116. A bacterial population can comprise ST65, ST23, ST81, and ST119. A bacterial population can comprise ST65, ST23, ST81, and ST37. A bacterial population can comprise ST65, ST23, ST81, and ST66. A bacterial population can comprise ST65, ST23, ST81, and ST116. A bacterial population can comprise ST65, ST23, ST119, and ST37. A bacterial population can comprise ST65, ST23, ST119, and ST66. A bacterial population can comprise ST65, ST23, ST119, and ST116. A bacterial population can comprise ST65, ST23, ST37, and ST66. A bacterial population can comprise ST65, ST23, ST37, and ST116. A bacterial population can comprise ST65, ST23, ST66, and ST116. A bacterial population can comprise ST65, ST81, ST119, and ST37. A bacterial population can comprise ST65, ST81, ST119, and ST66. A bacterial population can comprise ST65, ST81, ST119, and ST116. A bacterial population can comprise ST65, ST81, ST37, and ST66. A bacterial population can comprise ST65, ST81, ST37, and ST116. A bacterial population can comprise ST65, ST81, ST66, and ST116. A bacterial population can comprise ST65, ST119, ST37, and ST66. A bacterial population can comprise ST65, ST119, ST37, and ST116. A bacterial population can comprise ST65, ST119, ST66, and ST116. A bacterial population can comprise ST65, ST37, ST66, and ST116. A bacterial population can comprise ST101, ST56, ST71, and ST19. A bacterial population can comprise ST101, ST56, ST71, and ST23. A bacterial population can comprise ST101, ST56, ST71, and ST81. A bacterial population can comprise ST101, ST56, ST71, and ST119. A bacterial population can comprise ST101, ST56, ST71, and ST37. A bacterial population can comprise ST101, ST56, ST71, and ST66. A bacterial population can comprise ST101, ST56, ST71, and ST116. A bacterial population can comprise ST101, ST56, ST19, and ST23. A bacterial population can comprise ST101, ST56, ST19, and ST81. A bacterial population can comprise ST101, ST56, ST19, and ST119. A bacterial population can comprise ST101, ST56, ST19, and ST37. A bacterial population can comprise ST101, ST56, ST19, and ST66. A bacterial population can comprise ST101, ST56, ST19, and ST116. A bacterial population can comprise ST101, ST56, ST23, and ST81. A bacterial population can comprise ST101, ST56, ST23, and ST119. A bacterial population can comprise ST101, ST56, ST23, and ST37. A bacterial population can comprise ST101, ST56, ST23, and ST66. A bacterial population can comprise ST101, ST56, ST23, and ST116. A bacterial population can comprise ST101, ST56, ST81, and ST119. A bacterial population can comprise ST101, ST56, ST81, and ST37. A bacterial population can comprise ST101, ST56, ST81, and ST66. A bacterial population can comprise ST101, ST56, ST81, and ST116. A bacterial population can comprise ST101, ST56, ST119, and ST37. A bacterial population can comprise ST101, ST56, ST119, and ST66. A bacterial population can comprise ST101, ST56, ST119, and ST116. A bacterial population can comprise ST101, ST56, ST37, and ST66. A bacterial population can comprise ST101, ST56, ST37, and ST116. A bacterial population can comprise ST101, ST56, ST66, and ST116. A bacterial population can comprise ST101, ST71, ST19, and ST23. A bacterial population can comprise ST101, ST71, ST19, and ST81. A bacterial population can comprise ST101, ST71, ST19, and ST119. A bacterial population can comprise ST101, ST71, ST19, and ST37. A bacterial population can comprise ST101, ST71, ST19, and ST66. A bacterial population can comprise ST101, ST71, ST19, and ST116. A bacterial population can comprise ST101, ST71, ST23, and ST81. A bacterial population can comprise ST101, ST71, ST23, and ST119. A bacterial population can comprise ST101, ST71, ST23, and ST37. A bacterial population can comprise ST101, ST71, ST23, and ST66. A bacterial population can comprise ST101, ST71, ST23, and ST116. A bacterial population can comprise ST101, ST71, ST81, and ST119. A bacterial population can comprise ST101, ST71, ST81, and ST37. A bacterial population can comprise ST101, ST71, ST81, and ST66. A bacterial population can comprise ST101, ST71, ST81, and ST116. A bacterial population can comprise ST101, ST71, ST119, and ST37. A bacterial population can comprise ST101, ST71, ST119, and ST66. A bacterial population can comprise ST101, ST71, ST119, and ST116. A bacterial population can comprise ST101, ST71, ST37, and ST66. A bacterial population can comprise ST101, ST71, ST37, and ST116. A bacterial population can comprise ST101, ST71, ST66, and ST116. A bacterial population can comprise ST101, ST19, ST23, and ST81. A bacterial population can comprise ST101, ST19, ST23, and ST119. A bacterial population can comprise ST101, ST19, ST23, and ST37. A bacterial population can comprise ST101, ST19, ST23, and ST66. A bacterial population can comprise ST101, ST19, ST23, and ST116. A bacterial population can comprise ST101, ST19, ST81, and ST119. A bacterial population can comprise ST101, ST19, ST81, and ST37. A bacterial population can comprise ST101, ST19, ST81, and ST66. A bacterial population can comprise ST101, ST19, ST81, and ST116. A bacterial population can comprise ST101, ST19, ST119, and ST37. A bacterial population can comprise ST101, ST19, ST119, and ST66. A bacterial population can comprise ST101, ST19, ST119, and ST116. A bacterial population can comprise ST101, ST19, ST37, and ST66. A bacterial population can comprise ST101, ST19, ST37, and ST116. A bacterial population can comprise ST101, ST19, ST66, and ST116. A bacterial population can comprise ST101, ST23, ST81, and ST119. A bacterial population can comprise ST101, ST23, ST81, and ST37. A bacterial population can comprise ST101, ST23, ST81, and ST66. A bacterial population can comprise ST101, ST23, ST81, and ST116. A bacterial population can comprise ST101, ST23, ST119, and ST37. A bacterial population can comprise ST101, ST23, ST119, and ST66. A bacterial population can comprise ST101, ST23, ST119, and ST116. A bacterial population can comprise ST101, ST23, ST37, and ST66. A bacterial population can comprise ST101, ST23, ST37, and ST116. A bacterial population can comprise ST101, ST23, ST66, and ST116. A bacterial population can comprise ST101, ST81, ST119, and ST37. A bacterial population can comprise ST101, ST81, ST119, and ST66. A bacterial population can comprise ST101, ST81, ST119, and ST116. A bacterial population can comprise ST101, ST81, ST37, and ST66. A bacterial population can comprise ST101, ST81, ST37, and ST116. A bacterial population can comprise ST101, ST81, ST66, and ST116. A bacterial population can comprise ST101, ST119, ST37, and ST66. A bacterial population can comprise ST101, ST119, ST37, and ST116. A bacterial population can comprise ST101, ST119, ST66, and ST116. A bacterial population can comprise ST101, ST37, ST66, and ST116. A bacterial population can comprise ST56, ST71, ST19, and ST23. A bacterial population can comprise ST56, ST71, ST19, and ST81. A bacterial population can comprise ST56, ST71, ST19, and ST119. A bacterial population can comprise ST56, ST71, ST19, and ST37. A bacterial population can comprise ST56, ST71, ST19, and ST66. A bacterial population can comprise ST56, ST71, ST19, and ST116. A bacterial population can comprise ST56, ST71, ST23, and ST81. A bacterial population can comprise ST56, ST71, ST23, and ST119. A bacterial population can comprise ST56, ST71, ST23, and ST37. A bacterial population can comprise ST56, ST71, ST23, and ST66. A bacterial population can comprise ST56, ST71, ST23, and ST116. A bacterial population can comprise ST56, ST71, ST81, and ST119. A bacterial population can comprise ST56, ST71, ST81, and ST37. A bacterial population can comprise ST56, ST71, ST81, and ST66. A bacterial population can comprise ST56, ST71, ST81, and ST116. A bacterial population can comprise ST56, ST71, ST119, and ST37. A bacterial population can comprise ST56, ST71, ST119, and ST66. A bacterial population can comprise ST56, ST71, ST119, and ST116. A bacterial population can comprise ST56, ST71, ST37, and ST66. A bacterial population can comprise ST56, ST71, ST37, and ST116. A bacterial population can comprise ST56, ST71, ST66, and ST116. A bacterial population can comprise ST56, ST19, ST23, and ST81. A bacterial population can comprise ST56, ST19, ST23, and ST119. A bacterial population can comprise ST56, ST19, ST23, and ST37. A bacterial population can comprise ST56, ST19, ST23, and ST66. A bacterial population can comprise ST56, ST19, ST23, and ST116. A bacterial population can comprise ST56, ST19, ST81, and ST119. A bacterial population can comprise ST56, ST19, ST81, and ST37. A bacterial population can comprise ST56, ST19, ST81, and ST66. A bacterial population can comprise ST56, ST19, ST81, and ST116. A bacterial population can comprise ST56, ST19, ST119, and ST37. A bacterial population can comprise ST56, ST19, ST119, and ST66. A bacterial population can comprise ST56, ST19, ST119, and ST116. A bacterial population can comprise ST56, ST19, ST37, and ST66. A bacterial population can comprise ST56, ST19, ST37, and ST116. A bacterial population can comprise ST56, ST19, ST66, and ST116. A bacterial population can comprise ST56, ST23, ST81, and ST119. A bacterial population can comprise ST56, ST23, ST81, and ST37. A bacterial population can comprise ST56, ST23, ST81, and ST66. A bacterial population can comprise ST56, ST23, ST81, and ST116. A bacterial population can comprise ST56, ST23, ST119, and ST37. A bacterial population can comprise ST56, ST23, ST119, and ST66. A bacterial population can comprise ST56, ST23, ST119, and ST116. A bacterial population can comprise ST56, ST23, ST37, and ST66. A bacterial population can comprise ST56, ST23, ST37, and ST116. A bacterial population can comprise ST56, ST23, ST66, and ST116. A bacterial population can comprise ST56, ST81, ST119, and ST37. A bacterial population can comprise ST56, ST81, ST119, and ST66. A bacterial population can comprise ST56, ST81, ST119, and ST116. A bacterial population can comprise ST56, ST81, ST37, and ST66. A bacterial population can comprise ST56, ST81, ST37, and ST116. A bacterial population can comprise ST56, ST81, ST66, and ST116. A bacterial population can comprise ST56, ST119, ST37, and ST66. A bacterial population can comprise ST56, ST119, ST37, and ST116. A bacterial population can comprise ST56, ST119, ST66, and ST116. A bacterial population can comprise ST56, ST37, ST66, and ST116. A bacterial population can comprise ST71, ST19, ST23, and ST81. A bacterial population can comprise ST71, ST19, ST23, and ST119. A bacterial population can comprise ST71, ST19, ST23, and ST37. A bacterial population can comprise ST71, ST19, ST23, and ST66. A bacterial population can comprise ST71, ST19, ST23, and ST116. A bacterial population can comprise ST71, ST19, ST81, and ST119. A bacterial population can comprise ST71, ST19, ST81, and ST37. A bacterial population can comprise ST71, ST19, ST81, and ST66. A bacterial population can comprise ST71, ST19, ST81, and ST116. A bacterial population can comprise ST71, ST19, ST119, and ST37. A bacterial population can comprise ST71, ST19, ST119, and ST66. A bacterial population can comprise ST71, ST19, ST119, and ST116. A bacterial population can comprise ST71, ST19, ST37, and ST66. A bacterial population can comprise ST71, ST19, ST37, and ST116. A bacterial population can comprise ST71, ST19, ST66, and ST116. A bacterial population can comprise ST71, ST23, ST81, and ST119. A bacterial population can comprise ST71, ST23, ST81, and ST37. A bacterial population can comprise ST71, ST23, ST81, and ST66. A bacterial population can comprise ST71, ST23, ST81, and ST116. A bacterial population can comprise ST71, ST23, ST119, and ST37. A bacterial population can comprise ST71, ST23, ST119, and ST66. A bacterial population can comprise ST71, ST23, ST119, and ST116. A bacterial population can comprise ST71, ST23, ST37, and ST66. A bacterial population can comprise ST71, ST23, ST37, and ST116. A bacterial population can comprise ST71, ST23, ST66, and ST116. A bacterial population can comprise ST71, ST81, ST119, and ST37. A bacterial population can comprise ST71, ST81, ST119, and ST66. A bacterial population can comprise ST71, ST81, ST119, and ST116. A bacterial population can comprise ST71, ST81, ST37, and ST66. A bacterial population can comprise ST71, ST81, ST37, and ST116. A bacterial population can comprise ST71, ST81, ST66, and ST116. A bacterial population can comprise ST71, ST119, ST37, and ST66. A bacterial population can comprise ST71, ST119, ST37, and ST116. A bacterial population can comprise ST71, ST119, ST66, and ST116. A bacterial population can comprise ST71, ST37, ST66, and ST116. A bacterial population can comprise ST19, ST23, ST81, and ST119. A bacterial population can comprise ST19, ST23, ST81, and ST37. A bacterial population can comprise ST19, ST23, ST81, and ST66. A bacterial population can comprise ST19, ST23, ST81, and ST116. A bacterial population can comprise ST19, ST23, ST119, and ST37. A bacterial population can comprise ST19, ST23, ST119, and ST66. A bacterial population can comprise ST19, ST23, ST119, and ST116. A bacterial population can comprise ST19, ST23, ST37, and ST66. A bacterial population can comprise ST19, ST23, ST37, and ST116. A bacterial population can comprise ST19, ST23, ST66, and ST116. A bacterial population can comprise ST19, ST81, ST119, and ST37. A bacterial population can comprise ST19, ST81, ST119, and ST66. A bacterial population can comprise ST19, ST81, ST119, and ST116. A bacterial population can comprise ST19, ST81, ST37, and ST66. A bacterial population can comprise ST19, ST81, ST37, and ST116. A bacterial population can comprise ST19, ST81, ST66, and ST116. A bacterial population can comprise ST19, ST119, ST37, and ST66. A bacterial population can comprise ST19, ST119, ST37, and ST116. A bacterial population can comprise ST19, ST119, ST66, and ST116. A bacterial population can comprise ST19, ST37, ST66, and ST116. A bacterial population can comprise ST23, ST81, ST119, and ST37. A bacterial population can comprise ST23, ST81, ST119, and ST66. A bacterial population can comprise ST23, ST81, ST119, and ST116. A bacterial population can comprise ST23, ST81, ST37, and ST66. A bacterial population can comprise ST23, ST81, ST37, and ST116. A bacterial population can comprise ST23, ST81, ST66, and ST116. A bacterial population can comprise ST23, ST119, ST37, and ST66. A bacterial population can comprise ST23, ST119, ST37, and ST116. A bacterial population can comprise ST23, ST119, ST66, and ST116. A bacterial population can comprise ST23, ST37, ST66, and ST116. A bacterial population can comprise ST81, ST119, ST37, and ST66. A bacterial population can comprise ST81, ST119, ST37, and ST116. A bacterial population can comprise ST81, ST119, ST66, and ST116. A bacterial population can comprise ST81, ST37, ST66, and ST116. A bacterial population can comprise ST119, ST37, ST66, and ST116.

A bacterial population can comprise a bacterial strain of each of the following: Bifidobacterium adolescentis, Lactobacillus rhamnosus, Bifidobacterium longum, Bifidobacterium bifidum, and Bifidobacterium pseudocatenulatum. A bacterial population can comprise a bacterial strain of each of the following: Bifidobacterium adolescentis, Lactobacillus rhamnosus, Bifidobacterium longum, Bifidobacterium bifidum, and Bifidobacterium breve. A bacterial population can comprise a bacterial strain of each of the following: Bifidobacterium adolescentis, Lactobacillus rhamnosus, Bifidobacterium longum, Bifidobacterium bifidum, and Lactobacillus plantarum. A bacterial population can comprise a bacterial strain of each of the following: Bifidobacterium adolescentis, Lactobacillus rhamnosus, Bifidobacterium longum, Bifidobacterium pseudocatenulatum, and Bifidobacterium breve. A bacterial population can comprise a bacterial strain of each of the following: Bifidobacterium adolescentis, Lactobacillus rhamnosus, Bifidobacterium longum, Bifidobacterium pseudocatenulatum, and Lactobacillus plantarum. A bacterial population can comprise a bacterial strain of each of the following: Bifidobacterium adolescentis, Lactobacillus rhamnosus, Bifidobacterium longum, Bifidobacterium breve, and Lactobacillus plantarum. A bacterial population can comprise a bacterial strain of each of the following: Bifidobacterium adolescentis, Lactobacillus rhamnosus, Bifidobacterium bifidum, Bifidobacterium pseudocatenulatum, and Bifidobacterium breve. A bacterial population can comprise a bacterial strain of each of the following: Bifidobacterium adolescentis, Lactobacillus rhamnosus, Bifidobacterium bifidum, Bifidobacterium pseudocatenulatum, and Lactobacillus plantarum. A bacterial population can comprise a bacterial strain of each of the following: Bifidobacterium adolescentis, Lactobacillus rhamnosus, Bifidobacterium bifidum, Bifidobacterium breve, and Lactobacillus plantarum. A bacterial population can comprise a bacterial strain of each of the following: Bifidobacterium adolescentis, Lactobacillus rhamnosus, Bifidobacterium pseudocatenulatum, Bifidobacterium breve, and Lactobacillus plantarum. A bacterial population can comprise a bacterial strain of each of the following: Bifidobacterium adolescentis, Bifidobacterium longum, Bifidobacterium bifidum, Bifidobacterium pseudocatenulatum, and Bifidobacterium breve. A bacterial population can comprise a bacterial strain of each of the following: Bifidobacterium adolescentis, Bifidobacterium longum, Bifidobacterium bifidum, Bifidobacterium pseudocatenulatum, and Lactobacillus plantarum. A bacterial population can comprise a bacterial strain of each of the following: Bifidobacterium adolescentis, Bifidobacterium longum, Bifidobacterium bifidum, Bifidobacterium breve, and Lactobacillus plantarum. A bacterial population can comprise a bacterial strain of each of the following: Bifidobacterium adolescentis, Bifidobacterium longum, Bifidobacterium pseudocatenulatum, Bifidobacterium breve, and Lactobacillus plantarum. A bacterial population can comprise a bacterial strain of each of the following: Bifidobacterium adolescentis, Bifidobacterium bifidum, Bifidobacterium pseudocatenulatum, Bifidobacterium breve, and Lactobacillus plantarum. A bacterial population can comprise a bacterial strain of each of the following: Lactobacillus rhamnosus, Bifidobacterium longum, Bifidobacterium bifidum, Bifidobacterium pseudocatenulatum, and Bifidobacterium breve. A bacterial population can comprise a bacterial strain of each of the following: Lactobacillus rhamnosus, Bifidobacterium longum, Bifidobacterium bifidum, Bifidobacterium pseudocatenulatum, and Lactobacillus plantarum. A bacterial population can comprise a bacterial strain of each of the following: Lactobacillus rhamnosus, Bifidobacterium longum, Bifidobacterium bifidum, Bifidobacterium breve, and Lactobacillus plantarum. A bacterial population can comprise a bacterial strain of each of the following: Lactobacillus rhamnosus, Bifidobacterium longum, Bifidobacterium pseudocatenulatum, Bifidobacterium breve, and Lactobacillus plantarum. A bacterial population can comprise a bacterial strain of each of the following: Lactobacillus rhamnosus, Bifidobacterium bifidum, Bifidobacterium pseudocatenulatum, Bifidobacterium breve, and Lactobacillus plantarum. A bacterial population can comprise a bacterial strain of each of the following: Bifidobacterium longum, Bifidobacterium bifidum, Bifidobacterium pseudocatenulatum, Bifidobacterium breve, and Lactobacillus plantarum. For the bacterial composition comprising any 5 of Bifidobacterium adolescentis, Lactobacillus rhamnosus, Bifidobacterium longum, Bifidobacterium bifidum, Bifidobacterium pseudocatenulatum, Bifidobacterium breve, and Lactobacillus plantarum, Bifidobacterium adolescentis can comprise ST101; Lactobacillus rhamnosus can comprise ST116; Bifidobacterium longum can comprise ST119, ST19, ST81, or ST23; Bifidobacterium bifidum can comprise ST31 or ST80; Bifidobacterium breve can comprise ST56 or ST71; Bifidobacterium pseudocatenulatum can comprise ST37 or ST66; and Lactobacillus plantarum can comprise ST65.

A bacterial population can comprise a bacterial strain of each of the following: Bifidobacterium bifidum, Lactobacillus plantarum, Bifidobacterium longum, Bifidobacterium adolescentis, and Bifidobacterium breve. A bacterial population can comprise a bacterial strain of each of the following: Bifidobacterium bifidum, Lactobacillus plantarum, Bifidobacterium longum, Bifidobacterium adolescentis, and Bifidobacterium pseudocatenulatum. A bacterial population can comprise a bacterial strain of each of the following: Bifidobacterium bifidum, Lactobacillus plantarum, Bifidobacterium longum, Bifidobacterium adolescentis, and Lactobacillus rhamnosus. A bacterial population can comprise a bacterial strain of each of the following: Bifidobacterium bifidum, Lactobacillus plantarum, Bifidobacterium longum, Bifidobacterium breve, and Bifidobacterium pseudocatenulatum. A bacterial population can comprise a bacterial strain of each of the following: Bifidobacterium bifidum, Lactobacillus plantarum, Bifidobacterium longum, Bifidobacterium breve, and Lactobacillus rhamnosus. A bacterial population can comprise a bacterial strain of each of the following: Bifidobacterium bifidum, Lactobacillus plantarum, Bifidobacterium longum, Bifidobacterium pseudocatenulatum, and Lactobacillus rhamnosus. A bacterial population can comprise ST31, ST65, ST119, ST101, and ST56. A bacterial population can comprise ST31, ST65, ST119, ST101, and ST71. A bacterial population can comprise ST31, ST65, ST119, ST101, and ST37. A bacterial population can comprise ST31, ST65, ST119, ST101, and ST66. A bacterial population can comprise ST31, ST65, ST119, ST101, and ST116. A bacterial population can comprise ST31, ST65, ST119, ST56, and ST71. A bacterial population can comprise ST31, ST65, ST119, ST56, and ST37. A bacterial population can comprise ST31, ST65, ST119, ST56, and ST66. A bacterial population can comprise ST31, ST65, ST119, ST56, and ST116. A bacterial population can comprise ST31, ST65, ST119, ST71, and ST37. A bacterial population can comprise ST31, ST65, ST119, ST71, and ST66. A bacterial population can comprise ST31, ST65, ST119, ST71, and ST116. A bacterial population can comprise ST31, ST65, ST119, ST37, and ST66. A bacterial population can comprise ST31, ST65, ST119, ST37, and ST116. A bacterial population can comprise ST31, ST65, ST119, ST66, and ST116. In some cases, the bacterial population can comprise any of the aforementioned bacterial strains in addition to one or more additional bacterial strains from Bifidobacterium sp. or Lactobacillus sp. (or Vertebrate-Associated Lactobacillaceae).

### Collective Interactions

The bacterial population described herein can comprise a plurality of bacterial strains. In some cases, a first bacterial strain and a second bacterial strain different from the first bacterial strains of the plurality of bacterial strains can form a “collective interaction” or have a “collective effect”.

As used herein, a collective effect is an effect of a group of entities that, when present simultaneously, is greater than any individual effect of any individual entity of the group when present alone, individually, or not simultaneously. An interaction of the entities that facilitate the generation of the collective effect is referred to as the “collective interaction.” In some cases, a collective effect can comprise a synergistic effect. As used herein, a synergistic effect is an effect of a group of entities that, when present simultaneously, is greater than the sum of the effect of any individual entities when present alone, individually, or not simultaneously. An interaction of the entities that facilitate the generation of the synergistic effect is referred to as the “synergistic interaction.”

When referring to a plurality of bacterial strains of a bacterial population, a collective effect is an effect resulting from the interaction of at least two bacterial strains of the bacterial population that is greater than either or both effect of two control different bacterial populations each comprising only one of the two bacterial strains, wherein each of the two control bacterial populations has not been cultured or in contact with: (1) a cultured medium or supernatant of the other bacteria strain and/or (2) a bacterial product generated by the other bacteria strain. The interaction between the two bacterial strains that facilitates the collective effect is referred herein as the “collective interaction.” The collective effect can also comprise an effect resulting from the interaction of at least a first bacterial strain and: (1) a cultured medium of a second bacterial strain (different from the first bacterial strain) or a supernatant thereof; or (2) a bacterial product generated by the second bacterial strain that is greater than either or both effect of two control different bacterial populations each comprising only one of the two bacterial strains, wherein each of the two control bacterial populations has not been cultured or in contact with: (1) a cultured medium or supernatant of the other bacteria strain and/or (2) a bacterial product generated by the other bacteria strain. As used herein, when referring to multiple entities, the use of ordinal numbering is used to distinguish various distinguishable entities and should not be interpreted to limited to any one particular entity. A control composition or control formulation may comprise any control or control bacterial populations as described herein.

In some case, the collective interaction between two bacterial strains can encompass various mechanisms. For example, the collective interaction between two bacterial strains can comprise cross-feeding or syntrophy, as described here. Bacterial syntrophy occurs when one bacterial strains/species feeds off the bacterial product(s) (such as a metabolite) of another different bacterial strains/species. For example, during syntrophy, a first bacterial strain may use a second bacterial product (generated by a second bacterial strain different from the first bacterial strain) to generate a first bacterial product different from the second bacterial product. The first bacterial strain may use the second bacterial product as a carbon source. The first bacterial strain may use the second bacterial product as an energy source. As used herein, an energy source is a purified substance that acts as a source for energy production of a microbial organism added into the culture medium for culturing the microbial organism.

In some cases, the collective interaction between two bacterial strains can comprise modification(s) of at least one of the two bacterial strains. For example, a first bacterial strain may be modified (genetically, epigenetically, or metabolically) by a second bacterial product (generated by a second bacterial strain different from the first bacterial strain). Such modification may facilitate the modified first bacterial strain to exhibit an ability different from the unmodified bacteria, e.g., a collective effect in any one of the disease associated functions as described herein.

Two bacterial strains can exhibit a collective interaction when directly cultured together. Two bacterial strains can exhibit a collective interaction when directly cultured individually. When two collective bacterial stains cultured individually, the collective interaction can be mediated via a “conditioned medium” or “cultured medium.” As used herein, a cultured or conditioned medium comprises a culture medium that has been used to culture a bacterial strain. For example, in some case, the cultured medium may comprise a second bacterial product generated by the second bacterial strain. When used to culture a first bacterial strain different from the second bacterial strain, the first bacterial strain may feed off the second bacterial product or is modified by the second bacterial product. Bacterial syntrophy may occur via a medium comprising a carbon source or an energy source, for example a carbohydrate media or complete media, and a second bacterial strain or supernatant thereof.

In some cases, the bacterial population comprising at least two bacterial strains that exhibit a collective interaction can comprise any of the bacterial population as described herein. In some cases, the bacterial population comprising at least two bacterial strains that exhibit a collective interaction can also be extended to a composition comprising (1) any one of the bacterial strain and (2) the conditioned medium of the other bacterial strain or a supernatant thereof, and/or a bacterial product produced by the other bacterial strain. For example, a collective bacterial population comprising bacterial strain A and bacterial strain B can comprise: (1) A and (2) B, the conditioned medium of B or a supernatant thereof, and/or the bacterial product produced by B. In some cases, composition can also comprise: (1) the conditioned medium of A (or a supernatant thereof) or bacterial product produced by A and (2) the conditioned medium of B (or a supernatant thereof) or bacterial product produced by B. Thus, the disclosure of the bacterial population can be extended to the conditioned medium (or a supernatant thereof) by the bacterial strain and the bacterial product generated by the bacterial strain, as described in the bacterial population disclosed herein.

In some cases, for two bacterial strains exhibiting a collective interaction as described herein, a first bacterial strain of the two bacterial strains may be a donor bacterial strain and a second bacterial strain of the two bacterial strains different from the first bacterial strain may be a recipient strain, or vice versa. A “donor bacterial strain” or “donor strain,” as used herein, refers to a first bacterial strain that is capable of generating a bacterial product that can facilitate a collective interaction with a second bacterial strain. A “recipient bacterial strain” or “recipient strain,” as used herein, refers to a second bacterial strain that is capable of utilizing a bacterial product generated by a first bacterial strain to form the collective interaction as described herein. The methods to identify a donor or recipient strains are described herein, for example, in EXAMPLEs 4 and 7-8. Wherein when describing a cross-feeing of two bacterial strains that exhibit a collective interaction, the donor bacterial strain can cross-feed the recipient bacterial strain (i.e., the recipient bacterial strain is cross-fed). In some cases, as described herein, the cross-feeding can comprise inoculating/culturing the two bacterial strains together. In other cases, the cross-feeding can comprise inoculating the donor bacterial strain, generating a conditioned medium of the donor bacterial strain (or a supernatant thereof or a bacterial product generated by the donor bacterial strain), and inoculating/culturing the recipient strain with the conditioned medium of the donor bacterial strain (or a supernatant thereof or a bacterial product generated by the donor bacterial strain). In cross-feeding, the conditioned medium can be supplemented with a carbon source, as described herein.

In some cases, a first bacterial strain (such as any bacterial strain described herein) may have a sufficient ability in a first disease-associated function but does not have a sufficient ability in a second disease-associated function; a second bacterial strain different from the first bacterial strain (such as any bacterial strain described herein) may have a sufficient ability in a first disease-associated function but does not have a sufficient ability in the first disease-associated function. By generating a bacterial population comprising the first and second bacterial strains, the bacterial population can thus exhibit a collective effect or synergistic effect in the first and/or second disease-associated functions. In an illustrative example, a collective bacterial population may comprise at least one donor and one recipient bacterial strains. A donor strain may be capable of utilizing a vaginally (or infant gastrointestinally) relevant carbohydrate and convert the carbohydrate into a different bacterial product (such as a metabolite) but is incapable (or sufficiently capable) of inhibiting a growth of a vaginal (or infant gastrointestinal) pathogen. A recipient may be incapable of (or sufficiently capable) utilizing a vaginally (or infant gastrointestinally) relevant carbohydrate but is capable of utilizing the different bacterial product (such as a metabolite) and inhibiting a growth of a vaginal (or infant gastrointestinal) pathogen. Thus, while the bacterial populations comprising the individual donor or recipient bacterial strains may not be sufficient to inhibiting a growth of the vaginal (or infant gastrointestinal) pathogen within an environment comprising only the vaginally (or infant gastrointestinally) relevant carbohydrate as the nutrient source (since they cannot both proliferate and inhibit the growth of the pathogen within that environment), the bacterial population comprising both of the donor and recipient strains is capable of proliferating and inhibiting the growth of the pathogen within that environment. In another illustrative example, a bacterial population comprising the recipient bacterial strain and a cultured medium (of supernatant thereof) of the donor bacterial strain is also capable of proliferating and inhibiting the growth of the pathogen within that environment, since the cultured medium of the donor bacterial strain can contain the different bacterial product (such as a metabolite) generated by the donor bacterial strain such that the recipient bacterial strain can utilize and proliferate.

### Collective Effects

In some cases, a bacterial strain may exhibit sufficient abilities in the disease associated function as described herein using at least in part of the bacterial product. For example, a bacterial product may directly or indirectly inhibit the growth/biofilm formation of a vaginal pathogen; inhibit the growth of an infant gastrointestinal pathogen; facilitate the adherence of a bacterial strain to VEC/IEC; inhibit the immune response signaling pathway; increases the integrity of a barrier comprising IEC; or a combination thereof. In some cases, while a bacterial strain (or the cultured medium thereof; or a bacterial product encompassed within that cultured medium) may not have a sufficient ability in a first disease associated function, the bacterial strain (or the cultured medium thereof; or a bacterial product encompassed within that cultured medium) may have a sufficient ability in a second disease associated function. Thus, a bacterial population comprising the bacterial strain can be combined with another bacterial strain (or the cultured medium thereof; or a bacterial product encompassed within that cultured medium) to generate a collective effect in the first and second disease associated functions as described herein. In such case, the another bacterial strain may have a sufficient ability in the first disease associated function; not a sufficient ability in the first disease associated function; have a sufficient ability in the second disease associated function; and/or not have a sufficient ability in the second disease associated function.

In some instances, the bacterial population comprising the plurality of bacterial strains described herein (or a cultured medium thereof) can have a collective effect on the disease associated function as described herein. In some instances, the plurality of the bacterial population described herein (or a cultured medium thereof) can have a collective effect on the vaginal disease-associated function as described herein. For example, the bacterial population comprising the plurality of bacterial strains described herein (or a cultured medium thereof) can have a collective effect(s) on at least one of the adherence to a vaginal epithelial cell (VEC), the inhibition of growth of a vaginal pathogen, the inhibition of a biofilm formation of a vaginal pathogen, a utilization of a vaginally relevant carbohydrate, or a combination thereof.

In some cases, at least one of the bacterial strains (or the cultured medium thereof; or a bacterial product encompassed within that cultured medium) of the bacterial population comprising at least two different bacterial strains having a collective interaction can have an adherence to a VEC that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, at least one of the bacterial strains (or the cultured medium thereof; or a bacterial product encompassed within that cultured medium) of the bacterial population comprising at least two different bacterial strains having a collective interaction can have an adherence to a VEC that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at least two different bacterial strains.

In some cases, at least one of the bacterial strains (or the cultured medium thereof; or a bacterial product encompassed within that cultured medium) of the bacterial population comprising at least two different bacterial strains having a collective interaction can exhibit an inhibition of the growth of a vaginal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, at least one of the bacterial strains (or the cultured medium thereof; or a bacterial product encompassed within that cultured medium) of the bacterial population comprising at least two different bacterial strains having a collective interaction can exhibit an inhibition of the growth of a vaginal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at least two different bacterial strains.

In some cases, the bacterial population comprising at least two different bacterial strains having a collective interaction can exhibit an inhibition of the growth of a vaginal pathogen that is at least about 0.001%, at least about 0.001%, at least about 0.010%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial population comprising only one of the at least two different bacterial strains (or a bacterial population not comprising both of the two different bacterial strains). In some cases, the bacterial population comprising at least two different bacterial strains having a collective interaction having a collective interaction can exhibit an inhibition of the growth of a vaginal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher that of either bacterial population comprising only one of the at least two different bacterial strains (or a bacterial population not comprising both of the two different bacterial strains).

In some cases, at least one of the bacterial strains (or the cultured medium thereof; or a bacterial product encompassed within that cultured medium) of the bacterial population comprising at least two different bacterial strains having a collective interaction can exhibit an inhibition of the biofilm of a vaginal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, at least one of the bacterial strains (or the cultured medium thereof; or a bacterial product encompassed within that cultured medium) of the bacterial population comprising at least two different bacterial strains having a collective interaction can exhibit an inhibition of the biofilm of a vaginal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at least two different bacterial strains.

In some cases, the bacterial population comprising at least two different bacterial strains having a collective interaction can exhibit an inhibition of the biofilm formation of a vaginal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial population comprising only one of the at least two different bacterial strains (or a bacterial population not comprising both of the two different bacterial strains). In some cases, the bacterial population comprising at least two different bacterial strains having a collective interaction can exhibit an inhibition of the biofilm formation of a vaginal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher that of either bacterial population comprising only one of the at least two different bacterial strains (or a bacterial population not comprising both of the two different bacterial strains).

In some cases, at least one of bacterial strains of the bacterial population (or the cultured medium thereof; or a bacterial product encompassed within that cultured medium) comprising at least two different bacterial strains having a collective interaction can have a vaginally relevant carbohydrate growth (for example, for glycogen, bioglycogen, dextrin, or maltodextrin) ratio that is at least about 0.001%, at least about 0.001%, at least about 0.010%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain of two bacterial populations comprising only one of the at least two different bacterial strains not cultured with the other bacterial strain (or the cultured medium thereof; or a bacterial product encompassed within that cultured medium). In some cases, at least one of bacterial strains (or the cultured medium thereof; or a bacterial product encompassed within that cultured medium) of the bacterial population comprising at least two different bacterial strains having a collective interaction can have a vaginally relevant carbohydrate growth (for example, for glycogen, bioglycogen, dextrin, or maltodextrin) ratio that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain of two bacterial populations comprising only one of the at least two different bacterial strains not cultured with the other bacterial strain (or the cultured medium thereof; or a bacterial product encompassed within that cultured medium).

In some cases, at least one of bacterial strains (or the cultured medium thereof; or a bacterial product encompassed within that cultured medium) of the bacterial population comprising at least two different bacterial strains having a collective interaction can have a vaginal pH/physiological pH growth ratio that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain of two bacterial populations comprising only one of the at least two different bacterial strains not cultured with the other bacterial strain (or the cultured medium thereof; or a bacterial product encompassed within that cultured medium). In some cases, at least one of bacterial strains (or the cultured medium thereof; or a bacterial product encompassed within that cultured medium) of the bacterial population comprising at most two different bacterial strains having a collective interaction can have a vaginal pH/physiological pH growth ratio that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain of two bacterial populations comprising only one of the at least two different bacterial strains not cultured with the other bacterial strain (or the cultured medium thereof; or a bacterial product encompassed within that cultured medium).

In some cases, the bacterial population comprising at least two different bacterial strains having a collective interaction can generate at least about at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold more hydrogen peroxide than that of either bacterial population comprising only one of the at least two different bacterial strains (or a bacterial population not comprising both of the two different bacterial strains). In some cases, the bacterial population comprising at least two different bacterial strains having a collective interaction can exhibit an inhibition of the biofilm formation of a vaginal pathogen that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold more hydrogen peroxide than that of either bacterial population comprising only one of the at least two different bacterial strains (or a bacterial population not comprising both of the two different bacterial strains).

In some cases, at least one of bacterial strains (or the cultured medium thereof; or a bacterial product encompassed within that cultured medium) of the bacterial population comprising at least two different bacterial strains having a collective interaction can generate at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold more lactic acids than that (when measured by methods described in EXAMPLE 2) of either bacterial strain of two bacterial populations comprising only one of the at least two different bacterial strains not cultured with the other bacterial strain (or the cultured medium thereof; or a bacterial product encompassed within that cultured medium). In some cases, at least one of bacterial strains (or the cultured medium thereof; or a bacterial product encompassed within that cultured medium) of the bacterial population comprising at most two different bacterial strains having a collective interaction can generate at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold more lactic acids than that (when measured by methods described in EXAMPLE 2) of either bacterial strain of two bacterial populations comprising only one of the at least two different bacterial strains not cultured with the other bacterial strain (or the cultured medium thereof; or a bacterial product encompassed within that cultured medium).

In some instances, the plurality of the bacterial population described herein can have a collective effect on the infant gastrointestinal disease-associated function as described herein. For example, the bacterial population comprising at least two different bacterial strains having a collective interaction can have a collective effect(s) on at least one of the adherence to IEC, an integrity of a barrier comprising IEC, an inhibition of an infant gastrointestinal pathogen, a utilization of an infant-relevant carbohydrate, an inhibition of an immune response, or a combination thereof.

In some cases, one of the bacterial strains (or the cultured medium thereof; or a bacterial product encompassed within that cultured medium) of the bacterial population comprising at least two different bacterial strains having a collective interaction can have an adherence to an IEC that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, one of the bacterial strains (or the cultured medium thereof; or a bacterial product encompassed within that cultured medium) of the bacterial population comprising at least two different bacterial strains having a collective interaction can have an adherence to an IEC that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at least two different bacterial strains.

In some cases, one of the bacterial strains (or the cultured medium thereof; or a bacterial product encompassed within that cultured medium) of the bacterial population comprising at least two different bacterial strains having a collective interaction can exhibit an inhibition of the growth of an infant gastrointestinal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, one of the bacterial strains (or the cultured medium thereof; or a bacterial product encompassed within that cultured medium) of the bacterial population comprising at least two different bacterial strains having a collective interaction can exhibit an inhibition of the growth of an infant gastrointestinal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at most two different bacterial strains.

In some cases, the bacterial population comprising at least two different bacterial strains having a collective interaction can exhibit an inhibition of the growth of an infant gastrointestinal pathogen that is at least about 0.001%, at least about 0.001%, at least about 0.010%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial population comprising only one of the at least two different bacterial strains (or a bacterial population not comprising both of the two different bacterial strains). In some cases, the bacterial population comprising at least two different bacterial strains having a collective interaction can exhibit an inhibition of the growth of an infant gastrointestinal pathogen that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher that of either bacterial population comprising only one of the at least two different bacterial strains (or a bacterial population not comprising both of the two different bacterial strains).

In some cases, the bacterial population comprising at least two different bacterial strains having a collective interaction may increase the cellular impedance of a barrier comprising IEC by at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial population comprising only one of the at least two different bacterial strains (or a bacterial population not comprising both of the two different bacterial strains). In some cases, the bacterial population comprising at most two different bacterial strains having a collective interaction may increase the cellular impedance of a barrier comprising IEC by at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial population comprising only one of the at most two different bacterial strains (or a bacterial population not comprising both of the two different bacterial strains).

In some cases, one of the bacterial strains (or the cultured medium thereof; or a bacterial product encompassed within that cultured medium) of the bacterial population comprising at least two different bacterial strains having a collective interaction can increase the cellular impedance of a barrier comprising IEC by at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, one of the bacterial strains (or the cultured medium thereof; or a bacterial product encompassed within that cultured medium) of the bacterial population comprising at most two different bacterial strains having a collective interaction can increase the cellular impedance of a barrier comprising IEC by at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at most two different bacterial strains not cultured with the other bacterial strain or a cultured medium thereof.

In some cases, one of the bacterial strains (or the cultured medium thereof; or a bacterial product encompassed within that cultured medium) of the bacterial population comprising at least two different bacterial strains having a collective interaction can exhibit an inhibition of the immune response signaling pathway (for example, any immune response signaling pathway described herein or TLR4) that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain (or the cultured medium thereof; or a bacterial product encompassed within that cultured medium) of two bacterial populations comprising only one of the at least two different bacterial strains not cultured with the other bacterial strain or a cultured medium thereof. In some cases, one of the bacterial strains (or the cultured medium thereof; or a bacterial product encompassed within that cultured medium) of the bacterial population comprising at least two different bacterial strains having a collective interaction can exhibit an inhibition of the immune response signaling pathway that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain (or the cultured medium thereof; or a bacterial product encompassed within that cultured medium) of two bacterial populations comprising only one of the at most two different bacterial strains.

In some cases, the bacterial population comprising at least two different bacterial strains having a collective interaction can exhibit an inhibition of the immune response signaling pathway of an infant gastrointestinal pathogen that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial population comprising only one of the at least two different bacterial strains (or a bacterial population not comprising both of the two different bacterial strains). In some cases, the bacterial population comprising at least two different bacterial strains having a collective interaction can exhibit an inhibition of the immune response signaling pathway that is at most about 0.001%, at most about 0.001%, at most about 0.010%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher that of either bacterial population comprising only one of the at least two different bacterial strains (or a bacterial population not comprising both of the two different bacterial strains).

In some cases, at least one of bacterial strains of the bacterial population comprising at least two different bacterial strains having a collective interaction can have an infant gastrointestinally relevant carbohydrate growth (for example, any HMOs as described herein, 2′FL, or LNT) ratio that is at least about 0.001%, at least about 0.001%, at least about 0.01%, at least about 0.1%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than that of either bacterial strain of two bacterial populations comprising only one of the at least two different bacterial strains not cultured with the other bacterial strain. In some cases, at most one of bacterial strains of the bacterial population comprising at most two different bacterial strains having a collective interaction can have an infant gastrointestinally relevant carbohydrate growth (for example, any HMOs as described herein, 2′FL, or LNT) ratio that is at most about 0.001%, at most about 0.001%, at most about 0.01%, at most about 0.1%, at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than that of either bacterial strain of two bacterial populations comprising only one of the at most two different bacterial strains not cultured with the other bacterial strain.

In some cases, a first bacterial strain when present in a medium comprising the energy source and a second bacterial strain (different from the first bacterial strain) or its supernatant exhibits a growth of at least about 105% by weight as compared to the growth of the first bacterial strain when present in a medium comprising the energy source in an absence of the second bacterial strain or its supernatant. In some embodiments, the energy source for syntrophy can comprise starch. In some embodiments, the energy source for syntrophy does not comprise starch. The first bacterial strain may be present in the media with the energy source and second bacterial strain, or the supernatant of the second bacterial strain. In some embodiments, the supernatant derived from the bacterial strain can be free of cells, or it can contain a fermented derivative product of the bacterial strain. In some embodiments, the first strain can utilize the energy source to grow to at least 101%, 105%, 110%, 115%, 120%, 130%, 140% 150%, 160%, 170%, 180%, 190%, 200%, 250%, 300%, 350%, 400%, 450%, 500%, 550%, 600%, 650%, 700%, 750%, 800%, 850%, 900%, 950%, 999%, 1000%, 1100%, or more than 1100% by weight when compared to the growth of that first bacterial strain in medium with that energy source absent the bacterial strain or its supernatant for at most 48 hours, 24 hours, or 12 hours. In some embodiments, the first strain can utilize the energy source to grow to at least about 1200%, 1300%, 1400%, 1500%, 1600%, 1700%, 1800%, 1900%, 2000%, 3000%, 4000%, 5000%, 6000%, 7000%, 8000%, 9000%, 9999%, 10000% or more than about 10000% by weight when compared to the growth of that first bacterial strain in medium with that energy source absent the bacterial strain or its supernatant for at most 48 hours, 24 hours, or 12 hours.

In some embodiments, the energy source of the medium can include a modified starch, fermented starch, dextrin, maltodextrin, fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, or starch, or a combination thereof.

In some embodiments, the starch can comprise a modified starch, fermented starch, maltodextrin, or combination thereof.

In some embodiments, the first bacterial strain and/or the bacterial strain can include Lactobacillus, sp. and/or at least one or more strains of Bifidobacterium sp., and/or at least one strain of Akkermansia sp., and/or at least one or more strains of Blautia sp. and/or at least one or more strains of Clostridium sp., and/or at least one or more strains of Coprococcus sp., and/or at least one or more strains of Dorea sp., and/or at least one or more strains of Faecalibacterium sp., and/or at least one or more strains of Roseburia sp., and/or at least one or more strains of Ruminococcus sp., or a combination thereof.

In some embodiments, the first bacterial strain can proliferate for at least about 4 cell divisions within at most 72 hours, 60 hours, 48 hours, 36 hours, 24 hours, 12 hours, 6 hours, or 4 hours. In some embodiments, the supernatant can be cell-free. In some embodiments, the first bacterial strain can proliferate for at least about 8 cell divisions within at most 72 hours, 60 hours, 48 hours, 36 hours, 24 hours, 12 hours, 6 hours, or 4 hours. In some embodiments, the supernatant can be cell-free. In some embodiments, the first bacterial strain can proliferate for at least about 16 cell divisions within at most 72 hours, 60 hours, 48 hours, 36 hours, 24 hours, 12 hours, 6 hours, or 4 hours. In some embodiments, the supernatant can be cell-free. In some embodiments, the first bacterial strain can proliferate for at least about 32 cell divisions within at most 72 hours, 60 hours, 48 hours, 36 hours, 24 hours, 12 hours, 6 hours, or 4 hours.

In some embodiments, the pharmaceutical composition can comprise a different first and second bacterial strain, a medium that can include a secreted metabolite (or bacterial product) derived from the second bacterial strain, and a layer of epithelial cells. In some embodiments, the secreted metabolites (or bacterial product) derived from the second bacterial strain can decrease the gas permeability of the layer of epithelial cells by at least about 1%, 5%, 10%, 20%, 30%, 40%, 50%, 60%, 70%, 80%, 90%, or 99% compared to a layer of epithelial cells combined with a medium that can comprise the first bacterial strain in the absence of metabolites (or bacterial product) derived from the second bacterial strain. In some embodiments, the supernatant can be cell-free, or a fermentation product derived from the second bacterial strain. In some embodiments, the gas permeability of the epithelial cells can be measured by the transport of Fluorescein isothiocyanate (FITC)-conjugated dextran across the layer of epithelial cells.

In some embodiments, the epithelial cells can be mammalian epithelial cells or specifically human epithelial cells.

In some embodiments, the supernatant derived from the second bacterial strain can be cell-free, comprised of a fermentation product, or contain a secreted metabolite (or bacterial product) or inviable cell derived from the second bacterial strain. In some embodiments, the metabolite (or bacterial product) or inviable cell can be combined with an engineered cell comprising a reporter, and can decrease the signal of the reporter by at least about 1%, 5%, 10%, 20%, 30%, 40%, 50%, 60%, 70%, 80%, 90%, or 99% as compared to a signal of the reporter when the engineered cell is not combined with the metabolite (or bacterial product) or inviable cell derived from the second bacterial strain.

In some embodiments, a pharmaceutical composition comprising a bacterial population comprising a first bacterial strain and different second bacterial strain can be combined within a medium for at most 30 hours, 25 hours, 20 hours, 15 hours, 10 hours, 8 hours or 5 hours, can exhibit a growth of at least 101%, 105%, 110%, 115%, or 119% by colony-forming unit (CFU) as compared to a growth of the first bacterial strain when combined in a medium for at most about 15 hours not comprising the second bacterial strain or the supernatant thereof, which can include secreted metabolites (or bacterial products), fermentation products, and/or can be cell-free. In some embodiments, a pharmaceutical composition comprising a bacterial population comprising a first bacterial strain and different second bacterial strain can be combined within a medium for at most 30 hours, 25 hours, 20 hours, 15 hours, 10 hours, 8 hours or 5 hours, can exhibit a growth of at least 120%, 130%, 140%, or 149% by colony-forming unit (CFU) as compared to a growth of the first bacterial strain when combined in a medium for at most about hours not comprising the second bacterial strain or the supernatant thereof, which can include secreted metabolites (or bacterial products), fermentation products, and/or can be cell-free. In some embodiments, the media in which the first bacterial strain may be grown can comprise a secreted metabolite (or bacterial product) from the second bacterial strain or its supernatant. In some embodiments, the supernatant can be cell-free.

In some embodiments, a pharmaceutical composition comprising a bacterial population comprising a first bacterial strain and different second bacterial strain can be combined within a medium for at most 30 hours, 25 hours, 20 hours, 15 hours, 10 hours, 8 hours or 5 hours, can exhibit a growth of at least 150%, 160%, 170%, 180%, 190%, or 199% by colony-forming unit (CFU) as compared to a growth of the first bacterial strain when combined in a medium for at most about 15 hours not comprising the second bacterial strain or the supernatant thereof, which can include secreted metabolites (or bacterial products), fermentation products, and/or can be cell-free. In some embodiments, a pharmaceutical composition comprising a bacterial population comprising a first bacterial strain and different second bacterial strain can be combined within a medium for at most 30 hours, 25 hours, 20 hours, 15 hours, 10 hours, 8 hours or 5 hours, can exhibit a growth of at least 200%, 210%, 220%, 230%, 240%, 250%, or greater than 250% by colony-forming unit (CFU) as compared to a growth of the first bacterial strain when combined in a medium for at most about 15 hours not comprising the second bacterial strain or the supernatant thereof, which can include secreted metabolites (or bacterial products), fermentation products, and/or can be cell-free.

In some embodiments wherein a pharmaceutical composition comprising a bacterial population comprising a first bacterial strain and a different second bacterial strain, the first bacterial strain can be configured to utilize a metabolite (or bacterial product) derived from the bacterial strain as an energy source. In some embodiments, the metabolite (or bacterial product) is not a butyrate.

In some embodiments wherein a pharmaceutical composition comprising a bacterial population comprising at least one strain of Lactobacillus, sp. and/or at least one or more strains of Bifidobacterium sp., and/or at least one strain of Akkermansia sp., and/or at least one or more strains of Blautia sp. and/or at least one or more strains of Clostridium sp., and/or at least one or more strains of Coprococcus sp., and/or at least one or more strains of Dorea sp., and/or at least one or more strains of Faecalibacterium sp., and/or at least one or more strains of Roseburia sp., and/or at least one or more strains of Ruminococcus sp., and/or at least one or more strains of Anaerbutyricum sp., and/or at least one or more strains of Anaerostipes sp. and/or at least one or more strains of Anaerotignum sp., and/or at least one or more strains of Bacillus sp., and/or at least one or more strains of Bacteroides sp., and/or at least one or more strains of strain of Clostridium sp., and/or at least one or more strains of Collinsella sp., and/or at least one or more strains of Enterococcus sp., and/or at least one or more strains of Erysipelatoclostridium sp., and/or at least one or more strains of Escherichia sp., and/or at least one or more strains of Eubacterium sp. and/or at least one or more strains of Faecalicatena sp., and/or at least one or more strains of Holdemanella sp., and/or at least one or more strains of Lachnospira sp., and/or at least one or more strains of Longibaculum sp. and/or at least one or more strains of Paraprevotella sp., and/or at least one or more strains of Parabacteroides sp., and/or at least one or more strains of Pediococcus sp., and/or at least one or more strains of Veillonella sp., or a combination thereof, when combined within a medium comprising sialic acid, does not exhibit a growth of at least 101%, 105%, 110%, 115%, 120%, 130%, 140% 150%, 160%, 170%, 180%, 190%, 200%, 250%, 300%, 350%, 400%, 450%, 500%, 550%, 600%, 650%, 700%, 750%, 800%, 850%, 900%, 950%, 999%, 1000%, 1100%, or more than 1100% by weight as compared to the growth of a reference bacterial population when combined in a medium that can be comprised of glucose. In some embodiments, glucose can be a carbon source or the sole carbon source.

### Cultured Medium/Bacterial Products

In some cases, a bacterial strain or a bacterial population may exhibit a sufficient ability in a disease associated function using a bacterial product generated by the bacterial strain or the bacterial population. In some cases, two bacterial strains may exhibit a collective interaction using a bacterial product generated by at least one of the bacterial strains. In some cases, a bacterial strain or a bacterial population may exhibit a collective effect in a disease associated function using a bacterial product generated by the bacterial strain or the bacterial population.

The bacterial product may be present in a cultured medium of a bacterial strain. In some cases, the bacterial product may be present in a supernatant of the cultured medium. In some cases, when referring to a sufficient ability or collective effect of a bacterial strain on a disease associated function, the cultured medium of the bacterial strain, the supernatant thereof, or the bacterial product generated by the bacterial strain may have substantially the same or the same property or effect (i.e., the output of the property or effect being measured is within 80%, 85%, 90%, 91%, 92%, 93%, 94%, 95%, 96%, 97%, 98%, 99% or more). Thus, when referring to the sufficient ability or collective effect of the cultured medium of the bacterial strain, the supernatant thereof, or the bacterial product generated by the bacterial strain, the control can comprise the control described herein (such as the media control); or the cultured medium of the control bacterial strain, the supernatant thereof, or the bacterial product generated by the control bacterial strain.

In some cases, a first bacterial strain may convert a nutrient source into a first substance different from the nutrient source (such as by metabolic activity of the first bacterial strain) that is utilized by the second bacterial strain (different from the first bacterial strain). The second bacterial strain may use the first substance as a nutrient source (and for example, convert the first substance into a second substance different from the first substance and/or nutrient source). The second bacterial strain may not have a sufficient ability to utilize the nutrient source. Such interaction can allow the bacterial population comprising the first/second bacterial strains (or the cultured medium or the supernatant thereof) to exhibit a collective effect on a disease associated function as described herein. In another case, the generation of the second substance may only occur when the first bacterial population utilizes a nutrient source and convert to the first substance, whether the second bacterial strain can (or cannot) utilize the nutrient source. The second substance may facilitate the collective effect of the first/second bacterial strains (or the cultured medium or the supernatant thereof) on the disease associated function.

A supernatant of the cultured medium may comprise at least a fractionated or at least a partially purified derivative of the cultured medium. In some cases, the supernatant of the cultured medium may not comprise a bacterial cell. In some cases, the cultured medium may not comprise a bacterial cell. In some cases, the cultured medium or the supernatant thereof may comprise a bacterial cell. In some cases, the cultured medium or the supernatant thereof may comprise at most 1, 10, 100, 1000, or 10000 bacterial cells. In some cases, the cultured medium or the supernatant thereof having a sufficient ability a collective effect on a disease associated function may comprise at least 1, 2, 3, 4, 5 or more bacterial products.

In some cases, the pH of a cultured medium or a supernatant may be adjusted subsequent to culturing a bacterial strain with the cultured medium or a supernatant. For example, the pH may be lowered by at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7 or more. The pH may be lowered by at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, or 7. The pH may be increased by at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7 or more. The pH may be increased by at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, or 7. In some cases, the conditioned medium or supernatant may comprise a carbon source as described herein. The carbon source may comprise a vaginally-relevant carbohydrate or an infant gastrointestinally relevant carbohydrate. The conditioned medium with a carbon source can be used as a culture medium for culturing a recipient bacterial strain.

In some embodiments, the bacterial product may comprise a fermentation product of a bacterial strain. In some embodiments, the bacterial product may comprise a metabolite (or bacterial product). In some embodiments, the bacterial product may comprise a secreted metabolite (or bacterial product). In some embodiments, the bacterial product may comprise an organic compound. In some embodiments, the bacterial product may comprise an inorganic compound. In some embodiments, the bacterial product may comprise a peptide or polypeptide. In some embodiments, the bacterial product may comprise an amino acid. In some embodiments, the bacterial product may comprise a carbohydrate. In some embodiments, the bacterial product may comprise a derivative of a vaginally relevant carbohydrate. The bacterial product may comprise a derivative of glycogen, glucose, dextrin (such as maltodextrin), maltose, mucin, sialic acid, or a combination thereof. The bacterial product may comprise a derivative of glycogen. The bacterial product may comprise a derivative of glucose. The bacterial product may comprise a derivative of dextrin or maltodextrin. The bacterial product may comprise a derivative of maltose. The bacterial product may comprise a derivative of mucin. The bacterial product may comprise a derivative of sialic acid. The bacterial product may comprise a derivative of an infant gastrointestinally relevant carbohydrate. The bacterial product may comprise a derivative of HMOs. The bacterial product may have a sufficient ability in utilizing 2′FL. The bacterial product may have a sufficient ability in utilizing LNT. The bacterial product may have a sufficient ability in utilizing LNDFH-I. The bacterial product may have a sufficient ability in utilizing LNFP-I. The bacterial product may have a sufficient ability in utilizing LNFP-II. The bacterial product may have a sufficient ability in utilizing 3-FL. The bacterial product may have a sufficient ability in utilizing 6′-SL. The bacterial product may have a sufficient ability in utilizing DSLNT. The bacterial product may have a sufficient ability in utilizing LNnT. The bacterial product may have a sufficient ability in utilizing DFL. The bacterial product may have a sufficient ability in utilizing FDS-LNH. The bacterial product may have a sufficient ability in utilizing LNFP-III. The bacterial product may have a sufficient ability in utilizing 3′SL. In some embodiments, the bacterial product may comprise a lipid.

In some embodiments, the bacterial product may comprise lactic acids, hydrogen peroxide, cell wall components (such as lipoteichoic acid/LPS), proteinaceous or peptide products, short chain fatty acids, immunomodulatory lipids, bacteriocins or a combination thereof. In some embodiments, the bacterial product may comprise lactic acids. In some cases, the bacterial product may comprise hydrogen peroxide. In some embodiments, the bacterial product may comprise cell wall components. In some embodiments, the bacterial product may comprise LPS, In some embodiments, the bacterial product may comprise proteinaceous or peptide products. In some embodiments, the bacterial product may comprise short chain fatty acids. In some embodiments, the bacterial product may comprise immunomodulatory lipids. In some embodiments, the bacterial product may comprise bacteriocins.

In some embodiments, the bacterial product may not comprise a heterologous gene product. In some embodiments, the bacterial product may not comprise a recombinant gene product. In some embodiments, the bacterial product may not comprise a mammalian gene product. In some embodiments, the bacterial product may not comprise a synthetic gene product. In some embodiments, the bacterial product may not comprise a human cluster of differentiation 4 (CD4) peptide or a fragment thereof. In some embodiments, the bacterial product may not comprise a CD4 peptide or fragment thereof.

In some cases, the conditioned medium or supernatant of a bacterial strain described herein can have the sufficient ability in the disease associated function of the bacterial strain. In some cases, the bacterial product generated by a bacterial strain described herein can have the sufficient ability in the disease associated function of the bacterial strain. Thus, the sufficient ability of the bacterial strain described herein can be extend to the conditioned medium or supernatant of the bacterial strain or the bacterial product generated by the bacterial strain.

### Dosages

In some instances, a bacterial population may comprise a varying number of colony-forming units (CFU/dose) of each of the bacterial species and/or strain it contains. In some cases, a bacterial population may comprise from about 1×10\hat{ }3 CFU/dose to about 1×10\hat{ }12 CFU/dose of a bacterial species or strain. In some cases, a bacterial population may comprise from about 1×10\hat{ }3 CFU/dose to about 1×10\hat{ }11 CFU/dose of a bacterial species or strain. In some cases, a bacterial population may comprise from about 1×10\hat{ }3 CFU/dose to about 1×10\hat{ }10 CFU/dose of a bacterial species or strain. In some cases, a bacterial population may comprise from about 1×10\hat{ }3 CFU/dose to about 1×10\hat{ }9 CFU/dose of a bacterial species or strain. In some cases, a bacterial population may comprise from about 1×10\hat{ }3 CFU/dose to about 1×10\hat{ }8 CFU/dose of a bacterial species or strain. In some cases, a bacterial population may comprise from about 1×10\hat{ }4 CFU/dose to about 1×10\hat{ }12 CFU/dose of a bacterial species or strain. In some cases, a bacterial population may comprise from about 1×10\hat{ }4 CFU/dose to about 1×10\hat{ }11 CFU/dose of a bacterial species or strain. In some cases, a bacterial population may comprise from about 1×10\hat{ }4 CFU/dose to about 1×10\hat{ }10 CFU/dose of a bacterial species or strain. In some cases, a bacterial population may comprise from about 1×10\hat{ }4 CFU/dose to about 1×10\hat{ }9 CFU/dose of a bacterial species or strain. In some cases, a bacterial population may comprise from about 1×10\hat{ }4 CFU/dose to about 1×10\hat{ }8 CFU/dose of a bacterial species or strain. In some cases, a bacterial population may comprise from about 1×10\hat{ }5 CFU/dose to about 1×10\hat{ }12 CFU/dose of a bacterial species or strain. In some cases, a bacterial population may comprise from about 1×10\hat{ }5 CFU/dose to about 1×10\hat{ }11 CFU/dose of a bacterial species or strain. In some cases, a bacterial population may comprise from about 1×10\hat{ }5 CFU/dose to about 1×10\hat{ }10 CFU/dose of a bacterial species or strain. In some cases, a bacterial population may comprise from about 1×10\hat{ }5 CFU/dose to about 1×10\hat{ }9 CFU/dose of a bacterial species or strain. In some cases, a bacterial population may comprise from about 1×10\hat{ }5 CFU/dose to about 1×10\hat{ }8 CFU/dose of a bacterial species or strain. In some cases, a bacterial population may comprise from about 1×10\hat{ }6 CFU/dose to about 1×10\hat{ }12 CFU/dose of a bacterial species or strain. In some cases, a bacterial population may comprise from about 1×10\hat{ }6 CFU/dose to about 1×10\hat{ }11 CFU/dose of a bacterial species or strain. In some cases, a bacterial population may comprise from about 1×10\hat{ }6 CFU/dose to about 1×10\hat{ }10 CFU/dose of a bacterial species or strain. In some cases, a bacterial population may comprise from about 1×10\hat{ }6 CFU/dose to about 1×10\hat{ }9 CFU/dose of a bacterial species or strain. In some cases, a bacterial population may comprise from about 1×10\hat{ }6 CFU/dose to about 1×10\hat{ }8 CFU/dose of a bacterial species or strain. In some cases, a bacterial population may comprise from about 1×10\hat{ }7 CFU/dose to about 1×10\hat{ }12 CFU/dose of a bacterial species or strain. In some cases, a bacterial population may comprise from about 1×10\hat{ }7 CFU/dose to about 1×10\hat{ }11 CFU/dose of a bacterial species or strain. In some cases, a bacterial population may comprise from about 1×10\hat{ }7 CFU/dose to about 1×10\hat{ }10 CFU/dose of a bacterial species or strain. In some cases, a bacterial population may comprise from about 1×10\hat{ }7 CFU/dose to about 1×10\hat{ }9 CFU/dose of a bacterial species or strain. In some cases, a bacterial population may comprise from about 1×10\hat{ }7 CFU/dose to about 1×10\hat{ }8 CFU/dose of a bacterial species or strain. In some cases, a bacterial population may comprise from about 1×10\hat{ }8 CFU/dose to about 1×10\hat{ }12 CFU/dose of a bacterial species or strain. In some cases, a bacterial population may comprise from about 1×10\hat{ }8 CFU/dose to about 1×10\hat{ }11 CFU/dose of a bacterial species or strain. In some cases, a bacterial population may comprise from about 1×10\hat{ }8 CFU/dose to about 1×10\hat{ }10 CFU/dose of a bacterial species or strain. In some cases, a bacterial population may comprise from about 1×10\hat{ }8 CFU/dose to about 1×10\hat{ }9 CFU/dose of a bacterial species or strain. In some cases, a bacterial population may comprise from about 1×10\hat{ }9 CFU/dose to about 1×10\hat{ }12 CFU/dose of a bacterial species or strain. In some cases, a bacterial population may comprise from about 1×10\hat{ }9 CFU/dose to about 1×10\hat{ }11 CFU/dose of a bacterial species or strain. In some cases, a bacterial population may comprise from about 1×10\hat{ }9 CFU/dose to about 1×10\hat{ }10 CFU/dose of a bacterial species or strain. In some cases, such bacterial population may also comprise from about 1×10\hat{ }7 CFU/dose to about 1×10\hat{ }10 CFU/dose of a bacterial species or strain. In some instances, a bacterial population may comprise at least about 1×10\hat{ }3 CFU/dose, 5×10\hat{ }3 CFU/dose, 1×10\hat{ }4 CFU/dose, 5×10\hat{ }4 CFU/dose, 1×10\hat{ }5 CFU/dose, 5×10\hat{ }5 CFU/dose, 1×10\hat{ }6 CFU/dose, 5×10\hat{ }6 CFU/dose, 1×10\hat{ }7 CFU/dose, 5×10\hat{ }7 CFU/dose, 1×10\hat{ }8 CFU/dose, 5×10\hat{ }8 CFU/dose, 1×10\hat{ }9 CFU/dose, 5×10\hat{ }9 CFU/dose, 1×10\hat{ }10 CFU/dose, 5×10\hat{ }10 CFU/dose, 1×10\hat{ }11 CFU/dose, 5×10\hat{ }11 CFU/dose, or 1×10\hat{ }12 CFU/dose, but no more than about 5×10\hat{ }12 CFU/dose of a bacterial species or strain. The bacterial populations may also comprise from about 1×10\hat{ }6 to about 1×10\hat{ }11 CFU/dose per bacterial species or strain. In some cases, the bacterial populations may comprise from about 1×10\hat{ }3 to about 1×10\hat{ }12 CFU/dose per bacterial species or strain. In some instances, the bacterial populations may comprise from about 1×10\hat{ }8 to about 5×10\hat{ }10 CFU/dose per bacterial species or strain. In some instances, the bacterial populations may comprise from about 1×10\hat{ }7 to about 5×10\hat{ }10 CFU/dose per bacterial species or strain. In various cases, a bacterial population may comprise about 5×10\hat{ }8 CFU/dose per bacterial species or strain.

In some instances, a bacterial population may comprise a varying number of colony-forming units (CFU/dose) of bacterial cells. In some cases, a bacterial population may comprise from about 1×10\hat{ }3 CFU/dose to about 1×10\hat{ }12 CFU/dose of bacterial cells. In some cases, a bacterial population may comprise from about 1×10\hat{ }3 CFU/dose to about 1×10\hat{ }11 CFU/dose of bacterial cells. In some cases, a bacterial population may comprise from about 1×10\hat{ }3 CFU/dose to about 1×10\hat{ }10 CFU/dose of bacterial cells. In some cases, a bacterial population may comprise from about 1×10\hat{ }3 CFU/dose to about 1×10\hat{ }9 CFU/dose of bacterial cells. In some cases, a bacterial population may comprise from about 1×10\hat{ }3 CFU/dose to about 1×10\hat{ }8 CFU/dose of bacterial cells. In some cases, a bacterial population may comprise from about 1×10\hat{ }4 CFU/dose to about 1×10\hat{ }12 CFU/dose of bacterial cells. In some cases, a bacterial population may comprise from about 1×10\hat{ }4 CFU/dose to about 1×10\hat{ }11 CFU/dose of bacterial cells. In some cases, a bacterial population may comprise from about 1×10\hat{ }4 CFU/dose to about 1×10\hat{ }10 CFU/dose of bacterial cells. In some cases, a bacterial population may comprise from about 1×10\hat{ }4 CFU/dose to about 1×10\hat{ }9 CFU/dose of bacterial cells. In some cases, a bacterial population may comprise from about 1×10\hat{ }4 CFU/dose to about 1×10\hat{ }8 CFU/dose of bacterial cells. In some cases, a bacterial population may comprise from about 1×10\hat{ }5 CFU/dose to about 1×10\hat{ }12 CFU/dose of bacterial cells. In some cases, a bacterial population may comprise from about 1×10\hat{ }5 CFU/dose to about 1×10\hat{ }11 CFU/dose of bacterial cells. In some cases, a bacterial population may comprise from about 1×10\hat{ }5 CFU/dose to about 1×10\hat{ }10 CFU/dose of bacterial cells. In some cases, a bacterial population may comprise from about 1×10\hat{ }5 CFU/dose to about 1×10\hat{ }9 CFU/dose of bacterial cells. In some cases, a bacterial population may comprise from about 1×10\hat{ }5 CFU/dose to about 1×10\hat{ }8 CFU/dose of bacterial cells. In some cases, a bacterial population may comprise from about 1×10\hat{ }6 CFU/dose to about 1×10\hat{ }12 CFU/dose of bacterial cells. In some cases, a bacterial population may comprise from about 1×10\hat{ }6 CFU/dose to about 1×10\hat{ }11 CFU/dose of bacterial cells. In some cases, a bacterial population may comprise from about 1×10\hat{ }6 CFU/dose to about 1×10\hat{ }10 CFU/dose of bacterial cells. In some cases, a bacterial population may comprise from about 1×10\hat{ }6 CFU/dose to about 1×10\hat{ }9 CFU/dose of bacterial cells. In some cases, a bacterial population may comprise from about 1×10\hat{ }6 CFU/dose to about 1×10\hat{ }8 CFU/dose of bacterial cells. In some cases, a bacterial population may comprise from about 1×10\hat{ }7 CFU/dose to about 1×10\hat{ }12 CFU/dose of bacterial cells. In some cases, a bacterial population may comprise from about 1×10\hat{ }7 CFU/dose to about 1×10\hat{ }11 CFU/dose of bacterial cells. In some cases, a bacterial population may comprise from about 1×10\hat{ }7 CFU/dose to about 1×10\hat{ }10 CFU/dose of bacterial cells. In some cases, a bacterial population may comprise from about 1×10\hat{ }7 CFU/dose to about 1×10\hat{ }9 CFU/dose of bacterial cells. In some cases, a bacterial population may comprise from about 1×10\hat{ }7 CFU/dose to about 1×10\hat{ }8 CFU/dose of bacterial cells. In some cases, a bacterial population may comprise from about 1×10\hat{ }8 CFU/dose to about 1×10\hat{ }12 CFU/dose of bacterial cells. In some cases, a bacterial population may comprise from about 1×10\hat{ }8 CFU/dose to about 1×10\hat{ }11 CFU/dose of bacterial cells. In some cases, a bacterial population may comprise from about 1×10\hat{ }8 CFU/dose to about 1×10\hat{ }10 CFU/dose of bacterial cells. In some cases, a bacterial population may comprise from about 1×10\hat{ }8 CFU/dose to about 1×10\hat{ }9 CFU/dose of bacterial cells. In some cases, a bacterial population may comprise from about 1×10\hat{ }9 CFU/dose to about 1×10\hat{ }12 CFU/dose of bacterial cells. In some cases, a bacterial population may comprise from about 1×10\hat{ }9 CFU/dose to about 1×10\hat{ }11 CFU/dose of bacterial cells. In some cases, a bacterial population may comprise from about 1×10\hat{ }9 CFU/dose to about 1×10\hat{ }10 CFU/dose of bacterial cells. In some cases, such bacterial population may also comprise from about 1×10\hat{ }7 CFU/dose to about 1×10\hat{ }10 CFU/dose of bacterial cells. In some instances, a bacterial population may comprise at least about 1×10\hat{ }3 CFU/dose, 5×10\hat{ }3 CFU/dose, 1×10\hat{ }4 CFU/dose, 5×10\hat{ }4 CFU/dose, 1×10\hat{ }5 CFU/dose, 5×10\hat{ }5 CFU/dose, 1×10\hat{ }6 CFU/dose, 5×10\hat{ }6 CFU/dose, 1×10\hat{ }7 CFU/dose, 5×10\hat{ }7 CFU/dose, 1×10\hat{ }8 CFU/dose, 5×10\hat{ }8 CFU/dose, 1×10\hat{ }9 CFU/dose, 5×10\hat{ }9 CFU/dose, 1×10\hat{ }10 CFU/dose, 5×10\hat{ }10 CFU/dose, 1×10\hat{ }11 CFU/dose, 5×10\hat{ }11 CFU/dose, or 1×10\hat{ }12 CFU/dose, but no more than about 5×10\hat{ }12 CFU/dose of bacterial cells.

In some cases, at least one strain of Bifidobacterium sp. of a pharmaceutical composition may comprise from about 1×10\hat{ }6 CFU/dose to about 5×10\hat{ }9 CFU/dose. In some cases, at least one strain of Bifidobacterium sp. of a pharmaceutical composition may comprise from about 1×10\hat{ }6 CFU/dose to about 4×10\hat{ }9 CFU/dose. In some cases, at least one strain of Bifidobacterium sp. of a pharmaceutical composition may comprise from about 1×10\hat{ }6 CFU/dose to about 3×10\hat{ }9 CFU/dose. In some cases, at least one strain of Bifidobacterium sp. of a pharmaceutical composition may comprise from about 1×10\hat{ }6 CFU/dose to about 2×10\hat{ }9 CFU/dose. In some cases, at least one strain of Bifidobacterium sp. of a pharmaceutical composition may comprise from about 1×10\hat{ }6 CFU/dose to about 1×10\hat{ }9 CFU/dose. In some cases, at least one strain of Bifidobacterium sp. of a pharmaceutical composition may comprise from about 1×10\hat{ }6 CFU/dose to about 9×10\hat{ }8 CFU/dose. In some cases, at least one strain of Bifidobacterium sp. of a pharmaceutical composition may comprise from about 1×10\hat{ }6 CFU/dose to about 8×10\hat{ }8 CFU/dose. In some cases, at least one strain of Bifidobacterium sp. of a pharmaceutical composition may comprise from about 1×10\hat{ }6 CFU/dose to about 7×10\hat{ }8 CFU/dose. In some cases, at least one strain of Bifidobacterium sp. of a pharmaceutical composition may comprise from about 1×10\hat{ }6 CFU/dose to about 6×10\hat{ }8 CFU/dose. In some cases, at least one strain of Bifidobacterium sp. of a pharmaceutical composition may comprise from about 1×10\hat{ }6 CFU/dose to about 5×10\hat{ }8 CFU/dose. In some cases, at least one strain of Bifidobacterium sp. of a pharmaceutical composition may comprise from about 1×10\hat{ }6 CFU/dose to about 4×10\hat{ }8 CFU/dose. In some cases, at least one strain of Bifidobacterium sp. of a pharmaceutical composition may comprise from about 1×10\hat{ }6 CFU/dose to about 3×10\hat{ }8 CFU/dose. In some cases, at least one strain of Bifidobacterium sp. of a pharmaceutical composition may comprise from about 1×10\hat{ }6 CFU/dose to about 2×10\hat{ }8 CFU/dose. In some cases, at least one strain of Bifidobacterium sp. of a pharmaceutical composition may comprise from about 1×10\hat{ }6 CFU/dose to about 1×10\hat{ }8 CFU/dose. In some cases, at least one strain of Bifidobacterium sp. of a pharmaceutical composition may comprise from about 1×10\hat{ }6 CFU/dose to about 9×10\hat{ }7 CFU/dose. In some cases, at least one strain of Bifidobacterium sp. of a pharmaceutical composition may comprise from about 1×10\hat{ }6 CFU/dose to about 8×10\hat{ }7 CFU/dose. In some cases, at least one strain of Bifidobacterium sp. of a pharmaceutical composition may comprise from about 1×10\hat{ }6 CFU/dose to about 7×10\hat{ }7 CFU/dose. In some cases, at least one strain of Bifidobacterium sp. of a pharmaceutical composition may comprise from about 1×10\hat{ }6 CFU/dose to about 6×10\hat{ }7 CFU/dose. In some cases, at least one strain of Bifidobacterium sp. of a pharmaceutical composition may comprise from about 1×10\hat{ }6 CFU/dose to about 5×10\hat{ }7 CFU/dose. In some cases, at least one strain of Bifidobacterium sp. of a pharmaceutical composition may comprise from about 1×10\hat{ }6 CFU/dose to about 4×10\hat{ }7 CFU/dose. In some cases, at least one strain of Bifidobacterium sp. of a pharmaceutical composition may comprise from about 1×10\hat{ }6 CFU/dose to about 3×10\hat{ }7 CFU/dose. In some cases, at least one strain of Bifidobacterium sp. of a pharmaceutical composition may comprise from about 1×10\hat{ }6 CFU/dose to about 2×10\hat{ }7 CFU/dose. In some cases, at least one strain of Bifidobacterium sp. of a pharmaceutical composition may comprise from about 1×10\hat{ }6 CFU/dose to about 1×10\hat{ }7 CFU/dose. In some cases, at least one strain of Bifidobacterium sp. may comprise about 5×10\hat{ }9 CFU/dose. In some cases, at least one strain of Bifidobacterium sp. may comprise about 4×10\hat{ }9 CFU/dose. In some cases, at least one strain of Bifidobacterium sp. may comprise about 3×10\hat{ }9 CFU/dose. In some cases, at least one strain of Bifidobacterium sp. may comprise about 2×10\hat{ }9 CFU/dose. In some cases, at least one strain of Bifidobacterium sp. may comprise about 1×10\hat{ }9 CFU/dose. In some cases, at least one strain of Bifidobacterium sp. may comprise about 9×10\hat{ }8 CFU/dose. In some cases, at least one strain of Bifidobacterium sp. may comprise about 8×10\hat{ }8 CFU/dose. In some cases, at least one strain of Bifidobacterium sp. may comprise about 7×10\hat{ }8 CFU/dose. In some cases, at least one strain of Bifidobacterium sp. may comprise about 6×10\hat{ }8 CFU/dose. In some cases, at least one strain of Bifidobacterium sp. may comprise about 5×10\hat{ }8 CFU/dose. In some cases, at least one strain of Bifidobacterium sp. may comprise about 4×10\hat{ }8 CFU/dose. In some cases, at least one strain of Bifidobacterium sp. may comprise about 3×10\hat{ }8 CFU/dose. In some cases, at least one strain of Bifidobacterium sp. may comprise about 2×10\hat{ }8 CFU/dose. In some cases, at least one strain of Bifidobacterium sp. may comprise about 1×10\hat{ }8 CFU/dose. In some cases, at least one strain of Bifidobacterium sp. may comprise about 9×10\hat{ }7 CFU/dose. In some cases, at least one strain of Bifidobacterium sp. may comprise about 8×10\hat{ }7 CFU/dose. In some cases, at least one strain of Bifidobacterium sp. may comprise about 7×10\hat{ }7 CFU/dose. In some cases, at least one strain of Bifidobacterium sp. may comprise about 6×10\hat{ }7 CFU/dose. In some cases, at least one strain of Bifidobacterium sp. may comprise about 5×10\hat{ }7 CFU/dose. In some cases, at least one strain of Bifidobacterium sp. may comprise about 4×10\hat{ }7 CFU/dose. In some cases, at least one strain of Bifidobacterium sp. may comprise about 3×10\hat{ }7 CFU/dose. In some cases, at least one strain of Bifidobacterium sp. may comprise about 2×10\hat{ }7 CFU/dose. In some cases, at least one strain of Bifidobacterium sp. may comprise about 1×10\hat{ }7 CFU/dose.

In some cases, at least one strain of Vertebrate-Associated Lactobacillaceae of a pharmaceutical composition may comprise from about 1×10\hat{ }7 CFU/dose to about 5×10\hat{ }10 CFU/dose. In some cases, at least one strain of Vertebrate-Associated Lactobacillaceae of a pharmaceutical composition may comprise from about 1×10\hat{ }7 CFU/dose to about 4×10\hat{ }10 CFU/dose. In some cases, at least one strain of Vertebrate-Associated Lactobacillaceae of a pharmaceutical composition may comprise from about 1×10\hat{ }7 CFU/dose to about 3×10\hat{ }10 CFU/dose. In some cases, at least one strain of Vertebrate-Associated Lactobacillaceae of a pharmaceutical composition may comprise from about 1×10\hat{ }7 CFU/dose to about 2×10\hat{ }10 CFU/dose. In some cases, at least one strain of Vertebrate-Associated Lactobacillaceae of a pharmaceutical composition may comprise from about 1×10\hat{ }7 CFU/dose to about 1×10\hat{ }10 CFU/dose. In some cases, at least one strain of Vertebrate-Associated Lactobacillaceae of a pharmaceutical composition may comprise from about 1×10\hat{ }7 CFU/dose to about 9×10\hat{ }9 CFU/dose. In some cases, at least one strain of Vertebrate-Associated Lactobacillaceae of a pharmaceutical composition may comprise from about 1×10\hat{ }7 CFU/dose to about 8×10\hat{ }9 CFU/dose. In some cases, at least one strain of Vertebrate-Associated Lactobacillaceae of a pharmaceutical composition may comprise from about 1×10\hat{ }7 CFU/dose to about 7×10\hat{ }9 CFU/dose. In some cases, at least one strain of Vertebrate-Associated Lactobacillaceae of a pharmaceutical composition may comprise from about 1×10\hat{ }7 CFU/dose to about 6×10\hat{ }9 CFU/dose. In some cases, at least one strain of Vertebrate-Associated Lactobacillaceae of a pharmaceutical composition may comprise from about 1×10\hat{ }7 CFU/dose to about 5×10\hat{ }9 CFU/dose. In some cases, at least one strain of Vertebrate-Associated Lactobacillaceae of a pharmaceutical composition may comprise from about 1×10\hat{ }7 CFU/dose to about 4×10\hat{ }9 CFU/dose. In some cases, at least one strain of Vertebrate-Associated Lactobacillaceae of a pharmaceutical composition may comprise from about 1×10\hat{ }7 CFU/dose to about 3×10\hat{ }9 CFU/dose. In some cases, at least one strain of Vertebrate-Associated Lactobacillaceae of a pharmaceutical composition may comprise from about 1×10\hat{ }7 CFU/dose to about 2×10\hat{ }9 CFU/dose. In some cases, at least one strain of Vertebrate-Associated Lactobacillaceae of a pharmaceutical composition may comprise from about 1×10\hat{ }7 CFU/dose to about 1×10\hat{ }9 CFU/dose. In some cases, at least one strain of Vertebrate-Associated Lactobacillaceae of a pharmaceutical composition may comprise from about 1×10\hat{ }7 CFU/dose to about 9×10\hat{ }8 CFU/dose. In some cases, at least one strain of Vertebrate-Associated Lactobacillaceae of a pharmaceutical composition may comprise from about 1×10\hat{ }7 CFU/dose to about 8×10\hat{ }8 CFU/dose. In some cases, at least one strain of Vertebrate-Associated Lactobacillaceae of a pharmaceutical composition may comprise from about 1×10\hat{ }7 CFU/dose to about 7×10\hat{ }8 CFU/dose. In some cases, at least one strain of Vertebrate-Associated Lactobacillaceae of a pharmaceutical composition may comprise from about 1×10\hat{ }7 CFU/dose to about 6×10\hat{ }8 CFU/dose. In some cases, at least one strain of Vertebrate-Associated Lactobacillaceae of a pharmaceutical composition may comprise from about 1×10\hat{ }7 CFU/dose to about 5×10\hat{ }8 CFU/dose. In some cases, at least one strain of Vertebrate-Associated Lactobacillaceae of a pharmaceutical composition may comprise from about 1×10\hat{ }7 CFU/dose to about 4×10\hat{ }8 CFU/dose. In some cases, at least one strain of Vertebrate-Associated Lactobacillaceae of a pharmaceutical composition may comprise from about 1×10\hat{ }7 CFU/dose to about 3×10\hat{ }8 CFU/dose. In some cases, at least one strain of Vertebrate-Associated Lactobacillaceae of a pharmaceutical composition may comprise from about 1×10\hat{ }7 CFU/dose to about 2×10\hat{ }8 CFU/dose. In some cases, at least one strain of Vertebrate-Associated Lactobacillaceae of a pharmaceutical composition may comprise from about 1×10\hat{ }7 CFU/dose to about 1×10\hat{ }8 CFU/dose. In some cases, at least one strain of Vertebrate-Associated Lactobacillaceae may comprise about 5×10\hat{ }9 CFU/dose. In some cases, at least one strain of Vertebrate-Associated Lactobacillaceae may comprise about 4×10\hat{ }9 CFU/dose. In some cases, at least one strain of Vertebrate-Associated Lactobacillaceae may comprise about 3×10\hat{ }9 CFU/dose. In some cases, at least one strain of Vertebrate-Associated Lactobacillaceae may comprise about 2×10\hat{ }9 CFU/dose. In some cases, at least one strain of Vertebrate-Associated Lactobacillaceae may comprise about 1×10\hat{ }9 CFU/dose. In some cases, at least one strain of Vertebrate-Associated Lactobacillaceae may comprise about 9×10\hat{ }8 CFU/dose. In some cases, at least one strain of Vertebrate-Associated Lactobacillaceae may comprise about 8×10\hat{ }8 CFU/dose. In some cases, at least one strain of Vertebrate-Associated Lactobacillaceae may comprise about 7×10\hat{ }8 CFU/dose. In some cases, at least one strain of Vertebrate-Associated Lactobacillaceae may comprise about 6×10\hat{ }8 CFU/dose. In some cases, at least one strain of Vertebrate-Associated Lactobacillaceae may comprise about 5×10\hat{ }8 CFU/dose. In some cases, at least one strain of Vertebrate-Associated Lactobacillaceae may comprise about 4×10\hat{ }8 CFU/dose. In some cases, at least one strain of Vertebrate-Associated Lactobacillaceae may comprise about 3×10\hat{ }8 CFU/dose. In some cases, at least one strain of Vertebrate-Associated Lactobacillaceae may comprise about 2×10\hat{ }8 CFU/dose. In some cases, at least one strain of Vertebrate-Associated Lactobacillaceae may comprise about 1×10\hat{ }8 CFU/dose. In some cases, at least one strain of Vertebrate-Associated Lactobacillaceae may comprise about 9×10\hat{ }7 CFU/dose. In some cases, at least one strain of Vertebrate-Associated Lactobacillaceae may comprise about 8×10\hat{ }7 CFU/dose. In some cases, at least one strain of Vertebrate-Associated Lactobacillaceae may comprise about 7×10\hat{ }7 CFU/dose. In some cases, at least one strain of Vertebrate-Associated Lactobacillaceae may comprise about 6×10\hat{ }7 CFU/dose. In some cases, at least one strain of Vertebrate-Associated Lactobacillaceae may comprise about 5×10\hat{ }7 CFU/dose. In some cases, at least one strain of Vertebrate-Associated Lactobacillaceae may comprise about 4×10\hat{ }7 CFU/dose. In some cases, at least one strain of Vertebrate-Associated Lactobacillaceae may comprise about 3×10\hat{ }7 CFU/dose. In some cases, at least one strain of Vertebrate-Associated Lactobacillaceae may comprise about 2×10\hat{ }7 CFU/dose. In some cases, at least one strain of Vertebrate-Associated Lactobacillaceae may comprise about 1×10\hat{ }7 CFU/dose.

In some cases, identification of bacterial strains or isolates may be performed by sequencing of the full-length 16S rRNA gene. Such a method may use one or more amplification primer followed by nucleic acid sequencing. Full-length 16S rRNA gene sequence reads can be aligned in the Ribosomal Database Project (RDP), manually curated using nucleic acid analysis and sequencing programs (e.g., ARB, mother, etc.) to classify reads to operational taxonomic units (OTUs). The full-length 16S rRNA gene sequence of each species-level OTU can then be compared to the RDP reference database to assign taxonomic designations to the genus and/or strain level followed by a BLASTn search to either a characterized or candidate novel species.

The bacterial population may comprise a purified bacterial strain. The term “purified” or “substantially purified” as used herein when referring to a bacterial strain or a mixture of more than one bacterial strain, refers to the bacterial strain or bacterial strains that are substantially enriched in a sample. A purified or substantially purified bacterial strain(s) in sample may comprise at least about 50%, 60%, 70%, 80%, 85%, 90%, 95%, 99% or greater of the bacterial strain(s) in the sample. A purified or substantially purified bacterial strain(s) in sample may also comprise less than about 40%, 30%, 20%, 15%, 14%, 13%, 12%, 11%, 10%, 9%, 8%, 7%, 6%, 5%, 4%, 3%, 2%, 1% or less of the strains other than the bacterial strain(s) present in the sample. Such strain may comprise a bacterial strain. Such strain may also comprise any non-bacterial strains such as strains from other organisms.

Within a bacterial population comprising a first and second bacterial strains, the first bacterial strain is at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold higher than an amount of the second bacterial strain. Within a bacterial population comprising a first and second bacterial strains, the first bacterial strain is at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold higher than an amount of the second bacterial strain. Within a bacterial population comprising a first and second bacterial strains, the first bacterial strain is at least about 1%, at least about 2%, at least about 3%, at least about 4% at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 150%, at least about 2-fold, at least about 3-fold, at least about 4-fold, at least about 5-fold, at least about 6-fold, at least about 7-fold, at least about 8-fold, at least about 9-fold, at least about 10-fold, at least about 100-fold, at least about 1000-fold, at least about 10000-fold, at least about 100000-fold, or at least about 1000000-fold lower than an amount of the second bacterial strain. Within a bacterial population comprising a first and second bacterial strains, the first bacterial strain is at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100%, at most about 150%, at most about 2-fold, at most about 3-fold, at most about 4-fold, at most about 5-fold, at most about 6-fold, at most about 7-fold, at most about 8-fold, at most about 9-fold, at most about 10-fold, at most about 100-fold, at most about 1000-fold, at most about 10000-fold, at most about 100000-fold, or at most about 1000000-fold lower than an amount of the second bacterial strain.

In some cases, a bacterial population comprises a viable cell. In some cases, a bacterial population comprises an inviable cell. In some cases, a bacterial population comprises both a viable cell and an inviable cell. In some cases, the bacterial population may comprise at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100% viable cells. In some cases, the bacterial population may comprise at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100% viable cells. In some cases, the bacterial population may comprise at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100% inviable cells. In some cases, the bacterial population may comprise at most about 1%, at most about 2%, at most about 3%, at most about 4%, at most about 5%, at most about 6%, at most about 7%, at most about 8%, at most about 9%, at most about 10%, at most about 20%, at most about 30%, at most about 40%, at most about 50%, at most about 60%, at most about 70%, at most about 80%, at most about 90%, at most about 100% inviable cells.

In some cases, the composition or the bacterial population may be formulated into a therapeutically-effective amount. The therapeutically-effective amount may comprise any amount of bacteria as described herein, such that at least one of the disease associated functions (such as vaginal disease-associated function and NEC-associated function).

### Excipients

In some instances, a pharmaceutical composition may comprise a pharmaceutically acceptable excipient. The term “pharmaceutically acceptable excipient” or “excipient”, when referring to a pharmaceutical composition, refers to an excipient that does not produce an adverse, allergic, or other untoward reaction when administered to a subject, preferably a human. A pharmaceutically acceptable excipient may not produce the adverse, allergic, or other untoward reaction when administered to the subject when it is administered alone or in combination with the pharmaceutical composition. A pharmaceutically acceptable excipient may include any solvents, dispersion media, coatings, isotonic and absorption delaying agents and the like. A pharmaceutically acceptable excipient may be added to a pharmaceutical composition to stabilize the pharmaceutical composition. A pharmaceutically acceptable excipient may be added to a pharmaceutical composition to prevent the contamination of the pharmaceutical composition. A pharmaceutically acceptable excipient may be an inert substance added to a pharmaceutical composition to facilitate processing, handling, administration, etc. of the pharmaceutical composition.

In some cases, a pharmaceutically acceptable excipient may comprise at least a moisture absorbent material, an adjuvant, an antiadherent, a binder, a carrier, a disintegrant, a filler, a flavor, a color, a diluent, a lubricant, a glidant, a preservative, a sorbent, a solvent, a surfactant, a sweetener, a cryoprotectant, or any combination thereof. In some cases, a cryoprotectant may comprise a carbohydrate and an antioxidant. In some embodiments, the carbohydrate may comprise a saccharose, a trehalose, or a combination thereof. In some embodiments, the antioxidant may comprise amino acid. In some cases, a moisture absorbent material may comprise microcrystalline cellulose (MCC), hydroxypropyl methylcellulose, silicon dioxide (SiO₂), polyethylene glycol 8000, lactose, d-trehalose dihydrate, mannitol, calcium phosphate tribasic, calcium sulfate, corn starch, fructose, xylitol, maltitol, anhydrous lactose and dicalcium phosphate (DCP).

### Forms and Administrations

The composition described herein may be formulated into a pharmaceutical composition. The term “pharmaceutical composition” or “composition”, when referring to a pharmaceutical product, refers to a composition that can elicit or induce at least one physiological effect to a subject when administered by the subject, preferably a human. Such physiological effect may positively contribute to the overall health of a subject. In some cases, such physiological effect may also curb, inhibit, reduce, or decrease a negative physiological phenomenon of the subject.

In some instances, a pharmaceutical composition may be formulated into a suspension. In some cases, a pharmaceutical composition may be formulated into an oral dosage form or a vaginal dosage form. An oral dosage form of a pharmaceutical composition, in some cases, may comprise a capsule, tablet, emulsion, suspension, syrup, gel, gum, paste, herbal tea, drops, dissolving granules, powders, tablets, lyophilizate, a popsicle, or ice cream. In some cases, an oral dosage form of a pharmaceutical composition may comprise a capsule. In some cases, an oral dosage form of a pharmaceutical composition may comprise a dissolving granule. In some cases, an oral dosage form of a pharmaceutical composition may comprise a drop. In some cases, an oral dosage form of a pharmaceutical composition may comprise an emulsion. In some cases, an oral dosage form of a pharmaceutical composition may comprise a gel. In some cases, an oral dosage form of a pharmaceutical composition may comprise a gum. In some cases, an oral dosage form of a pharmaceutical composition may comprise an herbal tea. In some cases, an oral dosage form of a pharmaceutical composition may comprise an ice cream. In some cases, an oral dosage form of a pharmaceutical composition may comprise a lyophilizate. In some cases, an oral dosage form of a pharmaceutical composition may comprise a paste. In some cases, an oral dosage form of a pharmaceutical composition may comprise a popsicle. In some cases, an oral dosage form of a pharmaceutical composition may comprise a powder. In some cases, an oral dosage form of a pharmaceutical composition may comprise a suspension. In some cases, an oral dosage form of a pharmaceutical composition may comprise a syrup. In some cases, an oral dosage form of a pharmaceutical composition may comprise a tablet. In some cases, an oral dosage form of a pharmaceutical composition may comprise a pill, gel tab, sachet, a lozenge, or any other suitable oral dosage form. In some cases, a pharmaceutical composition in an oral dosage or suspension form may be administered alone. In other cases, a pharmaceutical composition in an oral dosage or suspension form may be mixed with a food product for administration to a subject. Such a food product may comprise baby formula, milk, or any derivatives thereof. In some cases, a pharmaceutical composition may be formulated into a parenteral administration form. A parenteral administration form, in some cases, may comprise various non-oral routes, e.g., in the form of a suppository.

A pharmaceutical composition for treating or preventing the vaginal or a complication of the vaginal disease may comprise a vaginal dosage form. A pharmaceutical composition for treating or preventing the vaginal or a complication of the vaginal disease may comprise a gel. Such a gel may comprise a hydrogel. A hydrogel can comprise a 3-dimensional polymer cross-linked with water soluble polymers. A hydrogel may comprise a thermo-responsive hydrogel. In some cases, the hydrogel may be mucoadhesive. A hydrogel can comprise a vaginally delivered aqueous hydrogel. A vaginal dosage form may comprise a suspension that is injectable to a vagina. The vaginal dosage form may comprise a suspension that is injectable to a vagina with the aid of a vaginal applicator.

A pharmaceutical composition for treating or preventing the infant gastrointestinal disease may comprise an oral dosage form. A pharmaceutical composition for treating or preventing the infant gastrointestinal disease may comprise a suspension of a bacterial population. Prior to forming the suspension, the bacterial population may be formulated into a solid or a liquid form as described herein. For example, the bacterial population may be formulated into a powder. In some cases, the pharmaceutical composition for treating or preventing the infant gastrointestinal disease may be mixed with HMOs. The pharmaceutical composition for treating or preventing the infant gastrointestinal disease may be mixed with baby formula. The pharmaceutical composition for treating or preventing the infant gastrointestinal disease may be mixed with human breast milk.

A composition for treating or preventing the vaginal disease may be mixed with a vaginally relevant carbohydrate as described herein. A pharmaceutical composition for treating or preventing the infant gastrointestinal disease may be mixed with an infant gastrointestinally relevant carbohydrate as described herein. Mixing the compositions with the carbohydrates (such as vaginally relevant carbohydrate/infant gastrointestinally relevant carbohydrate can increase the effect of the composition in the disease associated function, as described here.

In some instances, a pharmaceutical composition may be formulated into a storage form. The storage form may be encompassed within a container. The container may be a capsule or a tablet. For example, the pharmaceutical composition may be formulated into a powder stored within a capsule or a tablet. The pharmaceutical composition may be formulated into a suspension stored within a capsule or a tablet or a container capable of storing liquid.

In some instances, a pharmaceutical composition may be formulated into an enteral dosage form. An enteral dosage form, in some instances, may compromise a rectal dosage form, intragastric dosage form, or an oral dosage form. A rectal dosage form, in some instances, may comprise a suppository, gel, or enema suspension or solution. In some cases, an intragastric dosage form may comprise a dosage form that is configured to pass through a feeding tube. In some cases, an intragastric dosage form may comprise a gastroenteral solution, a gastroenteral suspension, or other gastroenteral liquid. In some cases, a pharmaceutical composition may be formulated into an injectable dosage form. A parenteral dosage form, in some instances, may comprise an injectable dosage form, an infusible dosage form, an emulsified injectable dosage form, or an emulsified infusible dosage form or a combination thereof. An injectable dosage pharmaceutical composition, in some instances, may comprise a liquid preparation. In some cases, an injectable dosage pharmaceutical composition may comprise a dry solid that, upon the addition of suitable vehicles, yield injectable solutions. In some cases, an injectable dosage pharmaceutical composition may comprise a liquid suspension of solids suspended in a suitable liquid medium. In some cases, an injectable dosage pharmaceutical composition may comprise an intramuscular or subcutaneous injection. An infusible dosage pharmaceutical composition, in some instances, may comprise a liquid preparation. In some cases, an infusible dosage pharmaceutical composition may comprise a dry solid that, upon the addition of suitable vehicles, yield infusible solutions. In some cases, an infusible dosage pharmaceutical composition may comprise a liquid suspension of solids suspended in a suitable liquid medium. In some cases, an infusible dosage pharmaceutical composition may comprise an intramuscular or subcutaneous infusion. In some instances, a pharmaceutical composition may be formulated into a topical dosage form. In some cases, a topical dosage pharmaceutical composition may comprise creams, foams, gels, lotions, ointments, pastes, powders, or a combination thereof.

In some cases, a pharmaceutical composition may be encompassed by a primary container. In some instances, a pharmaceutical composition may be encompassed by a capsule. In some cases, a capsule encompassing a pharmaceutical composition may comprise a plant-based capsule. In some cases, a capsule encompassing a pharmaceutical composition may comprise a vegan capsule. In some cases, a plant-based capsule may comprise a plant-derived material. The plant-derived material, in some cases, may comprise a cellulose-based polymer. In some cases, a plant-based capsule may comprise a hypromellose capsule. In some cases, a plant-based capsule may comprise a hydroxypropyl methylcellulose (HPMC) capsule. In some cases, a plant-based capsule may comprise a starch capsule. In some cases, a plant-based capsule may comprise a hydrolyzed plant-based collagen capsule. In some cases, a plant-based capsule may comprise a pullulan capsule. In some cases, a plant-based capsule may comprise a tapioca capsule. In some cases, a plant-based capsule may comprise the combinations of any plant-based materials described thereof. A primary container, in some cases, may comprise any capsules described herein and thereof and derivatives herein and thereof.

In some cases, a capsule may not be administered to a subject. In some cases, a capsule may not be administered to a subject alongside the pharmaceutical composition. In other cases, a capsule may be administered to a subject.

In some cases, a pharmaceutical composition may be lyophilized. In some cases, a pharmaceutical composition may be frozen. Such frozen or lyophilized formulations may be administered in a frozen or lyophilized state to a subject. In some instances, such frozen formulation may be a popsicle, an ice cream, or other frozen formulations.

In some cases, a liquid suspension may be aliquoted into certain volumes to provide a unit dose of such oral dosage form. Such unit dose may have a volume of about 0.25, 0.5, 1, 2, 3, 5, or 10 mL. In some instances, the unit dose of a pharmaceutical composition herein has a volume of about 1 mL. Such pharmaceutical composition may comprise a bacterial population, a cryoprotectant, an antioxidant, an aqueous buffer solution that may from a liquid cell suspension. Such cell suspension may be tested for quality control to ensure it contains a certain number of metabolically active cells per bacterial strain as described herein.

In some cases, a capsule, tablet or other solid or semisolid pharmaceutical composition may be administered as a vaginal suppository to the subject.

Provided herein are pharmaceutical compositions that can be designed and manufactured to allow storage and/or transport of the pharmaceutical compositions. In some instances, a pharmaceutical composition herein comprising a bacterial population can be designed such that the viability of the bacterial cells in the pharmaceutical composition is not or only minimally affected by storage and/or transport. In such instances, the viability of at least about 80%, 85%, 90%, 95%, 97%, or 99% of bacterial cells in the pharmaceutical composition is maintained during storage and/or transport.

In some instances, a pharmaceutical composition herein comprises a cryoprotectant to allow storage at low temperatures at about −70° C. or −80° C. to preserve viability of the bacterial cells. A pharmaceutical composition herein can further comprise an antioxidant that can preserve an anaerobic environment in the storage or transport vial and can protect the bacterial cells from reactive oxygen species. The term “oxygen-free” or “anaerobic” as used herein refers to a state with low oxygen or without oxygen. An oxygen-free or anaerobic environment may have 21%, 20%, 19%, 18%, 17%, 16%, 15%, 14%, 13%, 12%, 11%, 10%, 9%, 8%, 7%, 6%, 5%, 4%, 3%, 2%, 1%, 1×10\hat{ }-1%, 1×10\hat{ }-2%, 1×10\hat{ }-3%, 1×10\hat{ }-4%, 1×10\hat{ }-5%, 1×10\hat{ }-6%, 1×10\hat{ }-7%, 1×10\hat{ }-8%, 1×10\hat{ }-9%, 1×10\hat{ }-10%, 1×10\hat{ }-11%, or less oxygen, by volume, in the atmosphere of the environment.

In some instances, the antioxidants described herein and thereof may be used with the bacterial populations, the pharmaceutical compositions, the methods for producing pharmaceutical compositions, or the methods for large-scale growth of Vertebrate-Associated Lactobacillaceae or Bifidobacterium sp. described in this disclosure. In some cases, the antioxidants described herein and thereof may be used with the growth media or excipients described in this disclosure. In other cases, the antioxidants described herein and thereof may be used in any embodiments or examples described in this disclosure.

### Methods of Treatment

In some embodiments, a method of administering the pharmaceutical composition can comprise administering the pharmaceutical composition to a subject who has a disease or disease condition as described herein.

In some embodiments, a method of administering the pharmaceutical composition can comprise administering the pharmaceutical composition to a subject who has bacterial vaginosis (BV) and/or a risk of the BV, wherein the subject is at least about 5 years old, 10 years old or at least about 15 years old. In some embodiments, the subject is at most about 80 years old, about 70 years old, about 65 years old, about 60 years old, about 55 years old, or about 50 years old. In some embodiments, a method can comprise of administering the pharmaceutical composition to a subject who has necrotizing enterocolitis (NEC) and/or a risk of the NEC wherein the subject is at least about 1 day old and at most about 1 year old and the subject is a premature infant. In some embodiments, the pharmaceutical composition can be administered to the subjects who have microbial dysbiosis in the gastrointestinal (GI) tract or vagina.

In some instances, a pharmaceutical composition may be administered to a subject having or suspected of having a disease or disease condition. In some cases, a pharmaceutical composition may be administered to a subject having a disease or disease condition. In other cases, a pharmaceutical composition may be administered to a subject suspected of having a disease or disease condition. In some cases, a pharmaceutical composition may be administered to a subject to treat a disease or disease condition in the subject. In some cases, a pharmaceutical composition may be administered to a subject to prevent a disease or disease condition in the subject. In some cases, when used to prevent a disease, the subject may not have developed the disease or disease condition before being administered with the pharmaceutical composition.

In some instances, a disease or disease condition treated/prevented by a pharmaceutical composition may comprise a disease or disease condition associated with microbial dysbiosis. Microbial dysbiosis is a reduction in microbial diversity that creates an imbalance in the microbial community of certain areas of the body, including but not limited to the gut and the vagina, resulting in inflammatory disease or disease condition. In some cases, the disease can comprise a vaginal disease or disease condition or gastrointestinal disease or disease condition (such as an infant gastrointestinal disease or disease condition).

A disease or disease condition may comprise a disease or a complication associated with the disease. For example, the complication associated with the disease may comprise a symptom or condition that develops subsequently to or concurrently with a subject has developed the disease or disease condition. In some cases, the development of the disease may increase the risk of symptoms or conditions. The development of the disease or disease condition may directly facilitate the development of the complication. The development of the disease or disease condition may indirectly facilitate the development of the complication.

In some cases, a subject administered with the pharmaceutical compositions described herein and thereof may comprise a human subject.

For infant gastrointestinal diseases, a human subject that is administered with the pharmaceutical compositions described herein and thereof, in some cases, have an age of at least about 1 day old, at least about 2 days old, at least about 3 days old, at least about 4 days old, at least about 5 days old, at least about 6 days old, at least about 1 week old, at least about 2 weeks old, at least about 3 weeks old, at least about 4 weeks old, at least about 1 month old, at least about 2 months old, at least about 3 months old, at least about 4 months old, at least about 5 months old, at least about 6 months old, at least about 7 months old, at least about 8 months old, at least about 9 months old, at least about 10 months old, at least about 11 months old, or at most about 1 year old. For infant gastrointestinal disease or disease condition, a human subject may be an infant or a neonate. In some cases, the infant may be a preterm infant or a neonate.

For vaginal diseases, a human subject may be an infant of an adult. For vaginal disease or disease condition, a human subject may be at least about 2 years old, at least about 3 years old, at least about 4 years old, at least about 5 years old, at least about 6 years old, at least about 7 years old, at least about 8 years old, at least about 9 years old, at least about 10 years old, at least about 11 years old, at least about 12 years old, at least about 13 years old, at least about 14 years old, at least about 15 years old, at least about 16 years old, at least about 17 years old, at least about 18 years old, at least about 19 years old, at least about 20 years old, at least about 21 years old, at least about 22 years old, at least about 23 years old, at least about 24 years old, at least about 25 years old, at least about 26 years old, at least about 27 years old, at least about 28 years old, at least about 29 years old, at least about 30 years old, at least about 31 years old, at least about 32 years old, at least about 33 years old, at least about 34 years old, at least about 35 years old, at least about 36 years old, at least about 37 years old, at least about 38 years old, at least about 39 years old, at least about 40 years old, at least about 41 years old, at least about 42 years old, at least about 43 years old, at least about 44 years old, at least about 45 years old, at least about 46 years old, at least about 47 years old, at least about 48 years old, at least about 49 years old, at least about 50 years old, at least about 51 years old, at least about 52 years old, at least about 53 years old, at least about 54 years old, at least about 55 years old, at least about 56 years old, at least about 57 years old, at least about 58 years old, at least about 59 years old, at least about 60 years old, at least about 61 years old, at least about 62 years old, at least about 63 years old, at least about 64 years old, at least about 65 years old, at least about 66 years old, at least about 67 years old, at least about 68 years old, at least about 69 years old, at least about 70 years old, at least about 71 years old, at least about 72 years old, at least about 73 years old, at least about 74 years old, at least about 75 years old, at least about 76 years old, at least about 77 years old, at least about 78 years old, at least about 79 years old, at least about 80 years old, at least about 81 years old, at least about 82 years old, at least about 83 years old, at least about 84 years old, at least about 85 years old, at least about 86 years old, at least about 87 years old, at least about 88 years old, at least about 89 years old, at least about 90 years old, at least about 91 years old, at least about 92 years old, at least about 93 years old, at least about 94 years old, at least about 95 years old, at least about 96 years old, at least about 97 years old, at least about 98 years old, at least about 99 years old, or at least about 100 years old.

In some cases, a vaginal disease can comprise BV. A vaginal disease can comprise recurrent BV. A complication associated with a vaginal disease can comprise pre-term birth, birth/pregnancy complications, STIs (such as HIV Chlamydia, Gonorrhea, Suphilis, Trichomoniasis), pelvic inflammatory disease (PID), vulvovaginitis) or a combination thereof. The complication associated with BV can comprise pre-term birth. The complication associated with BV can comprise STIs or a risk thereof. The complication associated with BV can comprise birth/pregnancy complications. In some In some cases, BV can comprise recurrent BV. In some cases, the pharmaceutical composition described herein can treat or prevent morbidity associated with the BV or the complication associated with BV. The complication associated with BV can comprise PID. The complication associated with BV can comprise vulvovaginitis.

In some cases, a gastrointestinal disease can comprise infant gastrointestinal disease. An infant gastrointestinal disease can comprise NEC, infectious gastroenteritis, neonatal cholestasis, pediatric intestinal motility disorder, gastroenteritis, Inflammatory bowel disease (IBD), Irritable bowel syndrome (IBS), or a combination thereof. An infant gastrointestinal disease can comprise NEC. An infant gastrointestinal disease can comprise infectious gastroenteritis. An infant gastrointestinal disease can comprise neonatal cholestasis. An infant gastrointestinal disease can comprise intestinal motility disorder. An infant gastrointestinal disease can comprise gastroenteritis. An infant gastrointestinal disease can comprise Inflammatory bowel disease (IBD). An infant gastrointestinal disease can comprise Irritable bowel syndrome (IBS). An infant gastrointestinal disease can comprise morbidity associated with gastrointestinal tract complications or NEC.

In some instances, a disease or disease condition treated by a pharmaceutical composition may comprise an inflammatory disease. In some cases, an inflammatory disease treated by a pharmaceutical bacterial population may comprise bacterial vaginosis or necrotizing enterocolitis. In some cases, a disease treated by a pharmaceutical composition may comprise BV, an inflammatory disease of the vagina resulting from a dysbiosis of the vaginal microbiome, resulting in symptoms of vaginal discharge, vaginal odor, vaginal itching, and/or burning during urination. In some cases, an inflammatory disease treated by a pharmaceutical composition may comprise necrotizing enterocolitis, an inflammatory gastrointestinal disease associated with dysbiosis of the preterm gut microbiota, resulting in symptoms of abdominal pain and swelling, changes in heart rate, blood pressure, body temperature, and breathing, diarrhea with bloody stool, green or yellow vomit, lethargy, refusal to eat or lack of weight gain, and can be fatal.

The pharmaceutical composition described herein can be used to treat or prevent BV in a human subject. In some cases, the human subject has not been treated for BV. In some cases, the human subject has been treated for BV. For example, a human subject that has been for BV may have been administered for an antibiotic. The antibiotic may be used to inhibit or eliminate a vaginal pathogen as described herein. In some cases, the human subject that is being treated with the pharmaceutical composition as described herein has been treated with a medication that targets BV. In some cases, the human subject has recurrent BV (i.e., the human subject has suffered from BV previously). In some cases, the human subject has not suffered from BV previously.

A pharmaceutical composition herein can be administered for various periods of time according to different administration schedules. A treatment period may vary between subjects and individuals and can depend on various factors as described herein, e.g., disease state, age, etc. In some instances, a subject can be treated for one day to at least about one week, for about a week to about one month, or for about one month to about one year. In such instances, a subject can be treated for about one month, two months, or three months. In some cases, treatment can be performed on consecutive days, consecutive weeks, and/or consecutive months. In some embodiments, a pharmaceutical composition is administered for about 28, 29, or 30 consecutive days.

Methods of treatment herein can include administering a pharmaceutical composition of this disclosure once, two, three, four, five, six, seven, eight, nine, ten, eleven, or twelve times daily. In various instances, a pharmaceutical composition of this disclosure is administered twice daily. Such twice daily administration can be performed in the morning and in the evening, in the morning and in the afternoon, in the afternoon and the evening, in the morning and at night, in the afternoon and at night, and in the evening at night. In such cases, there can be a period of about less than 1, 1, 2, 3, 4, 5, 6, 7, 8, 9, 10, 11, 12, 13, 14, 15, 16, 17, 18, 19, 20, 21, 22, 23, or 24 hours between the first and the second administration of a given day.

### Method of Identifying a Bacterial Population

Provided herein are method of identifying bacterial strains described. In some cases, the method comprises providing a first bacterial strain. In some cases, the method comprises generating a plurality of cultures, In some cases, one of the plurality of cultures comprises the first bacterial strain and: (1) a given bacterial strain of a plurality of bacterial strains or (2) a metabolite generated by the given bacterial strain. In some cases, each of the plurality of cultures comprises the first bacterial strain and: (1) a given bacterial strain of a plurality of bacterial strains or (2) a metabolite generated by the given bacterial strain. In some cases, the method comprises determining a culture of the plurality of cultures that is capable of exhibiting a sufficient ability or a collective effect on the disease associated function as described herein.

In some cases, the method comprises: (a) providing a first bacterial strain; (b) generating a plurality of cultures in which a culture of the plurality of cultures comprises the first bacterial strain and: (1) a given bacterial strain of a plurality of bacterial strains or (2) a bacterial product generated by the given bacterial strain; and (c) determining a culture of the plurality of cultures of (b) that is capable of exhibiting a sufficient ability or a collective effect on the disease associated function as described herein, thereby identifying the given bacterial strain to generate the bacterial combination comprising the first bacterial strain and the given bacterial strain. In some cases, the method comprises: (a) providing a first bacterial strain; (b) generating a plurality of cultures, each comprising the first bacterial strain and: (1) a given bacterial strain of a plurality of bacterial strains or (2) a bacterial product generated by the given bacterial strain; and (c) determining a culture of the plurality of cultures of (b) that is capable of exhibiting a sufficient ability or a collective effect on the disease associated function as described herein, thereby identifying the given bacterial strain to generate the bacterial combination comprising the first bacterial strain and the given bacterial strain. In some cases, the first bacterial strain may comprise a recipient bacterial strain as described herein. In some cases, the plurality of bacterial strains of (b) may comprise a list of potential donor bacterial strains. In some cases, the culture of (b) may comprise the first bacterial strain and a cultured medium or supernatant thereof of a given bacterial strain of a plurality of bacterial strains. In some cases, the method comprises: (a) providing a first bacterial strain or a bacterial product generated by the first bacterial strain; (b) generating a plurality of cultures, each comprising: a given bacterial strain of a plurality of bacterial strains and (1) the first bacterial strain or (2) a bacterial product generated by the first bacterial strain; and (c) determining a culture of the plurality of cultures of (b) that is capable of exhibiting a sufficient ability or a collective effect on the disease associated function as described herein, thereby identifying the given bacterial strain to generate the bacterial combination comprising the first bacterial strain and the given bacterial strain. In some cases, the first bacterial strain may comprise a donor bacterial strain as described herein. In some cases, the plurality of bacterial strains of (b) may comprise a list of potential recipient bacterial strains. In some cases, the culture of (b) may comprise the second bacterial strain and a cultured medium or supernatant thereof of the first bacterial strain. In some cases, determining a culture of the plurality of cultures of (b) that is capable of exhibiting a sufficient ability or a collective effect on the disease associated function as described herein in process (c) may comprise determining whether a cultured medium of the culture of (b); supernatant thereof; or a bacterial product generated by the bacterial strains of the cultured medium of the culture of (b) exhibit a sufficient ability or an collective effect on the disease associated function as described herein. The identified bacterial population can be used to treat or prevent any disease or disease conditions as described herein. In some cases, methods for determining a bacterial population as described herein can comprise the methods described in EXAMPLEs 1-11.

As an illustrative example, for identifying the two bacterial strains for generating a collective bacterial population, the two bacterial strains may each have at least one sufficient ability in a disease associated function, as described herein (see, e.g., the candidate bacterial strains described in EXAMPLE 1). In some cases, each of the two bacterial strains may have at least one sufficient ability in a disease associated function different from that of the other.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains. In some embodiments, the bacterial strain, when combined within a medium comprising the carbon source and a second bacterial strain or a supernatant of the medium comprising the carbon source and the second bacterial strain, exhibits a growth of at least about 101%, 105%, 110%, 115%, 120%, 130%, 140% 150%, 160%, 170%, 180%, 190%, 200%, 250%, 300%, 350%, 400%, 450%, 500%, 550%, 600%, 650%, 700%, 750%, 800%, 850%, 900%, 950%, 999%, 1000%, or 1100% by weight as compared to growth of the first bacterial strain when combined with in a medium comprising the carbon source in an absence of the second bacterial strain or the supernatant of the medium comprising the carbon source and the second bacterial strain. In some embodiments, the carbon source does not comprise starch.

In some embodiments, the supernatant of the medium comprising the carbon source and second bacterial strain is cell-free or comprises a fermented product derived from the second bacterial strain.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains wherein the bacterial strain, when combined with a medium comprising a secreted metabolite (or bacterial product) derived from a second bacterial strain, proliferates for at least about 10 cell divisions. In some embodiments, the medium can comprise secreted metabolite (or bacterial product)s derived from the second bacterial strain which can consist of a supernatant derived from the growth culture of the second bacterial strain, which can be cell-free.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains wherein the bacterial strains, when combined with in a medium comprising a secreted metabolite (or bacterial product) derived from the second bacterial strain and a layer of epithelia cells, decreases a gas permeability of the layer of epithelial cells by at least about 1%, 2%, 3%, 4%, 5%, 10%, 20%, 30%, 40%, 50%, 60%, 70%, 80%, 90%, 99%, or 100% as compared to gas permeability of a layer of epithelial cells combined with in a medium comprising the bacterial strain in an absence of the secreted metabolite (or bacterial product), and wherein the gas permeability of the epithelial cells can be measured by transport of a Fluorescein isothiocyanate (FITC)-conjugated dextran across the layer of epithelial cells. In some embodiments, the mammalian epithelial cells can comprise mammalian epithelial cells, which can further be comprised of human epithelial cells. In some embodiments, the medium can comprise the secreted metabolite (or bacterial product) derived from the second bacterial strain, which can be cell-free or a fermentation product.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains wherein a secreted metabolite (or bacterial product) or an inviable cell derived from the bacterial strain, when combined with an engineered cell comprising a reporter, decreases a signal of the reporter by at least about 1%, 2%, 3%, 4%, 5%, 10%, 20%, 30%, 40%, 50%, 60%, 70%, 80%, 90%, 99%, or 100% as compared to a signal of the reporter when the engineered cell is not combined with the metabolite (or bacterial product) or the inviable cell derived from the bacterial strain. In some embodiments, the medium can comprise the secreted metabolite (or bacterial product) derived from the second bacterial strain, which can be cell-free or a fermentation product. In some embodiments, the engineered cell can comprise a macrophage. In some embodiments, the inviable cell can comprise a pasteurized cell. In some embodiments, the reporter can comprise a NFkB reporter or an interferon-sensitive response element reporter (ISRE). In some embodiments, the NFkB reporter comprises a secreted embryonic alkaline phosphatase (SEAP) reporter. In some embodiments, the ISRE reporter comprises a Lucia luciferase.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains wherein the bacterial strain, when combined with a medium for at most about 15 hours, comprising the second bacterial strain or a supernatant thereof, exhibits a growth of at least about 101%, 105%, 110%, 120%, 130%, 140%, 150%, 160%, 170%, 180%, 190%, 199%, 200%, or more than 200% by colony-forming unit (CFU) as compared to a growth of the bacterial strain when combined with in a medium for at most about 15 hours not comprising the second bacterial strain or the supernatant thereof. In some embodiments, the supernatant comprises a secreted metabolite (or bacterial product) derived from the second bacterial strain, or fermentation product, which can be cell-free.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains wherein the first bacterial strain is configured to utilize a metabolite (or bacterial product) derived from the second bacterial strain as an energy source. In some embodiments, the metabolite (or bacterial product) is not butyrate.

In some embodiments, a method of creating the pharmaceutical composition can comprise providing a plurality of bacterial strains, culturing a given bacterial strain of the plurality of bacterial strains in a carbon source of a plurality of carbon sources, measuring the growth of the plurality of bacterial strains, and selecting a bacterial strain of the plurality of bacterial strains wherein the bacterial strain is incapable of using sialic acid as the carbon source. In some embodiments, the bacterial population when combined in the medium comprising the sialic acid, may not exhibit a growth of at least about 101%, 105%, 110%, 120%, 130%, 140%, 150%, 160%, 170%, 180%, 190%, 199%, 200%, or more than 200% by weight as compared to the growth of the reference bacterial population when combined with in the medium comprising glucose as a carbon source or sole carbon source.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “a” or “an” as used herein refers to one or to more than one (i.e., to at least one) of the grammatical object of the article. By way of example, “an element” means one element or more than one element.

Percent (%) sequence identity or homology with respect to a reference polynucleotide sequence is the percentage of nucleotide residues in a candidate sequence that are identical with the nucleotide residues in the reference polynucleotide sequence, after aligning the sequences and introducing gaps, if necessary, to achieve the maximum percent sequence identity, and not considering any substitutions as part of the sequence identity. Alignment for purposes of determining percent polynucleotide sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % nucleotide sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program can be compiled for use on a UNIX operating system, including digital UNIX V4.0D. All sequence comparison parameters are set by the ALIGN-2 program and do not vary. In situations where ALIGN-2 is employed for polynucleotide sequence comparisons, the % nucleotide sequence identity of a given nucleotide sequence A to, with, or against a given nucleotide sequence B (which can alternatively be phrased as a given nucleotide sequence A that has or comprises a certain % nucleotide sequence identity to, with, or against a given nucleotide sequence B) is calculated as follows: 100 times the fraction X/Y, where X is the number of nucleotide residues scored as identical matches by the sequence alignment program ALIGN-2 in that program's alignment of A and B, and where Y is the total number of nucleotide residues in B. It will be appreciated that where the length of nucleotide sequence A is not equal to the length of nucleotide sequence B, the % nucleotide sequence identity of A to B will not equal the % nucleotide sequence identity of B to A. Unless specifically stated otherwise, all % nucleotide sequence identity values used herein are obtained as described in the immediately preceding paragraph using the ALIGN-2 computer program. % sequence identity can be converted into % sequence complementary based on canonical base-pairing.

Isolated strains disclosed herein have been deposited in the DSM Leibniz Institute DSMZ—German Collection of Microorganisms and Cell Cultures, Inhoffenstr. 7B, D-38124 Braunschweig, Germany in accordance with and under the provisions of the Budapest Treaty for the Deposit of Microorganisms, i.e., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which may issue disclosing the cultures plus five years after the last request for a sample from the deposit. The strains were tested by the DSMZ and determined to be viable. The DSMZ has assigned the following deposit accession numbers (in parentheses in the following) to the strains:
(1) Lactobacillus crispatus ST100 (DSM 33187), which was deposited on Jun. 27, 2019;
(2) Bifidobacterium bifidum ST31 (DSM 34533), Bifidobacterium bifidum ST80 (DSM 34534), Lactobacillus plantarum ST65 (DSM 34526), Lactobacillus crispatus ST20 (DSM 34527), Lactobacillus crispatus ST112 (DSM 34529), Lactobacillus gasseri ST105 (DSM 34528), Lactobacillus jensenii ST21 (DSM 34525), which were deposited on Jan. 26, 2023.

The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them. The deposits are available as required by foreign patent laws in countries wherein counterparts of the subject application, or its progeny, are filed. However, it should be understood that the availability of a deposit does not constitute a license to practice the subject matter disclosed herein in derogation of patent rights granted by governmental action.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### EXAMPLES

These examples are provided for illustrative purposes only and not to limit the scope of the claims provided herein.

### Example 1: Constructing a Collective Microbial Consortium

Provided herein are methods for evaluating individual microbial strains for building a collective microbial consortium, as described herein. A collective microbial consortium may be a bacterial population as described herein.

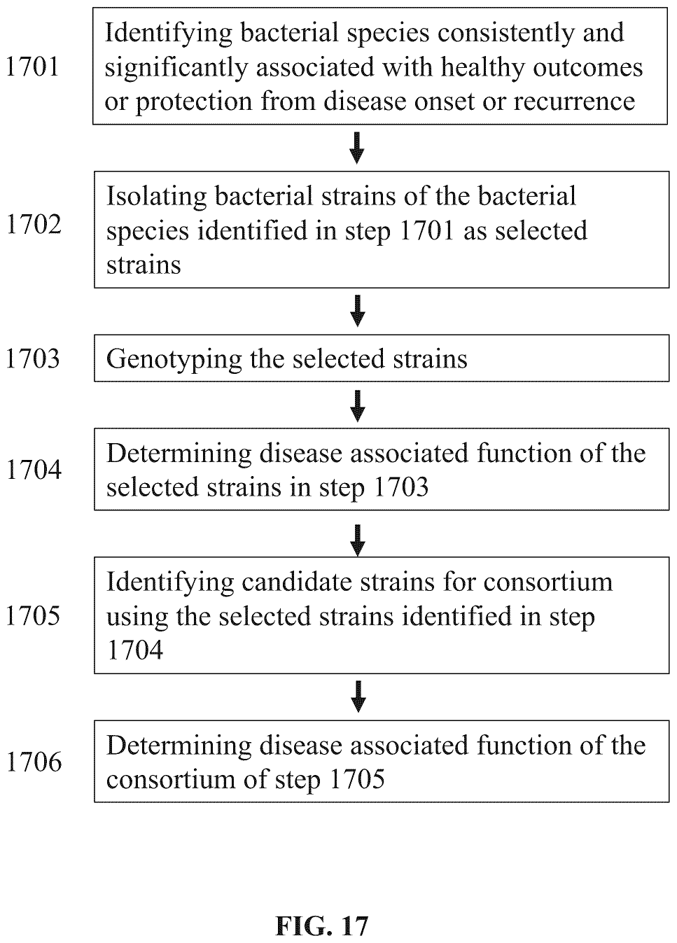

High-value strains underwent a strain selection process, as shown in FIG. 17, in which collective consortia with desirable mechanisms were generated. In process 1701, microbiome data from healthy individuals, public microbiome datasets (e.g., Olm et al., Sci Adv. 2019 Dec. 11; 5; Shao et al., Nature. 2019 October; 574(7776):117-121; Zhou et al., PLoS One. 2015 Mar. 5; 10(3):e0118632; Sobel et al., J Clin Microbiol. 2019 Apr. 26; 57(5):e00227-19; Tamraker et al., BMC Infect Dis. 2007 Nov. 7; 7:128; Ravel et al., Proc Natl Acad Sci USA. 2011 Mar. 15; 108 Suppl 1(Suppl 1):4680-7; France et al., Microbiome. 2020; 8: 166; and Munoz et al., Microbiome. 2021 Jul. 26; 9(1):163, each of which is incorporated by reference in its entirety), and microbiome profiles from clinical studies were analyzed to identify bacterial species consistently and significantly associated with healthy outcomes or protection from disease onset or recurrence. In process 1702, stool and vaginal microbiota samples were collected from healthy volunteers, and bacterial species and strains of interest identified in process 1701 were targeted for isolation and culture using enrichment and selective bacteriological culture media such as those described in EXAMPLE 5. In process 1703, bacterial isolates were genotyped using 16S rRNA gene sequencing and were selected based on in the selection criteria process 1701, such as those described in EXAMPLE 9. Additionally, and/or optionally, whole genome sequencing analysis was performed on the selected bacterial strains for functional capacity and genome-based risks factors, such as those described in EXAMPLE 9. In process 1704, individual selected bacterial strains were screened for disease-specific mode-of-actions (MOAs) and therapeutic functionality, such as those described in EXAMPLEs 2-3, 6, 8, and 10-11. selected bacterial strains that have sufficient ability when assessed using disease-specific MOA or functionality assays were identified as candidate bacterial strains for generating a collective bacterial consortium. In process 1705, the collective activities of individual candidate bacterial strains of interest were characterized through cross-feeding studies to identify candidate bacterial strains to function collectiveally in a collective microbial consortium, such as those described in EXAMPLEs 7-8. Such methods can also identify the bacterial strains as being a donor or recipient bacterial strain. In process 1706, consortia are evaluated for their ability to positively impact disease-specific MOAs and functionality, such as those described in EXAMPLE 4. Optionally, the consortia can be determined if they can treat the disease or alleviate the symptom(s) of the diseases.

### Example 2: BV-Specific Mode-Of-Actions (MOAs) and Functionality Assays

Provided herein are methods for determining BV-specific MOAs and functionality of the selected bacterial strains for identifying candidate bacterial strains for generating collective microbial consortium for treating or preventing BV.

selected bacterial strains were subjected to at least one of the following assays: (1) vaginal epithelia cell (VEC) adhesion assay; (2) vaginally relevant carbohydrate utilization assay; (3) vaginal pathogen growth/biofilm inhibition assay; (4) growth in vaginal pH; and (5) bacterial product generation that is relevant to treat or prevent BV, for identifying candidate bacterial strains with collective therapeutic potential for preventing and treating BV. The selected bacterial strains had sufficient ability to: adhere to VEC, utilize vaginally relevant carbohydrate, growing in vaginal pH, generating a bacterial product that is relevant to treat or prevent BV, and/or inhibit vaginal pathogen growth/biofilm formation.

### VEC Adhesion Assay

selected bacterial strains should have a sufficient ability to adhere to the VECs. Such ability can indicate that the bacterial strain is capable of long-term engraftment or colonization in the human vaginal tract of individuals, patients, or subjects being administered with a collective microbial consortium comprising the strains capable of sufficient VEC adherence. When a bacterial strain has a sufficient ability to adhere to VEC, it also can exclude vaginal pathogens from adhering to VEC.

In VEC adhesion assay, selected bacterial strains were individually tested for adherence to the VEC. The VECs (VK2/E6E7; ATCC, CRL-2616) were cultured in complete media (Keratinocyte-Serum Free medium (Gibco-BRL 17005-042) with 0.1 ng/ml human recombinant EGF, 0.05 mg/ml bovine pituitary extract, and calcium chloride 44.1 mg/L) with 1% Penicillin and Streptomycin (Gibco 15140-122)) at 37° C. in an atmosphere of 5% CO². The VECs were digested with 2 mL of 0.25% trypsin-EDTA (Gibco 25200056) for 5 mins and the digestion was terminated by adding 8 mL of 10% FBS DMEM-F12 (ATCC 30-2006). The supernatant was removed after the centrifugation for 5 mins at 150×g, and the cells were adjusted to a concentration to 2.5×10⁵ cells/mL using the complete media without Penicillin and Streptomycin. The VEC suspensions were aliquoted into individual wells of a 6-well-culture plate (2 mL/well) and incubated at 37° C. in an atmosphere of 5% CO² for 18 hours. After the incubation, when each culture well was about 50-60% confluent, the cells were washed with 10% FBS DMEM-F12 media, and the cells were co-incubated with the candidate bacteria.

The selected bacterial strains were cultured in vegan MRS media (HIMEDIA MV369-500) in for overnight in the anaerobic chamber incubator at 37° C. The bacteria were then pelleted and resuspended in 5 mL of PBS for washing at least twice. The bacteria were then resuspended in RPMI-1640 media (5 mL/culture; Sigma R8758-500 mL) and adjusted to 1.0×10\hat{ }8 CFU/mL using RPMI-1640 media.

1 mL of bacterial cell suspension was added to each well of 6-well-culture plates with monolayer of VECs, and the mixture of cells were incubated for 1 hour at 37° C. in an atmosphere of 5% CO². The mixture was then washed at least 5 times with PBS to remove non-adhering bacteria. At the last wash, the mixture was vigorously pipetted 20 times as a final wash. PBS was then removed, and 200 μL of 1% Triton X-100 was added to each culture. The mixture was well thoroughly suspended and incubated for 10 minutes at room temperature (RT). An additional 1800 μL of PBS was added to the mixture and vigorously pipetted for 20 times. The collected PBS-resuspended cells from were plated on to MRS plates with 10\hat{ }-1 to 10\hat{ }-7 dilutions. The number of bacterial cells adhered to the VEC was measured as plotted as the log [CFU of bacteria/9.5 cm\hat{ }2 VEC].

The summary of the result is shown in TABLE 8. As an illustrative example, the top 20% percentile of vaginal bacterial isolates (that were tested), as quantified with a greater than 2×10\hat{ }6 CFU/9.5 cm\hat{ }2 adherence to human VEC, were identified to have sufficient ability to adhere to VEC and identified as candidate bacterial strains for collective microbial consortium for treating or preventing BV.

TABLE 8
Summary of BV-specific MOA and functionality assays for exemplary strains and control

| | | % Growth compared | | | | | |
|---|---|---|---|---|---|---|---|
| | % Growth between | between conditioned | | | | | |
| | carbon source | medium tested and | | | | | |
| | tested and carbon | carbon source as | | | | | |
| | source as glucose | glucose | | | | | |
| | carbon source | ST100 conditioned | | | | | D-lactic acid |
| | tested: Glycogen | medium with carbon | | Vaginal | Acidic pH | | Production (RLU |
| | carbon source | source as glycogen | G. vaginalis | Epithelial | Tolerance | | [relative light |
| | tested: Maltose | ST112 conditioned | Biofilm | Cells | (Growth % | H2O2 | unit])/L-lactic |
| Bacterial | carbon source | medium with carbon | Inhibition | Adhesion | at pH 4.0 | Production | acid Production |
| strain | tested: Maltodextrin | source as glycogen | (% Reduction) | (log CFU/9.5 cm²) | vs pH 6.0) | (uM) | (RLU |
| L. | 60% | 0% | 40% | 5.6 | 67% | 650 | 2.00E+04 |
| crispatus | 100% | 50% | | | | | 1.00E+05 |
| ST100 | 90% | | | | | | |
| L. | 20% | 50% | 30% | 4.27 | 10.80% | 783 | 2.00E+04 |
| crispatus | 90% | 10% | | | | | 1.30E+05 |
| ST112 | 80% | | | | | | |
| L. | 0% | 10% | 40% | 6.00 | 1.25% | 1300 | 9.70E+04 |
| crispatus | 110% | 20% | | | | | 9.80E+03 |
| ST20 | 110% | | | | | | |
| L. | 10% | 90% | 50% | 4.8 | 70.50% | 1614 | 4.30E+04 |
| jensenii | 130% | 90% | | | | | 2.00E+04 |
| ST21 | 120% | | | | | | |
| L. | 0% | 70% | 40% | 6.58 | 40.70% | 799 | 1.80E+04 |
| gasseri | 80% | 100% | | | | | 1.40E+05 |
| ST105 | 0% | | | | | | |
| L. | 0% | Not tested | 0% | 5.68 | 3.20% | 741 | 6.20E+04 |
| jensenii | 70% | | | | | | 7.00E+03 |
| ST77 | 60% | | | | | | |
| L. | 0% | 200% | 20% | 5.41 | 52.50% | 1851 | 4.80E+04 |
| jensenii | 150% | 140% | | | | | 3.00E+04 |
| ST13 | 30% | | | | | | |
| L. | 40% | 90% | 10% | 6.48 | 26.40% | 856 | 2.00E+04 |
| crispatus | 100% | 100% | | | | | 1.50E+05 |
| ST43 | 90% | | | | | | |
| LP01 | Not tested | Not tested | 45% | Not tested | Not tested | Not tested | Not tested |
| | Not tested | Not tested | | | | | Not tested |
| | Not tested | | | | | | |
| LBV96 | Not tested | Not tested | 31% | Not tested | Not tested | Not tested | Not tested |
| | Not tested | Not tested | | | | | Not tested |
| | Not tested | | | | | | |
| LBV88 | Not tested | Not tested | 50% | Not tested | Not tested | Not tested | Not tested |
| | Not tested | Not tested | | | | | Not tested |
| | Not tested | | | | | | |

N/A: not applicable

### Vaginally Relevant Carbohydrate Utilization Assay

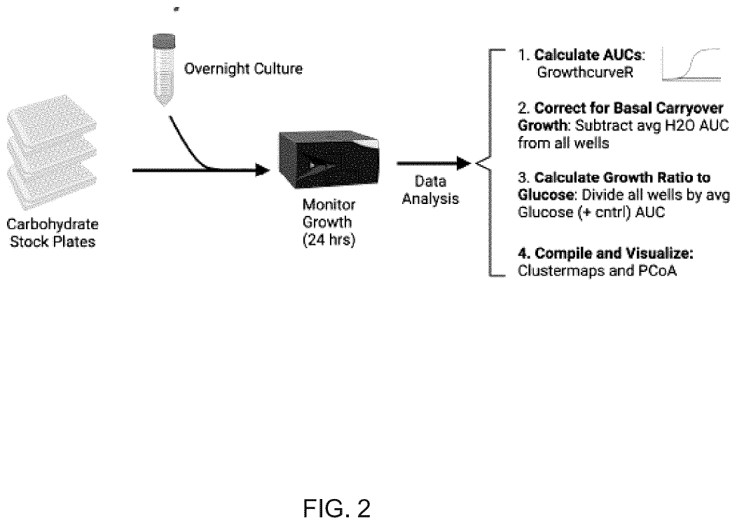

The candidate bacterial strains should have a sufficient ability to utilize vaginally relevant carbohydrate. Provided herein are methods for the preparation and execution of vaginally relevant carbohydrate utilization screening for identifying candidate bacterial strains with sufficient ability to utilize vaginal carbohydrates and growth factors that can promote host colonization and therapeutic efficacy. Such strains can be used as the candidate bacterial strains of a collective microbial consortium described herein. FIG. 2 illustrates the overall direct carbohydrate utilization screening method.

### 1. Preparation of the Carbohydrate Stock Solutions

Generally, the procedures for preparing carbohydrate stock solutions as described herein can be used for culturing and screening obligatory and facultative anaerobic bacterial strains. The exemplary bacterial strains in this example were isolated from vaginal swabs collected from healthy female donors and were grown on selective or enrichment culture media (e.g., see EXAMPLE 5).

First, the carbohydrate stock solutions were prepared by dissolving each carbohydrate listed in TABLE 9 (excluding mucin, guar gum, pectin, and inulin) in Milli-Q water to create a 20% carbohydrate solution, 10× the Final Concentration percentage listed in TABLE 9. Mucin was dissolved Milli-Q water to create a 3% stock solution, 10× the Final Concentration percentage.

TABLE 9
Carbohydrate Compound Library.

| | | Catalog | | Final % |
|---|---|---|---|---|
| Carbohydrate | Vendor | Number | Category | Concentration |
| Maltose | ThermoFisher | A16266.22 | Glycogen | 2 |
| | | | metabolite | |
| Mucin | Sigma | M1778-100G | Host | 0.3 |
| Glycogen | Sigma | G0885-5G | Host | 2 |
| Melibiose | Sigma | M5500-5G | Disaccharide | 2 |
| N- | Carbosynth | MA00746 | Deoxysugar | 2 |
| Acetylneuraminic | | | | |
| acid (Sialic Acid) | | | | |
| N-Acteyl | Sigma | A3286-100G | Amino sugar | 2 |
| Glucosamine | | | | |
| Maltodextrin | Spectrum | M1083 | Glycogen | 2 |
| | | | metabolite | |
| D-Glucose | Sigma | G7021 | Monosaccharide | 2 |
| D-Glucose + | Sigma | G7021 | Monosaccharide | 2 |
| 0.1% cysteine | VWR (cysteine) | J994-100G (cysteine) | Amino acid | |

Excluding mucin, the 10× carbohydrate stock solutions were filtered through a sterile 0.22 μm PES syringe filter (Millipore). Mucin was dissolved in Milli-Q water and autoclaved to attain sterility.

### 2. Stock Carbohydrate Plates Preparation

The stock carbohydrate plates were prepared by aliquoting 20 μL of each 10× carbohydrate stock solution individually into each well of the plate in a biosafety cabinet, as diagrammed in TABLE 10. Water was added to wells B10-12, D10-12, F10-12, and H9. Row H were media and carbohydrate stock solution sterile controls. The bottom row of the plate, row H, was negative media controls to evaluate media sterility. Upon completion of stock carbohydrate plate preparation, the sterile plates were sealed with foil and stored at −80′° C.

TABLE 10
Stock Carbohydrate Plate Layout

| | 1 | 2 | 3 | 4 | 5 | 6 | 7 | 8 | 9 | 10 | 11 | 12 |
|---|---|---|---|---|---|---|---|---|---|---|---|---|
| A | strain1 | strain1 | strain1 | strain1 | strain1 | strain1 | strain1 | strain1 | strain1 | strain1 | strain1 | strain1 |
| | glycogen | glycogen | glycogen | maltose | maltose | maltose | malto- | malto- | malto- | mucin | mucin | mucin |
| | | | | | | | dextrin | dextrin | dextrin | | | |
| B | strain1 | strain1 | strain1 | strain1 | strain1 | strain1 | strain1 | strain1 | strain1 | strain1 | strain1 | strain1 |
| | sialic | sialic | sialic | melibiose | melibiose | melibiose | glucose | glucose | glucose | water | water | water |
| | Acid | Acid | Acid | | | | | | | | | |
| C | strain2 | strain2 | strain2 | strain2 | strain2 | strain2 | strain2 | strain2 | strain2 | strain2 | strain2 | strain2 |
| | glycogen | glycogen | glycogen | maltose | maltose | maltose | malto- | malto- | malto- | mucin | mucin | mucin |
| | | | | | | | dextrin | dextrin | dextrin | | | |
| D | strain2 | strain2 | strain2 | strain2 | strain2 | strain2 | strain2 | strain2 | strain2 | strain2 | strain2 | strain2 |
| | sialic | sialic | sialic | melibiose | melibiose | melibiose | glucose | glucose | glucose | water | water | water |
| | Acid | Acid | Acid | | | | | | | | | |
| E | strain3 | strain3 | strain3 | strain3 | strain3 | strain3 | strain3 | strain3 | strain3 | strain3 | strain3 | strain3 |
| | glycogen | glycogen | glycogen | maltose | maltose | maltose | malto- | malto- | malto- | mucin | mucin | mucin |
| | | | | | | | dextrin | dextrin | dextrin | | | |
| F | strain3 | strain3 | strain3 | strain3 | strain3 | strain3 | strain3 | strain3 | strain3 | strain3 | strain3 | strain3 |
| | sialic | sialic | sialic | melibiose | melibiose | melibiose | glucose | glucose | glucose | water | water | water |
| | Acid | Acid | Acid | | | | | | | | | |
| G | strain1 | strain1 | strain1 | strain2 | strain2 | strain2 | strain3 | strain3 | strain3 | | | |
| | glucose Cys | glucose | glucose | glucose | glucose | glucose | glucose | glucose | glucose | | | |
| | (cysteine) | Cys | Cys | Cys | Cys | Cys | Cys | Cys | Cys | | | |
| H | glycogen | maltose | maltodextrin | mucin | sialic | melibiose | glucose | glucose | water | | | |
| | cntrl | cntrl | cntrl | cntrl | Acid | cntrl | cntrl | cys cntrl | cntrl | | | |
| | (control) | | | | cntrl | | | | | | | |

### 3. Inoculum and Culture

First, the frozen stock carbohydrate plates were prepared for inoculation by defrosting at 37 C for 1 hour, mixed for 1 min to resuspend any precipitated carbohydrate. Thawed and resuspended plates are place in the anaerobic chamber with a vented lid for two hours before use to ensure excess oxygen was removed. The previously isolated bacterial strains Lactobacillus were cultured overnight in complete media with glucose. A 1:100 dilution of the Lactobacilli cultures was prepared in a basal media without carbohydrates (TABLE 4).

TABLE 4
MRS Without Dextrose.

| Component | Amount (g/L) |
|---|---|
| MRS Broth w/o Dextrose (Alpha | 35.2 |
| Biosciences, cat# L12-104M) | |

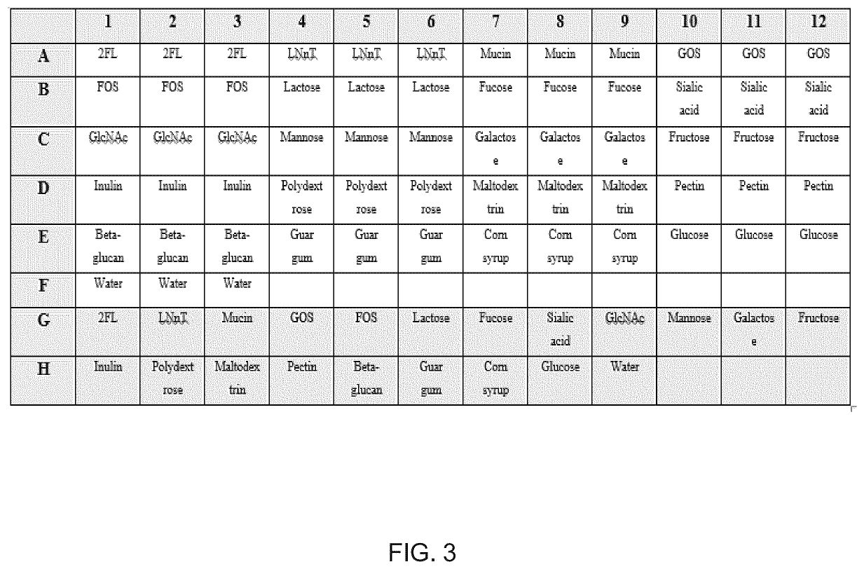

After dilution, 180 μL of three diluted bacterial strains were pipetted into wells of the defrosted and anaerobically stored stock carbohydrate plates as diagrammed in TABLE 10 where wells labeled “strain1” receive the first strain, wells labeled “strain2” receive the second strain, and wells labeled “strain3” receive the third strain. For the sterile media controls, 180 μL of basal media (TABLE 4) were pipetted into each well of row G and row H as diagrammed in FIG. 3. The OD₆₀₀ spectrophotometer measurement of each well of the stock carbohydrate plates was taken every 30 minutes for a 24-hour period.

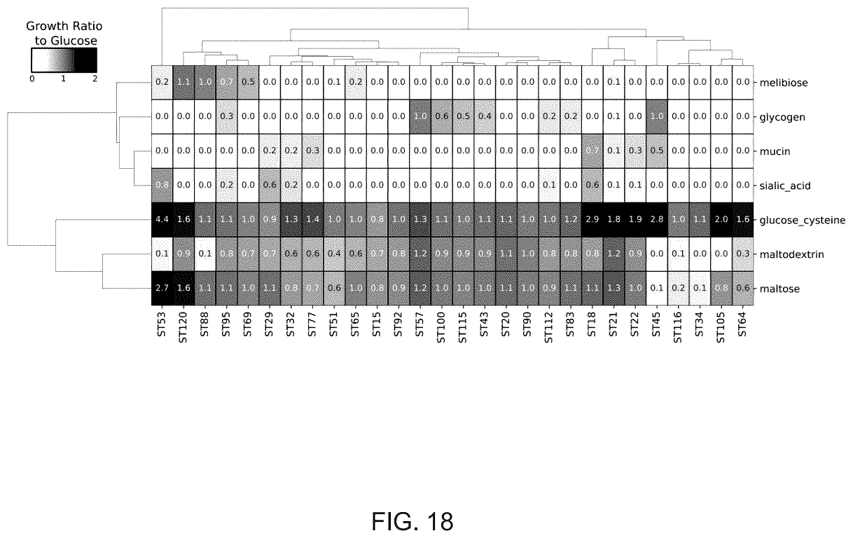

The summary of the result is shown in FIG. 18 with data processed as described in EXAMPLE 8 and summarized in TABLE 8. As an illustrative example, the candidate bacterial strains were identified as having the ability to utilize glycogen (>0.5 Growth Ratio in glycogen to in Glucose), and ability to utilize maltose or maltodextrin (>0.8 Growth Ratio in maltose or maltodextrin to in Glucose). Strains in the top 20% percentile of biomass accumulation (growth) on glycogen and/or maltose or maltodextrin were considered sufficient vaginally relevant carbohydrate utilizers and selected as candidate bacterial strains for collective microbial consortium for treating or preventing BV.

### Vaginal Pathogen Growth Biofilm Inhibition Assay

Biofilm formation by vaginal or BV-associated pathogens contributes to disease progression and treatment complications using the current standard of care. Candidate bacterial strains for collective microbial consortium for the treatment and prevention of BV are identified as having the ability to inhibit biofilm formation of G. vaginalis and prevented the expansion of L. iners biofilms.

The ability of selected bacterial strains to inhibit biofilm formation by BV pathogens was assessed as follows: Condition media comprising the bacterial products from selected bacterial strains were collected by growing the strains in vegan MRS media (HIMEDIA MV369-500) in an anaerobic chamber incubator at 37° C. overnight. The following day, the cells of selected bacterial strains from stationary phase cultures were pelleted, and the supernatant containing the bacterial products was collected and filtered through a 0.22 μm filter. Gardnerella vaginalis (ATCC 14018) and Lactobacillus iners (ATCC 55195) were grown overnight at 37° C., 5% CO2 in NYCIII media with 10% horse serum. The following day, overnight cultures were back diluted to 0.1 OD600 in NYCIII with 2% horse serum for G. vaginalis and NYCIII with 10% horse serum for L. iners. 200 μL of back diluted cultures were then seeded in 96 well plates and incubated for 24 hours at 37° C., 5% CO2. After incubation, media was removed and fresh media containing 10% spent media from selected bacterial strains was added in 200 μL. Treatments were performed in quadruplicate with matching media controls. Plates were then incubated for 24 hours at 37° C., 5% CO2. After incubation, the media was removed, and the biofilms were washed three times with 200 μL PBS. After the last wash, all liquid was removed from the well and the biofilms were stained with 200 μL of 0.1% w/v crystal violet solution for 3 minutes at room temperature. The crystal violet solution was removed, and the wells were washed 3 times with 200 μL PBS. After washing, all liquid was removed from the wells and the stained biofilms were left to air dry at room temperature for 30 minutes. The stained biofilms were then solubilized with 200 μL of 95% ethanol. Absorbance at 570 nm was measured with a plate reader. Biofilm Remaining Ratio was calculated by taking the ratio of the average absorbance of treatment wells versus media control wells.

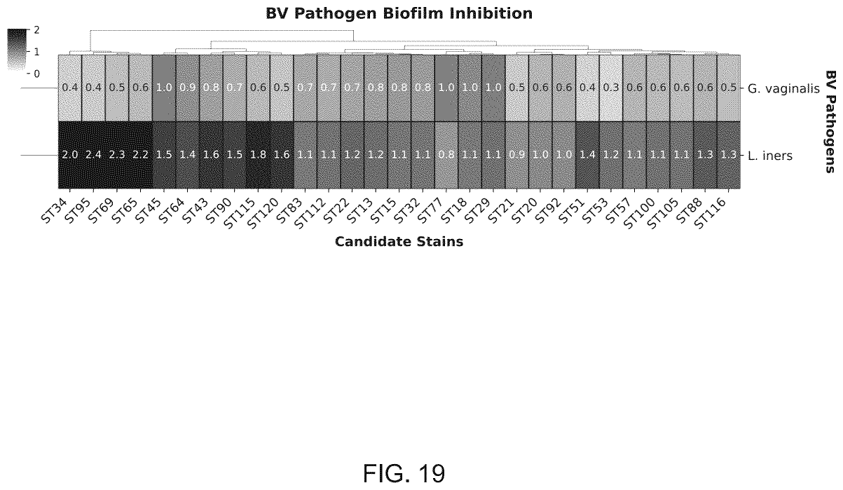

The effect of the conditioned media comprising the bacterial products from the selected bacterial strains on G. vaginalis and L. iners biofilm formation were represented as Biofilm Remaining Ratio values. These values were aggregated into a cluster map, an example of which shows various selected bacterial strains in FIG. 19 and TABLE 8. Condition media from individual strains had diverse effects on biofilm formation, clustering into different groups. One group of strains, including ST34, inhibit G. vaginalis biofilm formation by ˜60%, but increased L. iners biofilm formation by ˜100%. Another group of strains, including ST18, had no effect on either G. vaginalis or L. iners biofilm formation. Another group of strains, including ST20 (DSM34527), was capable of inhibiting G. vaginalis biofilm by 40% and did not exacerbate L. iners biofilm formation. These data were used to identify the candidate bacterial strains for the collective microbial consortium for treating or preventing BV, such as those described in TABLE 8.

As an illustrative example, the candidate bacterial strains could inhibit biofilm formation of G. vaginalis by 30% and prevent the expansion of L. iners biofilms by 20% relative to media control.

### Acidic pH Tolerance

Candidate bacterial strains should have a sufficient ability in growing in vaginal pH, which is acidic.

The ability of the selected bacterial strains to grow in acidic pH was measured using methods described in this EXAMPLE 2 or EXAMPLE 6, except that the tested and control condition is modified to be a medium with pH=4 and pH=6, respectively. The ability of the selected strain to grow in acidic pH was measured as the growth ratio of the bacterial strain between (1) media with pH=4 and (2) media with pH=6.

The result of the selected bacterial strains for growing in acid pH is summarized in TABLE 8. As an illustrative example, the candidate bacterial strains could generate at least about 650 μM hydrogen peroxide and about 1×10\hat{ }5 RLU of lactic acid when measured by the method described herein. While certain strains did not grow in acid pH significantly (such as ST77 or ST20), these strains could still be used in the collective microbial consortium as described herein when combined with the other candidate bacterial strains. In an illustrative example, the candidate bacterial strains had at least 30% growth ratio in media with pH=4/pH=6.

### Generations of Bacterial Products Relevant to BV

Candidate bacterial strains should have a sufficient ability to generate a bacterial product that can treat BV or alleviate the symptoms of BV. For example, the bacterial products can inhibit the growth of the BV pathogens. The bacterial products can comprise lactic acid or hydrogen peroxide (H₂O₂).

The ability of selected bacterial strains to generate lactic acids and hydrogen peroxide was assessed as follows: The hydrogen peroxide assay was performed as follows, in triplicate for each selected bacterial strain, using the Promega ROS-Glo hydrogen peroxide Assay Kit (Promega #G8820). Lactobacillus strains were grown anaerobically to stationary phase in vMRS and diluted to an OD600 of 0.3 in resuspension buffer (1×PBS+10 mM glucose+100 mM Tris-HCl pH 8). Cells were pelleted, supernatant removed, and resuspended in 1 mL resuspension buffer. 200 μL of each resuspended sample was incubated aerobically at 37° C., shaking at 100 rpm for 10 minutes to generate hydrogen peroxide. Cells were then pelleted and 10 μL of supernatant moved to opaque 96-well reaction plates containing 70 μL resuspension buffer per well. 20 μL of hydrogen peroxide Substrate solution (12.5 μL of 10 mM hydrogen peroxide Substrate per 1 mL Substrate Dilution Buffer, Promega #G8820) was added and plates incubated for 60 min at room temperature. 100 μL of ROS-Glo Detection Solution (1 mL Luciferin Detection Reagent, 10 μL D-Cysteine, 10 μL signal enchancer solution, Promega #G8820) was added, plates incubated for 20 minutes at room temperature, and luminesce read using a luminometer (and converted to molar concentration of hydrogen peroxide). The Lactic acid assay was performed as follows, in triplicate for each selected bacterial strain, using the Promega Lactate-Glo Assay Kit (J5021) with D-Lactic Dehydrogenase (Sigma, #L3888-500UN) and Sodium D-lactate (Sigma, #71716-1G) purchased separately to quantify D-Lactic acid. Supernatants from Lactobacillus strains grown to stationary phase were diluted 200× in PBS and 50 μL transferred to opaque 96-well reaction plates (separate 96-well plates for L and D-lactate). Lactate Detection Reagent was prepared for both L- and D-lactate (per reaction: 50 μL Luciferin Detection Solution, 0.25 μl Reductase, 0.25 μl Reductase Substrate, 0.25 μl Lactate Dehydrogenase, 0.25 μl NAD, Promega #J5021), and 50 μL of the Lactate Detection Reagent was added to each well. The plates were shaken for 1 minute to mix, incubated for 1 hour at room temperature, and luminesce read using a luminometer (measured as relative light unit/RLU).

The results of the selected bacterial strains for generating lactic acids and hydrogen peroxide are summarized in TABLE 8. As an illustrative example, the candidate bacterial strains could generate at least about 650 μM hydrogen peroxide and about 1×10\hat{ }5 RLU of lactic acid when measured by the method described herein.

Collectively, the candidate bacterial strains for collective microbial consortium for treating or preventing BV in the illustrative example showed sufficient ability to: adhere to VEC, utilize vaginally relevant carbohydrate, and/or inhibit vaginal pathogen growth/biofilm formation. Some of the exemplary strains are described in TABLE 8.

### Example 3: NEC-Specific Mode-Of-Actions (MOAs) and Functionality Assays

Provided herein are methods for determining NEC-specific MOAs and functionality for identifying candidate bacterial strains for generating collective microbial consortium for treating or preventing NEC.

Candidate bacterial strains were subjected to at least one of the following assays: (1) intestinal epithelial cell (IEC) adhesion assay; (2) Infant-relevant carbohydrate utilization; (3) NEC-pathogen inhibition; (4) TLR4 Receptor Inhibition; and (5) gastrointestinal barrier integrity. The selected bacterial strains had sufficient ability to: adhere to IEC, utilize infant-relevant carbohydrate, inhibit TLR4 receptor, inhibit NEC-pathogen growth and/or increase gastrointestinal barrier integrity.

### IEC Adhesion Assay

Selected bacterial strains are identified as having sufficient ability to adhere to the intestinal epithelial cells (IECs). Such ability can indicate that strain can show long-terms engraftments in the intestinal tract of the patients or subjects being administered with a collective microbial consortium comprising the strains. Strains that can adhere to IECs with a greater affinity than 1) the opportunistic pathogen strain E. coli; or 2) the comparator strains BG49 (Lactobacillus) or EV27 (Bifidobacterium) are identified as candidate bacterial strains for collective microbial consortium for treating or preventing NEC.

In intestinal epithelia cell adhesion assay, selected bacterial strains were individually tested for adherence to the IECs. The IECs (C2BBe1; ATCC, CRL-2102) were cultured in sterilized Biosafety cabinet (BSC) in complete media (DMEM (Gibco 11995) with 10% FBS and 1% Penicillin and Streptomycin (Gibco 15140-122)) at 37° C. in an atmosphere of 5% CO2. The IECs were digested with 0.25% trypsin-EDTA for 5 mins before the addition of complete media (for the T75 flask, use 2 mL of trypsin and 8 mL of complete media). The IECs were counted before being centrifuged at 2,700×g for 10 min, the media removed, and adjusted to a concentration to 1.0×10\hat{ }5 cells/mL in complete media (without Penicillin and Streptomycin). The IEC suspension was aliquoted into individual wells of a 6-well-culture plate (2 mL/well) and incubated at 37° C. in an atmosphere of 5% CO2 for 18 hours. After the incubation, the cells were washed with complete media (without Penicillin and Streptomycin), and the cells were co-incubated with the selected bacteria.

The selected bacterial strains were cultured in vegan media (in-house recipes) for overnight in the anaerobic chamber incubator at 37° C. The bacteria were then pelleted and resuspended in 5 mL of PBS for washing at least twice. The bacteria were then resuspended in DMEM (5 mL/culture; Gibco 11995).

1 mL of bacterial cell suspension was added to each well of 6-well-culture plates with monolayer of IECs, and the mixture of cells were incubated for 1 hour at 37° C. in an atmosphere of 50 CO2. The mixture was then washed at least 5 times with PBS to remove non-adhering bacteria. At the last wash, the mixture was vigorously pipetted 20 times as a final wash. PBS was then removed, and 1 mL of 0.25% trypsin-EDTA was added to each culture and incubated for 15 minutes at 37° C. in an atmosphere of 5% CO2. The detached cells were gently resuspended before plating on to YFAP or MRS plates (as appropriate) with 10\hat{ }-1 to 10\hat{ }-7 dilutions. The number of bacterial cells adhered to the IECs was measured and plotted as the log [CFU of bacteria/9.5 cm\hat{ }2 IEC].

The summary of the result is shown in TABLE 11-1 and TABLE 11-2.

TABLE 11-1
First summary of NEC-specific MOA and functionality
assays for exemplary strains and control

| | | Top | Pathobiont | E. coli | TLR4 |
|---|---|---|---|---|---|
| | IEC | Carbohydrate | growth | growth | inhibition |
| Strain ID | adherence | utilization | inhibition* | inhibition (%) | (%) |
| ST19 | 1.2 log 4 | FOS, Galactose, | Tier 1 | 65 | 5 |
| | | Maltodextrin | | | |
| ST23 | 1.8 log 6 | FOS, GlcNAc, | Tier 1 | 71 | 12 |
| | | Mannose | | | |
| ST24 | not tested | FOS, Inulin, | Tier 1 | 99 | 13 |
| | | Maltodextrin, | | | |
| | | Galactose | | | |
| ST27 | 1.0 log 7 | FOS, Galactose, | Tier 1 | 68 | 0 |
| | | Maltodextrin | | | |
| ST31 | 1.6 log 7 | 2FL, LNnT, | Tier 1 | 7 | 6 |
| | | GlcNAc | | | |
| ST34 | 4.3 log 5 | not tested | Tier 1 | 63 | 0 |
| ST37 | 7.3 log 4 | FOS, 2FL, | Tier 1 | 76 | 0 |
| | | Maltodextrin | | | |
| ST51 | 2.8 log 4 | not tested | Tier 1 | 73 | 0 |
| ST56 | 1.0 log 5 | FOS | Tier 1 | | 1 |
| ST61 | 2.6 log 4 | FOS, Inulin, | Tier 1 | 90 | 16 |
| | | Maltodextrin | | | |
| ST65 | 5.9 log 6 | FOS, GlcNAc, | Tier 1 | 91 | 20 |
| | | Mannose | | | |
| ST66 | 1.1 log 5 | FOS, | Tier 1 | 98 | 6 |
| | | Maltodextrin | | | |
| ST71 | 1.4 log 5 | FOS | Tier 1 | 86 | 18 |
| ST80 | 1.9 log 7 | 2FL, Galactose | Tier 2 | 17 | 3 |
| ST81 | 2.9 log 4 | FOS, Galactose | Tier 1 | 82 | 13 |
| ST96 | 9.1 log 4 | FOS, Galactose, | Tier 1 | 85 | −20 |
| | | Maltodextrin | | | |
| ST101 | 3.3 log 4 | FOS, Inulin, | Tier 1 | 78 | 16 |
| | | Maltodextrin, | | | |
| | | Galactose | | | |
| ST102 | 2.1 log 6 | FOS, Mucin, | Tier 1 | 68 | 12 |
| | | Maltodextrin | | | |
| ST116 | 1.6 log 5 | GlcNAc, | Tier 1 | 37 | 0 |
| | | Mannose, | | | |
| | | Galactose | | | |
| ST119 | 3.6 log 5 | Galactose, | Tier 2 | 89 | 14 |
| | | 2FL, FOS | | | |
| L. reuteri BG49 | 9.1 log 5 | Galactose | not tested | 51 | 0 |
| B. longum EV27 | 6.2 log 4 | FOS, 2FL, | Tier 2 | 80 | 0 |
| | | LNnT, Galactose | | | |
| E. coli | 1.0 log 6 | N/A | N/A | N/A | −40 |
| notes | (log 10 [CFU/ | *all use | 4 Tiers | in glucose | in glucose |
| | 9.5 cm\hat{ }2]) | Lactose | | rich media | rich media |

*Tier 1 bacterial strains inhibited the growth of all 6 pathogens and at least 2 of these pathogens were inhibited by greater than 50%. Tier 2 bacterial strains inhibited the growth of at least 4 pathogens, and the inhibition of these pathogens was less than 50%. Tier 3 bacterial strains inhibited the growth of at least 3 pathogens but promoted the growth of at least 1 pathogen. Tier 4 bacterial strains promoted the growth of at least 3 pathogens.
N/A: not applicable

TABLE 11-2
Second summary of NEC-specific MOA and functionality
assays for exemplary strains and control

| | | | | E. coli | |
|---|---|---|---|---|---|
| | Growth in 50% | Growth in 50% | Barrier | growth | TLR4 |
| | BMWL (% | milk FWL (% | function (% | inhibition | inhibition |
| | relative to | relative to | relative to | (%) when | (%) when |
| | glucose | glucose | media | grown in | grown in |
| Strain ID | media) | media) | control) | BMWL | BMWL |
| ST19 | 94 | 110 | not tested | 89 | not tested |
| ST23 | 123 | 103 | 120 | 86 | 94 |
| ST24 | 119 | 88 | not tested | not tested | not tested |
| ST27 | not tested | not tested | 99 | not tested | not tested |
| ST31 | 269 | 177 | 89 | 85 | 51 |
| ST34 | 80 | 94 | not tested | not tested | not tested |
| ST37 | 96 | 101 | 120 | 90 | 93 |
| ST51 | 79 | 94 | not tested | not tested | not tested |
| ST56 | | | not tested | not tested | not tested |
| ST61 | 174 | 118 | 112 | not tested | not tested |
| ST65 | 115 | 96 | 112 | 98 | 26 |
| ST66 | 105 | 114 | 128 | not tested | not tested |
| ST71 | 146 | 138 | 113 | 89 | 70 |
| ST80 | 59 | 124 | 115 | 10 | 0 |
| ST81 | 130 | 108 | 107 | 92 | 92 |
| ST96 | 138 | 207 | 130 | not tested | not tested |
| ST101 | 151 | 160 | 123 | 90 | 98 |
| ST102 | 114 | 106 | not tested | 91 | 53 |
| ST116 | 122 | 112 | 99 | 96 | 90 |
| ST119 | 95 | 94 | 115 | 87 | 97 |
| L. reuteri BG49 | 86 | 111 | not tested | not tested | not tested |
| B. longum EV27 | 175 | 154 | not tested | not tested | not tested |
| E. coli | N/A | N/A | N/A | N/A | N/A |
| notes | breast milk | milk formula | 40 h | 50% BMWL | 50% BMWL |
| | whey liquid | whey liquid | impedance | media | media |

N/A: not applicable

As an illustrative example, the, candidate bacterial strains for the collective consortia for treating or preventing NEC had at least about 1×10\hat{ }7 [CFU of bacteria/9.5 cm\hat{ }2 IEC's plasma membrane], adhere to IECs with an affinity 10× greater than that of E. coli or the comparator strain BG49 and 100× greater than the comparator strain EV27. The lowest percentile of bacteria, which had at least about 1×10\hat{ }4 [CFU of bacteria/9.5 cm\hat{ }2 IEC's plasma membrane], adhere to IECs at least as well as the comparator strain EV27. These selected bacterial strains were identified as candidate bacterial strains for collective microbial consortium for treating or preventing NEC.

### Infant Relevant Carbohydrate Utilization

selected bacterial strains are identified as having the ability to utilize 1) lactose (the major carbohydrates present in human breast milk or milk formula products) as a sole carbon source and 2) at least one other carbohydrate predicted to be present in the intestinal tract. Such abilities can indicate that strain can show long-terms engraftments in the intestinal tract of the patients or subjects being administered with a collective microbial consortium comprising the strains. An ability to utilize a unique carbohydrate can allow a selected strain to establish a niche within the intestinal tract of the patient or subject by providing a competitive nutrient advantage.

Strains were evaluated on their ability to utilize a carbohydrate as a sole carbon source by measuring their growth rate in media containing the carbohydrate as the sole carbohydrate source and determining the growth ratio relative to the strain's growth in the same media containing glucose. selected bacterial strains that could utilize lactose and at least one other unique carbohydrate source to grow to a cell density equal to ˜70% of the growth observed in glucose media were identified as candidate bacterial strains for the collective microbial consortium for treating or preventing NEC. Additional selection criteria of the candidate bacterial strain can comprise those that could also utilize additional carbohydrates to grow to a biomass equal to or greater than 50% of that in glucose. Candidate bacterial strains were additionally selected for use in a collective microbial consortium that did not share 100% similarities in carbohydrate utilization profiles.

Provided herein are methods for the preparation and execution of direct carbohydrate utilization screening for the purpose of screening selected bacterial isolates for their ability to utilize carbohydrates and growth factors that may promote host colonization and therapeutic efficiency. Such strains may be used as part of a collective microbial consortium described herein. FIG. 2 illustrates the overall direct carbohydrate utilization screening method.

### 1. Preparation of the Carbohydrate Stock Solutions

Generally, the procedures for preparing carbohydrate stock solutions as described herein can be used for culturing and screening obligatory and facultative anaerobic bacterial strains. The exemplary bacterial strains in this example were derived from a human fecal, vaginal, or gastrointestinal sample and were grown on selective media.

First, the carbohydrate stock solutions were prepared by dissolving each carbohydrate listed in TABLE 6, excluding mucin, guar gum, pectin, and inulin, in Milli-Q water to create a 20% carbohydrate solution, 10× the Final Concentration percentage listed in TABLE 6. Mucin, guar gum, pectin, and inulin were not dissolved in Milli-Q water.

selected bacterial strains were subjected to at least three assays: (1) intestinal epithelial cell (IEC) adhesion assay; (2) Infant-relevant carbohydrate utilization; (3) NEC-pathogen inhibition; (4) TLR4 Receptor Inhibition; and (5) barrier integrity.

TABLE 6
Carbohydrate Compound Library.

| | | Catalog | | Final % |
|---|---|---|---|---|
| Carbohydrate | Vendor | Number | Category | Concentration |
| 2FL | DSM | 5016552174 | HMO | 2 |
| LNnT | DSM | 5016561174 | HMO | 2 |
| Mucin | Sigma | M1778-100G | Host | 0.3 |
| Galactooligosaccharides | Carbosynth | OG32134 | oligosaccharides | 2 |
| (GOS) | | | | |
| Fructooligosaccharide | Carbosynth | OF05207 | oligosaccharides | 2 |
| (FOS) | | | | |
| D-Lactose monohydrate | Sigma | 61339-25G | disaccharide | 2 |
| L-Fucose | Carbosynth | MF06710 | deoxy sugar | 2 |
| N-Acetylneuraminic acid | Carbosynth | MA00746 | deoxysugar | 2 |
| (Sialic Acid) | | | | |
| N-Acteyl Glucosamine | Sigma | A3286-100G | amino sugar | 2 |
| D-Mannose | Carbosynth | MM06704 | monosaccharide | 2 |
| D-Galactose | Carbosynth | MG05201 | monosaccharide | 2 |
| D-Fructose | Carbosynth | MF00781 | monosaccharide | 2 |
| Inulin | Sigma | I2255 | prebiotic | 0.3 |
| polydextrose | Carbosynth | YP29207 | prebiotic | 2 |
| maltodextrin | Spectrum | M1083 | prebiotic | 2 |
| pectin | Hoosier Hill | Hoosier Hill Fruit | prebiotic | 0.3 |
| | Farms | Pectin, 2 Lb | | |
| Guar gum | Bob's Red | Bob's Red Mill Guar | prebiotic | 0.05 |
| | Mill | Gum, 08 Oz | | |
| Corn Syrup | Golden | Golden Barrel Corn | prebiotic | 2 |
| | Barrel | Syrup (32 fl. oz.) | | |
| D-Glucose | Sigma | G7021 | monosaccharide | 2 |

Excluding mucin, guar gum, pectin, and inulin, the 10× carbohydrate stock solutions were filtered through a sterile 0.22 μm PES syringe filter (Millipore). Mucin, guar gum, pectin, and inulin were dissolved in water and autoclaved to attain sterility.

### 2. Stock Carbohydrate Plates Preparation

The stock carbohydrate plates were prepared by aliquoting 20 μL of each 10× carbohydrate stock solution individually into each well of the plate in a biosafety cabinet, as diagrammed in FIG. 3. Stock carbohydrate solutions were added into all wells except F1-3 and H9, into which water was pipetted. Rows G and H were media and carbohydrate stock solution sterile controls. Bacteria culture was pipetted into the wells in rows A-F. The bottom rows of the plate, row G and H, are negative media controls to evaluate media sterility. Upon completion of stock carbohydrate plate preparation, the plates were wrapped in a seal of parafilm and frozen at −20° C.

### 3. Inoculum and Culture

First, the frozen stock carbohydrate plates were prepared for inoculation by being defrosted, centrifuged at 400 rpm for 1 minute, and placed in an anaerobic chamber for one hour before use.

The previously isolated bacterial strains Bifidobacteria and Lactobacillus were cultured overnight in complete media with glucose. A 1:100 dilution of the Bifidobacteria and Lactobacilli cultures were prepared in a basal media without carbohydrates (TABLEs 3-4).

After dilution, 180 μL of the diluted bacterial strain was pipetted into each well of row A, row B, row C, row D, row E, and row F of the defrosted and anaerobically stored stock carbohydrate plates as diagrammed in FIG. 3. For the sterile media controls, 180 μL of basal media (TABLEs 3-4) were pipetted into each well of row G and row H as diagrammed in FIG. 3.

The OD₆₀₀ spectrophotometer measurement of each well of the stock carbohydrate plates was taken every 30 minutes for a 24-hour time period.

The summary of the result is shown in TABLEs 11-1 and 11-2. All strains could utilize lactose as a sole carbohydrate source. As an illustrative example, the top 20 percentile of strains that were tested could also utilize at least one other infant-relevant carbohydrate source that was expected to be abundant in human breast milk or infant milk formula (such as 2FL, LNnT). Candidate bacterial strains for collective microbial consortium for treating or preventing NEC could utilize lactose and at least 2 of these other infant-relevant carbohydrates to grow to a biomass equal to or greater than 70% of that in glucose. Many of these strains could also utilize additional carbohydrates to grow to a biomass equal to or greater than 50% of that in glucose.

### NEC-Pathogen Growth Inhibition

Candidate bacterial strains for collective microbial consortium for the treatment and prevention of NEC are identified as having the ability to inhibit NEC pathogen growth.

Methods for determining the inhibitions are outlined in EXAMPLE 11.

The summary of the result is shown in TABLEs 11-1 and 11-2. Most of the candidate strains could inhibit the growth of E. coli by more than 70%.

### TLR4 Receptor Inhibition

Candidate bacterial strains can be identified having sufficient ability to inhibit innate immune response (such as TLR4) activation if culture supernatants from their growth can reduce LPS-stimulated TLR4 activation by at least 50%. Such an ability can indicate that strain can provide protection against an over-reactive and inappropriate innate immune response. Such an immune response often precedes overt NEC disease onset.

Strains were compared against 1) a commercially available TLR antagonist (LPS purified from R. sphaeroides) available from Invivogen (tlrl-rslps) and 2) the comparator strains BG49 (Lactobacillus) or EV27 (Bifidobacterium), which are single strain products of other companies.

In the TLR4 receptor inhibition assay, the LPS used for activation of TLR4 was purified from gastrointestinal pathogen (such as E. coli and is commercially available from Invivogen (tlrl-b5lps)) The. selected bacterial strains were individually tested for their ability to inhibit LPS-stimulated TLR4 activation. HEK-Blue™ hTLR4 cells having the SEAP reporter (Invivogen, hkb-htlr4) were cultured in a sterilized Biosafety cabinet (BSC) in complete media (DMEM (Gibco 11995) with 10% FBS, 1% Penicillin and Streptomycin (Gibco 15140-122), 1% Normocin, and 1% HEK-Blue™ Selection) at 37° C. in an atmosphere of 5% CO². The HEK-Blue™ hTLR4 cells were incubated in pre-warmed sterile PBS for 5 mins before being pipet mixed and transferred to a 15 mL tube (for the T175 flask, use 10 mL of PBS). The cells were counted and diluted in treatment media (DMEM (Gibco 11995) with 10% heat-inactivated FBS, 1% Penicillin and Streptomycin (Gibco 15140-122)) to a concentration of 138,889 cells per mL and then seeded into a 96-well plate at a concentration of 25,000 cells per well and incubated at 37° C. in an atmosphere of 5% CO².

Next, culture supernatants were prepared from each selected strain for testing—each supernatant was diluted to 80% in treatment media (60 uL supernatant and 15 uL treatment media). Each mix was vortexed and 10 uL were added to each of 4 replicate wells in the 96-well plate. The plate was incubated for 5-h before the addition of LPS. LPS was added to a final concentration of 0.1 ng/mL (10 uL of 2 ng/mL stock prepared in treatment media). Media controls, with and without LPS addition, were included on each plate for signal normalization. Once LPS was added, the 96-well plate containing the treated HEK-Blue™ hTLR4 cells was incubated at 37° C. in an atmosphere of 5% CO² for 18-h. After 18-h, 10 uL of supernatant was collected from each well into a new 96-well plate and added with 90 μL of HEK-Blue™ Detection media for 4 hours protected from light at room temperature for determining TLR4 activation. TLR4 activation is determined by reading the colorimetric absorbance at 630 nm on a standard plate reader.

The summary of the result is shown in TABLE 11. No individual strain grown in glucose rich culture media was able to generate supernatant that could inhibit LPS-stimulated TLR4 activation. Thus, all the selected strains were used as potential candidate strains in a collective consortium for to test for their ability to inhibit TLR4 activation.

### Barrier Integrity

Candidate bacterial strains for the collective microbial consortium for the treatment and prevention of NEC should be able to maintain or improve intestinal barrier integrity, in some cases, via extracellular products.

The ability of bacterial strains to affect epithelial barrier integrity in vitro was assessed using an impedance-based real-time cell monitoring system. The conditioned media were from selected bacterial strains were harvested as previously described (see EXAMPLES 3 or 7). The cellular impedance of c2BBe1 intestinal epithelial cells was measured using the xCELLigence Real-Time Cell Analysis system (Agilent) as follows. Baseline impedance measurements were recorded by adding 50 μl of cell culture media (DMEM w/ 10% FBS+30 mM HEPES) to a 96-well E plate (PET). The plate was loaded in an xCELLigence MP cradle housed in a tissue culture incubator at 37° C., 5% CO₂, and baseline impedance was measured. After the measurements, the plate was removed from the cradle, and each well was seeded with 49,000 cells/well in 50 μl of cell culture media. After seeding the plate, it was incubated under normal atmospheric conditions at room temperature for 30 minutes. The plate was then loaded in the MP cradle and incubated for hour with impedance measurements recorded every 15 minutes. In a separate 96-well plate, treatments of bacterial products from select strains were prepared. In triplicate, 22 μl of media controls or bacterial products were mixed with 88 μl of cell culture media. 100 μl of this mixture was then added to the E plate, which was loaded back into the MP cradle. The plate was incubated for 72 hours with measurements recorded every 5 minutes. Data were analyzed by normalizing the Cell Index (cellular impedance) to the time of treatment addition. The average Normalized Cell Index was plotted for the entire time course of the experiment. Intestinal epithelial barrier integrity was assessed in 2 ways: as the cellular impedance achieved after 40 hours of treatment (when the cellular impedance reached a steady state plateau) and as the area under the curve of the average Normalized Cell Index line.

The effect of extracellular bacterial products on intestinal epithelial barrier formation in vitro are represented as the change in the Cell Index over time, normalized to the time of treatment addition. Extracellular bacterial products from individual strains did not increase the rate at which cellular impedance increased but did affect the maximum cellular impedance achieved. Quantification of the 40-hour cellular impedance between treatment and control conditions are shown in TABLE 11. Extracellular bacterial products from individual strains had diverse effects on the maximum cellular impedance of the c2BBe1 intestinal epithelial cells, ranging from 89-130% of the media control. Some strains had no effect on epithelial barrier formation, such as ST27, ST81, and ST116. Other strains increased the maximum cellular impedance greater than 20% (relative to a media-only control), such as ST23, ST37, ST66, ST96, and ST101. None of the extracellular bacterial products tested drastically decreased intestinal epithelial barrier formation. The candidate bacterial strains that could improve barrier function by at least 10% over the control, as measured by increased cellular impedance after 40 hours of treatment or by an increase in the area under the curve calculated from recording cellular impedance over time, were identified as candidate bacterial strains.

Collectively, the candidate strains for collective microbial consortium for treating or preventing NEC showed sufficient ability to: The selected bacterial strains had sufficient ability to: adhere to IEC, utilize infant-relevant carbohydrate, inhibit NEC-pathogen growth, and increase gastrointestinal barrier integrity.

### Example 4: Collective BV- or NEC-Specific Functionality Assays

Provided herein are methods for evaluating collective effects of individual selected microbial strains within a collective microbial consortium.

Individual candidate bacterial strains were first evaluated for having the ability to be a donor or recipient strains when combined with other candidate bacterial strains, such as modified using the methods described in EXAMPLEs 7-8. the identified donor and recipient strains were then subjected to disease specific assays to identify collective microbial consortium, such as using the methods of this EXAMPLE or modified from those described herein (such as in EXAMPLEs 2-3, 6, 8, and 10-11).

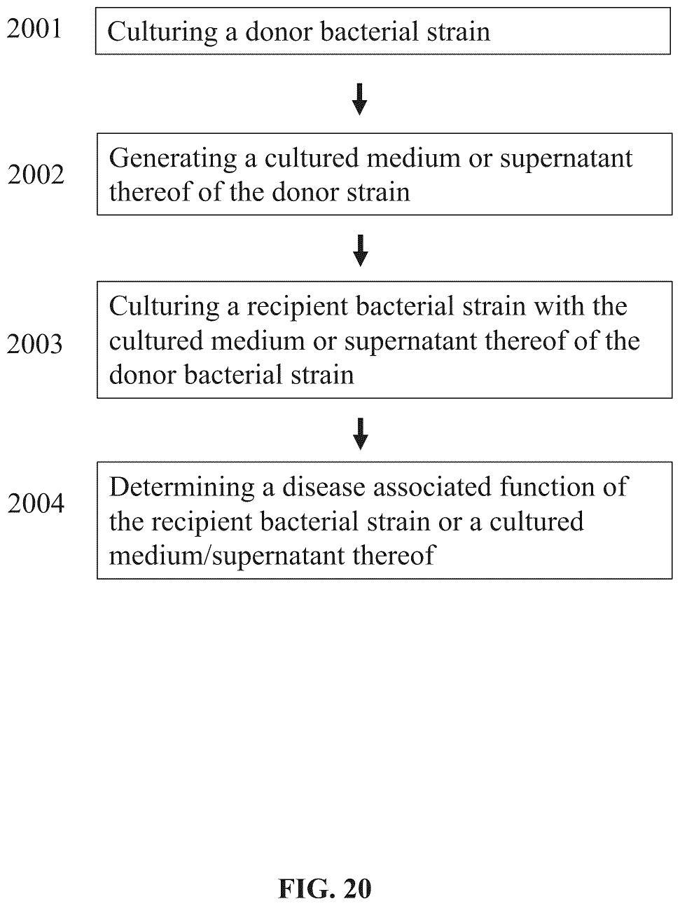

FIG. 20 depicts a cartoon schematic of the method for evaluating collective effects of individual candidate microbial strains within a collective microbial consortium. In process 2001, a donor bacterial strain is inoculated in a culture medium for 24-36 hours. In process 2002, the cell-free supernatant of the donor bacterial culture is harvested from the donor bacterial culture by pelleting and removing the donor bacterial cells from the cultured medium. Cell-free supernatants are further filtered using a 0.2 um filter to remove cells and cellular debris. Optionally, 9 parts of the supernatant of the donor bacterial culture and 1 part of 10× growth media without a sugar source are mixed and use to inoculate a recipient bacterial strain for about 24 hours. In process 2003, the cell-free supernatant of the recipient bacterial culture is harvested from the recipient bacterial culture by pelleting and removing the recipient bacterial cells from the cultured medium. In process 2004, the cell-free of the recipient bacterial culture is used to test for various diseases-specific functionality/assays, such as those described herein, including those described in this example (e.g., growth, pathogen-inhibition, and/or immune response reporter) or those modified from EXAMPLEs 2-3, 6, 8, and 10-11.

### Vaginal Pathogen Inhibition

Methods for determining the inhibitions are outlined in EXAMPLE 2. Supernatants of all strains of interest were used at a final dilution of 1/10 in the assay.

The result of the exemplary consortium is summarized in TABLE 12. For example, most collective consortium, such as those comprising ST105 or ST21, inhibited the growth of BV pathogen significantly better than those of the individual strain when cultured alone (i.e., without the conditioned medium) or controls (see a comparison of TABLEs 8 and 11), suggesting these collective consortia could be more effective in treating or preventing BV compared to the individual bacterial strain.

TABLE 12
Summary of BV-specific MOA and functionality assays
for exemplary collective combinations and control

| | % Growth between (1) ST100 | G. vaginalis Biofilm Inhibition |
|---|---|---|
| | conditioned medium with | (% Reduction) when grown on |
| | carbon source as glucose | ST100 conditioned medium with |
| Bacterial strain(s) | and (2) MRS medium | carbon source as glucose |
| L. jensenii ST21 | 68% | 47% |
| L. gasseri ST105 | 69% | 50% |
| L. crispatus ST20 | 0% | 6% |
| L. crispatus ST43 | 0% | 13% |
| L. crispatus ST92 | 7% | 13% |
| L. jensenii ST21 + | N/A | 55% |
| L. gasseri ST105 | | |
| L. crispatus ST20, | N/A | 54% |
| L. jensenii ST21, | | |
| L. gasseri ST105 | | |
| L. crispatus ST20, | N/A | 13% |
| L. crispatus ST43, | | |
| L. crispatus ST92 | | |

N/A: not applicable

### NEC Pathogen Inhibition

Candidate bacterial strains for collective microbial consortium for the treatment and prevention of NEC can inhibit the growth of NEC pathogen (such as E. coli) by at least 5%. As an illustrative example, the candidate bacterial strains can inhibit the growth of E. coli by at least 20% compared to the media control.

Methods for determining the inhibitions are outlined in EXAMPLEs 3 and 11. Supernatants of all strains of interest were used at a final dilution of 1/10 in the assay.

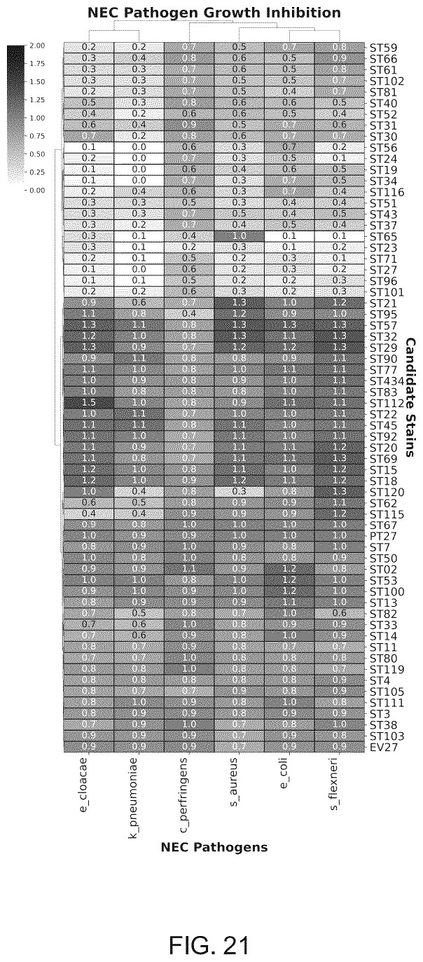

The effect of the conditioned media comprising the extracellular products from the selected bacterial strains on planktonic growth of NEC pathogens as Pathogen Growth Ratio values. These values were aggregated into a cluster map, an example of which shows various selected bacterial strains in FIG. 21 and TABLE 13.

TABLE 13
Summary of NEC-specific MOA and functionality assays
for exemplary collective combinations and control

| | Growth ratio (%) between | TLR4 inhibition | Barrier function |
|---|---|---|---|
| | (1) ST31 conditioned | with cultured | with cultured |
| | medium with carbon source | in ST31 | in ST31 |
| | as glucose and (2) non- | conditioned | conditioned |
| Bacterial | nonconditioned medium with | medium (% | medium (% over |
| strains | carbon source as glucose | inhibition) | control AUC) |
| ST19 | not tested | not tested | not tested |
| ST23 | 70 | 61 | 109 |
| ST24 | not tested | not tested | not tested |
| ST27 | 70 | 25 | 137 |
| ST31 | N/A | N/A | N/A |
| ST34 | 90 | not tested | not tested |
| ST37 | not tested | not tested | 97 |
| ST51 | 85 | not tested | not tested |
| ST56 | not tested | not tested | not tested |
| ST61 | 80 | 79 | 143 |
| ST65 | 85 | not tested | not tested |
| ST66 | 75 | 86 | 105 |
| ST71 | not tested | 46 | 132 |
| ST80 | not tested | 0 | 127 |
| ST81 | 75 | 89 | 150 |
| ST96 | not tested | not tested | 140 |
| ST101 | 75 | 75 | 152 |
| ST102 | not tested | not tested | not tested |
| ST116 | 80 | not tested | not tested |
| ST119 | not tested | 94 | 145 |
| L. reuteri BG49 | N/A | N/A | N/A |
| B. longum EV27 | N/A | N/A | N/A |
| E. coli | N/A | N/A | N/A |
| notes | ST31 grown in | ST31 grown in | ST31 grown in |
| | 2FL or LNnT | glucose | glucose |

N/A: not applicable

Condition media from individual strains had diverse effects on the planktonic growth of NEC-associated pathogens, clustering into different groups. One group of strains, including ST23, inhibits the growth of 5 out of 6 pathogens by at least 30%. Another group of strains, including ST119, inhibited the growth of 3 out of 6 pathogens by 20%. Another group of strains, including ST57, promoted the growth of some pathogens by 30%. These data were used to identify the selected bacterial strains for the collective microbial consortium for treating or preventing NEC, such as those described in TABLE 13.

### TLR4 Receptor Inhibition

Methods for determining the inhibitions of the innate immune response activation are outlined in EXAMPLE 3. Supernatants of all strains of interest were used at a final dilution of 1/10 in the assay.

The result is summarized in TABLE 13. While no individual strain grown in glucose rich culture media was able to generate supernatant that could inhibit LPS-stimulated TLR4 activation (see EXAMPLE 3 or TABLE 11), when grown in the same media in which glucose was replaced with 50% breast milk whey liquid, 6 strains were identified whose supernatants could inhibit LPS-stimulated TLR4 activation by at least 50%. In addition, 7 strains were identified whose supernatants could inhibit LPS-stimulated TLR4 activation by at least 50% when cross-fed. The power of cross-feeding was apparent in the observation that the culture media used for cross-feeding experiments was the same glucose rich media used for growing individual strains. Thus, significant TLR4 inhibition functionality was discovered when one strain was grown in the presence of the bacterial products generated from another strain. As described herein, NEC is associated with activation of innate immune response (such as those regulated by TLR4). Candidate bacterial strains for collective microbial consortium for the treatment and prevention of NEC should inhibit the activation of TLR4 signaling by at least 1%.

### Barrier Integrity

While none of the extracellular bacterial products tested drastically decreased intestinal epithelial barrier formation (see EXAMPLE 3 or TABLE 11), these extracellular products from cross-fed strains increased the rate at which cellular impedance increased for the c2BBe1 intestinal epithelial cells, as shown by the increased areas under the curve described in TABLE 13. Strains such as ST61, ST81, ST96, ST101, and 119 were able to increase epithelial barrier formation by 40% or greater when cross-fed by ST31.

Collectively, the data showed that collective consortium could inhibit TLR4 activation or increase barrier significantly better than those of individual strains alone (i.e., without the conditioned medium) or controls (see a comparison of TABLEs 11-1/11-2 and 13), suggesting these collective consortia could be more effective in treating or preventing NEC compared to the individual bacterial strain.

### Example 5: Bacterial Strain Repository Culturing and Expansion

Provided herein are methods for the isolation and culturing of bacterial strains for screening and collective microbial consortium construction. Such strains may be used as part of a collective microbial consortium described herein.

Therapeutic bacterial selected bacterial strains in the genera Bifidobacterium and Lactobacillus were cultured from donor samples using vegan MRS media at pH 5.75 and pH 6.75, with and without the addition of L-cysteine. Using a sterile disposable spatula or loop, 10-20 mg of sample were transferred into 900 μL of sterile PBS-C, mixed, and serially diluted to 1E-10. Using a 1 mL pipette, 100 μL of dilutions 1E-8-1E-10 were added to MRS agar growth media (with or without cysteine) and spread across the agar plate in a circular fashion using a sterile disposable hockey stick spreader. Sealed plates were placed inverted (agar on top) in the 37° C. incubator for growth.

After 48-72 hours of growth, parafilm was gently removed from the exterior of the plates and the plates were arranged in order of increasing dilution. Plates with clearly isolated colonies were chosen for strain isolation. The goal was to isolate a diversity of strains by selecting isolated colonies that vary in morphology (size, shape, color, margin, etc.). Using a sterile loop, colonies of interest were selected and streaked for isolation using a 4-quadrant method. After 48 hours incubation at 37 C, isolated colonies were re-streaked on selective media for purification. This process was completed twice. After the second round of colony purification, the isolated colony was transferred to 15 mL of liquid culture media. After 24 hours, cultures were harvested and mixed with glycerol (final 15% concentration) and frozen at −80 C to generate research cell banks. Strain identification was accomplished through Sanger sequencing of the 16SrRNA gene using PCR amplicons generated from the 27F and 1492R primers.

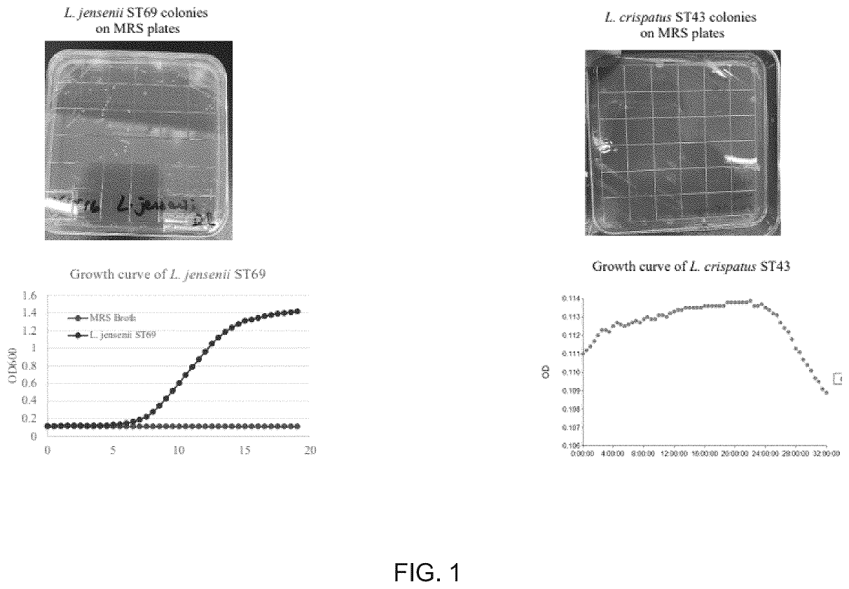

The OD₆₀₀ of the culture was measured periodically and were plotted onto growth curves shown in FIG. 1. The selected media pH and cysteine content were determined by the growth curves for each strain of interest, results of which are shown in TABLE 2. For example, the growth curves demonstrate that L. jensenii exited the log phase and entered the stationary phase at about 15 hours post-inoculation, with an OD₆₀₀ of about 1.4. The growth curves also show that L. crispatus entered the stationary phase at less than about 4 hours with an OD₆₀₀ of about 0.11, and cell death began at about 22 hours post-inoculation.

TABLE 2
Strain Repository Expansion Media Preparation.

| | pH 5.75 | pH 5.75 + cys | pH 6.75 | pH 6.75 + cys |
|---|---|---|---|---|
| VST11 | X | X | — | X |
| (Frozen) | | | | |
| VST15 | X | X | — | L. crispatus |
| (Frozen) | | | | (1 isolate) |
| VST17 | X | X | — | X |
| (Frozen) | | | | |
| VST19 | L. crispatus | L. crispatus | X | X |
| VST22 | X | X | — | X |
| (Frozen) | | | | |
| VST23 | X | X | — | X |
| VST24 | L. crispatus | L. crispatus | — | X |
| VST25 | X | L. gasseri | X | X |
| VST26 | X | X | — | X |
| VST27 | X | L. crispatus | — | X |
| | | L. jensenii | | |
| VST28 | X | X | — | X |
| (Frozen) | | | | |
| VST31 | L. paracasei | L. paracasei | X | X |
| VST32 | L. fermentum | X | X | X |

Media of the pH and cysteine content (1 mM) shown in TABLE 2 were prepared for enrichment of bacterial isolates from the genera Bifidobacterium and Lactobacillus. The media were sterile filtered through a sterile 0.22 μm PES syringe filter (Millipore). The sterile media was then placed in an anaerobic chamber with a loose cap for 24 hours prior to use for inoculation.

The previously isolated bacterial strains Bifidobacteria and Lactobacilli were cultured overnight in complete MRS media with glucose at the pH indicated in TABLE 2.

The cultured bacterial strains of interest were isolated. Examples of an identified selection of these strains is summarized in TABLE 5.

TABLE 5
Vaginal Swab and Infant Stool Sample Isolations Summary.

| | | | | | | | Third | |
|---|---|---|---|---|---|---|---|---|
| | | | PCR & | Dominant | Other | Second | Round | |
| | Colony | Isolates | Sanger | Lactobacillus | Dominant | Round of | of | MCB |
| Samples | Morphology | Selected | Sequencing | Strains | Strains | Isolation | Isolation | Manufacture |
| 25 | Single- | 1221 | 642 | 25 unique | Enterococcus | 17 | 14 | 13 |
| | Multiple | | | Lactobacillus | faecalis | strains | strains | completed |
| | | | | strains | Escherichia | completed | completed | |
| | | | | L. crispatus (12 | fergusonii | | | |
| | | | | unique strains) | Escherichia | | | |
| | | | | L. jensenii (5 | coli | | | |
| | | | | unique strains) | | | | |
| | | | | L. gasseri (2 | | | | |
| | | | | unique strains) | | | | |
| | | | | L. fermentum (2 | | | | |
| | | | | unique strains) | | | | |
| | | | | L. Plantarum (1 | | | | |
| | | | | unique strain) | | | | |
| | | | | L. paracasei (1 | | | | |
| | | | | unique strain) | | | | |
| | | | | L. vaginalis (1 | | | | |
| | | | | unique strain) | | | | |
| | | | | Bifidobacterium | | | | |
| | | | | breve (1 unique | | | | |
| | | | | strain) | | | | |
| n/a | n/a | 1065 | 945 | B. longum (177) | n/a | n/a | n/a | 30 |
| | | | | B. fragilis (124) | | | | |
| | | | | B. bifidum (71) | | | | |
| | | | | E. ramosum (62) | | | | |
| | | | | B. | | | | |
| | | | | pseudocatenulatum | | | | |
| | | | | (50) | | | | |
| | | | | B. breve (31) | | | | |
| | | | | B. stercoris (28) | | | | |

### Example 6: Direct Carbohydrate Utilization Screening

Provided herein are methods for the preparation and execution of direct carbohydrate utilization screening for the purpose of screening selected bacterial isolates for their ability to utilize carbohydrates and growth factors that may promote host colonization and therapeutic efficiency. Such strains may be used as part of a microbial consortium described herein. FIG. 2 illustrates the overall direct carbohydrate utilization screening method.

### 1. Preparation of the Carbohydrate Stock Solutions

Generally, the procedures for preparing carbohydrate stock solutions as described herein can be used for culturing and screening obligatory and facultative anaerobic bacterial strains. The exemplary bacterial strains in this example were derived from a human fecal, vaginal, or gastrointestinal sample and were grown on selective media.

First, the carbohydrate stock solutions were prepared by dissolving each carbohydrate listed in TABLE 6, excluding mucin, guar gum, pectin, and inulin, in Milli-Q water to create a 20% carbohydrate solution, 10× the Final Concentration percentage listed in TABLE 6. Mucin, guar gum, pectin, and inulin were not dissolved in Milli-Q water.

TABLE 6
Carbohydrate Compound Library.

| | | Catalog | | Final % |
|---|---|---|---|---|
| Carbohydrate | Vendor | Number | Category | Concentration |
| 2FL | DSM | 5016552174 | HMO | 2 |
| LNnT | DSM | 5016561174 | HMO | 2 |
| Mucin | Sigma | M1778-100G | Host | 0.3 |
| Galactooligosaccharides | Carbosynth | OG32134 | oligosaccharides | 2 |
| (GOS) | | | | |
| Fructooligosaccharide | Carbosynth | OF05207 | oligosaccharides | 2 |
| (FOS) | | | | |
| D-Lactose monohydrate | Sigma | 61339-25G | disaccharide | 2 |
| L-Fucose | Carbosynth | MF06710 | deoxy sugar | 2 |
| N-Acetylneuraminic | Carbosynth | MA00746 | deoxysugar | 2 |
| acid (Sialic Acid) | | | | |
| N-Acteyl | Sigma | A3286-100G | amino sugar | 2 |
| Glucosamine | | | | |
| D-Mannose | Carbosynth | MM06704 | monosaccharide | 2 |
| D-Galactose | Carbosynth | MG05201 | monosaccharide | 2 |
| D-Fructose | Carbosynth | MF00781 | monosaccharide | 2 |
| Inulin | Sigma | I2255 | prebiotic | 0.3 |
| polydextrose | Carbosynth | YP29207 | prebiotic | 2 |
| maltodextrin | Spectrum | M1083 | prebiotic | 2 |
| pectin | Hoosier Hill | Hoosier Hill Fruit | prebiotic | 0.3 |
| | Farms | Pectin, 2 Lb | | |
| Guar gum | Bob's Red | Bob's Red Mill Guar | prebiotic | 0.05 |
| | Mill | Gum, 08 Oz | | |
| Corn Syrup | Golden | Golden Barrel Corn | prebiotic | 2 |
| | Barrel | Syrup (32 fl. oz.) | | |
| D-Glucose | Sigma | G7021 | monosaccharide | 2 |
| Maltose | ThermoFisher | A16266.22 | Glycogen | 2 |
| | | | metabolite | |
| Mucin | Sigma | M1778-100G | Host | 0.3 |
| Glycogen | Sigma | G0885-5G | Host | 2 |
| Melibiose | Sigma | M5500-5G | Disaccharide | 2 |
| N-Acetylneuraminic | Carbosynth | MA00746 | Deoxysugar | 2 |
| acid (Sialic Acid) | | | | |
| N-Acteyl | Sigma | A3286-100G | Amino sugar | 2 |
| Glucosamine | | | | |
| Maltodextrin | Spectrum | M1083 | Glycogen | 2 |
| | | | metabolite | |
| D-Glucose + | Sigma | G7021 | Monosaccharide | 2 |
| 0.1% cysteine | VWR | J994-100G | Amino acid | |
| | (cysteine) | (cysteine) | | |

Excluding mucin, guar gum, pectin, and inulin, the 10× carbohydrate stock solutions were filtered through a sterile 0.22 μm PES syringe filter (Millipore). Mucin, guar gum, pectin, and inulin were dissolved in water and autoclaved to attain sterility.

### 2. Stock Carbohydrate Plates Preparation

The stock carbohydrate plates were prepared by aliquoting 20 μL of each 10× carbohydrate stock solution individually into each well of the plate in a biosafety cabinet, as diagrammed in FIG. 3. Stock carbohydrate solutions were added into all wells except F1-3 and H9, into which water was pipetted. Rows G and H were media and carbohydrate stock solution sterile controls. Bacteria culture was pipetted into the wells in rows A-F. The bottom rows of the plate, row G and H, are negative media controls to evaluate media sterility. Upon completion of stock carbohydrate plate preparation, the plates were wrapped in a seal of parafilm and frozen at −20° C.

### 3. Inoculum and Culture

First, the frozen stock carbohydrate plates were prepared for inoculation by being defrosted, centrifuged at 400 rpm for 1 minute, and placed in an anaerobic chamber for one hour before use.

The previously isolated bacterial strains Bifidobacteria and Lactobacillus were cultured overnight in complete media with glucose.

A 1:100 dilution of the Bifidobacteria and Lactobacilli cultures were prepared in a basal media without carbohydrates (TABLEs 3-4).

After dilution, 180 μL of the diluted bacterial strain was pipetted into each well of row A, row B, row C, row D, row E, and row F of the defrosted and anaerobically stored stock carbohydrate plates as diagrammed in FIG. 3. For the sterile media controls, 180 μL of basal media (TABLEs 3-4) were pipetted into each well of row G and row H as diagrammed in FIG. 3.

Inoculated culture plates were incubated anaerobically in a spectrophotometer at 37° C. with the lid temperature set to +2° C. to reduce condensation during optical density readings.

The OD₆₀₀ spectrophotometer measurement of each well of the stock carbohydrate plates was taken every 30 minutes, after a 30 second orbital shaking period, for a 24-48-hour time period.

### Example 7: Cross-Feeding Assay

Provided herein are methods for the identifying donor and recipient bacterial strains for generation of a collective microbial consortium. The methods comprise preparation and testing of cross-feeding capabilities for the purpose of screening candidate bacterial isolates with complementary MOAs for their ability to utilize carbohydrates and growth factors that may promote host colonization, and their ability to secrete growth-supporting compounds. Donor strains were screened for their ability to produce bacterial products that support the growth of recipient strains that are incapable of directly using the initial niche-relevant carbohydrate. Such strains may be used as part of a microbial consortium described herein. FIG. 2 illustrates the overall direct carbohydrate utilization screening method.

### 1) Selection of Donor Strains and Recipient Strains

Candidate therapeutic selected bacterial strains of interest belonging to Lactobacillus and Bifidobacterium genera were selected and screened for their ability to utilize an array of distinct, niche-relevant carbohydrates. (A subset shown in TABLE 6). Primary strong consumers of carbohydrates of particular relevance for engraftment in the vaginal canal (glycogen, maltose, maltodextrin) or in the infant gastro-intestinal tract (HMOs, lactose) were prioritized as donor strains. Donor strains were screened for their ability to produce bacterial products that support the growth of recipient strains that are incapable of directly using the initial niche-relevant carbohydrate. Recipient strains were chosen based on strong performance in additional disease-relevant MOA studies, such as those in EXAMPLES 2-3, 6, 8, and 10-11.

### 2. Production and Collection of Donor Strain Metabolic Products

For generation of the donor strain (donor) metabolic products, 5 mL of desired growth media was prepared in a 15 mL culture tube under anaerobic conditions. Culture media from TABLES 3 and 4 were used for donor strain growth and were supplemented with niche-relevant carbohydrates of interest as outlined in TABLE 6. The sterile culture media were inoculated at a 1% v/v using a culture of the donor test strains grown in rich media for 24-36 h at 37° C. under anaerobic or microaerophilic atmosphere. Donor strain cultures were grown in single carbon source media for 24-36 h to stationary phase at 37° C. under anaerobic or microaerophilic atmosphere. Culture growth was monitored by OD600 in a 96-well plate spectrophotometer. After entering stationary phase, cells were centrifuged for 10 min at 2,700×g and cell-free supernatant containing metabolic products of the donor strain selected were collected under sterile conditions. All donor selected supernatants were collected and pH adjusted to 6.0 using sodium hydroxide, followed by filter sterilization using a 0.22 um syringe filter. Sterile supernatants containing donor strain selected metabolic products were allowed to fully reduce in the anaerobic chamber for 18-24 h prior to subsequent use. Control media that were not inoculated with any donor strain selected were also processed as negative controls. Purity of donor cultures was evaluated at each process using microscopy and 16S rRNA gene sequencing. Metabolic products from donor strains were stored at −80° C. for subsequent use when not immediately utilized.

### Evaluation of Recipient Strain Growth on Donor Strain Metabolic Products

The ability of donor strains to produce bacterial products

that support the growth of recipient strains was subsequently evaluated using sterile donor strain metabolic products. An overnight culture of the test recipient selected was grown in rich media for 24-36 h to stationary phase at 37° C. under anaerobic or microaerophilic atmosphere and served as an inoculum culture. Once in stationary phase, 1 mL of the culture was separated by centrifugation for 10 min at 2,700×g. The culture media and supernatant were removed, and the cell pellet was resuspended in 1 mL of mBasal media (TABLE 3) with no carbohydrate source.

TABLE 3
mBasal Media.

| Component | Amount (g/L) |
|---|---|
| Peptone (BBL Trypticase | 10 |
| Peptone BD 211921) | |
| Yeast Extract | 2 |
| NaCl | 5 |
| Magnesium sulfate | 0.2 |
| Dipotassium hydrogen | 2 |
| phosphate | |
| L-cysteine | 0.5 |

Donor test media were prepared in 5 mL volumes in 15 culture tubes. Test media consisting of 9-parts donor selected cell free supernatant and 1-part 10× growth media (-glucose). Growth media was composed of 10×YFAP (for Bifidobacterium strains) or 10×vMRS (for Lactobacillus strains). The donor test culture media were inoculated with 1% v/v of the recipient selected strain resuspended in minimal media (such as mBasal) containing donor selected metabolic products as a media base. Recipient strains were grown in donor test media for 24-36 h at 37° C. under anaerobic atmosphere (90% N₂, 5% CO₂, 5% H₂). Growth was monitored by OD₆₀₀ in an incubated spectrophotometer.

### Example 8: Analysis of Data for Direct Carbohydrate Utilization Screening and Cross-Feeding Assay

Provided herein are methods for growth curve area analysis to determine the growth response of each bacterial strain of interest to various carbohydrate medias. Such strains may be used as part of a microbial consortium described herein.

A growth curve can be plotted for each inoculated well using the R package Growthcurver. Growthcurver can then be used to calculate the area under the growth curve (AUC) for each well. The Growthcurver program may be run with all default parameters, including subtracting the OD₆₀₀ measurement of water or sterile media in each row, which can be the minimum OD₆₀₀ value, from each well. The growth curves may be manually inspected for bubbles and inconsistencies, and if bubbles or inconsistencies are found, these wells may be excluded from analysis.

The average AUC may be computed for each carbohydrate type as listed in TABLE 6, and the AUC value for the water control may be subtracted from every AUC value to factor in baseline growth. The AUC obtained for each carbohydrate can be divided by the AUC obtained with glucose media to obtain the AUC ratio (AUC_{carbohydrate}/AUC_{glucose}). An AUC ratio at or close to 1 indicates the carbohydrate may have been used efficiently as a growth factor by the bacteria.

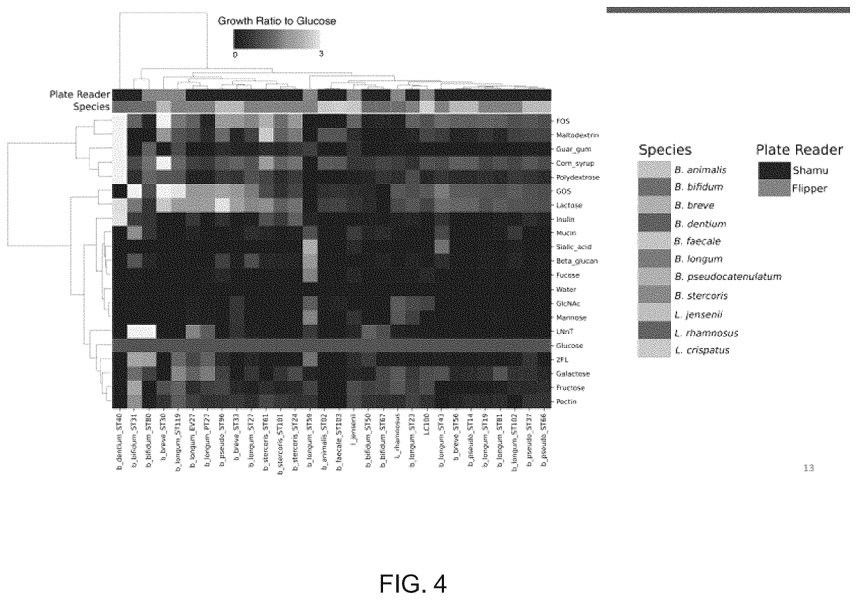

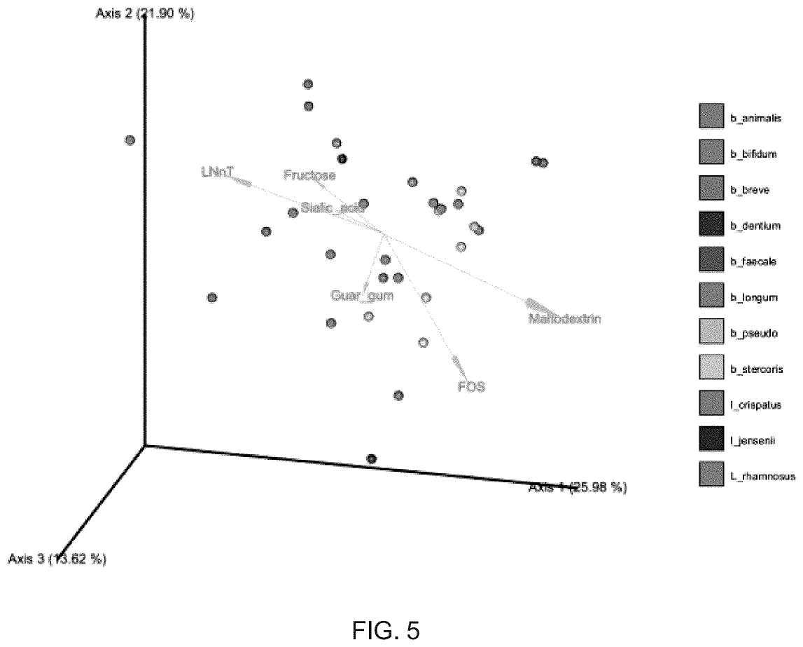

To visualize the data, the Python libraries matplotlib and seaborn may be used to generate growth curve plots and visualize AUC ratios as shown in FIG. 4 and FIG. 5. Carbohydrate utilization patterns across strains may be visualized by clustermaps as demonstrated in FIG. 4, and principal coordinates analysis are exemplified in FIG. 5.

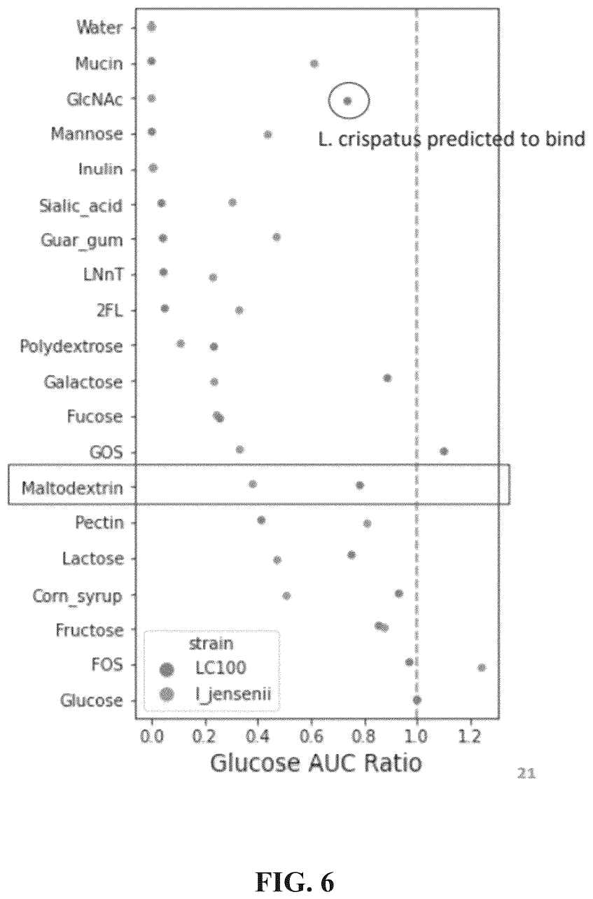

Glucose AUC ratios can be plotted for each type of carbohydrate and used to predict the glucose AUC ratio of additional Lactobacillus strains, as illustrated in FIG. 6 and TABLE 7.

TABLE 7
Expected Lactobacillus Growth on Carbohydrate Medias

| | Carbohydrate | Expected Outcome |
|---|---|---|
| 1 | Glycogen | Growth |
| 2 | Maltose | Growth |
| 3 | Maltodextrin | Growth |
| 4 | Mucin | No Growth |
| 5 | N-Acetylneuraminic | No Growth |
| | Acid (Sialic Acid) | |
| 6 | Melibiose | Growth |
| 7 | Glucose | Growth |
| 8 | Water | No Growth |

### Example 9: Bifidobacteria and Lactobacillus Screening

Provided herein are methods for Bifidobacteria screening to determine the identity of strains of interest isolated from human samples. Such strains may be used as part of a microbial consortium described herein. Exemplary genome sequences are described in SEQ ID NOs: 1-30.

### 1. Bifidobacteria Sequencing

Bifidobacteria cells were cultured overnight in mBasal media. The culture was diluted 1× and grown to an OD₆₀₀ of 1.0 as 15 mL aliquots. The culture was then aliquoted into 1 mL sample aliquots and centrifuged until a cell pellet was formed and bound to a magnetic, thermoplastic disk made with silica nanomaterial. The supernatant was poured off and the pellets were frozen at −20° C.

The Bifidobacteria cells were lysed by adding to the cell pellet 20 mg/mL of lysozyme and 250 U/mL or about 100 μL of mutanolysin, allowing the mixture to sit at room temperature for 1 hour. After 1 hour of lysing, the 1 mL tube was gently inverted to gently mix the cell pellet and lysing solution while keeping the pellet intact.

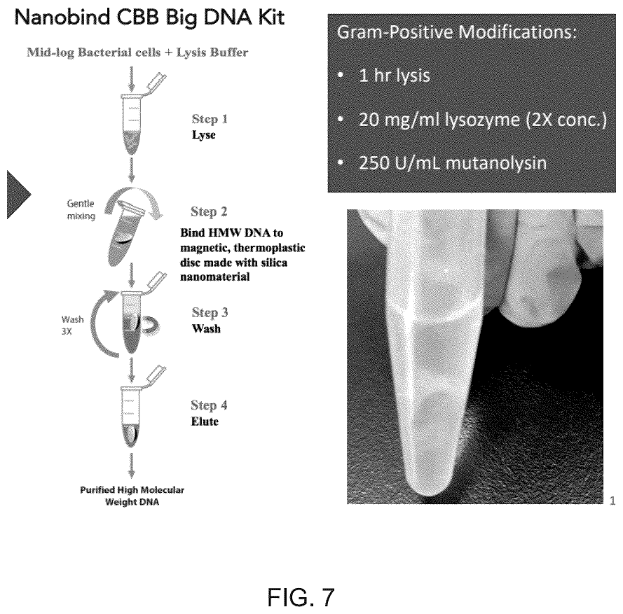

Following lysis, the cell pellet was washed three times by adding wash buffer solution and gently mixing the pellet and wash solution, then disposing of the wash solution. The cellular purification and DNA extraction method is illustrated in FIG. 7.

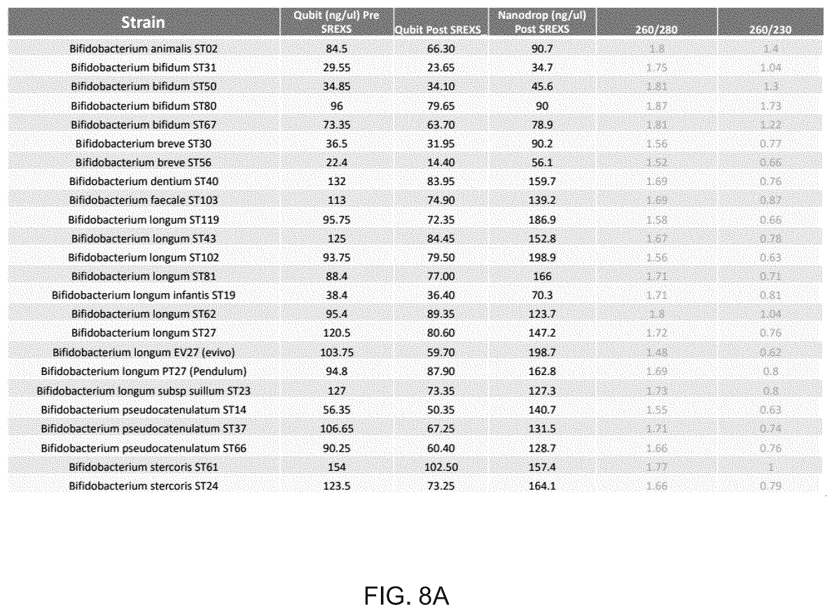

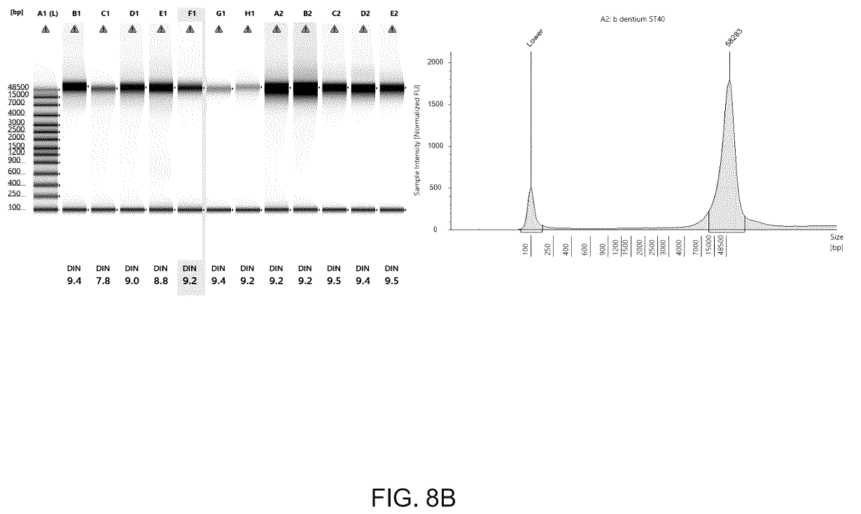

The eluate was then sequenced using Qubit fluorometer and Nanodrop spectrophotometer to detect the ng/μL measurements of the sample and optical density 260/280 and 260/230 of the eluate, example results are shown in FIGS. 8A and 8B.

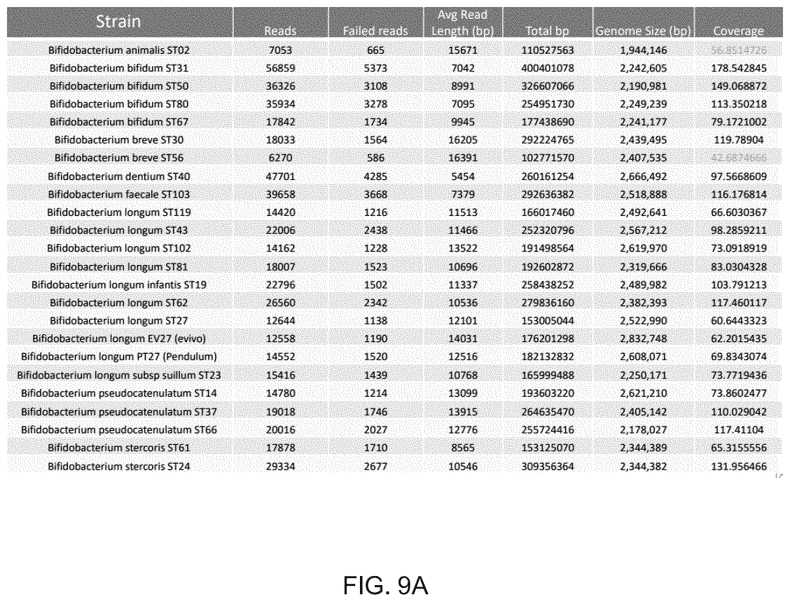

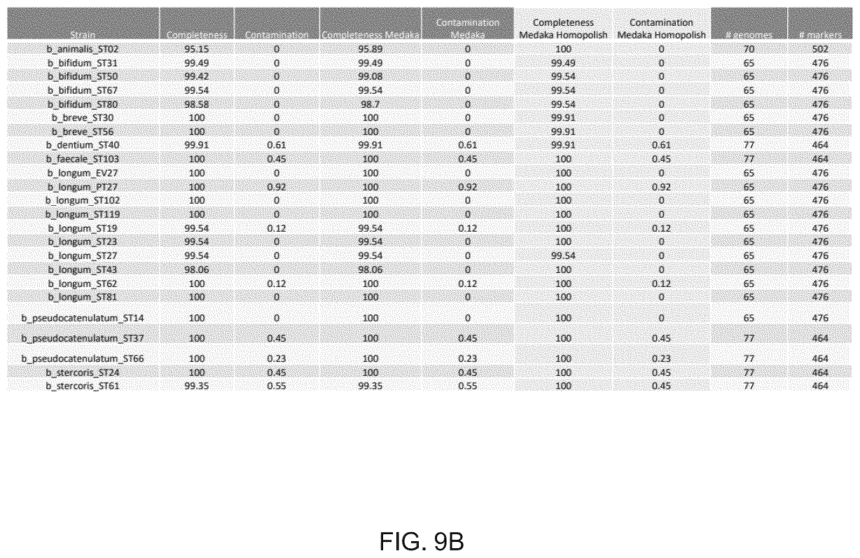

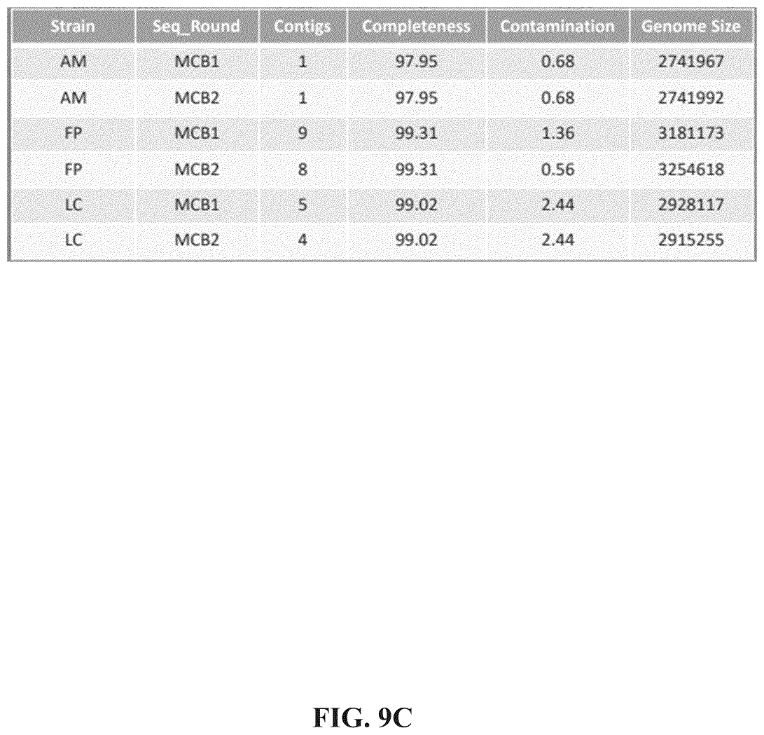

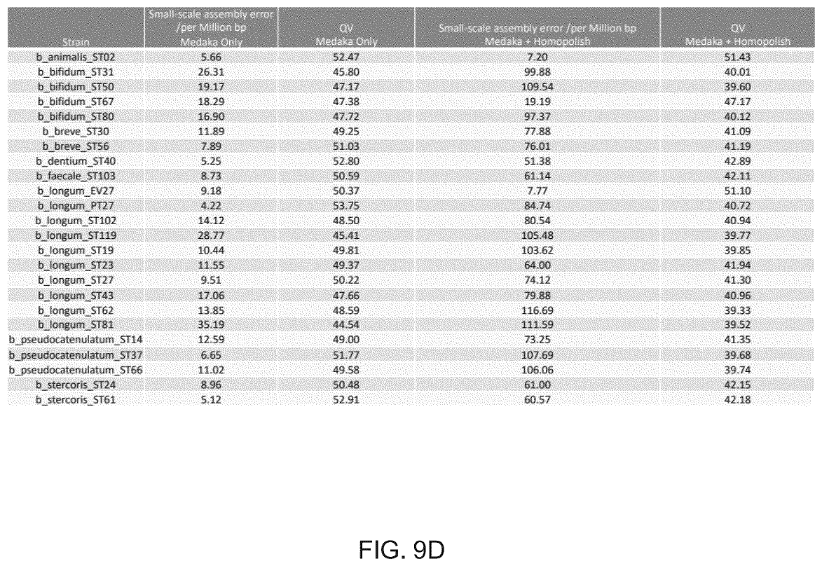

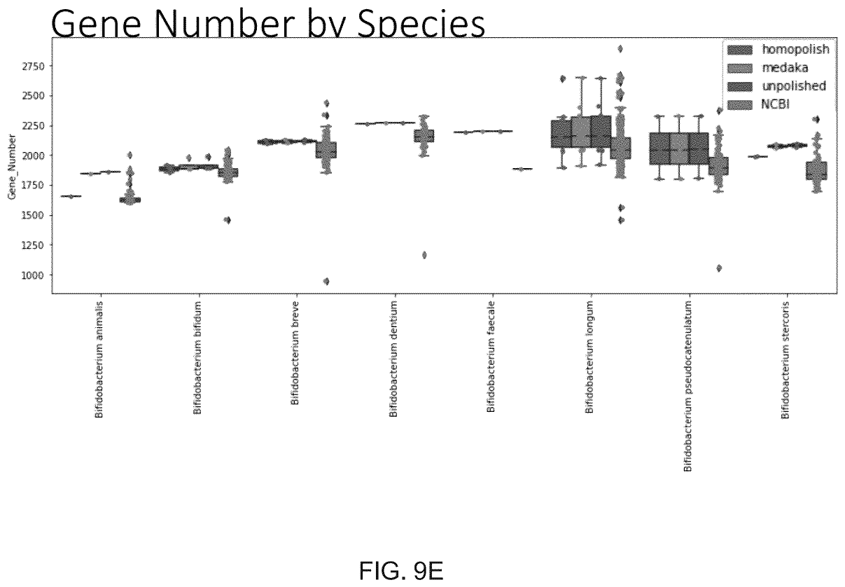

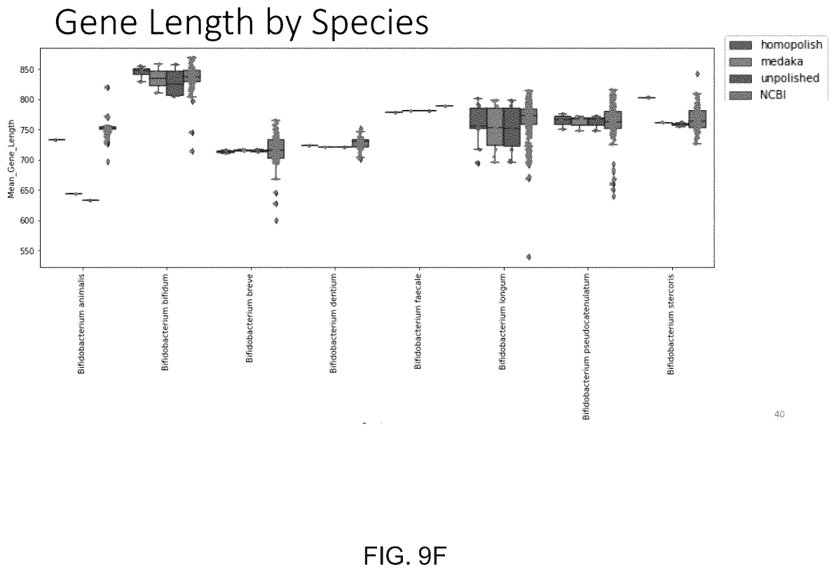

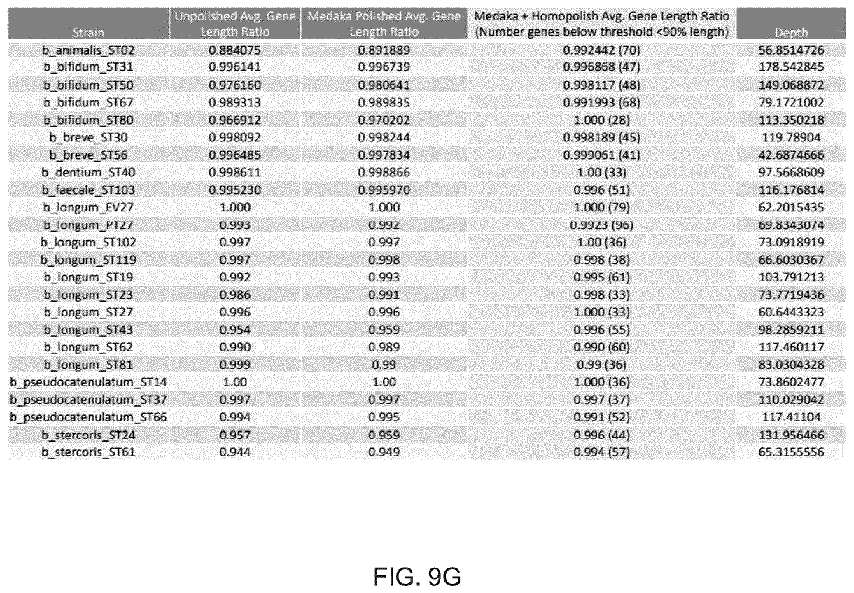

The eluate was also sequenced using nanopore sequencing to analyze the length and purity of the DNA strands. Example results are shown in FIGS. 9A, 9B-9D and 9G.

The nanopore sequencing data was analyzed using prokka software to determine the number of genes and the average gene lengths for each species both polished and unpolished, the example results of which are shown in FIG. 9D-9G.

### 2. Lactobacillus Sequencing

Lactobacillus cells may be cultured overnight in mBasal media. The culture can be diluted 1× and grown to an OD₆₀₀ of 1.0 as 15 mL aliquots. The culture may then be aliquoted into 1 mL sample aliquots and centrifuged until a cell pellet forms and can be bound to a magnetic, thermoplastic disk made with silica nanomaterial. The supernatant may be poured off and the pellets can be frozen at −20° C.

The Lactobacillus cells may be lysed by adding to the cell pellet 20 mg/mL of lysozyme and 250 U/mL, or about 100 μL of mutanolysin, allowing the mixture to sit at room temperature for 1 hour.

Following binding of DNA to the thermoplastic, the DNA may be washed three times in wash buffer solution. The cellular purification and DNA extraction method is illustrated in FIG. 7.

The eluate may then be sequenced using Qubit fluorometer and Nanodrop spectrophotometer to detect the ng/μL measurements of the sample and optical density 260/280 and 260/230 of the eluate.

The nanopore sequencing data can also be analyzed using prokka software to determine the number of genes and the average gene lengths for each species both polished and unpolished.

### Example 10: Additional Assays

### Membrane Integrity Screen

Provided herein are various assays for determining additional functionality of bacterial strains. Such strains may be used as part of a microbial consortium described herein.

### Inducing Membrane Permeability Using TNFα

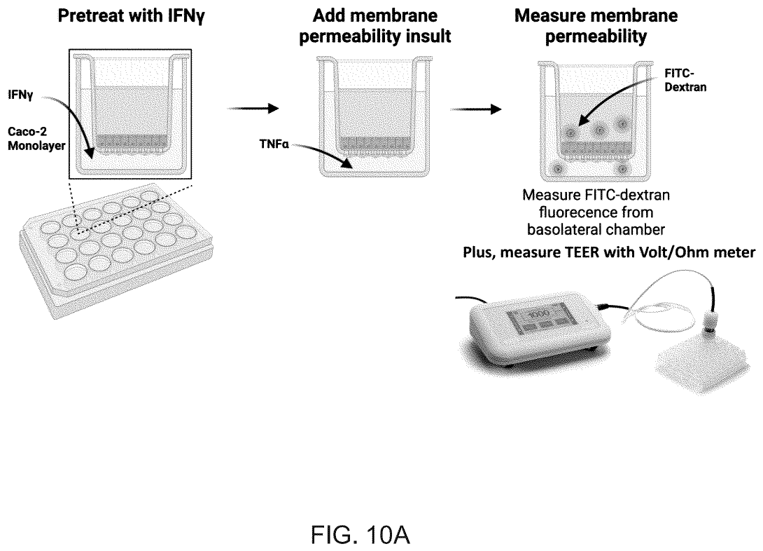

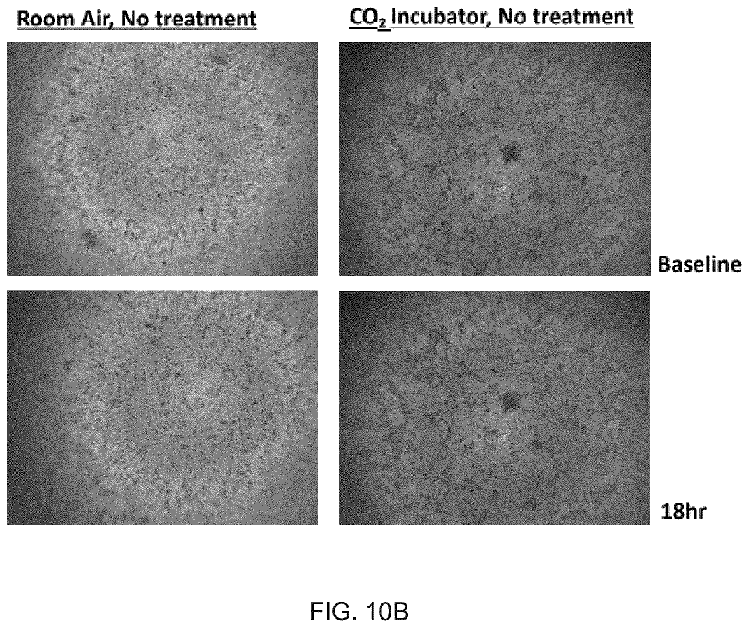

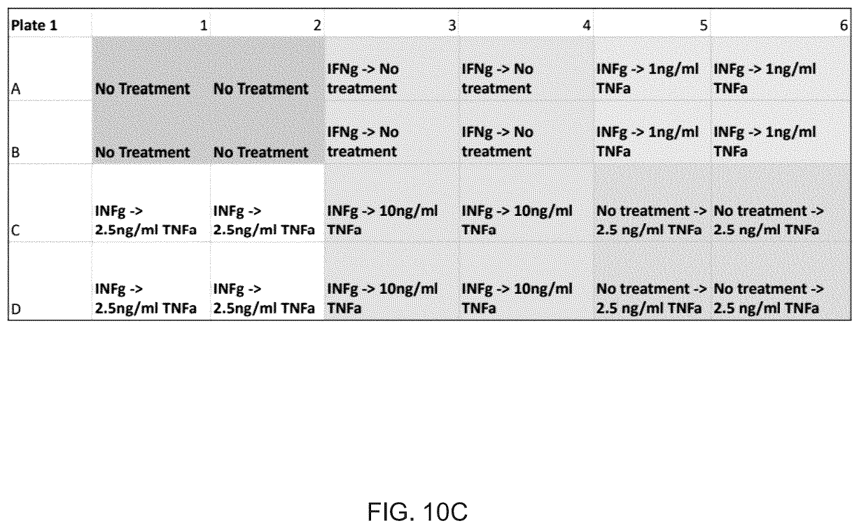

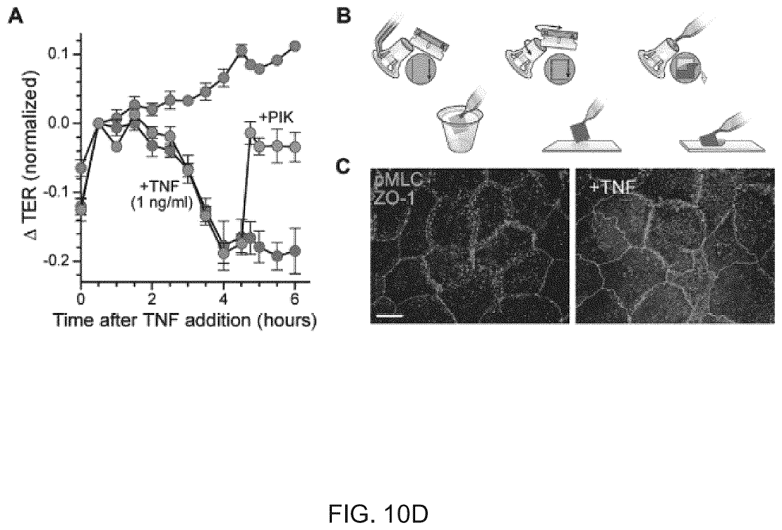

The method for the membrane integrity screen is shown in FIG. 10A. A monolayer of Caco-2 cells pre-treated with Interferon Gamma (IFNγ), except for the control wells, adhered to each well of a 24-well plate were incubated with pasteurized bacterial strains of interest. The monolayer of Caco-2 cells was cultured in either room air or CO₂ incubator conditions, the microscopy examples of which are shown in FIG. 10B. The plate map is shown in FIG. 10C. Tumor Necrosis Factor alpha (TNFα) solution was pipetted into each well as a membrane permeability insult. About 25 μL of FITC-Dextran-4 (FD4) solution was pipetted into each well as a fluorescent tag. The control wells were untreated with either IFNγ or TNFα. Another group received only the IFNγ, another group received only TNFα, and the remaining two groups received either 1 ng/mL or 10 ng/mL of TNFα. Example data of TNFα induced membrane permeability at various hours with the addition of TNF and PIK, demonstrating various amounts of membrane permeability over multiple hours is shown in FIG. 10D.

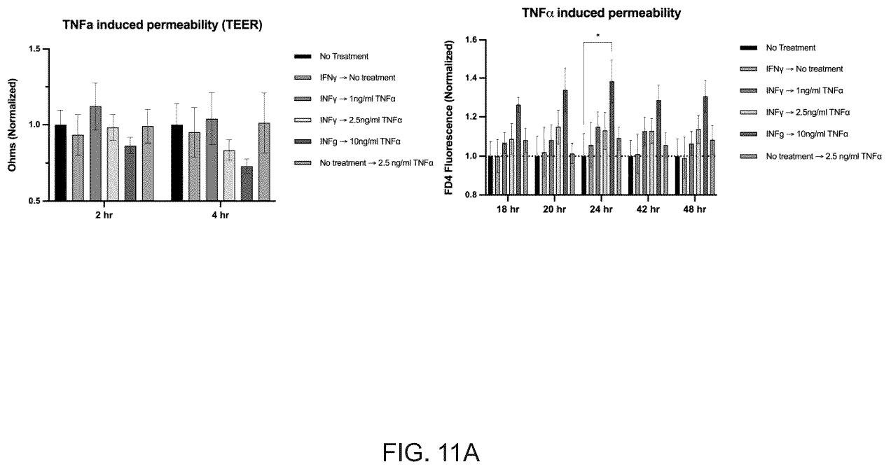

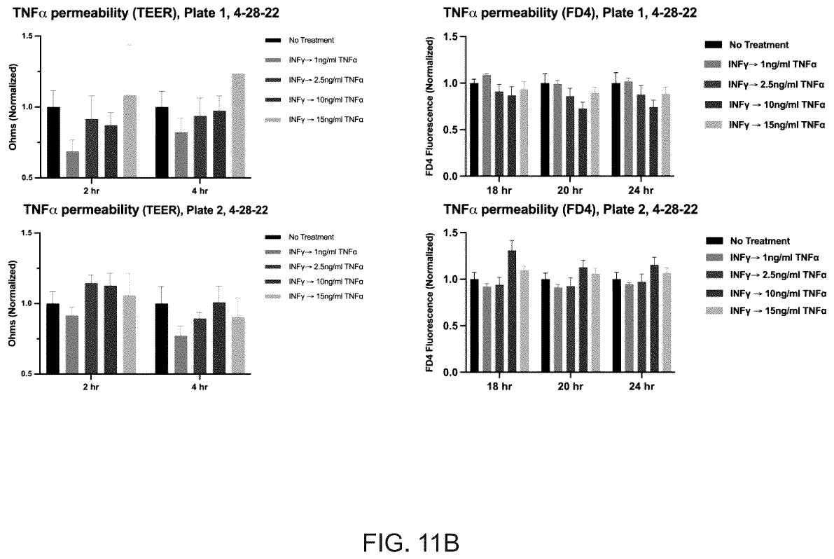

Transepithelial Electrical Resistance (TEER) was measured with a voltmeter or ohmmeter periodically for 4 hours post-TNFα treatment to determine membrane permeability of the Caco-2 monolayer cells, of which result examples are shown in FIGS. 11A and 11B. FD4 was added immediately after the 4-hour TEER measurement was taken. The membrane permeability was also measured via spectroscopy to determine the OD₆₀₀ of the FD4 fluorescence from the basolateral chamber, examples of the results of which are shown in FIGS. 11A and 11B.

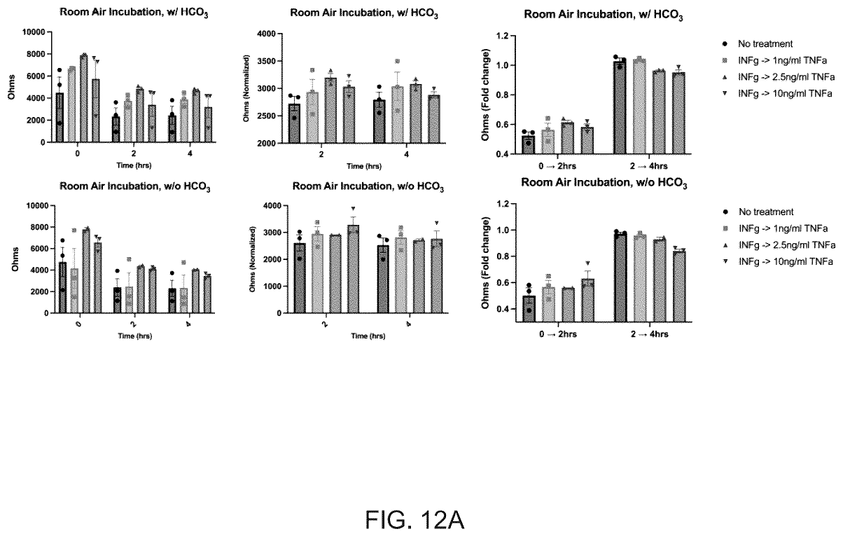

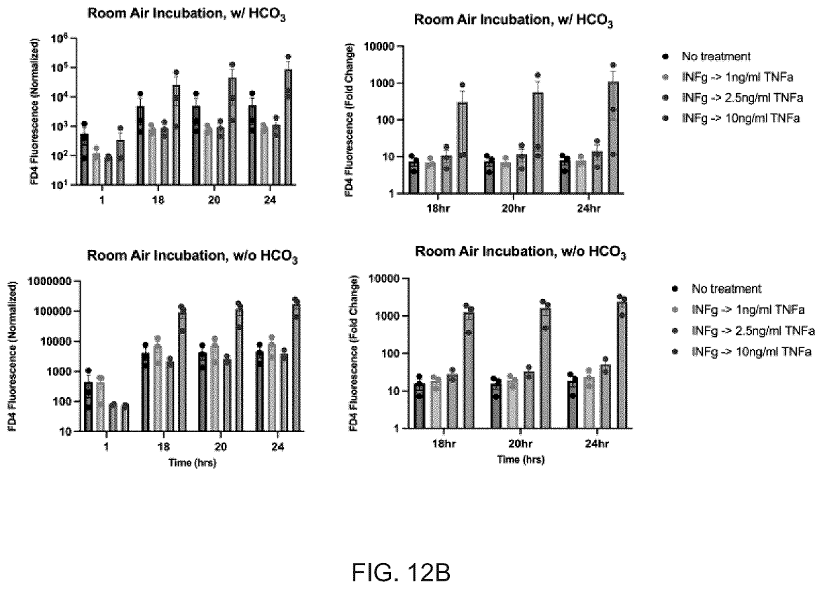

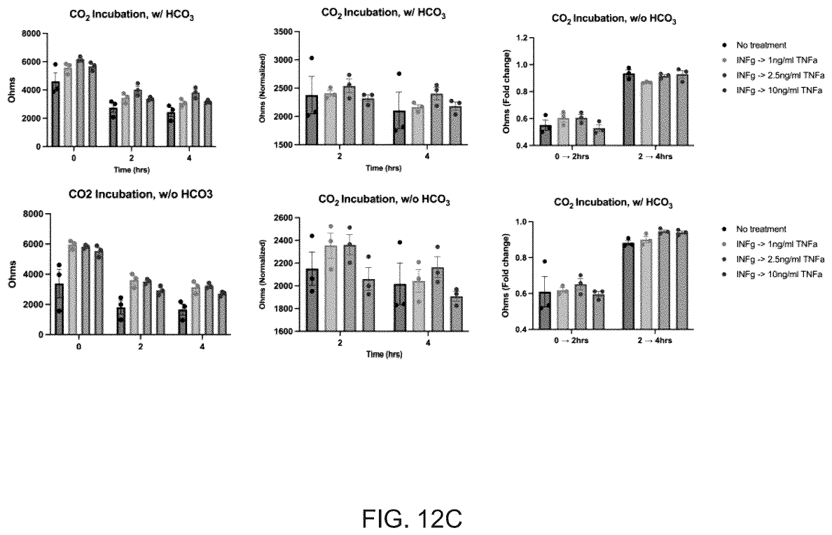

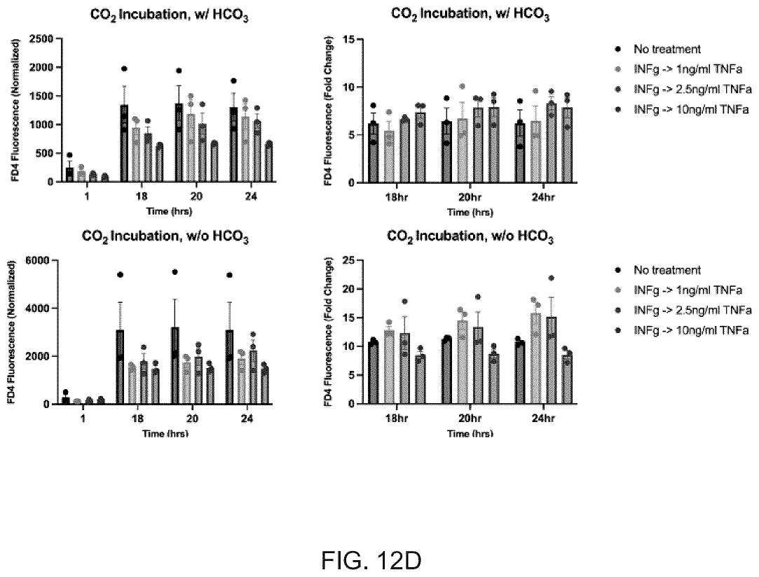

TEER was also measured under various incubation conditions, for example the conditions of the Caco-2 epithelial cells cultured with room air incubation or CO₂ incubation, as well as with and without the addition of 20 μL of HCO₃ into the wells. The results of these conditions are exemplified in FIG. 12A-12D.

### RAW-Dual Reporter

### 1. SEAP Detection Assay

Screening for strains that alter production of immune response factor Nuclear Factor Kappa B (NFκB) was performed via SEAP detection assay. The RAW-Dual immortalized macrophage cell line with stable expression of NFκB and Interferon-Sensitive Response Element (ISRE) was pre-incubated with pasteurized bacterial strains of interest. About 20 μL of bacterial supernatant or pasteurized bacterial cells of interest was added to each well of a 96 well plate, and endotoxin-free water was added as a negative control. About 180 μL of cell suspension was added to each well, and the 96 well plate was incubated in an anaerobic chamber at about 37° C. for about 18-24 hours. About 180 μL of resuspended QUANTI-Blue™ solution was then added in each well, and about 20 μL of RAW cell supernatant was added.

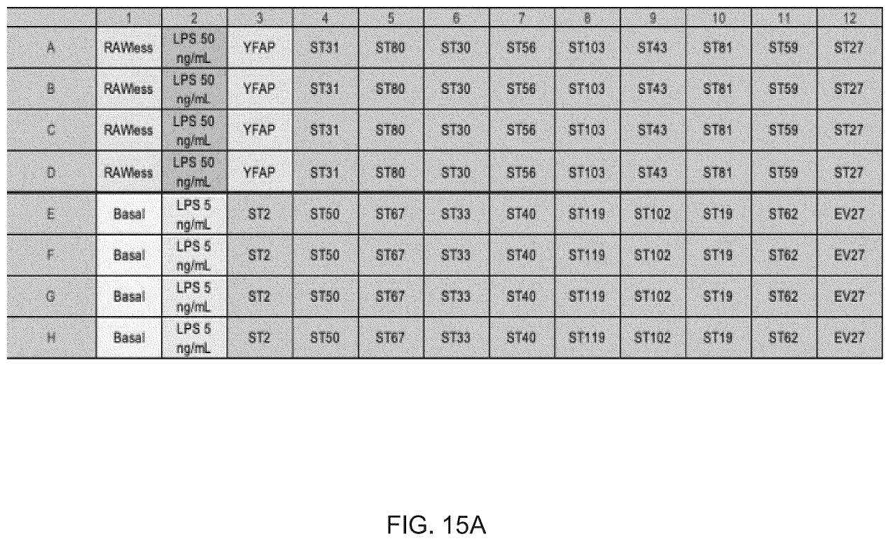

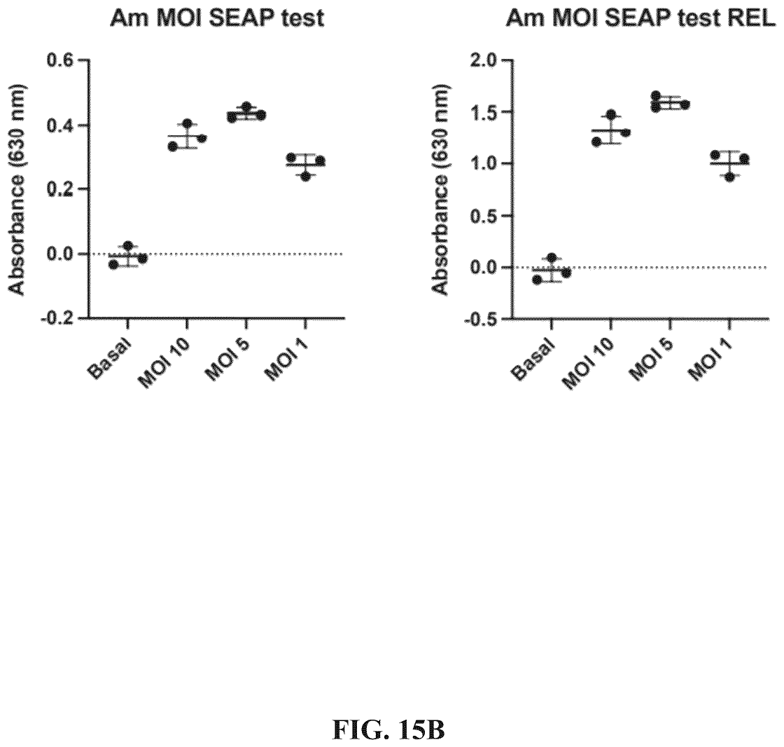

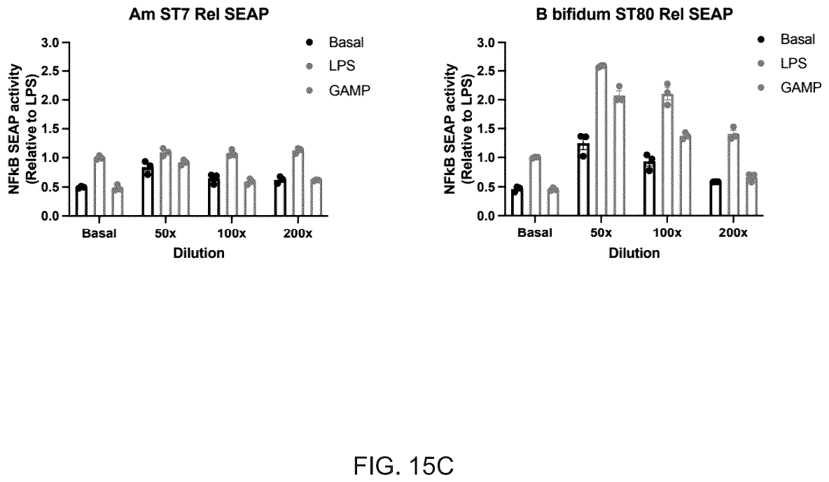

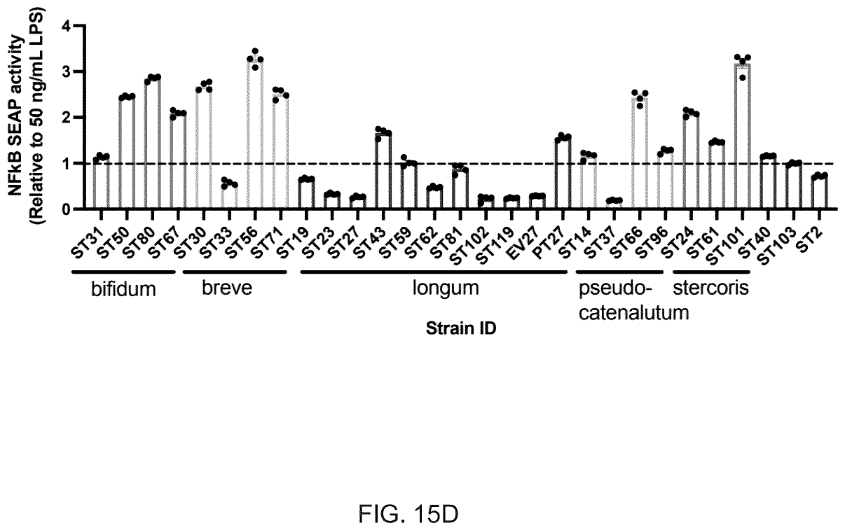

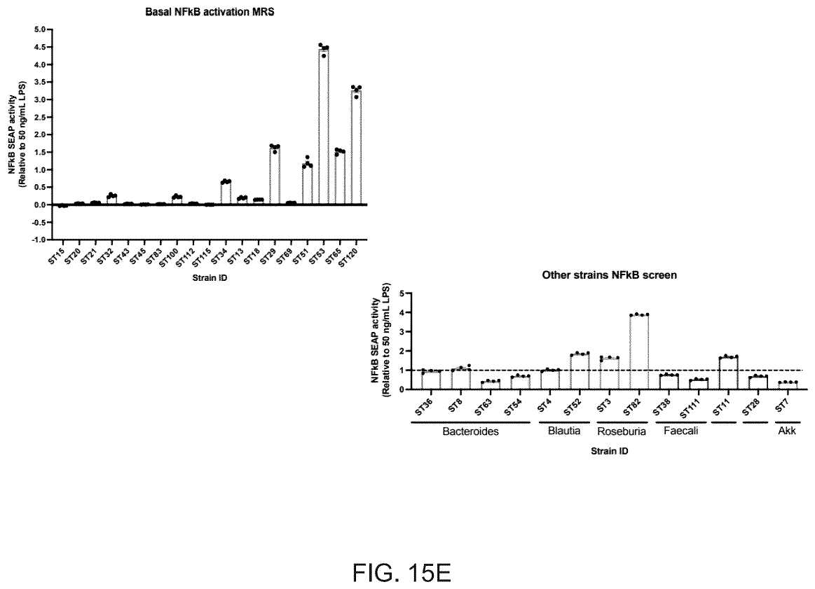

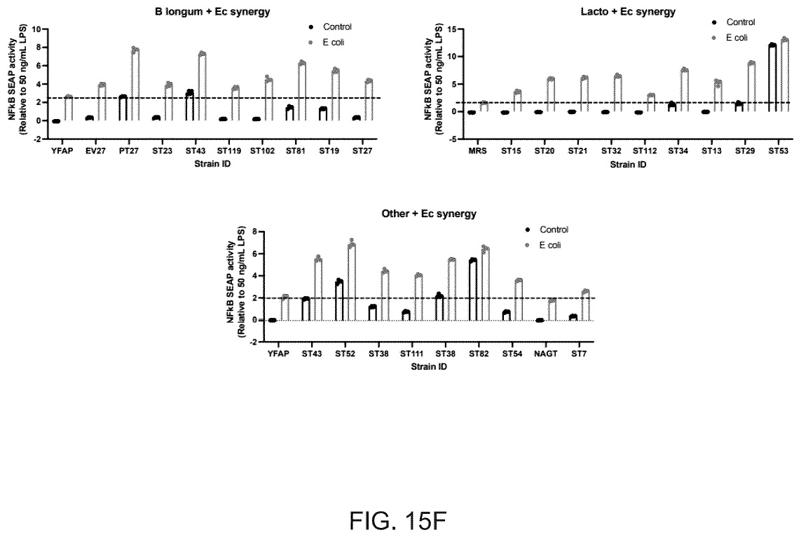

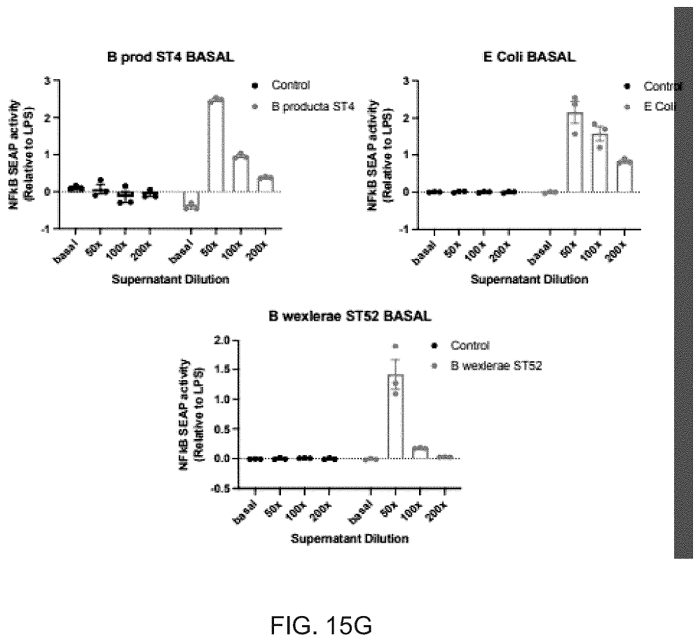

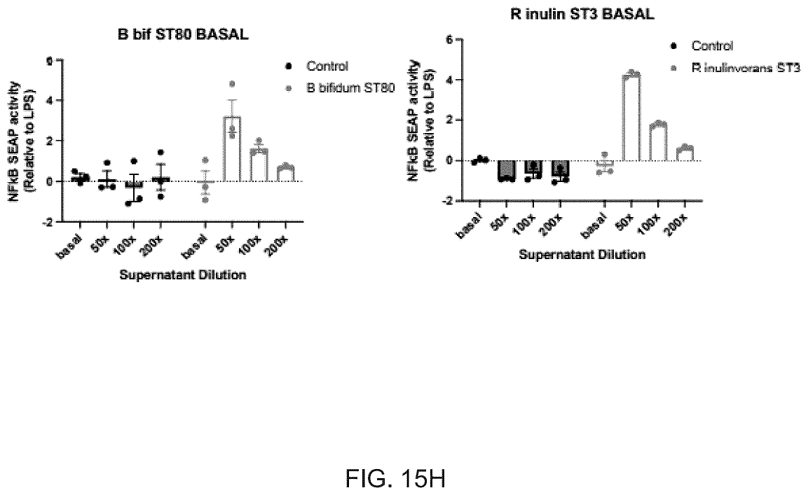

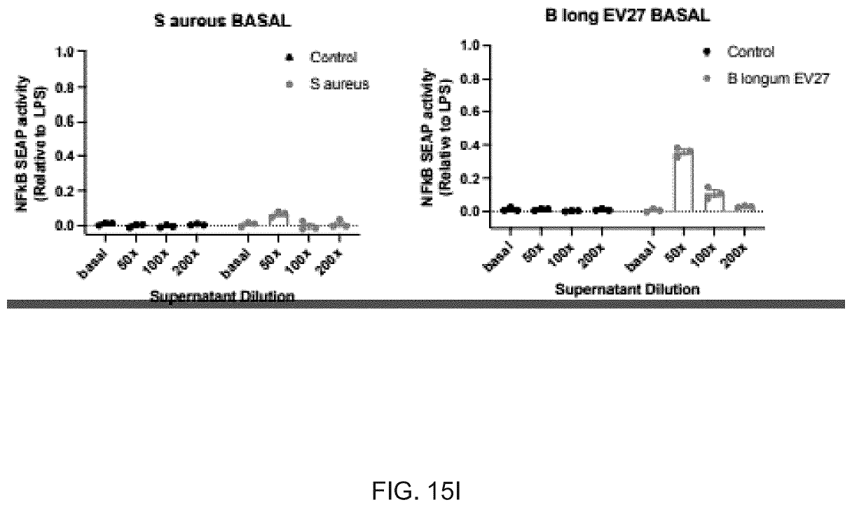

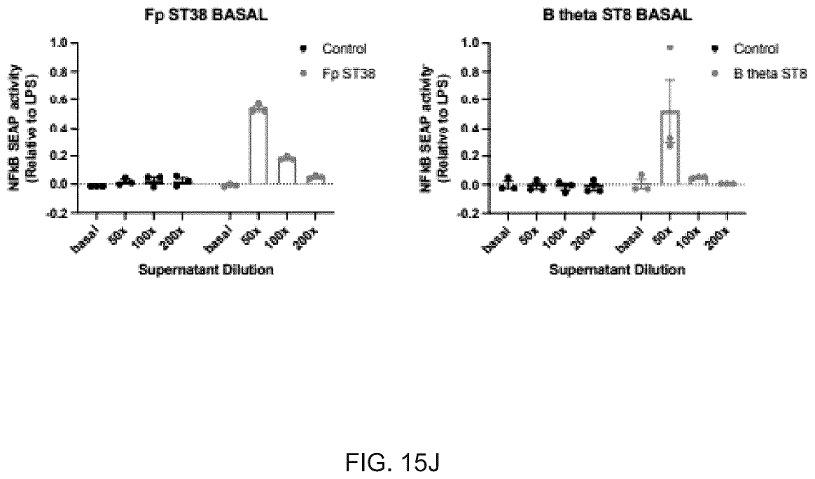

The plate setup for the SEAP detection assay is illustrated in FIG. 15A, wherein about LPS of either 50 ng/mL or 5 ng/mL were added to either Basal or RAWless cells in addition to the bacterial products of the strains of interest. SEAP levels were determined via spectrophotometer measurement of each well of the 96 well plate at about 620-655 nm. The results of the SEAP test were plotted using PRISM software. An example in which the SEAP test was performed at Basal, MOI 10, MOI 5, and MOI 1 to determine the best MOI for the SEAP test are shown in FIG. 15B. The selected bacterial products dilution of the SEAP test was tested at Basal, 50×, 100×, and 200×, example results for AM ST7 are shown in FIG. 15C. SEAP testing was performed on various bacterial products derived from the bacterial strains of interest, example results of which are shown in FIG. 15D-15J.

### 2. Lucia Luciferase Detection Assay

Screening for strains that alter production of transcription-regulating Interferon Regulatory Factor (IRF) was performed via Lucia Luciferase (LUC) assay. The RAW-Dual immortalized macrophage cell line with stable expression of NFκB and Interferon-Sensitive Response Element (ISRE) was pre-incubated with pasteurized bacterial strains of interest. About 20 μL of bacterial supernatant or pasteurized bacterial cells of interest was added to each well of a 96 well plate, and endotoxin-free water was added as a negative control. About 180 μL of cell suspension was added to each well, and the 96 well plate was incubated in an anaerobic chamber at about 37° C. for about 18-24 hours. About 180 μL of resuspended QUANTI-Blue™ solution was then added in each well, and about 20 μL of RAW cell supernatant was added.

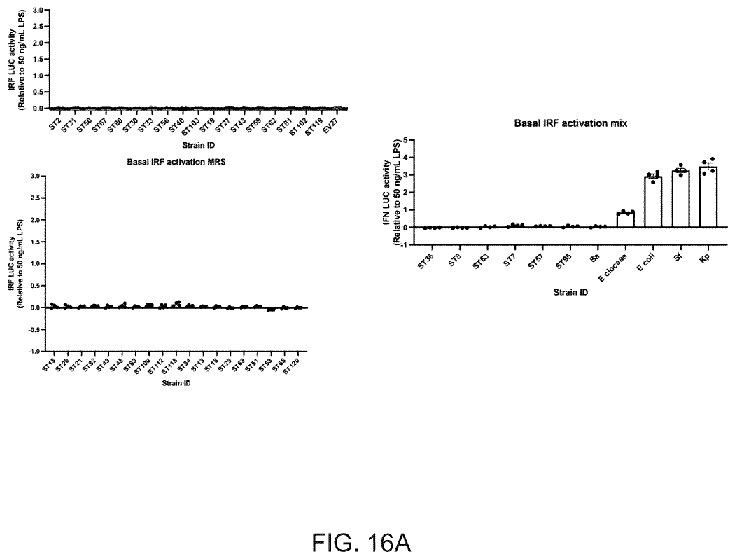

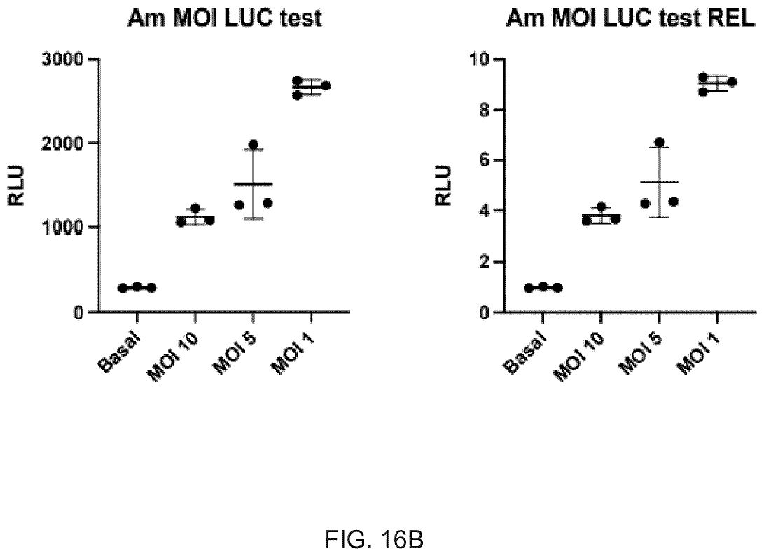

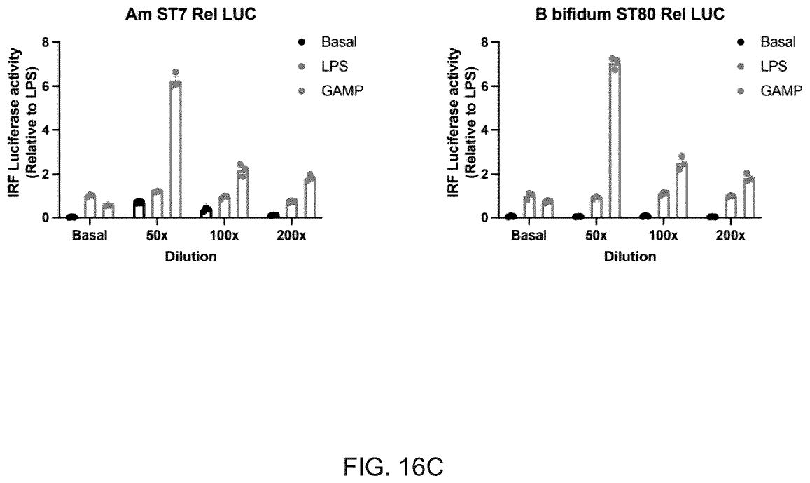

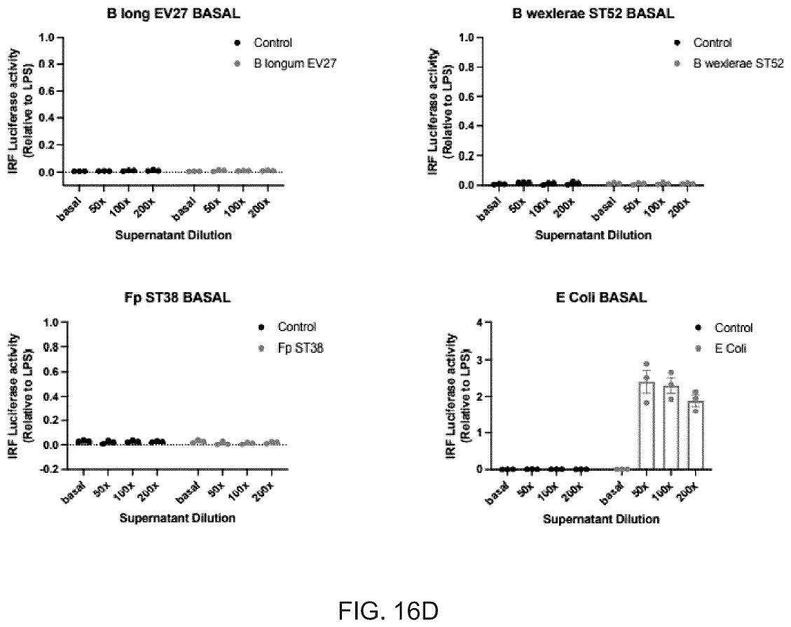

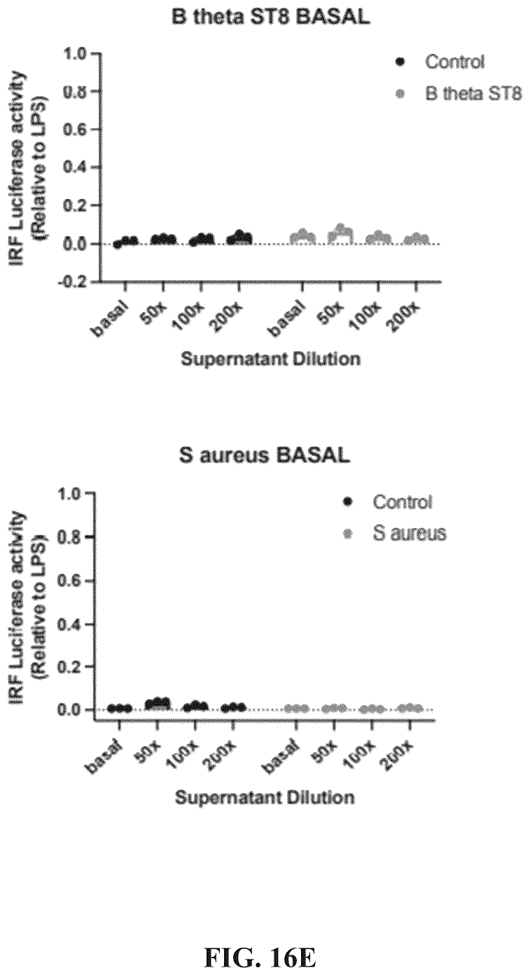

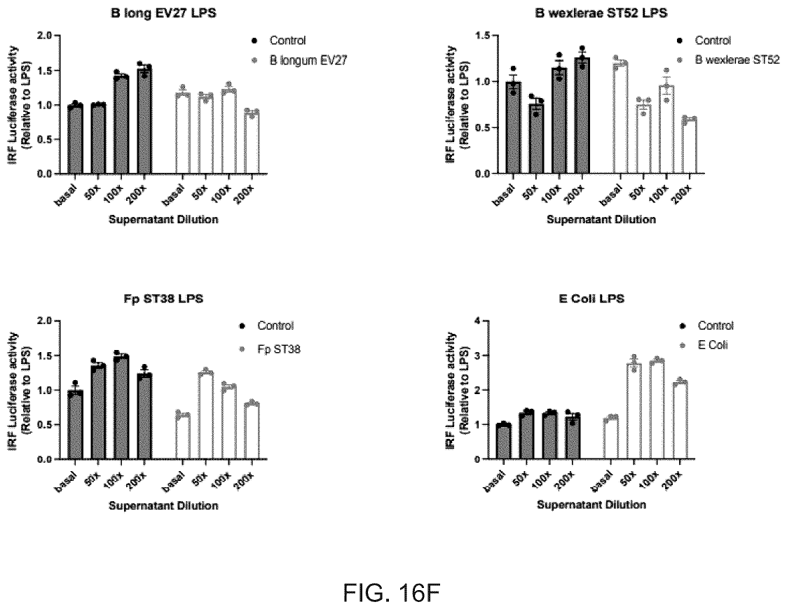

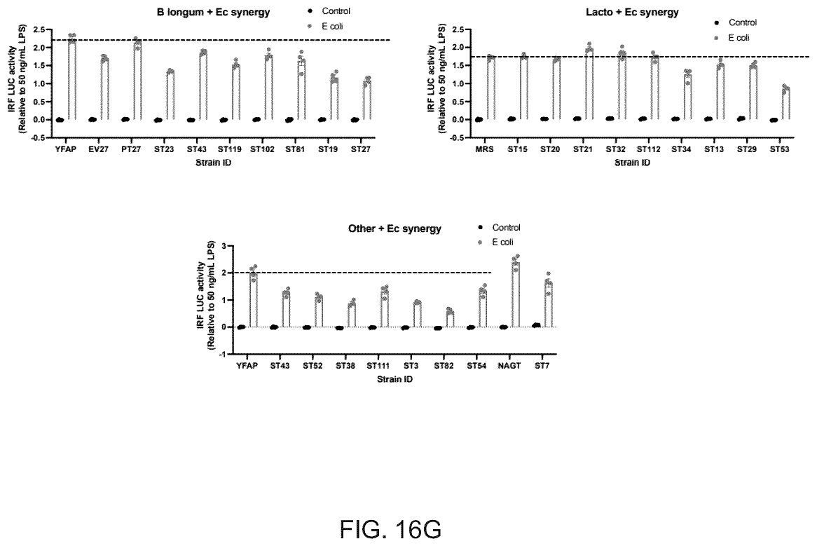

The plate setup for the LUC assay is illustrated in FIG. 15A, wherein LPS of either 50 ng/mL or 5 ng/mL were added to either Basal or RAWless cells in addition to the bacterial products of the strains of interest. LUC levels were immediately determined using luminometry. The results of the LUC test were plotted using GEN5 software, and examples of basal IRF activation data for various bacterial products of the strains of interest are shown in FIG. 16A. An example in which the LUC test was performed at Basal, MOI 10, MOI 5, and MOI 1 to determine the best MOI for the LUC test are shown in FIG. 16B. The ideal bacterial product dilution of the LUC test was tested at Basal, 50×, 100×, and 200×, example results for AM ST7 are shown in FIG. 16C. The examples of LUC test results for various bacterial products derived from bacterial strains of interest relative to differing levels of LPS are shown in FIG. 16D-16H. The LUC detection can be used to determine whether potential therapeutic bacterial strains activate inflammatory transcription factor (IRF) production.

### Example 11: Pathogen Assays

Provided herein are methods for executing pathogen assays to assess if bacterial products from bacterial strains of interest may inhibit the growth of pathogenic bacteria and the effects of those bacterial products on pathogen adherence to cells. Such strains may be used as part of a microbial consortium described herein.

### In Vitro Growth Inhibition

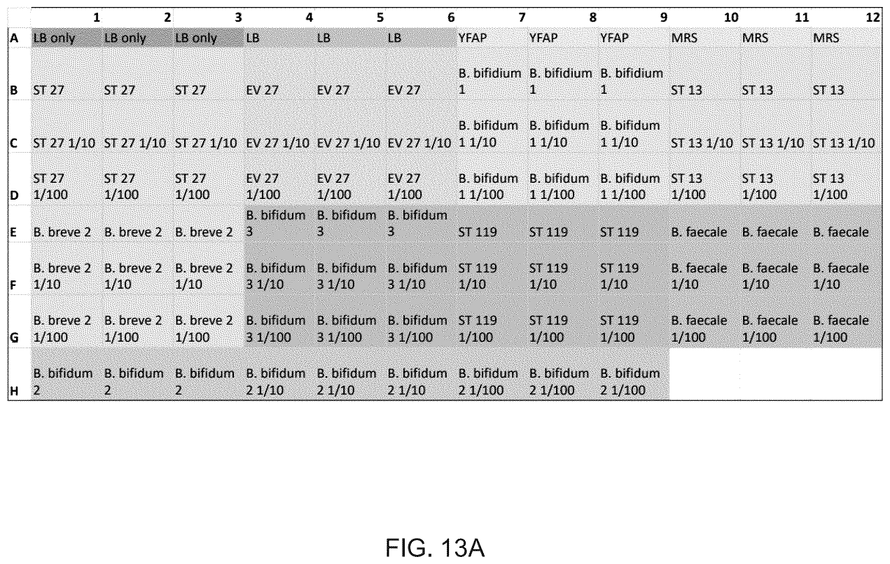

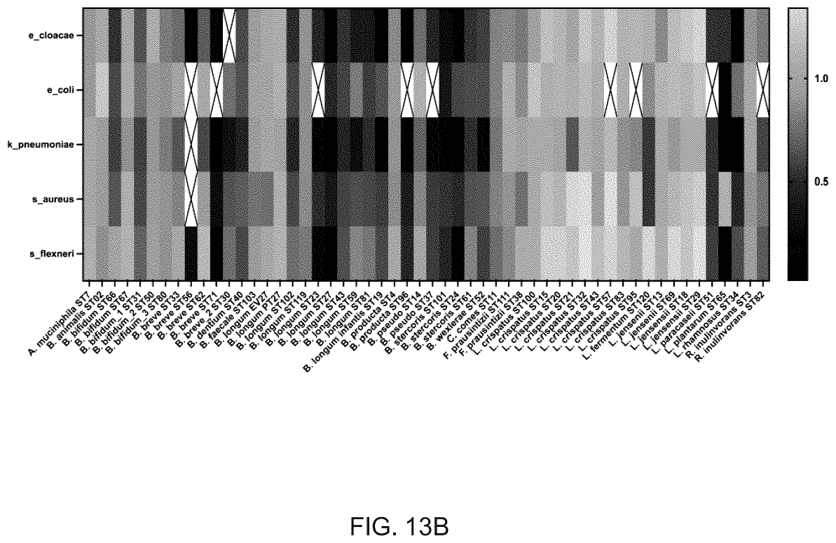

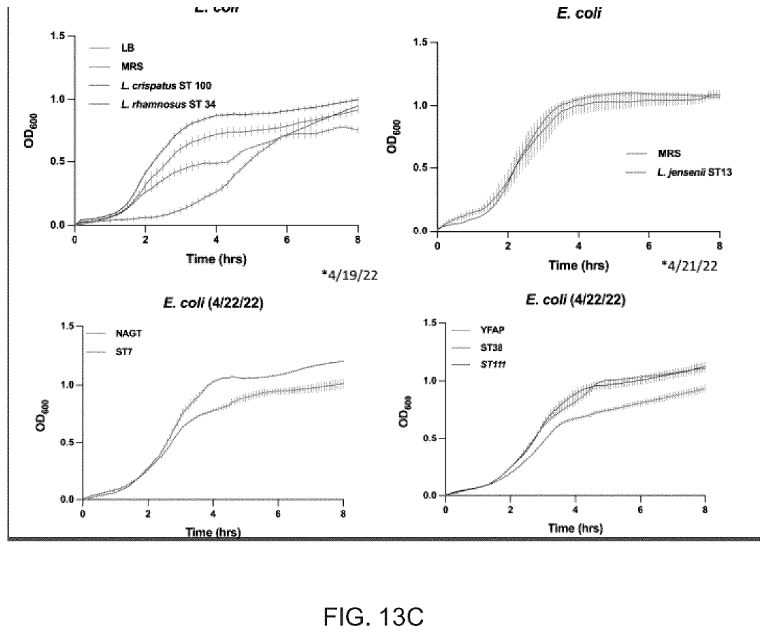

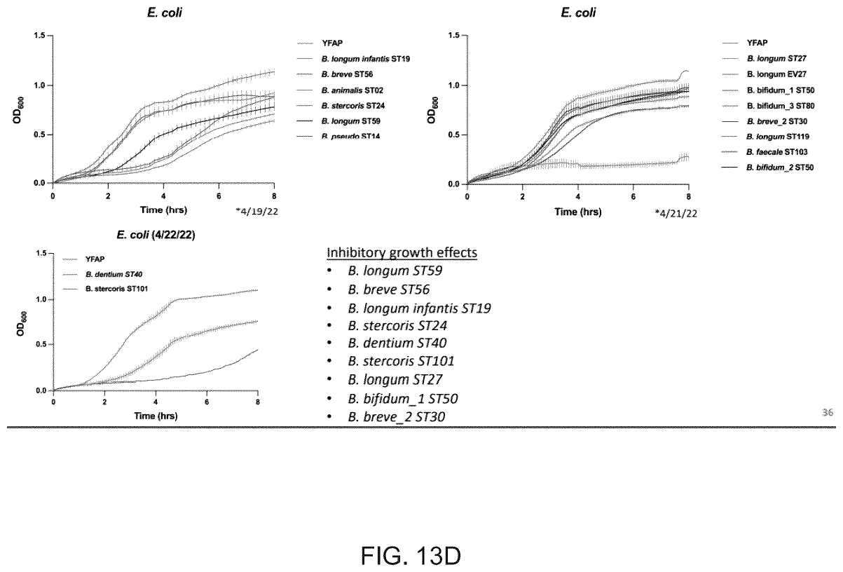

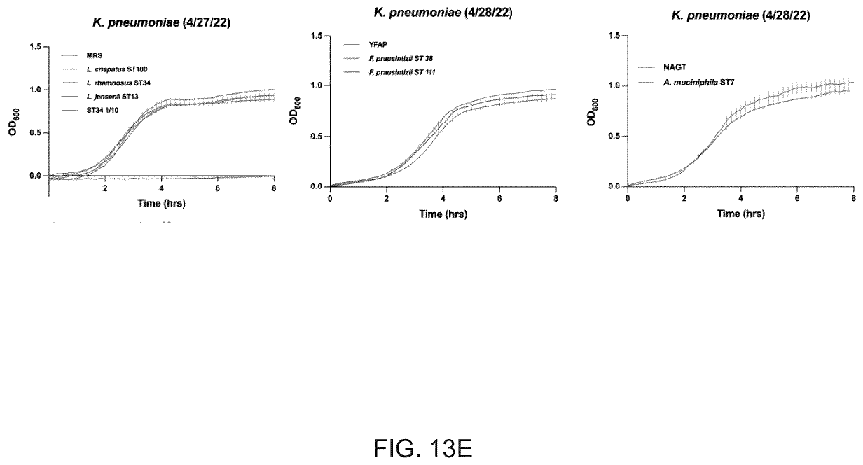

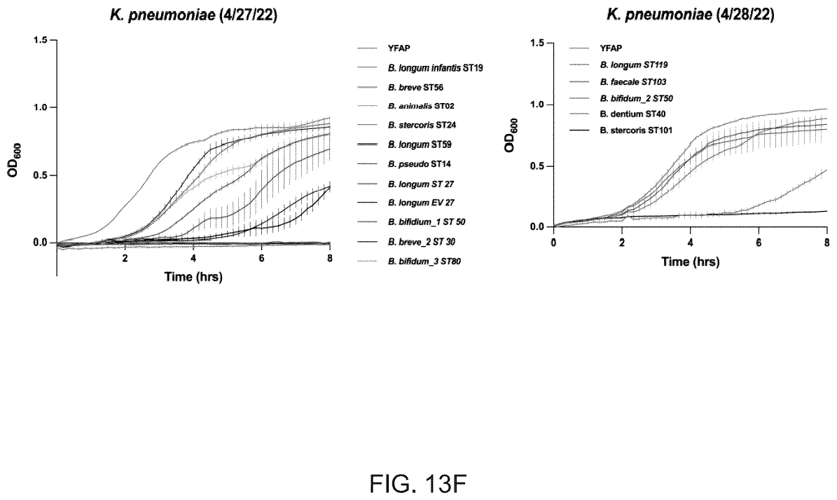

The ability of bacteria of interest to inhibit NEC pathogen growth via production of extracellular bacterial products was assessed by pathogen growth inhibition assay. The pathogens of interest were cultured in appropriate growth media. The growth of cultures was measured by spectroscopy and were back-diluted to achieve an OD₆₀₀ of 0.1 in the appropriate growth media. was used to measure the diluted culture and achieve an OD₆₀₀ of 0.01. The bacteria of interest were cultured in appropriate growth media, as shown in TABLEs 3-4. The cultures of the bacteria of interest were centrifuged to form a cell pellet. The supernatant was harvested and filter-sterilized using 0.22 μm filters. 20 μL of filtered cell-free supernatants were pipetted into wells of the 96-well plate as shown in FIG. 13A at a final concentration of 1/10 or 1/100 as indicated. Following the addition of the supernatants to the 96-well plate, 180 μL of each diluted pathogen culture of interest was pipetted into wells of the 96-well plate as shown in FIG. 13A.

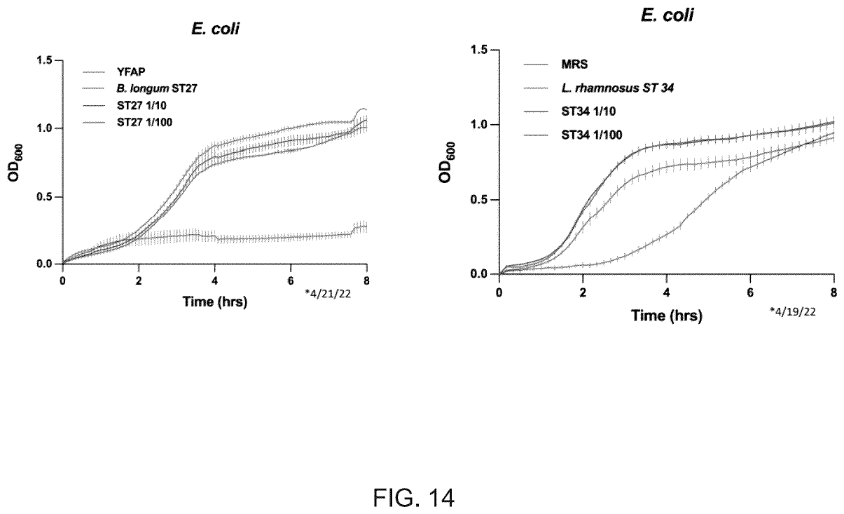

The pathogen growth was monitored by measuring the OD₆₀₀ of the culture in each well of the 96-well plate for 8 hours. The effect of supernatants from bacteria of interest on pathogen growth is represented as the Pathogen Growth Ratio, which is the average AUC of treatment over the average AUC of a media control, an example of which showing various strains of interest is shown in FIG. 13B. The data was plotted to form a growth curve for the supernatant well of each bacterial strain of interest, examples of which are shown in FIG. 13A-13F. Dilutions of supernatants of bacteria of interest at 1/10 and 1/100 were also plotted with examples as shown in FIG. 14. For example, dose-dependent inhibition of E. coli was observed, with a 1:10 dilution of some bacterial products, for example ST27, exhibiting a greater inhibitory effect on E. coli growth than a 1:100 dilution of those bacterial products. For screening purposes, supernatants of strains of interest were tested at 1/10, as stronger inhibitory phenotypes were observed.

### Example 12: Methods for Treating BV or NEC

Provided herein are methods for treating BV or NEC using the compositions as described herein.

The composition described herein is administer to a subject. The subject can be a human subject with the vaginal diseases or infant gastrointestinal disease. For BV, the subject can be a human subject with BV. The subject can be a human subject with recurrent BV. For NEC, the subject can be a human subject with NEC. The subject can be an infant. The subject can be a mouse model that models the vaginal disease or infant gastrointestinal disease.

For treating BV, the subject is administered with the composition described herein vaginally. The composition can comprise a hydrogel. The composition can also be administered without the hydrogel. After the administration, the amount of the vaginal pathogen in the vagina of the subject is determined and compared to the control not administrated with the composition. Subjects can have a lower amount of vaginal pathogens subsequent to being administered with the composition.

For treating NEC, the subject is administered with the composition described herein orally. The composition can be formulated into a suspension. After the administration, the amount of the infant gastrointestinal pathogen in the gastrointestinal tract of the subject is determined and compared to the control not administrated with the composition. Subjects can have a lower amount of infant gastrointestinal pathogens subsequent to being administered with the composition. Additionally, the TLR4 signaling pathway activation can be determined. Subjects can have a lower activation of the TLR4 signaling pathway subsequent to being administered with the composition. For measuring TLR4 activation, a mouse model can comprise a TLR4 reporter, such as the IRES or SEAP reporter as described herein.

Other symptoms of BV or NEC can also be assessed to determine the therapeutic effect of the composition.

### NUMBERED EMBODIMENTS

Embodiment 1. A pharmaceutical composition comprising a bacterial population comprising a first bacterial strain and a second bacterial strain,
wherein said first bacterial strain and said second bacterial strain are different from one another,
wherein said bacterial population comprises Bifidobacterium sp. or Vertebrate-Associated Lactobacillaceae,
wherein said first bacterial strain, when present in a medium comprising an energy source and said second bacterial strain or a supernatant of said medium comprising said energy source and said second bacterial strain, exhibits a growth of at least about 105% by weight as compared to growth of said first bacterial strain when present in a medium comprising said energy source in an absence of said second bacterial strain or said supernatant of said medium comprising said energy source and said second bacterial strain, and
wherein said energy source does not comprise starch.
   Embodiment 2. The pharmaceutical composition of embodiment 1, wherein said supernatant of said medium comprising said energy source and said second bacterial strain is cell-free.
   Embodiment 3. The pharmaceutical composition of embodiment 1 or 2, wherein said supernatant of said medium comprising said energy source and said second bacterial strain comprises a fermentation product derived from said second bacterial strain.
   Embodiment 4. The pharmaceutical composition of any one of embodiments 1-3, wherein said first bacterial strain, when present within said medium comprising said energy source and said second bacterial strain or said supernatant of said medium comprising said energy source and said second bacterial strain, exhibits a growth for at most about 48 hours of at least about 105% by weight as compared to a growth of said first bacterial strain when present within said medium comprising said energy source in an absence of said second bacterial strain or said supernatant of said medium comprising said energy source and said second bacterial strain for at most about 48 hours.
   Embodiment 5. The pharmaceutical composition of embodiment 4, wherein said first bacterial strain, when present within said medium comprising said energy source and said second bacterial strain or said supernatant of said medium comprising said energy source and said second bacterial strain, exhibits a growth for at most about 24 hours of at least about 105% by weight as compared to a growth of said first bacterial strain when present within said medium comprising said energy source in an absence of said second bacterial strain or said supernatant of said medium comprising said energy source and said second bacterial strain for at most about 24 hours.
   Embodiment 6. The pharmaceutical composition of embodiment 5, wherein said first bacterial strain, when present within said medium comprising said energy source and said second bacterial strain or said supernatant of said medium comprising said energy source and said second bacterial strain, exhibits a growth for at most about 12 hours of at least about 105% by weight as compared to a growth of said first bacterial strain when present within said medium comprising said energy source in an absence of said second bacterial strain or said supernatant of said medium comprising said energy source and said second bacterial strain for at most about 12 hours.
   Embodiment 7. The pharmaceutical composition of any one of embodiments 1-6, wherein said first bacterial strain, when present within said medium comprising said energy source and said second bacterial strain or said supernatant of said medium comprising said energy source and said second bacterial strain, exhibits said growth of at least about 150% by weight as compared to said growth of said first bacterial strain when present within said medium comprising said energy source in an absence of said second bacterial strain or said supernatant of said medium comprising said energy source and said second bacterial strain.
   Embodiment 8. The pharmaceutical composition of embodiment 7, wherein said first bacterial strain, when present within said medium comprising said energy source and said second bacterial strain or said supernatant of said medium comprising said energy source and said second bacterial strain, exhibits said growth of at least about 1000% by weight as compared to said growth of said first bacterial strain when present within said medium comprising said energy source in an absence of said second bacterial strain or said supernatant of said medium comprising said energy source and said second bacterial strain.
   Embodiment 9. The pharmaceutical composition of embodiment 8, wherein said first bacterial strain, when present within said medium comprising said energy source and said second bacterial strain or said supernatant of said medium comprising said energy source and said second bacterial strain, exhibits said growth of at least about 10000% by weight as compared to said growth of said first bacterial strain when present within said medium comprising said energy source in an absence of said second bacterial strain or said supernatant of said medium comprising said energy source and said second bacterial strain.
   Embodiment 10. The pharmaceutical composition of any one of embodiments 1-9, wherein said pharmaceutical composition is formulated in an enteral dosage form, an injectable dosage form, a parenteral dosage form, a topical dosage form, or a combination thereof.
   Embodiment 11. The pharmaceutical composition of embodiment 10, wherein said enteral dosage form comprises an oral dosage form, an intragastric dosage form, or a rectal dosage form.
   Embodiment 12. The pharmaceutical composition of embodiment 11, wherein said intragastric dosage form comprises a dosage form that is configured to pass through a feeding tube.
   Embodiment 13. The pharmaceutical composition of any one of embodiments 1-12, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 14. The pharmaceutical composition of any one of embodiments 1-13, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 15. The pharmaceutical composition of any one of embodiments 1-14, wherein said Bifidobacterium sp. comprises B. animalis, B. pseudocatenulatum, B. bifidum, B. breve, B. dentium, B. faecale, B. longum, or a combination thereof.
   Embodiment 16. The pharmaceutical composition of any one of embodiments 1-15, wherein said first bacterial strain comprises said Bifidobacterium sp. or said Vertebrate-Associated Lactobacillaceae.
   Embodiment 17. The pharmaceutical composition of embodiment 16, wherein said first bacterial strain comprises said Bifidobacterium sp.
   Embodiment 18. The pharmaceutical composition of embodiment 16, wherein said first bacterial strain comprises said Vertebrate-Associated Lactobacillaceae.
   Embodiment 19. The pharmaceutical composition of embodiment 18, wherein second bacterial strain comprises said Bifidobacterium sp. or said Vertebrate-Associated Lactobacillaceae.
   Embodiment 20. The pharmaceutical composition of embodiment 19, wherein said second bacterial strain comprises said Bifidobacterium sp.
   Embodiment 21. The pharmaceutical composition of embodiment 19, wherein said second bacterial strain comprises said Vertebrate-Associated Lactobacillaceae.
   Embodiment 22. The pharmaceutical composition of any one of embodiments 1-21, wherein said starch is not a modified starch.
   Embodiment 23. The pharmaceutical composition of embodiment 22, wherein said starch is not a fermented starch.
   Embodiment 24. The pharmaceutical composition of embodiment 23, wherein said fermented starch is not a dextrin.
   Embodiment 25. The pharmaceutical composition of embodiment 24, wherein said dextrin is not a maltodextrin.
   Embodiment 26. The pharmaceutical composition of embodiment 25, wherein said energy source comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, or a combination thereof.
   Embodiment 27. The pharmaceutical composition of any one of embodiments 1-26, wherein said pharmaceutical composition further comprises a pharmaceutically acceptable excipient, a cryoprotectant, or a combination thereof.
   Embodiment 28. The pharmaceutical composition of any one of embodiments 1-27, wherein said pharmaceutical composition further comprises Akkermansia sp., Anaerbutyricum sp., Anaerostipes sp., Anaerotignum sp., Bacillus sp., Bacteroides sp., Blautia sp., Clostridium sp., Collinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 29. A pharmaceutical composition comprising a bacterial population comprising a first bacterial strain and a second bacterial strain,
wherein said first bacterial strain and said second bacterial strain are different from one another,
wherein said bacterial population comprises Bifidobacterium sp. or Vertebrate-Associated Lactobacillaceae,
wherein said first bacterial strain, when present within a medium comprising a secreted metabolite derived from said second bacterial strain, proliferates for at least about 10 cell divisions.
   Embodiment 30. The pharmaceutical composition of embodiment 29, wherein said medium comprising said secreted metabolite derived from said second bacterial strain comprises a supernatant derived from a growth culture of said second bacterial strain.
   Embodiment 31. The pharmaceutical composition of embodiment 30, wherein said supernatant derived from said growth culture of said second bacterial strain is cell-free.
   Embodiment 32. The pharmaceutical composition of any one of embodiments 29-31, wherein said first bacterial strain, when present within said medium comprising said secreted metabolite derived from said second bacterial strain, proliferates for at least about 10 cell divisions within at most about 48 hours.
   Embodiment 33. The pharmaceutical composition of embodiment 32, wherein said first bacterial strain, when present within said medium comprising said secreted metabolite derived from said second bacterial strain, proliferates for at least about 10 cell divisions within at most about 24 hours.
   Embodiment 34. The pharmaceutical composition of embodiment 33, wherein said first bacterial strain, when present within said medium comprising said secreted metabolite derived from said second bacterial strain, proliferates for at least about 10 cell divisions within at most about 12 hours.
   Embodiment 35. The pharmaceutical composition of any one of embodiments 29-34, wherein said first bacterial strain, when present within said medium comprising said secreted metabolite derived from said second bacterial strain, proliferates for at least about 15 cell divisions.
   Embodiment 36. The pharmaceutical composition of embodiment 35, wherein said first bacterial strain, when present within said medium comprising said secreted metabolite derived from said second bacterial strain, proliferates for at least about 20 cell divisions.
   Embodiment 37. The pharmaceutical composition of embodiment 36, wherein said first bacterial strain, when present within said medium comprising said secreted metabolite derived from said second bacterial strain, proliferates for at least about 32 cell divisions.
   Embodiment 38. The pharmaceutical composition of any one of embodiments 29-37, wherein said pharmaceutical composition is formulated in an enteral dosage form, an injectable dosage form, a parenteral dosage form, a topical dosage form, or a combination thereof.
   Embodiment 39. The pharmaceutical composition of embodiment 38, wherein said enteral dosage form comprises an oral dosage form, an intragastric dosage form, or a rectal dosage form.
   Embodiment 40. The pharmaceutical composition of embodiment 39, wherein said intragastric dosage form comprises a dosage form that is configured to pass through a feeding tube.
   Embodiment 41. The pharmaceutical composition of any one of embodiments 29-40, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 42. The pharmaceutical composition of any one of embodiments 29-41, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 43. The pharmaceutical composition of any one of embodiments 29-42 wherein said Bifidobacterium sp. comprises B. animalis, B. pseudocatenulatum, B. bifidum, B. breve, B. dentium, B. faecale, B. longum, or a combination thereof.
   Embodiment 44. The pharmaceutical composition of any one of embodiments 29-43, wherein said first bacterial strain comprises said Bifidobacterium sp. or said Vertebrate-Associated Lactobacillaceae.
   Embodiment 45. The pharmaceutical composition of embodiment 44, wherein said first bacterial strain comprises said Bifidobacterium sp.
   Embodiment 46. The pharmaceutical composition of embodiment 44, wherein said first bacterial strain comprises said Vertebrate-Associated Lactobacillaceae.
   Embodiment 47. The pharmaceutical composition of any one of embodiments 29-46, wherein second bacterial strain comprises said Bifidobacterium sp. or said Vertebrate-Associated Lactobacillaceae.
   Embodiment 48. The pharmaceutical composition of embodiment 47, wherein said second bacterial strain comprises said Bifidobacterium sp.
   Embodiment 49. The pharmaceutical composition of embodiment 47, wherein said second bacterial strain comprises said Vertebrate-Associated Lactobacillaceae.
   Embodiment 50. The pharmaceutical composition of any one of embodiments 29-49, wherein said energy source comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof.
   Embodiment 51. The pharmaceutical composition of embodiment 50, wherein said starch comprises a modified starch.
   Embodiment 52. The pharmaceutical composition of embodiment 51, wherein said starch comprises a fermented starch.
   Embodiment 53. The pharmaceutical composition of any one of embodiments 29-52, wherein said fermented starch comprises a dextrin.
   Embodiment 54. The pharmaceutical composition of embodiment 53, wherein said dextrin comprises a maltodextrin.
   Embodiment 55. The pharmaceutical composition of any one of embodiments 29-54, wherein said pharmaceutical composition further comprises a pharmaceutically acceptable excipient, a cryoprotectant, or a combination thereof.
   Embodiment 56. The pharmaceutical composition of any one of embodiments 29-55, wherein said pharmaceutical composition further comprises Akkermansia sp., Anaerbutyricum sp., Anaerostipes sp., Anaerotignum sp., Bacillus sp., Bacteroides sp., Blautia sp., ClostridiumCollinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 57. A pharmaceutical composition comprising a bacterial population comprising a first bacterial strain and a second bacterial strain,
wherein said first bacterial strain and said second bacterial strain are different from one another,
wherein said bacterial population comprises Bifidobacterium sp. or Vertebrate-Associated Lactobacillaceae,
wherein said first bacterial strain, when present within a medium comprising a secreted metabolite derived from said second bacterial strain and a layer of epithelial cells, decreases permeability of said layer of epithelial cells by at least about 5%, as compared to permeability of a layer of epithelial cells present within a medium comprising said first bacterial strain in an absence of said secreted metabolite, and
   wherein said permeability of said layer of epithelial cells is measured by transport of a Fluorescein isothiocyanate (FITC)-conjugated dextran or by transepithelial electrical resistance across said layer of epithelial cells.
   Embodiment 58. The pharmaceutical composition of embodiment 57, wherein said medium comprising said secreted metabolite derived from said second bacterial strain comprises a supernatant derived from a growth culture of said second bacterial strain.
   Embodiment 59. The pharmaceutical composition of embodiment 58, wherein said supernatant derived from said growth culture of said second bacterial strain is cell-free.
   Embodiment 60. The pharmaceutical composition of any one of embodiments 57-59, wherein said secreted metabolite derived from said second bacterial strain comprises a fermentation product derived from said second bacterial strain.
   Embodiment 61. The pharmaceutical composition any one of embodiments 57-60, wherein said first bacterial strain, when present within said medium comprising said secreted metabolite derived from said second bacterial strain, decreases said permeability of said layer of epithelial cells by at least about 10%, as compared to said permeability of said layer of epithelial cells present within said medium comprising said first bacterial strain in said absence of said secreted metabolite.
   Embodiment 62. The pharmaceutical composition of embodiment 61, wherein said first bacterial strain, when present within said medium comprising said secreted metabolite derived from said second bacterial strain, decreases said permeability of said layer of epithelial cells by at least about 20%, as compared to said permeability of said layer of epithelial cells present within said medium comprising said first bacterial strain in said absence of said secreted metabolite.
   Embodiment 63. The pharmaceutical composition of embodiment 62, wherein said first bacterial strain, when present within said medium comprising said secreted metabolite derived from said second bacterial strain, decreases said permeability of said layer of epithelial cells by at least about 50%, as compared to said permeability of said layer of epithelial cells present within said medium comprising said first bacterial strain in said absence of said secreted metabolite.
   Embodiment 64. The pharmaceutical composition of any one of embodiments 57-63, wherein said epithelial cells comprise mammalian epithelial cells.
   Embodiment 65. The pharmaceutical composition of embodiment 64, wherein said mammalian epithelial cells comprises human epithelial cells.
   Embodiment 66. The pharmaceutical composition of any one of embodiments 57-65, wherein said pharmaceutical composition is formulated in an enteral dosage form, an injectable dosage form, a parenteral dosage form, a topical dosage form, or a combination thereof.
   Embodiment 67. The pharmaceutical composition of embodiment 65, wherein said enteral dosage form comprises an oral dosage form, an intragastric dosage form, or a rectal dosage form.
   Embodiment 68. The pharmaceutical composition of embodiment 66, wherein said intragastric dosage form comprises a dosage form that is configured to pass through a feeding tube.
   Embodiment 69. The pharmaceutical composition of any one of embodiments 57-68, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 70. The pharmaceutical composition of any one of embodiments 57-69, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 71. The pharmaceutical composition of any one of embodiments 57-70, wherein said Bifidobacterium sp. comprises B. animalis, B. pseudocatenulatum, B. bifidum, B. breve, B. dentium, B. faecale, B. longum, or a combination thereof.
   Embodiment 72. The pharmaceutical composition of any one of embodiments 57-71, wherein said first bacterial strain comprises said Bifidobacterium sp. or said Vertebrate-Associated Lactobacillaceae.
   Embodiment 73. The pharmaceutical composition of embodiment 72, wherein said first bacterial strain comprises said Bifidobacterium sp.
   Embodiment 74. The pharmaceutical composition of embodiment 72, wherein said first bacterial strain comprises said Vertebrate-Associated Lactobacillaceae.
   Embodiment 75. The pharmaceutical composition of any one of embodiments 57-58, wherein said second bacterial strain comprises said Bifidobacterium sp. or said Vertebrate-Associated Lactobacillaceae.
   Embodiment 76. The pharmaceutical composition of embodiment 75, wherein said second bacterial strain comprises said Bifidobacterium sp.
   Embodiment 77. The pharmaceutical composition of embodiment 75, wherein said second bacterial strain comprises Vertebrate-Associated Lactobacillaceae.
   Embodiment 78. The pharmaceutical composition of any one of embodiments 57-77, wherein said energy source comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof.
   Embodiment 79. The pharmaceutical composition of embodiment 78, wherein said starch comprises a modified starch.
   Embodiment 80. The pharmaceutical composition of embodiment 79, wherein said starch comprises a fermented starch.
   Embodiment 81. The pharmaceutical composition of embodiment 80, wherein said fermented starch comprises a dextrin.
   Embodiment 82. The pharmaceutical composition of embodiment 81, wherein said dextrin comprises a maltodextrin.
   Embodiment 83. The pharmaceutical composition of any one of embodiments 57-82, wherein said pharmaceutical composition further comprises a pharmaceutically acceptable excipient, a cryoprotectant, or a combination thereof.
   Embodiment 84. The pharmaceutical composition of any one of embodiments 57-83, wherein said pharmaceutical composition further comprises Akkermansia sp., Anaerbutyricum sp., Anaerostipes sp., Anaerotignum sp., Bacillus sp., Bacteroides sp., Blautia sp., ClostridiumCollinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 85. A pharmaceutical composition comprising a bacterial population comprising a bacterial strain,
wherein said bacterial population comprises Bifidobacterium sp. or Vertebrate-Associated Lactobacillaceae, and
wherein a secreted metabolite or an inviable cell derived from said bacterial strain, when combined with an engineered cell comprising a reporter, decreases a signal of said reporter by at least about 5% as compared to a signal of said reporter when said engineered cell is not combined with said metabolite or said inviable cell derived from said bacterial strain.
   Embodiment 86. The pharmaceutical composition of embodiment 85, wherein said medium comprising said secreted metabolite or said inviable cells derived from said bacterial strain comprises a supernatant derived from a growth culture of said bacterial strain.
   Embodiment 87. The pharmaceutical composition of embodiment 86, wherein said supernatant derived from said growth culture of said bacterial strain is cell-free.
   Embodiment 88. The pharmaceutical composition of any one of embodiments 85-87, wherein said medium comprising said secreted metabolite or said inviable cells derived from said bacterial strain comprises a fermentation product derived from a growth culture of said bacterial strain.
   Embodiment 89. The pharmaceutical composition of any one of embodiments 85-88, wherein said secreted metabolite derived from said bacterial strain comprises a fermentation product derived from said bacterial strain.
   Embodiment 90. The pharmaceutical composition of any one of embodiments 85-89, wherein said engineered cell comprises a macrophage.
   Embodiment 91. The pharmaceutical composition of any one of embodiments 85-90, wherein said inviable cell comprises a pasteurized cell.
   Embodiment 92. The pharmaceutical composition of any one of embodiments 85-91, wherein said reporter comprises a nuclear factor kappa-light-chain-enhancer of activated B cells (NFkB) reporter or an interferon-sensitive response element reporter (ISRE).
   Embodiment 93. The pharmaceutical composition of embodiment 92, wherein said NFkB reporter comprises a secreted embryonic alkaline phosphatase (SEAP) reporter.
   Embodiment 94. The pharmaceutical composition of embodiment 92, wherein said ISRE reporter comprises a Lucia luciferase.
   Embodiment 95. The pharmaceutical composition of any one of embodiments 85-94, wherein said secreted metabolite or said inviable cell derived from said bacterial strain, when combined with said engineered cell comprising said reporter, decreases said signal of said reporter by at least about 10% as compared to when said engineered cell is not combined with said metabolite or said inviable cell derived from said bacterial strain.
   Embodiment 96. The pharmaceutical composition of embodiment 95, wherein said secreted metabolite or said inviable cell derived from said bacterial strain, when combined with said engineered cell comprising said reporter, decreases said signal of said reporter by at least about 50% as compared to when said engineered cell is not combined with said metabolite or said inviable cell derived from said bacterial strain.
   Embodiment 97. The pharmaceutical composition of any one of embodiments 85-96, wherein said pharmaceutical composition is formulated in an enteral dosage form, an injectable dosage form, a parenteral dosage form, a topical dosage form, or a combination thereof.
   Embodiment 98. The pharmaceutical composition of embodiment 97, wherein said enteral dosage form comprises an oral dosage form, an intragastric dosage form, or a rectal dosage form.
   Embodiment 99. The pharmaceutical composition of embodiment 98, wherein said intragastric dosage form comprises a dosage form that is configured to pass through a feeding tube.
   Embodiment 100. The pharmaceutical composition of any one of embodiments 85-99, wherein said bacterial strain comprises said Bifidobacterium sp. or said Vertebrate-Associated Lactobacillaceae.
   Embodiment 101. The pharmaceutical composition of embodiment 100, wherein said bacterial strain comprises said Bifidobacterium sp.
   Embodiment 102. The pharmaceutical composition of embodiment 101, wherein said Bifidobacterium sp. comprises B. animalis, B. pseudocatenulatum, B. bifidum, B. breve, B. dentium, B. faecale, B. longum, or a combination thereof.
   Embodiment 103. The pharmaceutical composition of embodiment 100, wherein said bacterial strain comprises said Vertebrate-Associated Lactobacillaceae.
   Embodiment 104. The pharmaceutical composition of embodiment 103, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 105. The pharmaceutical composition of any one of embodiments 85-104, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 106. The pharmaceutical composition of any one of embodiments 85-105, wherein said energy source comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof.
   Embodiment 107. The pharmaceutical composition of embodiment 106, wherein said starch comprises a modified starch.
   Embodiment 108. The pharmaceutical composition of embodiment 107, wherein said starch comprises a fermented starch.
   Embodiment 109. The pharmaceutical composition of embodiment 108, wherein said fermented starch comprises a dextrin.
   Embodiment 110. The pharmaceutical composition of embodiment 109, wherein said dextrin comprises a maltodextrin.
   Embodiment 111. The pharmaceutical composition of any one of embodiments 85-110, wherein said pharmaceutical composition further comprises a pharmaceutically acceptable excipient, a cryoprotectant, or a combination thereof.
   Embodiment 112. The pharmaceutical composition of any one of embodiments 85-111, wherein said pharmaceutical composition further comprises Akkermansia sp., Anaerbutyricum sp., Anaerostipes sp., Anaerotignum sp., Bacillus sp., Bacteroides sp., Blautia sp., ClostridiumCollinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 113. A pharmaceutical composition comprising a bacterial population comprising a first bacterial strain and a second bacterial strain,
wherein said first bacterial strain and said second bacterial strains are different from one another,
wherein said bacterial population comprises Bifidobacterium sp. or Vertebrate-Associated Lactobacillaceae,
wherein said first bacterial strain, when present within a medium comprising said second bacterial strain or a supernatant thereof for at most about 15 hours, exhibits a growth of at least about 105% by colony-forming unit (CFU) as compared to a growth of said first bacterial strain when present within a medium not comprising said second bacterial strain or said supernatant thereof for at most about 15 hours.
   Embodiment 114. The pharmaceutical composition of embodiment 113, wherein said medium comprising said second bacterial strain or said supernatant thereof comprises a secreted metabolite derived from said second bacterial strain.
   Embodiment 115. The pharmaceutical composition of embodiment 114, wherein said secreted metabolite derived from said second bacterial strain comprises a fermentation product of derived from said second bacterial strain.
   Embodiment 116. The pharmaceutical composition of any one of embodiments 113-115, wherein said supernatant of said medium comprising said second bacterial strain is cell-free.
   Embodiment 117. The pharmaceutical composition of any one of embodiments 113-116, wherein said first bacterial strain, when present within said medium comprising said second bacterial strain or a supernatant thereof for at most about 10 hours, exhibits a growth of at least about 105% by CFU as compared to a growth of said first bacterial strain when present within said medium not comprising said second bacterial strain or said supernatant thereof for at most about 10 hours.
   Embodiment 118. The pharmaceutical composition of embodiment 117, wherein said first bacterial strain, when present within said medium comprising said second bacterial strain or a supernatant thereof for at most about 8 hours, exhibits a growth of at least about 105% by CFU as compared to a growth of said first bacterial strain when present within said medium for not comprising said second bacterial strain or said supernatant thereof for at most about 8 hours.
   Embodiment 119. The pharmaceutical composition of any one of embodiments 113-118, wherein said first bacterial strain, when present within said medium comprising said second bacterial strain or a supernatant thereof for at most about 15 hours, exhibits a growth of at least about 120% by CFU as compared to a growth of said first bacterial strain when present within said medium not comprising said second bacterial strain or said supernatant thereof for at most about 15 hours.
   Embodiment 120. The pharmaceutical composition of embodiment 119, wherein said first bacterial strain, when present within said medium comprising said second bacterial strain or a supernatant thereof for at most about 15 hours, exhibits a growth of at least about 150% by CFU as compared to a growth of said first bacterial strain when present within said medium not comprising said second bacterial strain or said supernatant thereof for at most about 15 hours.
   Embodiment 121. The pharmaceutical composition of embodiment 120, wherein said first bacterial strain, when present within said medium comprising said second bacterial strain or a supernatant thereof for at most about 15 hours, exhibits a growth of at least about 200% by CFU as compared to a growth of said first bacterial strain when present within said medium not comprising said second bacterial strain or said supernatant thereof for at most about 15 hours.
   Embodiment 122. The pharmaceutical composition of any one of embodiments 113-121, wherein said bacterial strain comprises said Bifidobacterium sp. or said Vertebrate-Associated Lactobacillaceae.
   Embodiment 123. The pharmaceutical composition of embodiment 122, wherein said bacterial strain comprises said Bifidobacterium sp.
   Embodiment 124. The pharmaceutical composition of embodiment 123, wherein said Bifidobacterium sp. comprises B. animalis, B. pseudocatenulatum, B. bifidum, B. breve, B. dentium, B. faecale, B. longum, or a combination thereof.
   Embodiment 125. The pharmaceutical composition of embodiment 123, wherein said bacterial strain comprises said Vertebrate-Associated Lactobacillaceae.
   Embodiment 126. The pharmaceutical composition of embodiment 125, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 127. The pharmaceutical composition of any one of embodiments 113-126, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 128. The pharmaceutical composition of any one of embodiments 113-127, wherein said energy source comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof.
   Embodiment 129. The pharmaceutical composition of embodiment 128, wherein said starch comprises a modified starch.
   Embodiment 130. The pharmaceutical composition of embodiment 129, wherein said starch comprises a fermented starch.
   Embodiment 131. The pharmaceutical composition of any one of embodiments 113-130, wherein said fermented starch comprises a dextrin.
   Embodiment 132. The pharmaceutical composition of embodiment 131, wherein said dextrin comprises a maltodextrin.
   Embodiment 133. The pharmaceutical composition of any one of embodiments 113-132, wherein said pharmaceutical composition further comprises a pharmaceutically acceptable excipient, a cryoprotectant, or a combination thereof.
   Embodiment 134. The pharmaceutical composition of any one of embodiments 113-133, wherein said pharmaceutical composition further comprises Akkermansia sp., Anaerbutyricum sp., Anaerostipes sp., Anaerotignum sp., Bacillus sp., Bacteroides sp., Blautia sp., ClostridiumCollinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 135. A pharmaceutical composition comprising a bacterial population comprising a first bacterial strain and a second bacterial strain,
wherein said first bacterial strain and said second bacterial strain are different from one another,
wherein said bacterial population comprises Bifidobacterium sp. or Vertebrate-Associated Lactobacillaceae,
wherein said first bacterial strain is configured to utilize a metabolite derived from said second bacterial strain as a growth promoter, and
wherein said metabolite is not a butyrate, a vitamin B12, or an ammonia (NH3).
   Embodiment 136. The pharmaceutical composition of embodiment 135, wherein said metabolite is a secreted metabolite derived from said bacterial strain.
   Embodiment 137. The pharmaceutical composition of embodiment 135 or 136, wherein said metabolite is not a derivative or a combination of said butyrate, said vitamin B12, or said NH3.
   Embodiment 138. The pharmaceutical composition of any one of embodiments 135-137, wherein said pharmaceutical composition is formulated in an enteral dosage form, an injectable dosage form, a parenteral dosage form, a topical dosage form, or a combination thereof.
   Embodiment 139. The pharmaceutical composition of embodiment 138, wherein said enteral dosage form comprises an oral dosage form, an intragastric dosage form, or a rectal dosage form.
   Embodiment 140. The pharmaceutical composition of embodiment 139, wherein said intragastric dosage form comprises a dosage form that is configured to pass through a feeding tube.
   Embodiment 141. The pharmaceutical composition of any one of embodiments 135-140, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 142. The pharmaceutical composition of any one of embodiments 135-141, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 143. The pharmaceutical composition of any one of embodiments 135-142, wherein said Bifidobacterium sp. comprises B. animalis, B. pseudocatenulatum, B. bifidum, B. breve, B. dentium, B. faecale, B. longum, or a combination thereof.
   Embodiment 144. The pharmaceutical composition of any one of embodiments 135-143, wherein said first bacterial strain comprises said Bifidobacterium sp. or said Vertebrate-Associated Lactobacillaceae.
   Embodiment 145. The pharmaceutical composition of embodiment 144, wherein said first bacterial strain comprises said Bifidobacterium sp.
   Embodiment 146. The pharmaceutical composition of embodiment 144, wherein said first bacterial strain comprises said Vertebrate-Associated Lactobacillaceae.
   Embodiment 147. The pharmaceutical composition of any one of embodiments 135-146, wherein said second bacterial strain comprises said Bifidobacterium sp. or said Vertebrate-Associated Lactobacillaceae.
   Embodiment 148. The pharmaceutical composition of embodiment 147, wherein said second bacterial strain comprises said Bifidobacterium sp.
   Embodiment 149. The pharmaceutical composition of embodiment 147, wherein said second bacterial strain comprises said Vertebrate-Associated Lactobacillaceae.
   Embodiment 150. The pharmaceutical composition of any one of embodiments 135-149, wherein said growth promoter comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof.
   Embodiment 151. The pharmaceutical composition of embodiment 150, wherein said starch comprises a modified starch.
   Embodiment 152. The pharmaceutical composition of embodiment 151, wherein said starch comprises a fermented starch.
   Embodiment 153. The pharmaceutical composition of embodiment 152, wherein said fermented starch comprises a dextrin.
   Embodiment 154. The pharmaceutical composition of embodiment 153, wherein said dextrin comprises a maltodextrin.
   Embodiment 155. The pharmaceutical composition of any one of embodiments 135-154, wherein said pharmaceutical composition further comprises a pharmaceutically acceptable excipient, a cryoprotectant, or a combination thereof.
   Embodiment 156. The pharmaceutical composition of em any one of embodiments 135-155, wherein said pharmaceutical composition further comprises Akkermansia sp., Anaerbutyricum sp., Anaerostipes sp., Anaerotignum sp., Bacillus sp., Bacteroides sp., Blautia sp., ClostridiumCollinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 157. A pharmaceutical composition comprising a bacterial population comprising at least one strain of Bifidobacterium sp. or Vertebrate-Associated Lactobacillaceae,
wherein said bacterial population, when present within a medium comprising sialic acid, does not exhibit a growth of at least about 105% by weight as compared to a growth of a reference bacterial population when present within a medium comprising glucose.
   Embodiment 158. The pharmaceutical composition of embodiment 157, wherein said bacterial population, when present within said medium comprising said sialic acid, does not exhibit said growth of at least about 105% by weight as compared to said growth of said reference bacterial population when present within said medium comprising said glucose as a carbon source.
   Embodiment 159. The pharmaceutical composition of embodiment 158, wherein said bacterial population, when present within said medium comprising said sialic acid, does not exhibit said growth of at least about 105% by weight as compared to said growth of said reference bacterial population when present within said medium comprising said glucose as a sole carbon source.
   Embodiment 160. The pharmaceutical composition of any one of embodiments 157-159, wherein said bacterial population, when present within said medium comprising said sialic acid, does not exhibit a growth of at least about 120% by weight as compared to said growth of said reference bacterial population when present within said medium comprising said glucose.
   Embodiment 161. The pharmaceutical composition of embodiment 160, wherein said bacterial population, when present within said medium comprising said sialic acid, does not exhibit a growth of at least about 150% by weight as compared to said growth of said reference bacterial population when present within said medium comprising said glucose.
   Embodiment 162. The pharmaceutical composition of embodiment 161, wherein said bacterial population, when present within said medium comprising said sialic acid, does not exhibit a growth of at least about 1000% by weight as compared to said growth of said reference bacterial population when present within said medium comprising said glucose.
   Embodiment 163. The pharmaceutical composition of any one of embodiments 157-162, wherein said pharmaceutical composition is formulated in an enteral dosage form, an injectable dosage form, a parenteral dosage form, a topical dosage form, or a combination thereof.
   Embodiment 164. The pharmaceutical composition of embodiment 163, wherein said enteral dosage form comprises an oral dosage form, an intragastric dosage form, or a rectal dosage form.
   Embodiment 165. The pharmaceutical composition of embodiment 164, wherein said intragastric dosage form comprises a dosage form that is configured to pass through a feeding tube.
   Embodiment 166. The pharmaceutical composition of any one of embodiments 157-165, wherein said Vertebrate-Associated Lactobacillaceae comprises Vertebrate-Associated Lactobacillaceae, LimosiVertebrate-Associated Lactobacillaceae, LigiVertebrate-Associated Lactobacillaceae, Lacticaseibacillus sp., or a combination thereof.
   Embodiment 167. The pharmaceutical composition of any one of embodiments 157-166, wherein said Vertebrate-Associated Lactobacillaceae comprises Lactobacillus crispatus, Lactobacillus gasseri, Limosilactobacillus vaginalis, Limosilactobacillus fermentum, Lactobacillus plantarum, Lactobacillus jensenii, Lacticaseibacillus paracasei, or a combination thereof.
   Embodiment 168. The pharmaceutical composition of any one of embodiments 157-167, wherein said Bifidobacterium sp. comprises B. animalis, B. pseudocatenulatum, B. bifidum, B. breve, B. dentium, B. faecale, B. longum, or a combination thereof.
   Embodiment 169. The pharmaceutical composition of any one of embodiments 157-168, wherein said first bacterial strain comprises said Bifidobacterium sp. or said Vertebrate-Associated Lactobacillaceae.
   Embodiment 170. The pharmaceutical composition of embodiment 169, wherein said first bacterial strain comprises said Bifidobacterium sp.
   Embodiment 171. The pharmaceutical composition of embodiment 169, wherein said first bacterial strain comprises said Vertebrate-Associated Lactobacillaceae.
   Embodiment 172. The pharmaceutical composition of any one of embodiments 157-171, wherein second bacterial strain comprises said Bifidobacterium sp. or said Vertebrate-Associated Lactobacillaceae.
   Embodiment 173. The pharmaceutical composition of embodiment 172, wherein said second bacterial strain comprises said Bifidobacterium sp.
   Embodiment 174. The pharmaceutical composition of embodiment 172, wherein said second bacterial strain comprises said Vertebrate-Associated Lactobacillaceae.
   Embodiment 175. The pharmaceutical composition of any one of embodiments 157-174, wherein said energy source comprises fructooligosaccharides (FOS), guar gum, corn syrup, polydextrose, Galacto-Oligosaccharides (GOS), lactose, inulin, mucin, sialic acid, glucan, fructose, N-Acetylglucosamine (GlcNAc), mannose, Lacto-N-neotetraose (LNnT), glucose, 2′-Fucosyllactose (2′-FL), galactose, fructose, pectin, starch, or a combination thereof.
   Embodiment 176. The pharmaceutical composition of embodiment 175, wherein said starch comprises a modified starch.
   Embodiment 177. The pharmaceutical composition of embodiment 176, wherein said starch comprises a fermented starch.
   Embodiment 178. The pharmaceutical composition of embodiment 177, wherein said fermented starch comprises a dextrin.
   Embodiment 179. The pharmaceutical composition of embodiment 178, wherein said dextrin comprises a maltodextrin.
   Embodiment 180. The pharmaceutical composition of any one of embodiments 157-179, wherein said pharmaceutical composition further comprises a pharmaceutically acceptable excipient, a cryoprotectant, or a combination thereof.
   Embodiment 181. The pharmaceutical composition of any one of embodiments 157-180, wherein said pharmaceutical composition further comprises Akkermansia sp., Anaerbutyricum sp., Anaerostipes sp., Anaerotignum sp., Bacillus sp., Bacteroides sp., Blautia sp., ClostridiumCollinsella sp., Coprococcus sp., Dorea sp., Enterococcus sp., Erysipelatoclostridium sp., Escherichia sp., Eubacterium sp., Faecalibacterium sp., Faecalicatena sp., Holdemanella sp., Lachnospira sp., Longibaculum sp., Paraprevotella sp., Parabacteroides sp., Pediococcus sp., Roseburia sp., Ruminococcus sp., Veillonella sp., or a combination thereof.
   Embodiment 182. A method, comprising administering the pharmaceutical composition of any one of embodiments 1-181 to a subject.
   Embodiment 183. The method of embodiment 182, wherein said subject has bacterial vaginosis BV or necrotizing enterocolitis (NEC) or a risk of said BV or a risk of said NEC.
   Embodiment 184. The method of embodiment 183, wherein said subject has said BV or said risk of said BV.
   Embodiment 185. The method of embodiment 184, wherein said subject has said BV.
   Embodiment 186. The method of embodiment 184, wherein said subject has said risk of BV.
   Embodiment 187. The method of embodiment 184, wherein said subject is at least about 10 years old.
   Embodiment 188. The method of embodiment 187, wherein said subject is at least about 15 years old.
   Embodiment 189. The method of embodiment 187, wherein said subject is at most about 120 years old.
   Embodiment 190. The method of embodiment 183, wherein said subject has said NEC or said risk of said NEC.
   Embodiment 191. The method of embodiment 190, wherein said subject has said NEC.
   Embodiment 192. The method of embodiment 190, wherein said subject has said risk of said NEC.
   Embodiment 193. The method of embodiment 190, wherein said subject is at most about 1 year old.
   Embodiment 194. The method of embodiment 193, wherein said subject is at least about 1 day old.
   Embodiment 195. The method of embodiment 190, wherein said subject is a premature infant.
   Embodiment 196. The method of embodiment 182, wherein said subject has a microbial dysbiosis.
   Embodiment 197. The method of embodiment 196, wherein said subject has said microbial dysbiosis in a gastrointestinal (GI) tract or vagina of said subject.
   Embodiment 198. The method of embodiment 197, wherein said subject has said microbial dysbiosis in said GI tract of said subject.
   Embodiment 199. The method of embodiment 197, wherein said subject has said microbial dysbiosis in said vagina of said subject.
   Embodiment 200. A method, comprising:
(a) providing a plurality of bacterial strains;
(b) culturing a given bacterial strain of said plurality of bacterial strains in a carbon source or a plurality of carbon sources;
(c) measuring growth of said plurality of bacterial strains; and
(d) selecting a bacterial strain of said plurality of bacterial strains,
   wherein said plurality of bacterial strains comprises Bifidobacterium sp. or Vertebrate-Associated Lactobacillaceae, wherein said bacterial strain, when present within a medium comprising said carbon source and a second bacterial strain or a supernatant of said medium comprising said carbon source and said second bacterial strain, exhibits a growth of at least about 105% by weight as compared to a growth of said first bacterial strain when present within a medium comprising said carbon source in an absence of said second bacterial strain or said supernatant of said medium comprising said carbon source and said second bacterial strain, and
wherein said carbon source does not comprise starch.
   Embodiment 201. The method of embodiment 200, wherein said supernatant of said medium comprising said carbon source and said second bacterial strain is cell-free.
   Embodiment 202. The method of embodiment 200 or 201, wherein said supernatant of said medium comprising said carbon source and said second bacterial strain comprises a fermentation product derived from said second bacterial strain.
   Embodiment 203. A method, comprising:
(a) providing a plurality of bacterial strains;
(b) culturing a given bacterial strain of said plurality of bacterial strains in a carbon source of a plurality of carbon sources;
(c) measuring growth of said plurality of bacterial strains;
(d) selecting a bacterial strain of said plurality of bacterial strains,
   wherein said plurality of bacterial strains comprises Bifidobacterium sp. or Vertebrate-Associated Lactobacillaceae, wherein said bacterial strain, when present within a medium comprising a secreted metabolite derived from a second bacterial strain, proliferates for at least about 10 cell divisions.
   Embodiment 204. The method of embodiment 203, wherein said medium comprising said secreted metabolite derived from said second bacterial strain comprises a supernatant derived from a growth culture of said second bacterial strain.
   Embodiment 205. The method of embodiment 204, wherein said supernatant derived from said growth culture of said second bacterial strain is cell-free.
   Embodiment 206. A method, comprising:
(a) providing a plurality of bacterial strains;
(b) culturing a given bacterial strain of said plurality of bacterial strains in a carbon source of a plurality of carbon sources;
(c) measuring growth of said plurality of bacterial strains; and
(d) selecting a bacterial strain of said plurality of bacterial strains,
   wherein said plurality of bacterial strains comprises Bifidobacterium sp. or Vertebrate-Associated Lactobacillaceae, wherein said bacterial strain, when present within a medium comprising a secreted metabolite derived from said second bacterial strain and a layer of epithelial cells, decreases permeability of said layer of epithelial cells by at least about 5%, as compared to permeability of a layer of epithelial cells present within a medium comprising said bacterial strain in an absence of said secreted metabolite, and
wherein said permeability of said layer of epithelial cells is measured by transport of a Fluorescein isothiocyanate (FITC)-conjugated dextran or by transepithelial electrical resistance across said layer of epithelial cells.
   Embodiment 207. The method of embodiment 206, wherein said medium comprising said secreted metabolite derived from said second bacterial strain comprises a supernatant derived from a growth culture of said second bacterial strain.
   Embodiment 208. The method of embodiment 207, wherein said supernatant derived from said growth culture of said second bacterial strain is cell-free.
   Embodiment 209. The method of any one of embodiments 206-208, wherein said secreted metabolite derived from said second bacterial strain comprises a fermentation product derived from said second bacterial strain.
   Embodiment 210. The method of any one of embodiments 206-209, wherein said epithelial cells comprise mammalian epithelial cells.
   Embodiment 211. The method of embodiment 210, wherein said mammalian epithelial cells comprises human epithelial cells.
   Embodiment 212. A method, comprising:
(a) providing a plurality of bacterial strains;
(b) culturing a given bacterial strain of said plurality of bacterial strains in a carbon source of a plurality of carbon sources;
(c) measuring growth of said plurality of bacterial strains; and
(d) selecting a bacterial strain of said plurality of bacterial strains,
wherein said plurality of bacterial strains comprises Bifidobacterium sp. or Vertebrate-Associated Lactobacillaceae, wherein a secreted metabolite or an inviable cell derived from said bacterial strain, when combined with an engineered cell comprising a reporter, decreases a signal of said reporter by at least about 5% as compared to a signal of said reporter when said engineered cell is not combined with said metabolite or said inviable cell derived from said bacterial strain.
   Embodiment 213. The method of embodiment 212, wherein said medium comprising said secreted metabolite or said inviable cells derived from said second bacterial strain comprises a supernatant derived from a growth culture of said second bacterial strain.
   Embodiment 214. The method of embodiment 213, wherein said supernatant derived from said growth culture of said second bacterial strain is cell-free.
   Embodiment 215. The method of any one of embodiments 212-214, wherein said medium comprising said secreted metabolite or said inviable cells derived from said second bacterial strain comprises a fermentation product derived from a growth culture of said second bacterial strain.
   Embodiment 216. The method of any one of embodiments 212-215, wherein said secreted metabolite derived from said second bacterial strain comprises a fermentation product derived from said second bacterial strain.
   Embodiment 217. The method of any one of embodiments 212-216, wherein said engineered cell comprises a macrophage.
   Embodiment 218. The method of any one of embodiments 212-217, wherein said inviable cell comprises a pasteurized cell.
   Embodiment 219. The method of any one of embodiments 212-218, wherein said reporter comprises a nuclear factor kappa-light-chain-enhancer of activated B cells (NFkB) reporter or an interferon-sensitive response element reporter (ISRE).
   Embodiment 220. The method of embodiment 219, wherein said NFkB reporter comprises a secreted embryonic alkaline phosphatase (SEAP) reporter.
   Embodiment 221. The method of embodiment 219 or 220, wherein said ISRE reporter comprises a Lucia luciferase.
   Embodiment 222. A method, comprising:
(a) providing a plurality of bacterial strains;
(b) culturing a given bacterial strain of said plurality of bacterial strains in a carbon source of a plurality of carbon sources;
(c) measuring growth of said plurality of bacterial strains; and
(d) selecting a bacterial strain of said plurality of bacterial strains,
wherein said plurality of bacterial strains comprises Bifidobacterium sp. or Vertebrate-Associated Lactobacillaceae, wherein said bacterial strain, when present within a medium comprising said second bacterial strain or a supernatant thereof for at most about 15 hours, exhibits a growth of at least about 105% by colony-forming unit (CFU) as compared to a growth of said bacterial strain when present within a medium not comprising said second bacterial strain or said supernatant thereof for at most about 15 hours.
   Embodiment 223. The method of embodiment 222, wherein said medium comprising said second bacterial strain or said supernatant thereof comprises a secreted metabolite derived from said second bacterial strain.
   Embodiment 224. The method of embodiment 223, wherein said secreted metabolite derived from said second bacterial strain comprises a fermentation product of derived from said second bacterial strain.
   Embodiment 225. The method of any one of embodiments 222-224, wherein said supernatant of said medium comprising said second bacterial strain is cell-free.
   Embodiment 226. A method, comprising:
(a) providing a plurality of bacterial strains;
(b) culturing a given bacterial strain of said plurality of bacterial strains in a carbon source of a plurality of carbon sources;
(c) measuring growth of said plurality of bacterial strains; and
(d) selecting a bacterial strain of said plurality of bacterial strains,
wherein said plurality of bacterial strains comprises Bifidobacterium sp. or Vertebrate-Associated Lactobacillaceae, wherein said first bacterial strain is configured to utilize a metabolite derived from said second bacterial strain as a growth promoter, and
wherein said metabolite is not a butyrate, a vitamin B12, or an ammonia (NH3).
   Embodiment 227. The method of embodiment 226, wherein said metabolite is a secreted metabolite derived from said second bacterial strain.
   Embodiment 228. The method of embodiment 226 or 227, wherein said metabolite is not a derivative or a combination of said butyrate, said vitamin B12, or said NH3.
   Embodiment 229. A method, comprising:
(a) providing a plurality of bacterial strains;
(b) culturing a given bacterial strain of said plurality of bacterial strains in a carbon source of a plurality of carbon sources;
(c) measuring growth of said plurality of bacterial strains;
(d) selecting a bacterial strain of said plurality of bacterial strains,
   wherein said plurality of bacterial strains comprises Bifidobacterium sp. or Vertebrate-Associated Lactobacillaceae, wherein said bacterial strain is incapable of using sialic acid as said carbon source.
   Embodiment 230. The method of embodiment 229, wherein said bacterial population, when present within said medium comprising said sialic acid, does not exhibit a growth of at least about 105% by weight as compared to said growth of a reference bacterial population when present within said medium comprising glucose as a carbon source for said reference bacterial population.
   Embodiment 231. The method of embodiment 230, wherein said bacterial population, when present within said medium comprising said sialic acid, does not exhibit a growth of at least about 105% by weight as compared to said growth of said reference bacterial population when present within said medium comprising said glucose as a sole carbon source for said reference bacterial population.

1.-240. (canceled)

241. A composition comprising:
a bacterial population comprising a first bacterial strain and a second bacterial strain different from said first bacterial strain, wherein said bacterial population is for use in treating a disease or disease condition in a subject in need thereof,
wherein said first bacterial strain and said second bacterial strain comprise Bifidobacterium sp. or Vertebrate-Associated Lactobacillaceae, and
wherein said composition is configured to treat said disease or disease condition at least in part by:
(1) inhibiting a growth of a pathogen in said subject by at least 0.1%, relative to a growth of said pathogen in said subject when inhibited by a control composition comprising a control bacterial population that does not comprise said first bacterial strain and said second bacterial strain;
(2) inhibiting a biofilm formation of said pathogen in said subject by at least 0.1%, relative to a biofilm formation of said pathogen in said subject when inhibited by said control composition;
(3) inhibiting an immune response signaling pathway of a cell of said subject by at least 0.1%, relative to said immune response signaling pathway of said cell of said subject when inhibited by said control composition; or
(4) any combinations of (1)-(3).

242. The composition of claim 241, wherein said composition is configured to treat said disease or disease condition at least in part by inhibiting said growth of said pathogen in said subject by at least 0.1%, relative to said growth of said pathogen in said subject when inhibited by said control composition.

243. The composition of claim 241, wherein said disease or disease condition comprises an infant gastrointestinal disease.

244. The composition of claim 243, wherein said infant gastrointestinal disease comprises necrotizing enterocolitis (NEC).

245. The composition of claim 241, wherein said pathogen comprises E. coli, K. pneumoniae, C. perfringens, S. aureus, S. flexneri, or a combination thereof.

246. The composition of claim 241, wherein said composition is configured to inhibit said immune response signaling pathway of said cell of said subject by at least 0.1%, relative to said immune response signaling pathway of said cell of said subject when inhibited by said control composition.

247. The composition of claim 246, wherein said immune response signaling pathway comprises an inflammatory immune response signaling pathway, an innate immune response signaling pathway, or a combination thereof.

248. The composition of claim 241, wherein said bacterial population comprises Bifidobacterium bifidum, Bifidobacterium adolescentis, Bifidobacterium breve, Bifidobacterium longum, Bifidobacterium pseudocatenulatum, Lactobacillus plantarum, Lactobacillus rhamnosus, or a combination thereof.

249. The composition of claim 241, wherein said bacterial population comprises at least two of Bifidobacterium longum, Bifidobacterium bifidum, Bifidobacterium breve, or Lactobacillus plantarum.

250. The composition of claim 241, wherein said bacterial population comprises (1) Bifidobacterium longum, Bifidobacterium bifidum, and Lactobacillus plantarum or (2) Bifidobacterium longum, Bifidobacterium bifidum and Bifidobacterium breve.

251. The composition of claim 241, wherein said composition is formulated in a solid or liquid dosage form.

252. The composition of claim 241, wherein said disease or disease condition comprises a vaginal disease or a complication associated with said vaginal disease.

253. The composition of claim 252, (1) wherein said vaginal disease comprises bacterial vaginosis (BV), recurrent BV, or a combination thereof; or (2) wherein said complication associated with said vaginal disease comprises preterm birth, pelvic inflammatory disease (PID), vulvovaginitis, sexually transmitted infections (STIs), or a combination thereof.

254. The composition of claim 241, wherein said composition is configured to treat said disease or disease condition at least in part by:
inhibiting said biofilm formation of said pathogen in said subject by at least 0.1%, relative to said biofilm formation of said pathogen in said subject when inhibited by said control composition.

255. The composition of claim 241, wherein said pathogen comprises Prevotella bivia, Atopobium vaginae, Sneathia spp., G. vaginalis, L. iners, or a combination thereof

256. The composition of claim 241, wherein bacterial population comprises Lactobacillus jensenii, Lactobacillus gasseri, Lactobacillus crispatus, or a combination thereof.

257. The composition of claim 241, wherein said bacterial population comprises at least two of Lactobacillus jensenii, Lactobacillus gasseri, or Lactobacillus crispatus.

258. The composition of claim 241, wherein said bacterial population comprises Lactobacillus jensenii and Lactobacillus crispatus.

259. The composition of claim 241, wherein said composition is formulated in a vaginal dosage form.

260. A method of treating a disease or disease condition in a subject in need thereof, comprising:
administering to said subject a therapeutically-effective amount of a composition, wherein said composition comprises a bacterial population comprising a first bacterial strain and a second bacterial strain different from said first bacterial strain,
wherein said first bacterial strain and said second bacterial strain comprise Bifidobacterium sp. or Vertebrate-Associated Lactobacillaceae, and
wherein said composition:
(1) inhibits a growth of a pathogen in said subject by at least 0.1%, relative to a growth of said pathogen in said subject when inhibited by a control composition not comprising said first bacterial strain and said second bacterial strain;
(2) inhibits a biofilm formation of said pathogen in said subject by at least 0.1%, relative to a biofilm formation of said pathogen in said subject when inhibited by said control composition;
(3) inhibits an immune response signaling pathway of a cell of said subject by at least 0.1%, relative to said immune response signaling pathway of said cell of said subject when inhibited by said control composition; or
(4) any combinations of (1)-(3).
